(19) United States
(12) Patent Application Publication
(10) Pub. No.: US 2022/0152031 A1
(43) Pub. Date: May 19, 2022

(54) PYRIDAZINONES AND METHODS OF USE THEREOF

(71) Applicant: Goldfinch Bio, Inc., Cambridge, MA (US)

(72) Inventors: John Francis Reilly, Framingham, MA (US); Liron Walsh, Newton, MA (US); Peter H. Mundel, Brookline, MA (US); Amy Kieu Duyen Westerling-Bui, Cambridge, MA (US); Matthew H. Daniels, Somerville, MA (US); Maolin Yu, West Roxbury, MA (US); Mark W. Ledeboer, Acton, MA (US); Jean-Christophe P. Harmange, Andover, MA (US); Marie-Francoise Yveline Coeffet-Le Gal, Arlington, MA (US); Michael Broxson, Concord, MA (US)

(21) Appl. No.: 17/440,928

(22) PCT Filed: Mar. 18, 2020
(86) PCT No.: PCT/US20/23369

§ 371 Date: Sep. 20, 2021

Related U.S. Application Data
(60) Provisional application No. 62/821,178, filed on Mar. 20, 2019.

(51) Int. Cl. A61K 31/519; A61K 45/06

(52) U.S. Cl. CPC A61K 31/519; A61K 45/06

(57) ABSTRACT

Disclosed are therapeutic methods, e.g., of treating kidney diseases, using compounds of Formula (A) in combination with a second therapeutic agent.

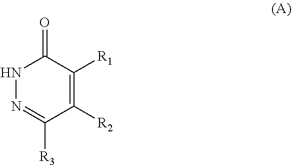

### RELATED APPLICATIONS

This application claims the benefit of priority to U.S. Provisional Application for Patent Ser. No. 62/821,178, filed Mar. 20, 2019.

### BACKGROUND

Proteinuria is a condition in which an excessive amount of protein in the blood leaks into the urine. Proteinuria can progress from a loss of 30 mg of protein in the urine over a 24-hour period (called microalbuminuria) to >300 mg/day (called macroalbuminuria), before reaching levels of 3.5 grams of protein or more over a 24-hour period, or 25 times the normal amount. Proteinuria occurs when there is a malfunction in the kidney's glomeruli, causing fluid to accumulate in the body (edema). Prolonged protein leakage has been shown to result in kidney failure. Nephrotic Syndrome (NS) disease accounts for approximately 12% of prevalent end stage renal disease cases at an annual cost in the United States of more than $3 billion. Approximately 5 out of every 100,000 children are diagnosed with NS every year and 15 out of every 100,000 children are living with it today. For patients who respond positively to treatment, the relapse frequency is extremely high. Ninety % of children with Nephrotic Syndrome will respond to treatment, however, an estimated 75% will relapse. There is a need for more effective methods of treating, or reducing risk of developing, kidney disease, e.g., proteinuria.

Mammalian TRP channel proteins form six-transmembrane cation-permeable channels which may be grouped into six subfamilies on the basis of amino acid sequence homology (TRPC, TRPV, TRPM, TBPA, TRPP, and TRPML). Recent studies of TRP channels indicate that they are involved in numerous fundamental cell functions and are considered to play an important role in the pathophysiology of many diseases. Many TRPs are expressed in kidney along different parts of the nephron and growing evidence suggest that these channels are involved in hereditary, as well as acquired kidney disorders. TRPC6, TRPM6, and TRPP2 have been implicated in hereditary focal segmental glomerulosclerosis (FSGS), hypomagnesemia with secondary hypocalcemia (HSH), and polycystic kidney disease (PKI), respectively.

TRPC5 has also been reported to contribute to the mechanisms underlying regulation of innate fear responses. (J Neurosci. 2014 Mar. 5; 34(10): 3653-3667).

### SUMMARY

One aspect of the invention is methods of treating kidney disease comprising the step of co-administering to a subject in need thereof a TRPC5 inhibitory compound and a second therapeutic agent. In some embodiments, the method of the invention comprises the step of co-administering to a subject in need thereof:

a. a TRPC5 inhibitory compound of structural Formula (A), or a tautomer or a pharmaceutically acceptable salt thereof:

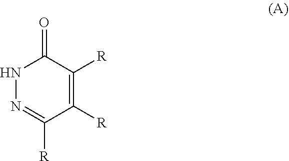

wherein

each R is independently selected from the group consisting of H, alkyl, alkenyl, alkynyl, aryl, heterocyclyl, heteroaryl, halogen, —OH, CN, cycloalkyl, —O-alkyl, —O— cycloalkyl, —O-aryl, -aryl-O-aryl, —CF₃, —C(H)F₂, alkylene-CF₃, alkylene-C(H)F₂, —SO₂-alkyl, —O-alkylene-O-alkyl, -heterocyclyl-L-R⁴, and heteroaryl-L-R⁴;

R⁴ is absent or selected from the group consisting of alkyl, cycloalkyl, polycyclyl, aryl, heterocyclyl, heteroaryl, —C(O)N(R⁵)₂, and CF₃;

R⁵ is independently H or alkyl;

R⁶ is selected from the group consisting of alkyl, cycloalkyl, aryl, heterocyclyl, heteroaryl, alkylene-aryl, —C(O)N(R⁵)₂, and CF₃;

L is absent or selected from the group consisting of methylene, —C(O)—, —SO₂—, —CH₂N(Me)-, —N(R⁵)(R⁶)—, —C(R⁵)(R⁶)—, and —O—R⁶; and

one and only one R is -heterocyclyl-L-R⁴ or -heteroaryl-L-R⁴; and

b. a second therapeutic agent selected from an immunomodulator, a calcineurin inhibitor, a renin angiotensin aldosterone system inhibitor, an antiproliferative agent, an alkylating agent, a corticosteroid, an angiotensin converting enzyme inhibitor, an adrenocorticotropic hormone stimulant, an angiotensin receptor blocker, a sodium-glucose transport protein 2 inhibitor, a dual sodium-glucose transport protein 1/2 inhibitor, a nuclear Factor-1 (erythroid-derived 2)-like 2 agonist, a chemokine receptor 2 inhibitor, a chemokine receptor 5 inhibitor, an endothelin 1 receptor antagonist, a beta blocker, a mineralocorticoid receptor antagonist, a loop or thiazide diuretic, a calcium channel blocker, a statin, a short-intermediate or long-acting insulin, a dipeptidyl peptidase 4 inhibitor, a glucagon-like peptide 1 receptor agonist, a sulfonylurea, an apoptosis signal-regulating kinase-1, a chymase inhibitor, a selective glycation inhibitor, a renin inhibitor, an interleukin-33 inhibitor, a farnesoid X receptor agonist, a soluble guanylate cyclase stimulator, a thromboxane receptor antagonist, a xanthine oxidase inhibitor, an erythropoietin receptor agonist, a cannabinoid receptor type 1 inverse agonist, a NADPH oxidase inhibitor, an anti-vascular endothelial growth factor B, an anti-fibrotic agent, a neprilysin inhibitor, a dual CD80/CD86 inhibitor, a CD40 antagonist, a cellular cholesterol and lipid blocker, a PDGFR antagonist, a Slit guidance ligand 2, an APOL1 inhibitor, an Nrl2 activator/NF-κB inhibitor, a somatostatin receptor agonist, a PPAR gamma agonist, a AMP activated protein kinase stimulator, a tyrosine kinase inhibitor, a glucosylceramide synthase inhibitor, an arginine vasopressin receptor 2 antagonist, a xanthine oxidase inhibitor, and a vasopressin receptor 2 antagonist.

In some embodiments, the TRPC5 inhibitor and the second therapeutic agent are administered as separate dosage forms.

In alternate embodiments, the TRPC5 inhibitor and the second therapeutic agent are administered together as a fixed dose combination (i.e., a single formulation).

In some embodiments, the second therapeutic agent is an immunomodulator, a calcineurin inhibitor, a renin angiotensin aldosterone system inhibitor, an antiproliferative agent, a corticosteroid, an angiotensin converting enzyme inhibitor, an angiotensin receptor blocker, a sodium-glucose transport protein 2 inhibitor, a nuclear Factor-1 (erythroid-derived 2)-like 2 agonist, a chemokine receptor 2 inhibitor, a chemokine receptor 5 inhibitor, or an endothelin 1 receptor antagonist.

In some embodiments, the TRPC5 inhibitory compound is represented by structural Formula (A-I), (A-II), or (A-III), or a tautomer or a pharmaceutically acceptable salt thereof;

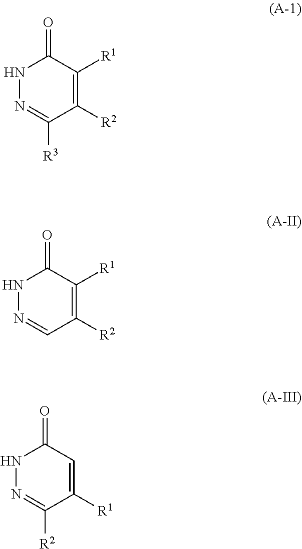

wherein

R¹ and R³ are independently selected from the group consisting of H, alkyl, alkenyl, alkynyl, aryl, heterocyclyl, heteroaryl, halogen, —OH, —CN, -cycloalkyl, —O-alkyl, —O-cycloalkyl, —O-aryl, -aryl-O-aryl —CF₃, —C(H)F₂, alkylene-CF₃, alkylene-C(H)F₂, —SO₂-alkyl, and —O-alkylene-O-alkyl, -heterocyclyl-L-R⁴, and -heteroaryl-L-R⁴;

R² is -heterocyclyl-L-R⁴;

R⁴ is absent or selected from the group consisting of alkyl, cycloalkyl, aryl, alkylene-aryl, alkylene-heteroaryl, heteroaryl, heterocyclyl, —C(O)N(R⁵)₂, and CF₃;

R⁵ is independently H or alkyl;

R⁶ is selected from the group consisting of alkyl, cycloalkyl, aryl, heterocyclyl, heteroaryl, alkylene-aryl, —C(O)N(R⁵)₂, and CF₃;

L is absent or selected from the group consisting of methylene, —C(O)—, —SO₂—, —CH₂N(Me)-, —N(R⁵)(R⁶)—, —C(R⁵)(R⁶)—, and —O—R⁶; and

one and only one of R¹, R², and R³ is -heterocyclyl-L-R⁴ or -heteroaryl-L-R⁴.

In some embodiments, the TRPC5 inhibitory compound has structural formula (I):

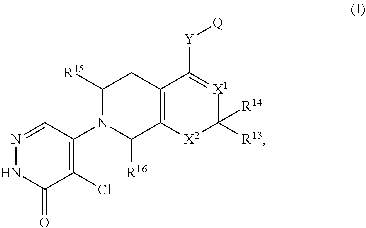

or a pharmaceutically acceptable salt thereof;
wherein:

“ [symbol] ” is a single bond or a double bond

X¹ is CH or N;

when “ [symbol] ” is a double bond, X² is CH or N;

when “ [symbol] ” is a single bond, X² is N(CH₃),

when X¹ is CH, X² is N or N(CH₃);

Y is —O—, —N(CH₃)—, —N(CH₂CH₂OH)—, cyclopropan-1,1-diyl, or —CH(CH₃)—;

Q is 2-trifluoromethyl-4-fluorophenyl, 2-difluoromethyl-4-fluorophenyl, 2-trifluoromethylphenyl, 2-methyl-4-fluorophenyl, 2-chloro-4-fluorophenyl, 2-chlorophenyl, 1-(benzyl)-4-methylpiperidin-3-yl, 4-trifluoromethylpyridin-3-yl, 2-trifluoromethyl-6-fluorophenyl, 2-trifluoromethyl-3-cyanophenyl, 2-ethyl-3-fluorophenyl, 2-chloro-3-cyanophenyl, 2-trifluoromethyl-5-fluorophenyl, or 2-difluoromethylphenyl;

when “ [symbol] ” is a double bond, R¹³ is hydrogen, —CH₂OH, —CH(OH)—CH₂OH, —NH₂, —CH(OH)CH₃, —OCH₃, or —NH—(CH₂)₂OH; and R¹⁴ is absent; or

when “ [symbol] ” is a single bond, R¹³ and R¹⁴ are taken together to form ═O; and

each of R⁵ and R⁶ is independently hydrogen or —CH₃.

In some embodiments, the TRPC5 inhibitory compound has the structural formula (II):

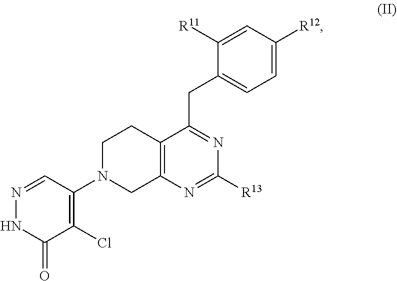

or a pharmaceutically acceptable salt thereof;
wherein:

R¹¹ is chloro, —CF₃, —CHF₂, or —CH₃;

R¹² is hydrogen or fluoro; and

R¹³ is hydrogen, —NH₂, —CH₂OH, or CH(OH)—CH₂OH.

In some embodiments, the immunomodulator is rituximab. In some embodiments, the angiotensin converting enzyme inhibitor is captopril, zofenopril, enalapril, ramipril, quinapril, perindopril, lisinopril, benazepril, imidapril, trandolapril, or cilazapril.

In some embodiments, the angiotensin receptor blocker is losartan, candesartan, valsartan, irbesartan, telmisartan, eprosartan, olmesartan, azilsartan, or fimasartan.

In some embodiments, the renin angiotensin aldosterone system inhibitor is aliskiren.

In some embodiments, the endothelin 1 receptor antagonist is ambrisentan, atrasentan, bosentan, or sparsentan. In some additional embodiments, the endothelin 1 receptor antagonist is macitentan.

In some embodiments, the anti-proliferative agent is mycophenolate mofetil. In some additional embodiments, the anti-proliferative agent is mycophenolate sodium, or azathioprine.

In some embodiments, the SGLT2 inhibitor is canagliflozin, dapagliflozin, empagliflozin, a combination of empagliflozin and linagliptin, a combination of empagliflozin and metformin, or a combination of dapagliflozin and metformin. In some additional embodiments, the SGLT2 inhibitor also inhibits SGLT1. In some aspects of these embodiments, that SGLT1/2 inhibitor is sotagliflozin.

In some embodiments, the calcineurin inhibitor is cyclosporine A or tacrolimus. In some additional embodiments, the calcineurin inhibitor is voclosporin.

In some embodiments, the nuclear Factor-1 (erythroid-derived 2)-like 2 agonist is bardoxolone or CXA-10.

In some embodiments, the chemokine receptor 2 inhibitor is PF-04136309 or ccx140. In some additional embodiments, the chemokine receptor 2 inhibitor is propagemanium (DMX-200).

In some embodiments, the beta blocker is a beta blocker is metoprolol succinate, metoprolol tartrate, propranolol, or carvedilol.

In some embodiments, the mineralocorticoid receptor antagonist is spironolactone, eplerenone, finerenone, esaxerenone, or apararenone.

In some embodiments, the loop or thiazide diuretic is furosemide, bumetanide, torsemide, or Bendroflumethiazide.

In some embodiments, the calcium channel blocker is verapamil, diltiazem, amlodipine, or nifedipine.

In some embodiments, the statin is atorvastatin, pravastatin, fluvastatin, lovastatin, rosuvastatin, simvastatin, or pitavastatin.

In some embodiments, the short-intermediate or long-acting insulin is NPH insulin (Humulin®, Novolin®, or biosimilars), Insulin Lispro (Humalog®), Insulin glulisine, Insulin glargine (Basaglar®, Lantus®), Insulin Detemir (Levemir®), or Insulin degludec (Tresiba®)).

In some embodiments, the dipeptidyl peptidase 4 inhibitor is sitagliptin, saxagliptin, linagliptin, or vildagliptin

In some embodiments, the glucagon-like peptide 1 receptor agonist is exenatide, liraglutide, dulaglutide, lixisenatide, albiglutide, or semaglutide.

In some embodiments, the sulfonylurea is glimepiride, glipizide, glyburide, glibenclamide, chlorpropamide, tolazamide or tolbutamide.

In some embodiments, the apoptosis signal-regulating kinase-1 is selonsertib.

In some embodiments, the chymase inhibitor is fulacimstat (BAY1142524).

In some embodiments, the selective glycation inhibitor is GLY-230.

In some embodiments, the renin inhibitor is SCO-272.

In some embodiments, the interleukin-33 inhibitor is MEDI-3506.

In some embodiments, the farnesoid X receptor agonist is nidufexor (LMB763)

In some embodiments, the soluble guanylate cyclase stimulator is praliciguat, olinciguat, IW-6463, vericiguat, or riociguat.

In some embodiments, the thromboxane receptor antagonist is SER150.

In some embodiments, the xanthine oxidase inhibitor is TMX-049.

In some embodiments, the erythropoietin receptor agonist is cibinetide (ARA-290).

In some embodiments, the cannabinoid receptor type 1 inverse agonist is nimacimab, GFB-024, or CRB-4001.

In some embodiments, the NADPH oxidase inhibitor is APX-115.

In some embodiments, the anti-vascular endothelial growth factor B is CSL-346.

In some embodiments, the anti-fibrotic agent is FT011.

In some embodiments, the neprilysin inhibitor is TD-1439, TD-0714, or sacubitril

In some embodiments, the a dual CD80/CD86 inhibitor is abatacept.

In some embodiments, the CD40 antagonist is bleselumab (ASKP1240).

In some embodiments, the cellular cholesterol and lipid blocker is VAR-200.

In some embodiments, the PDGFR antagonist is ANG 3070.

In some embodiments, the Slit guidance ligand 2 is PF-06730512.

In some embodiments, the APOL1 inhibitor is VX-147.

In some embodiments, the Nrl2 activator/NF-κB inhibitor is bardoxolone.

In some embodiments, the somatostatin receptor agonist is lanreotide.

In some embodiments, the PPAR gamma agonist is pioglitazone.

In some embodiments, the AMP activated protein kinase stimulator is metformin.

In some embodiments, the tyrosine kinase inhibitor is tesevatinib.

In some embodiments, the glucosylceramide synthase inhibitor is venglustat malate.

In some embodiments, the arginine vasopressin receptor 2 antagonist is lixivaptan.

In some embodiments, the xanthine oxidase inhibitor is oxypurinol.

In some embodiments, the vasopressin receptor 2 antagonist is tolvaptan.

In some embodiments, the second therapeutic agent is tacrolimus, cyclosporine A, rituximab, mycophenolate mofetil, a corticosteroid, sparsentan, enalapril, or losartan.

In some embodiments, the disease or condition is Focal Segmental Glomerulosclerosis (FSGS), Primary Focal Segmental Glomerulosclerosis, genetic Focal Segmental Glomerulosclerosis, secondary Focal Segmental Glomerulosclerosis, Diabetic nephropathy, Alport syndrome, hypertensive kidney disease, nephrotic syndrome, steroid-resistant nephrotic syndrome, minimal change disease, membranous nephropathy, idiopathic membranous nephropathy, membranoproliferative glomerulonephritis (MPGN), immune complex-mediated MPGN, complement-mediated MPGN, Lupus nephritis, postinfectious glomerulonephritis, thin basement membrane disease, mesangial proliferative glomerulonephritis, amyloidosis (primary), c1q nephropathy, rapidly progressive GN, anti-GBM disease, C3 glomerulonephritis, hypertensive nephrosclerosis, or IgA nephropathy. In some embodiments, the disease or condition is focal segmental glomerulosclerosis.

The methods are effective for a variety of subjects including mammals, e.g., humans and other animals, such as laboratory animals, e.g., mice, rats, rabbits, or monkeys, or domesticated and farm animals, e.g., cats, dogs, goats, sheep, pigs, cows, or horses. In some embodiments, the subject is a human.

The invention provides several advantages. The prophylactic and therapeutic methods described herein are effective in treating kidney disease, e.g., proteinuria, and have minimal, if any, side effects. Further, methods described herein are effective to identify compounds that treat or reduce risk of developing a kidney disease, anxiety, depression, or cancer.

Unless otherwise defined, all technical and scientific terms used herein have the same meaning as commonly understood by one of ordinary skill in the art to which this invention belongs. Although methods and materials similar or equivalent to those described herein can be used in the practice or testing of the present invention, suitable methods and materials are described below. All publications, patent applications, patents, and other references mentioned herein are incorporated by reference in their entirety. In case of conflict, the present specification, including definitions, will control. In addition, the materials, methods, and examples are illustrative only and not intended to be limiting,

Other features, objects, and advantages of the invention will be apparent from the detailed description, and from the claims.

### BRIEF DESCRIPTION OF THE DRAWINGS

FIG. 1 shows albumin excretion in PAN-injured rats treated with compound 100 or mizoribine.

FIG. 2 shows vascularization of human kidney organoids when transplanted under the rat kidney capsule.

FIG. 3 shows oral dosing of compound 100 results in drug exposure in an implanted organoid.

FIG. 4 shows a plot of the effect of compound AO on alumbin excretion in DOCA-salt hypertensive rats.

FIGS. 5A-5F show confocal microscopy images (FIGS. 5A, 5B, 5D, 5E, 5F) of murine podocytes pretreated with compound AO or DMSO, and then insulted with protamine sulfate (PS), and quantitation of treated podocytes with collapsed actin cytoplasm (FIG. 5C).

FIGS. 6A-6F show confocal microscopy images (FIGS. 6A, 6B, 6D, 6E, 6F) of human iPSC derived kidney organoids pretreated with compound AO or DMSO, and then insulted with prolamine sulfate (PS), and quantitation of mean phalloidin intensity per organoid (FIG. 6C).

### DETAILED DESCRIPTION

### Definitions

The term “acyl” is art-recognized and refers to a group represented by the general formula hydrocarbylC(O)—, preferably alkylC(O)—.

The term “acylamino” is art-recognized and refers to an amino group substituted with an acyl group and may be represented, for example, by the formula hydrocarbylC(O)NH—.

The term “acyloxy” is art-recognized and refers to a group represented by the general formula hydrocarbylC(O)O—, preferably alkylC(O)O—.

The term “alkoxy” refers to an alkyl group, preferably a lower alkyl group, having an oxygen attached thereto. Representative alkoxy groups include methoxy, trifluoromethoxy, ethoxy, propoxy, tert-butoxy and the like.

The term “alkoxyalkyl” refers to an alkyl group substituted with an alkoxy group and may be represented by the general formula alkyl-O-alkyl.

The term “alkenyl”, as used herein, refers to an aliphatic group containing at least one double bond and is intended to include both “unsubstituted alkenyls” and “substituted alkenyls”, the latter of which refers to alkenyl moieties having substituents replacing a hydrogen on one or more carbons of the alkenyl group. Such substituents may occur on one or more carbons that are included or not included in one or more double bonds. Moreover, such substituents include all those contemplated for alkyl groups, as discussed below, except where stability is prohibitive. For example, substitution of alkenyl groups by one or more alkyl, carbocyclyl, aryl, heterocyclyl, or heteroaryl groups is contemplated.

An “alkyl” group or “alkane” is a straight chained or branched non-aromatic hydrocarbon which is completely saturated. Typically, a straight chained or branched alkyl group has from 1 to about 20 carbon atoms, preferably from 1 to about 10 unless otherwise defined. Examples of straight chained and branched alkyl groups include methyl, ethyl, n-propyl, iso-propyl, n-butyl, sec-butyl, tert-butyl, pentyl, hexyl, pentyl and octyl. A C1-C6 straight chained or branched alkyl group is also referred to as a “lower alkyl” group.

Moreover, the term “alkyl” (or “lower alkyl”) as used throughout the specification, examples, and claims is intended to include both “unsubstituted alkyls” and “substituted alkyls”, the latter of which refers to alkyl moieties having substituents replacing a hydrogen on one or more carbons of the hydrocarbon backbone. Such substituents, if not otherwise specified, can include, for example, a halogen (e.g., fluoro), a hydroxyl, a carbonyl (such as a carboxyl, an alkoxycarbonyl, a formyl, or an acyl), a thiocarbonyl (such as a thioester, a thioacetate, or a thioformate), an alkoxy, a phosphoryl, a phosphate, a phosphonate, a phosphinate, an amino, an amido, an amidine, an imine, a cyano, a nitro, an azido, a sulfhydryl, an alkylthio, a sulfate, a sulfonate, a sulfamoyl, a sulfonamido, a sulfonyl, a heterocyclyl, an aralkyl, or an aromatic or heteroaromatic moiety. In preferred embodiments, the substituents on substituted alkyls are selected from C₁₋₆ alkyl, C₃₋₆ cycloalkyl, halogen, carbonyl, cyano, or hydroxyl. In more preferred embodiments, the substituents on substituted alkyls are selected from fluoro, carbonyl, cyano, or hydroxyl. It will be understood by those skilled in the art that the moieties substituted on the hydrocarbon chain can themselves be substituted, if appropriate. For instance, the substituents of a substituted alkyl may include substituted and unsubstituted forms of amino, azido, imino, amido, phosphoryl (including phosphonate and phosphinate), sulfonyl (including sulfate, sulfonamido, sulfamoyl and sulfonate), and silyl groups, as well as ethers, alkylthios, carbonyls (including ketones, aldehydes, carboxylates, and esters), —CF₃, —CN and the like. Exemplary substituted alkyls are described below. Cycloalkyls can be further substituted with alkyls, alkenyls, alkoxys, alkylthios, aminoalkyls, carbonyl-substituted alkyls, —CF₃, —CN, and the like.

Unless otherwise specified, “alkylene” by itself or as part of another substituent refers to a saturated straight-chain or branched divalent group having the stated number of carbon atoms and derived from the removal of two hydrogen atoms from the corresponding alkane. Examples of straight chained and branched alkylene groups include —CH₂— (methylene), —CH₂—CH₂— (ethylene), —CH₂—CH₂—CH₂-(propylene), —C(CH₃)₂—, —CH₂—CH(CH₃)—, —CH₂—CH₂—CH₂—CH₂—, —CH₂—CH₂—CH₂—CH₂—CH₂— (pentylene), —CH₂—CH(CH₃)—CH₂—, and —CH₂—C(CH₃)₂—CH₂—.

The term “C_{x-y}” when used in conjunction with a chemical moiety, such as, acyl, acyloxy, alkyl, alkenyl, alkynyl, or alkoxy is meant to include groups that contain from x to y carbons in the chain. For example, the term “C_{x-y} alkyl” refers to substituted or unsubstituted saturated hydrocarbon groups, including straight-chain alkyl and branched-chain alkyl groups that contain from x to y carbons in the chain, including haloalkyl groups. Preferred haloalkyl groups include trifluoromethyl, difluoromethyl, 2,2,2-trifluoroethyl, and pentafluoroethyl. C₀ alkyl indicates a hydrogen where the group is in a terminal position, a bond if internal. The terms “C_{2-y} alkenyl” and “C_{2-y} alkynyl” refer to substituted or unsubstituted unsaturated aliphatic groups analogous in length and possible substitution to the alkyls described above, but that contain at least one double or triple bond respectively.

The term “alkylamino”, as used herein, refers to an amino group substituted with at least one alkyl group.

The term “alkylthio”, as used herein, refers to a thiol group substituted with an alkyl group and may be represented by the general formula alkylS-.

The term “alkynyl”, as used herein, refers to an aliphatic group containing at least one triple bond and is intended to include both “unsubstituted alkynyls” and “substituted alkynyls”, the latter of which refers to alkynyl moieties having substituents replacing a hydrogen on one or more carbons of the alkynyl group. Such substituents may occur on one or more carbons that are included or not included in one or more triple bonds. Moreover, such substituents include all those contemplated for alkyl groups, as discussed above, except where stability is prohibitive. For example, substitution of alkynyl groups by one or more alkyl, carbocyclyl, aryl, heterocyclyl, or heteroaryl groups is contemplated.

The term “amide”, as used herein, refers to a group

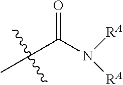

wherein each R^{A} independently represent a hydrogen or hydrocarbyl group, or two R^{A} are taken together with the N atom to which they are attached complete a heterocycle having from 4 to 8 atoms in the ring structure.

The terms “amine” and “amino” are art-recognized and refer to both unsubstituted and substituted amines and salts thereof, e.g., a moiety that can be represented by

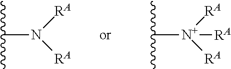

wherein each R^{A} independently represents a hydrogen or a hydrocarbyl group, or two R^{A} are taken together with the N atom to which they are attached complete a heterocycle having from 4 to 8 atoms in the ring structure.

The term “aminoalkyl”, as used herein, refers to an alkyl group substituted with an amino group.

The term “aralkyl”, as used herein, refers to an alkyl group substituted with an aryl group.

The term “aryl” as used herein include substituted or unsubstituted single-ring aromatic groups in which each atom of the ring is carbon. Preferably, the ring is a 6- or 10-membered ring, more preferably a 6-membered ring. The term “aryl” also includes polycyclic ring systems having two or more cyclic rings in which two or more carbons are common to two adjoining rings wherein at least one of the rings is aromatic, e.g., the other cyclic rings can be cycloalkyls, cycloalkenyls, aryls, heteroaryls, and/or heterocyclyls. Aryl groups include benzene, naphthalene, phenanthrene, phenol, aniline, and the like.

The term “carbamate” is art-recognized and refers to a group

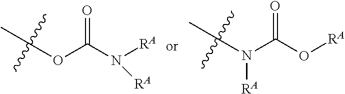

wherein each R^{A} independently represent hydrogen or a hydrocarbyl group, such as an alkyl group, or both R^{A} taken together with the intervening atom(s) complete a heterocycle having from 4 to 8 atoms in the ring structure.

The terms “carbocycle”, and “carbocyclic”, as used herein, refers to a saturated or unsaturated ring in which each atom of the ring is carbon. The term carbocycle includes both aromatic carbocycles and non-aromatic carbocycles. Non-aromatic carbocycles include both cycloalkane rings, in which all carbon atoms are saturated, and cycloalkene rings, which contain at least one double bond. “Carbocycle” includes 5-7 membered monocyclic and 8-12 membered bicyclic rings. Each ring of a bicyclic carbocycle may be selected from saturated, unsaturated and aromatic rings. Carbocycle includes bicyclic molecules in which one, two or three or more atoms are shared between the two rings. The term “fused carbocycle” refers to a bicyclic carbocycle in which each of the rings shares two adjacent atoms with the other ring. Each ring of a fused carbocycle may be selected from saturated, unsaturated and aromatic rings. In an exemplary embodiment, an aromatic ring, e.g., phenyl, may be fused to a saturated or unsaturated ring, e.g., cyclohexane, cyclopentane, or cyclohexene. Any combination of saturated, unsaturated and aromatic bicyclic rings, as valence permits, is included in the definition of carbocyclic. Exemplary “carbocycles” include cyclopentane, cyclohexane, bicyclo[2.2.1]heptane, 1,5-cyclooctadiene, 1,2,3,4-tetrahydronaphthalene, bicyclo[4.2.0]oct-3-ene, naphthalene and adamantane. Exemplary fused carbocycles include decalin, naphthalene, 1,2,3,4-tetrahydronaphthalene, bicyclo[4.2.0]octane, 4,5,6,7-tetrahydro-1H-indene and bicyclo[4.1.0]hept-3-ene. “Carbocycles” may be substituted at any one or more positions capable of bearing a hydrogen atom.

A “cycloalkyl” group is a cyclic hydrocarbon which is completely saturated. “Cycloalkyl” includes monocyclic and bicyclic rings. Typically, a monocyclic cycloalkyl group has from 3 to about 10 carbon atoms, more typically 3 to 8 carbon atoms unless otherwise defined. The second ring of a bicyclic cycloalkyl may be selected from saturated, unsaturated and aromatic rings. Cycloalkyl includes bicyclic molecules in which one, two or three or more atoms are shared between the two rings. The term “fused cycloalkyl” refers to a bicyclic cycloalkyl in which each of the rings shares two adjacent atoms with the other ring. The second ring of a fused bicyclic cycloalkyl may be selected from saturated, unsaturated and aromatic rings. A “cycloalkenyl” group is a cyclic hydrocarbon containing one or more double bonds.

The term “carbocyclylalkyl”, as used herein, refers to an alkyl group substituted with a carbocycle group.

The term “carbonate” is art-recognized and refers to a group —OCO₂—R^{A}, wherein R^{A} represents a hydrocarbyl group.

The term “carboxy”, as used herein, refers to a group represented by the formula —CO₂H.

The term “ester”, as used herein, refers to a group —C(O)OR^{A} wherein R^{A} represents a hydrocarbyl group.

The term “ether”, as used herein, refers to a hydrocarbyl group linked through an oxygen to another hydrocarbyl group. Accordingly, an ether substituent of a hydrocarbyl group may be hydrocarbyl-O—. Ethers may be either symmetrical or unsymmetrical. Examples of ethers include, but are not limited to, heterocycle-O-heterocycle and aryl-O-heterocycle. Ethers include “alkoxyalkyl” groups, which may be represented by the general formula alkyl-O-alkyl.

The terms “halo” and “halogen” as used herein means halogen and includes chloro, fluoro, bromo, and iodo.

The terms “hetaralkyl” and “heteroaralkyl”, as used herein, refers to an alkyl group substituted with a hetaryl group.

The term “heteroalkyl”, as used herein, refers to a saturated or unsaturated chain of carbon atoms and at least one heteroatom, wherein no two heteroatoms are adjacent.

The terms “heteroaryl” and “hetaryl” include substituted or unsubstituted aromatic single ring structures, preferably 5- to 7-membered rings, more preferably 5- to 6-membered rings, whose ring structures include at least one heteroatom, preferably one to four heteroatoms, more preferably one or two heteroatoms. The terms “heteroaryl” and “hetaryl” also include polycyclic ring systems having two or more cyclic rings in which two or more carbons are common to two adjoining rings wherein at least one of the rings is heteroaromatic, e.g., the other cyclic rings can be cycloalkyls, cycloalkenyls, aryls, heteroaryls, and/or heterocyclyls. Heteroaryl groups include, for example, pyrrole, furan, thiophene, imidazole, oxazole, thiazole, pyrazole, pyridine, pyrazine, pyridazine, and pyrimidine, and the like.

The term “heteroatom” as used herein means an atom of any element other than carbon or hydrogen. Preferred heteroatoms are nitrogen, oxygen, and sulfur.

The terms “heterocyclyl”, “heterocycle”, and “heterocyclic” refer to substituted or unsubstituted non-aromatic ring structures, preferably 3- to 10-membered rings, more preferably 3- to 7-membered rings, whose ring structures include at least one heteroatom, preferably one to four heteroatoms, more preferably one or two heteroatoms. The terms “heterocyclyl” and “heterocyclic” also include polycyclic ring systems having two or more cyclic rings in which two or more carbons are common to two adjoining rings wherein at least one of the rings is heterocyclic, e.g., the other cyclic rings can be cycloalkyls, cycloalkenyls, aryls, heteroaryls, and/or heterocyclyls. Heterocyclyl groups include, for example, piperidine, piperazine, pyrrolidine, tetrahydropyran, tetrahydrofuran, morpholine, lactones, lactams, and the like.

The term “heterocyclylalkyl” or “heterocycloalkyl”, as used herein, refers to an alkyl group substituted with a heterocycle group.

The term “hydrocarbyl”, as used herein, refers to a group that is bonded through a carbon atom that does not have a ═O or ═S substituent, and typically has at least one carbon-hydrogen bond and a primarily carbon backbone, but may optionally include heteroatoms. Thus, groups like methyl, ethoxyethyl, 2-pyridyl, and trifluoromethyl are considered to be hydrocarbyl for the purposes of this application, but substituents such as acetyl (which has a ═O substituent on the linking carbon) and ethoxy (which is linked through oxygen, not carbon) are not. Hydrocarbyl groups include, but are not limited to aryl, heteroaryl, carbocycle, heterocyclyl, alkyl, alkenyl, alkynyl, and combinations thereof.

The term “hydroxyalkyl”, as used herein, refers to an alkyl group substituted with a hydroxy group.

The term “lower” when used in conjunction with a chemical moiety, such as, acyl, acyloxy, alkyl, alkenyl, alkynyl, or alkoxy is meant to include groups where there are ten or fewer non-hydrogen atoms in the substituent, preferably six or fewer. A “lower alkyl”, for example, refers to an alkyl group that contains ten or fewer carbon atoms, preferably six or fewer. In certain embodiments, acyl, acyloxy, alkyl, alkenyl, alkynyl, or alkoxy substituents defined herein are respectively lower acyl, lower acyloxy, lower alkyl, lower alkenyl, lower alkynyl, or lower alkoxy, whether they appear alone or in combination with other substituents, such as in the recitations hydroxyalkyl and aralkyl (in which case, for example, the atoms within the aryl group are not counted when counting the carbon atoms in the alkyl substituent).

The terms “polycyclyl”, “polycycle”, and “polycyclic” refer to two or more rings (e.g., cycloalkyls, cycloalkenyls, aryls, heteroaryls, and/or heterocyclyls) in which two or more atoms are common to two adjoining rings, e.g., the rings are “fused rings”. Each of the rings of the polycycle can be substituted or unsubstituted. In certain embodiments, each ring of the polycycle contains from 3 to 10 atoms in the ring, preferably from 5 to 7.

The term “silyl” refers to a silicon moiety with three hydrocarbyl moieties attached thereto.

The term “substituted” refers to moieties having substituents replacing a hydrogen on one or more carbons of the backbone. It will be understood that “substitution” or “substituted with” includes the implicit proviso that such substitution is in accordance with permitted valence of the substituted atom and the substituent, and that the substitution results in a stable compound, e.g., which does not spontaneously undergo transformation such as by rearrangement, cyclization, elimination, etc. As used herein, the term “substituted” is contemplated to include all permissible substituents of organic compounds. In a broad aspect, the permissible substituents include acyclic and cyclic, branched and unbranched, carbocyclic and heterocyclic, aromatic and non-aromatic substituents of organic compounds. The permissible substituents can be one or more and the same or different for appropriate organic compounds. For purposes of this invention, the heteroatoms such as nitrogen may have hydrogen substituents and/or any permissible substituents of organic compounds described herein which satisfy the valences of the heteroatoms. Substituents can include any substituents described herein, for example, a halogen, a hydroxyl, a carbonyl (such as a carboxyl, an alkoxycarbonyl, a formyl, or an acyl), a thiocarbonyl (such as a thioester, a thioacetate, or a thioformate), an alkoxy, a phosphoryl, a phosphate, a phosphonate, a phosphinate, an amino, an amido, an amidine, an imine, a cyano, a nitro, an azido, a sulfhydryl, an alkylthio, a sulfate, a sulfonate, a sulfamoyl, a sulfonamido, a sulfonyl, a heterocyclyl, an aralkyl, or an aromatic or heteroaromatic moiety. In preferred embodiments, the substituents on substituted alkyls are selected from C₁₋₆ alkyl, C₃₋₆ cycloalkyl, halogen, carbonyl, cyano, or hydroxyl. In more preferred embodiments, the substituents on substituted alkyls are selected from fluoro, carbonyl, cyano, or hydroxyl. It will be understood by those skilled in the art that substituents can themselves be substituted, if appropriate. Unless specifically stated as “unsubstituted,” references to chemical moieties herein are understood to include substituted variants. For example, reference to an “aryl” group or moiety implicitly includes both substituted and unsubstituted variants.

The term “sulfate” is art-recognized and refers to the group —OSO₃H, or a pharmaceutically acceptable salt thereof.

The term “sulfonamide” is art-recognized and refers to the group represented by the general formulae

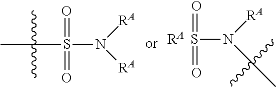

wherein each R^{A} independently represents hydrogen or hydrocarbyl, such as alkyl, or both R^{A} taken together with the intervening atom(s) complete a heterocycle having from 4 to 8 atoms in the ring structure.

The term “sulfoxide” is art-recognized and refers to the group —S(O)—R^{A}, wherein R^{A} represents a hydrocarbyl.

The term “sulfonate” is art-recognized and refers to the group SO₃H, or a pharmaceutically acceptable salt thereof.

The term “sulfone” is art-recognized and refers to the group —S(O)₂—R^{A}, wherein R^{A} represents a hydrocarbyl.

The term “thioalkyl”, as used herein, refers to an alkyl group substituted with a thiol group.

The term “thioester”, as used herein, refers to a group —C(O)SR^{A} or —SC(O)R^{A} wherein R^{A} represents a hydrocarbyl.

The term “thioether”, as used herein, is equivalent to an ether, wherein the oxygen is replaced with a sulfur.

The term “urea” is art-recognized and may be represented by the general formula

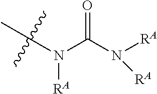

wherein each R^{A} independently represents hydrogen or a hydrocarbyl, such as alkyl, or any occurrence of R^{A} taken together with another and the intervening atom(s) complete a heterocycle having from 4 to 8 atoms in the ring structure.

“Protecting group” refers to a group of atoms that, when attached to a reactive functional group in a molecule, mask, reduce or prevent the reactivity of the functional group. Typically, a protecting group may be selectively removed as desired during the course of a synthesis. Examples of protecting groups can be found in Greene and Wuts, Protective Groups in Organic Chemistry, 3^{rd} Ed., 1999, John Wiley & Sons, NY and Harrison et al., Compendium of Synthetic Organic Methods, Vols. 1-8, 1971-1996, John Wiley & Sons, NY. Representative nitrogen protecting groups include, but are not limited to, formyl, acetyl, trifluoroacetyl, benzyl, benzyloxycarbonyl (“CBZ”), tert-butoxycarbonyl (“Boc”), trimethylsilyl (“TMS”), 2-trimethylsilyl-ethanesulfonyl (“TES”), trityl and substituted trityl groups, allyloxycarbonyl, 9-fluorenylmethyloxycarbonyl (“FMOC”), nitro-veratryloxycarbonyl (“NVOC”) and the like. Representative hydroxyl protecting groups include, but are not limited to, those where the hydroxyl group is either acylated (esterified) or alkylated such as benzyl and trityl ethers, as well as alkyl ethers, tetrahydropyranyl ethers, trialkylsilyl ethers (e.g., TMS or TIPS groups), glycol ethers, such as ethylene glycol and propylene glycol derivatives and allyl ethers.

As used herein, a therapeutic that “prevents” or “reduces the risk of developing” a disease, disorder, or condition refers to a compound that, in a statistical sample, reduces the occurrence of the disease, disorder, or condition in the treated sample relative to an untreated control sample, or delays the onset or reduces the severity of one or more symptoms of the disorder or condition relative to the untreated control sample.

The term “treating” includes prophylactic and/or therapeutic treatments. The term “prophylactic or therapeutic” treatment is art-recognized and includes administration to the host of one or more of the subject compositions. If it is administered prior to clinical manifestation of the unwanted condition (e.g., disease or other unwanted state of the host animal) then the treatment is prophylactic (i.e., it protects the host against developing the unwanted condition), whereas if it is administered after manifestation of the unwanted condition, the treatment is therapeutic, (i.e., it is intended to diminish, ameliorate, or stabilize the existing unwanted condition or side effects thereof).

The phrases “conjoint administration” and “administered conjointly” refer to any form of administration of two or more different therapeutic compounds such that the second compound is administered while the previously administered therapeutic compound is still effective in the body (e.g., the two compounds are simultaneously effective in the patient, which may include synergistic effects of the two compounds). For example, the different therapeutic compounds can be administered either in the same formulation or in a separate formulation, either concomitantly or sequentially. In certain embodiments, the different therapeutic compounds can be administered within one hour, 12 hours, 24 hours, 36 hours, 48 hours, 72 hours, or a week of one another. Thus, an individual who receives such treatment can benefit from a combined effect of different therapeutic compounds.

The term “prodrug” is intended to encompass compounds which, under physiologic conditions, are converted into the therapeutically active agents of the present invention. A common method for making a prodrug is to include one or more selected moieties which are hydrolyzed under physiologic conditions to reveal the desired molecule. In other embodiments, the prodrug is converted by an enzymatic activity of the host animal. For example, esters or carbonates (e.g., esters or carbonates of alcohols or carboxylic acids) are preferred prodrugs of the present invention. In certain embodiments, some or all of the compounds of the invention in a formulation represented above can be replaced with the corresponding suitable prodrug, e.g., wherein a hydroxyl in the parent compound is presented as an ester or a carbonate or carboxylic acid present in the parent compound is presented as an ester.

As used herein, “small molecules” refers to small organic or inorganic molecules of molecular weight below about 3,000 Daltons in general, small molecules useful for the invention have a molecular weight of less than 3,000 Daltons (Da). The small molecules can be, e.g., from at least about 100 Da to about 3,000 Da (e.g., between about 100 to about 3,000 Da, about 100 to about 2500 Da, about 100 to about 2,000 Da, about 100 to about 1,750 Da, about 100 to about 1,500 Da, about 100 to about 1,250 Da, about 100 to about 1,000 Da, about 100 to about 750 Da, about 100 to about 500 Da, about 200 to about 1500, about 500 to about 1000, about 300 to about 1000 Da, or about 100 to about 250 Da).

In some embodiments, a “small molecule” refers to an organic, inorganic, or organometallic compound typically having a molecular weight of less than about 1000. In some embodiments, a small molecule is an organic compound, with a size on the order of 1 nm. In some embodiments, small molecule drugs of the invention encompass oligopeptides and other biomolecules having a molecular weight of less than about 1000.

An “effective amount” is an amount sufficient to effect beneficial or desired results. For example, a therapeutic amount is one that achieves the desired therapeutic effect. This amount can be the same or different from a prophylactically effective amount, which is an amount necessary to prevent onset of disease or disease symptoms. An effective amount can be administered in one or more administrations, applications or dosages. A therapeutically effective amount of a composition depends on the composition selected. The compositions can be administered from one or more times per day to one or more times per week; including once every other day. The skilled artisan will appreciate that certain factors may influence the dosage and timing required to effectively treat a subject, including but not limited to the severity of the disease or disorder, previous treatments, the general health and/or age of the subject, and other diseases present. Moreover, treatment of a subject with a therapeutically effective amount of the compositions described herein can include a single treatment or a series of treatments.

Compounds of the Invention

One aspect of the invention provides methods of treating a kindey disease comprising the step of co-administering to a subject in need thereof a TRPC5 inhibitory compound and a second therapeutic agent. In some embodiments, the TRPC5 inhibitory compound is a small molecule inhibitor of TRPC5.

Small Molecule Inhibitors of TRPC5

In some embodiments, the TRPC5 inhibitory compound is a compound of structural formula (A), or a tautomer or a pharmaceutically acceptable salt thereof,

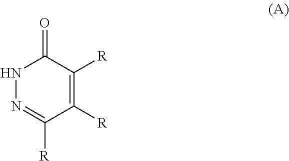

wherein

each R is independently selected from the group consisting of H, alkyl, alkenyl, alkynyl, aryl, heterocyclyl, heteroaryl, halogen, —OH, CN, cycloalkyl, —O-alkyl, —O— cycloalkyl, —O-aryl, -aryl-O-aryl, —CF₃, —C(H)F₂, alkylene-CF₃, alkylene-C(H)F₂, —SO₂-alkyl, —O-alkylene-O-alkyl, -heterocyclyl-L-R⁴, and heteroaryl-L-R⁴;

R⁴ is absent or selected from the group consisting of alkyl, cycloalkyl, polycyclyl, aryl, heterocyclyl, heteroaryl, —C(O)N(R⁵)₂, and CF₃;

R⁵ is independently H or alkyl;

R⁶ is selected from the group consisting of alkyl, cycloalkyl, aryl, heterocyclyl, heteroaryl, alkylene-aryl, —C(O)N(R⁵)₂, and CF₃;

L is absent or selected from the group consisting of methylene, —C(O)—, —SO₂—, —CH₂N(Me)-, —N(R⁵)(R⁶)—, —C(R⁵)(R⁶)—, and —O—R⁶; and

one and only one R is -heterocyclyl-L-R⁴ or -heteroaryl-L-R⁴.

In some embodiments, the TRPC5 inhibitory compound is represented by structural Formula (A-I), (A-II), or (A-III), or a tautomer or a pharmaceutically acceptable salt thereof;

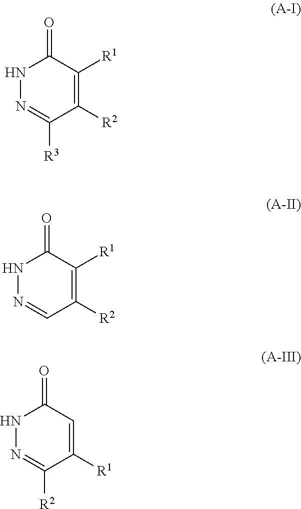

wherein

R¹ and R³ are independently selected from the group consisting of H, alkyl, alkenyl, alkynyl, aryl, heterocyclyl, heteroaryl, halogen, —OH, —CN, -cycloalkyl, —O-alkyl, —O-cycloalkyl, —O-aryl, -aryl-O-aryl, —CF₃, —C(H)F₂, alkylene-CF₃, alkylene-C(H)F₂, —SO₂-alkyl, and —O-alkylene-O-alkyl, -heterocyclyl-L-R⁴, and -heteroaryl-L-R⁴;

R² is -heterocyclyl-L-R⁴;

R⁴ is absent or selected from the group consisting of alkyl, cycloalkyl, aryl, alkylene-aryl, alkylene-heteroaryl, heteroaryl, heterocyclyl, —C(O)N(R⁵)₂, and CF₃;

R⁵ is independently H or alkyl;

R⁶ is selected from the group consisting of alkyl, cycloalkyl, aryl, heterocyclyl, heteroaryl, alkylene-aryl, —C(O)N(R⁵)₂, and CF₃;

L is absent or selected from the group consisting of methylene, —C(O)—, —SO₂—, —CH₂N(Me)-, —N(R5)(R6)-, —C(R5)(R6)-, and —O—R⁶; and

one and only one of R¹, R², and R³ is -heterocyclyl-L-R⁴ or -heteroaryl-L-R⁴.

In some embodiments, the TRPC5 inhibitory compound is a compound disclosed in International Patent Application No. PCT/US18/51465, filed Sep. 18, 2018, which is hereby incorporated by reference herein in its entirety.

In some embodiments, the TRPC5 inhibitory compound is selected from any one of the following compounds, or a pharmaceutically acceptable salt thereof:

| Compound | Structure | |
|---|---|---|
| MS | 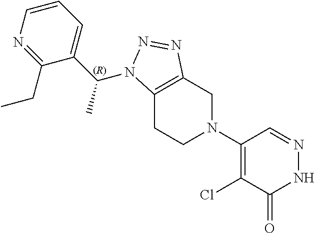 | MS |
| MX | 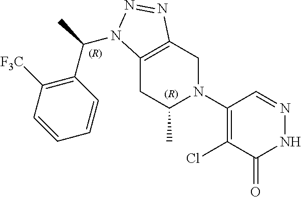 | MX |
| LY | 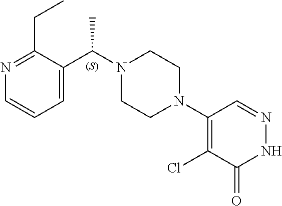 | LY |
| LW | 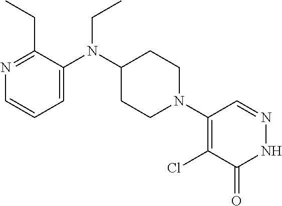 [SMILES: CCc1ncccc1N(CC)C1CCN(c2cn[nH]c(=O)c2Cl)CC1] | LW |
| OM | 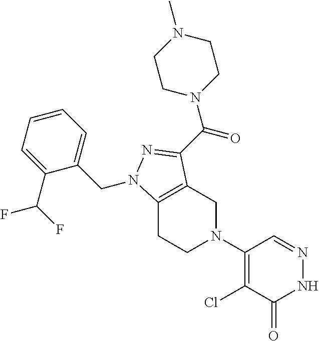 [SMILES: CN1CCN(C(=O)c2nn(Cc3ccccc3C(F)F)c3c2CN(c2cn[nH]c(=O)c2Cl)CC3)CC1] | OM |
| OU | 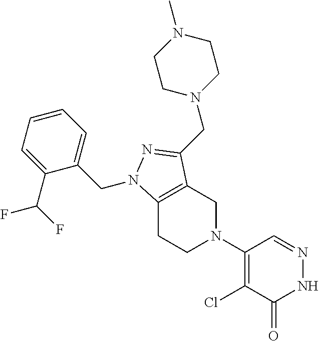 [SMILES: CN1CCN(Cc2nn(Cc3ccccc3C(F)F)c3c2CN(c2cn[nH]c(=O)c2Cl)CC3)CC1] | OU |
| OP | 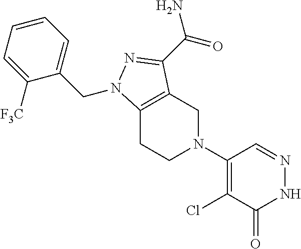 [SMILES: NC(=O)c1nn(Cc2ccccc2C(F)(F)F)c2c1CN(c1cn[nH]c(=O)c1Cl)CC2] | OP |
| NL | 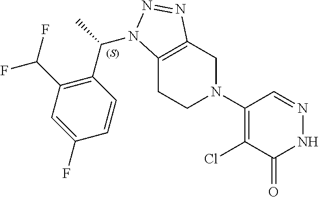 | NL |
| QM | 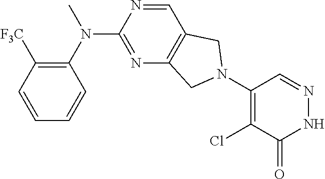 [SMILES: CN(c1ncc2c(n1)CN(c1cn[nH]c(=O)c1Cl)C2)c1ccccc1C(F)(F)F] | QM |
| MD (single enantiomer; absolute stereochemistry at benzylic methine not yet assigned) | 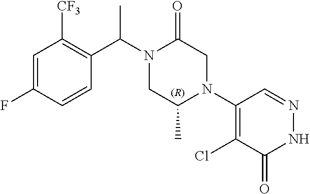 | MD |
| MF (single enantiomer; absolute stereochemistry not yet assigned) | 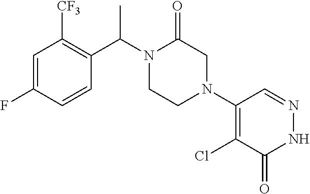 [SMILES: CC(c1ccc(F)cc1C(F)(F)F)N1CCN(c2cn[nH]c(=O)c2Cl)CC1=O] | |
| PW | 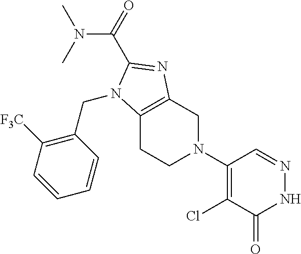 [SMILES: CN(C)C(=O)c1nc2c(n1Cc1ccccc1C(F)(F)F)CCN(c1cn[nH]c(=O)c1Cl)C2] | PW |
| PR | 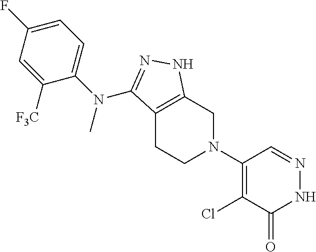 [SMILES: CN(c1ccc(F)cc1C(F)(F)F)c1n[nH]c2c1CCN(c1cn[nH]c(=O)c1Cl)C2] | PR |
| AO | 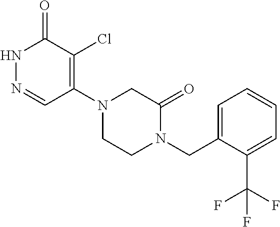 [SMILES: O=C1CN(c2cn[nH]c(=O)c2Cl)CCN1Cc1ccccc1C(F)(F)F] | |
| JX | 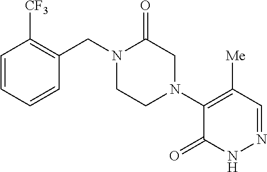 [SMILES: Cc1cn[nH]c(=O)c1N1CCN(Cc2ccccc2C(F)(F)F)C(=O)C1] | JX |
| KX | 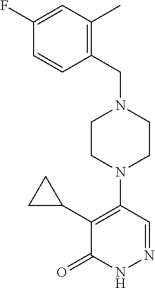 [SMILES: Cc1cc(F)ccc1CN1CCN(c2cn[nH]c(=O)c2C2CC2)CC1] | KX |
| FA | 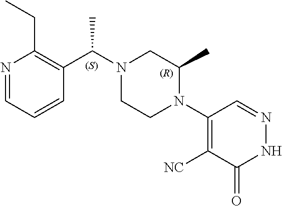 | FA |

In some embodiments, the TRPC5 inhibitory compound has structural formula

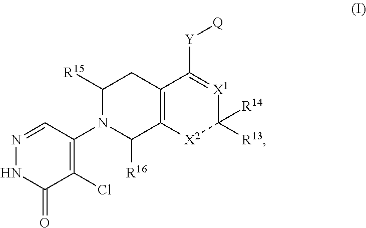

or a pharmaceutically acceptable salt thereof;
wherein:

“ [symbol] ” is a single bond or a double bond

X¹ is CH or N;

when “ [symbol] ” is a double bond, X² is CH or N;

when “ [symbol] ” is a single bond, X² is N(CH₃),

when X¹ is CH, X² is N or N(CH₃);

Y is —O—, —N(CH₃)—, —N(CH₂CH₂OH)—, cyclopropan-1,1-diyl, or —CH(CH₃)—;

Q is 2-trifluoromethyl-4-fluorophenyl, 2-difluoromethyl-4-fluorophenyl, 2-trifluoromethylphenyl, 2-methyl-4-fluorophenyl, 2-chloro-4-fluorophenyl, 2-chlorophenyl, 1-(benzyl)-4-methylpiperidin-3-yl, 4-trifluoromethylpyridin-3-yl, 2-trifluoromethyl-6-fluorophenyl, 2-trifluoromethyl-3-cyanophenyl, 2-ethyl-3-fluorophenyl, 2-chloro-3-cyanophenyl, 2-trifluoromethyl-5-fluorophenyl, or 2-difluoromethylphenyl;

when “ [symbol] ” is a double bond, le is hydrogen, —CH₂OH, —CH(OH)—CH₂OH, —NH₂, —CH(OH)CH₃, —OCH₃, or —NH—(CH₂)₂OH; and R¹⁴ is absent; or

when “ [symbol] ” is a single bond, R¹³ and R¹⁴ are taken together to form ═O; and

each of R¹⁵ and R¹⁶ is independently hydrogen or —CH₃. In some embodiments, if X¹ is N, X² is N, Y is —O— or —N(CH₃)—, and Q is 2-trifluoromethylphenyl, then at least one of R¹³, R¹⁵, and R¹⁶ is not hydrogen.

In some embodiments, the TRPC5 inhibitory compound has the structural formula

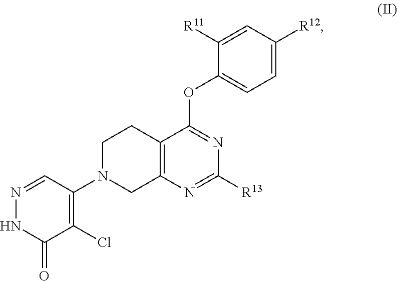

or a pharmaceutically acceptable salt thereof;
wherein:

R¹¹ is chloro, —CF₃, —CHF₂, or —CH₃;

R¹² is hydrogen or fluoro; and

R¹³ is hydrogen, —NH₂, —CH₂OH, or CH(OH)—CH₂OH.

In some embodiments, R¹¹ is —CHF₂; and R¹² is fluoro.

In some embodiments, the TRPC5 inhibitory compound is selected from any one of the following compounds, or a pharmaceutically acceptable salt thereof:

| Com- | |
|---|---|
| pound | Structure |
| 100 | 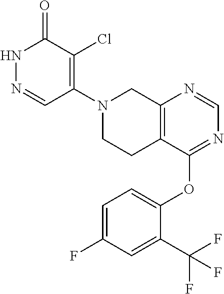 [SMILES: O=c1[nH]ncc(N2CCc3c(ncnc3Oc3ccc(F)cc3C(F)(F)F)C2)c1Cl] |
| 101 | 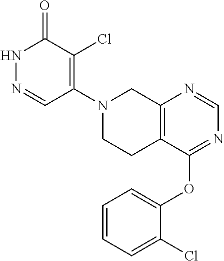 [SMILES: O=c1[nH]ncc(N2CCc3c(ncnc3Oc3ccccc3Cl)C2)c1Cl] |
| 102 | 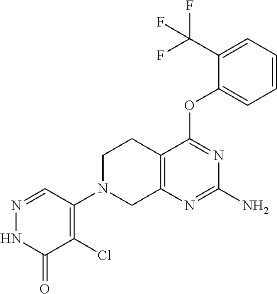 [SMILES: Nc1nc2c(c(Oc3ccccc3C(F)(F)F)n1)CCN(c1cn[nH]c(=O)c1Cl)C2] |
| 103 | 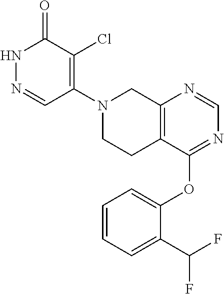 [SMILES: O=c1[nH]ncc(N2CCc3c(ncnc3Oc3ccccc3C(F)F)C2)c1Cl] |
| 104 | 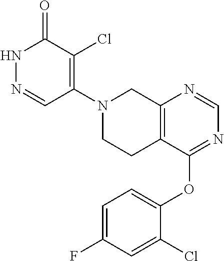 [SMILES: O=c1[nH]ncc(N2CCc3c(ncnc3Oc3ccc(F)cc3Cl)C2)c1Cl] |
| 105 | 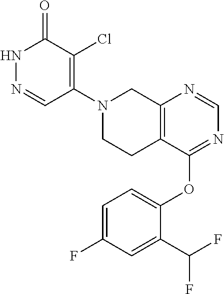 [SMILES: O=c1[nH]ncc(N2CCc3c(ncnc3Oc3ccc(F)cc3C(F)F)C2)c1Cl] |
| 106 | 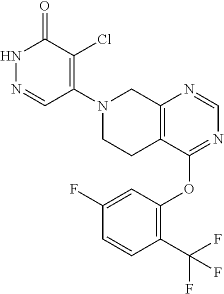 [SMILES: O=c1[nH]ncc(N2CCc3c(ncnc3Oc3cc(F)ccc3C(F)(F)F)C2)c1Cl] |
| 107 | 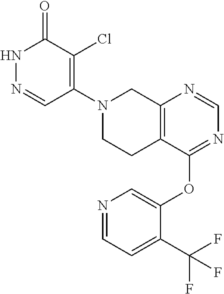 [SMILES: O=c1[nH]ncc(N2CCc3c(ncnc3Oc3cnccc3C(F)(F)F)C2)c1Cl] |
| 108 | 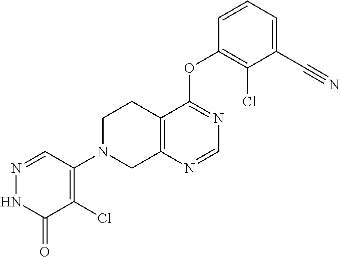 [SMILES: N#Cc1cccc(Oc2ncnc3c2CCN(c2cn[nH]c(=O)c2Cl)C3)c1Cl] |
| 109 | 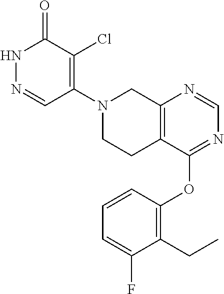 [SMILES: CCc1c(F)cccc1Oc1ncnc2c1CCN(c1cn[nH]c(=O)c1Cl)C2] |
| 110 | 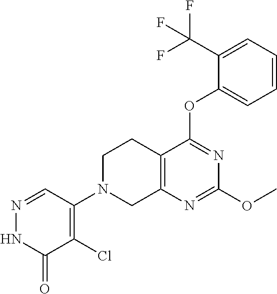 [SMILES: COc1nc2c(c(Oc3ccccc3C(F)(F)F)n1)CCN(c1cn[nH]c(=O)c1Cl)C2] |
| 111 | 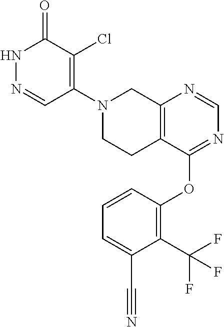 [SMILES: N#Cc1cccc(Oc2ncnc3c2CCN(c2cn[nH]c(=O)c2Cl)C3)c1C(F)(F)F] |
| 112 | 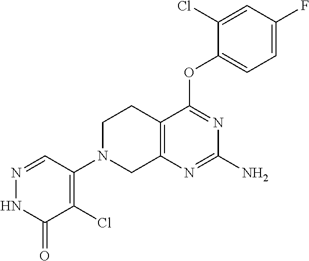 [SMILES: Nc1nc2c(c(Oc3ccc(F)cc3Cl)n1)CCN(c1cn[nH]c(=O)c1Cl)C2] |
| 113 | 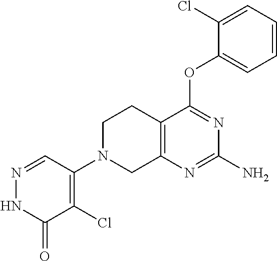 [SMILES: Nc1nc2c(c(Oc3ccccc3Cl)n1)CCN(c1cn[nH]c(=O)c1Cl)C2] |
| 114 | 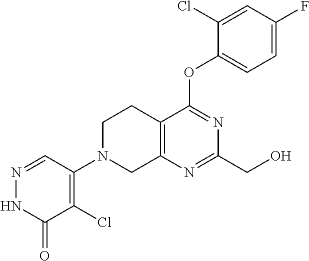 [SMILES: O=c1[nH]ncc(N2CCc3c(nc(CO)nc3Oc3ccc(F)cc3Cl)C2)c1Cl] |
| 115 | 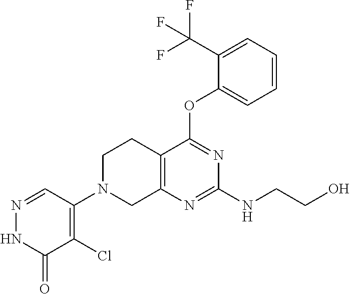 [SMILES: O=c1[nH]ncc(N2CCc3c(nc(NCCO)nc3Oc3ccccc3C(F)(F)F)C2)c1Cl] |
| 116 | 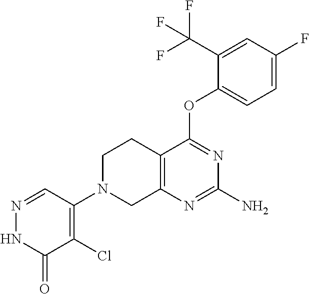 [SMILES: Nc1nc2c(c(Oc3ccc(F)cc3C(F)(F)F)n1)CCN(c1cn[nH]c(=O)c1Cl)C2] |
| 117 | 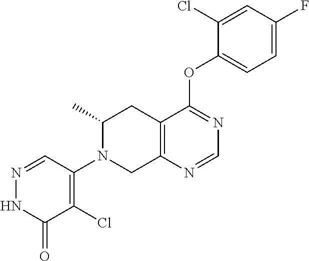 [SMILES: C[C@@H]1Cc2c(ncnc2Oc2ccc(F)cc2Cl)CN1c1cn[nH]c(=O)c1Cl] |
| 117a | 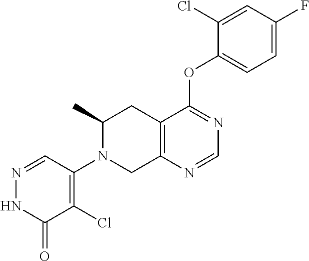 [SMILES: C[C@H]1Cc2c(ncnc2Oc2ccc(F)cc2Cl)CN1c1cn[nH]c(=O)c1Cl] |
| 118 | 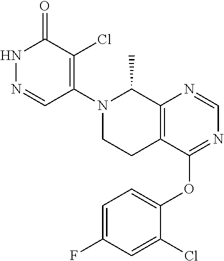 [SMILES: C[C@@H]1c2ncnc(Oc3ccc(F)cc3Cl)c2CCN1c1cn[nH]c(=O)c1Cl] |
| 119 | 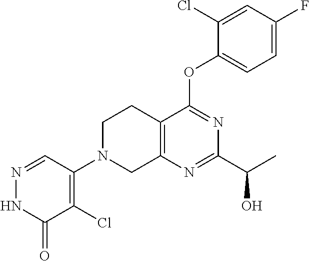 [SMILES: C[C@@H](O)c1nc2c(c(Oc3ccc(F)cc3Cl)n1)CCN(c1cn[nH]c(=O)c1Cl)C2] |
| 120 | 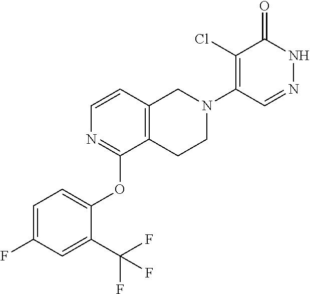 [SMILES: O=c1[nH]ncc(N2CCc3c(ccnc3Oc3ccc(F)cc3C(F)(F)F)C2)c1Cl] |
| 121 | 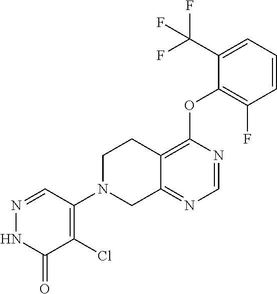 [SMILES: O=c1[nH]ncc(N2CCc3c(ncnc3Oc3c(F)cccc3C(F)(F)F)C2)c1Cl] |
| 122 | 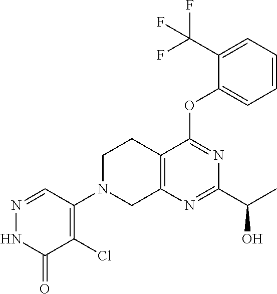 [SMILES: C[C@@H](O)c1nc2c(c(Oc3ccccc3C(F)(F)F)n1)CCN(c1cn[nH]c(=O)c1Cl)C2] |
| 123 | 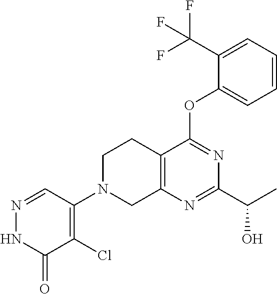 [SMILES: C[C@H](O)c1nc2c(c(Oc3ccccc3C(F)(F)F)n1)CCN(c1cn[nH]c(=O)c1Cl)C2] |
| 124 | 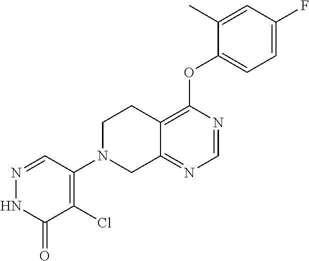 [SMILES: Cc1cc(F)ccc1Oc1ncnc2c1CCN(c1cn[nH]c(=O)c1Cl)C2] |
| 125 | 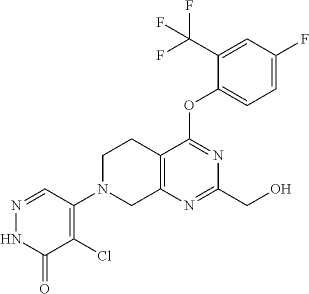 [SMILES: O=c1[nH]ncc(N2CCc3c(nc(CO)nc3Oc3ccc(F)cc3C(F)(F)F)C2)c1Cl] |
| 126 | 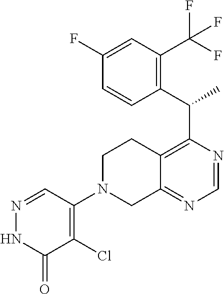 [SMILES: C[C@@H](c1ccc(F)cc1C(F)(F)F)c1ncnc2c1CCN(c1cn[nH]c(=O)c1Cl)C2] |
| 126a | 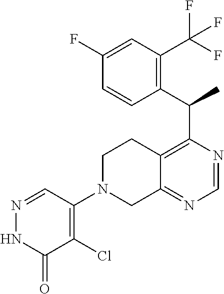 [SMILES: C[C@H](c1ccc(F)cc1C(F)(F)F)c1ncnc2c1CCN(c1cn[nH]c(=O)c1Cl)C2] |
| 127 | 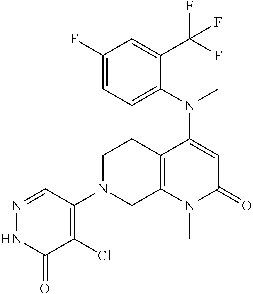 [SMILES: CN(c1ccc(F)cc1C(F)(F)F)c1cc(=O)n(C)c2c1CCN(c1cn[nH]c(=O)c1Cl)C2] |
| 128 | 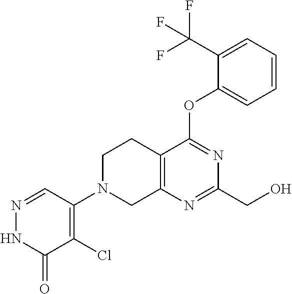 [SMILES: O=c1[nH]ncc(N2CCc3c(nc(CO)nc3Oc3ccccc3C(F)(F)F)C2)c1Cl] |
| 129 | 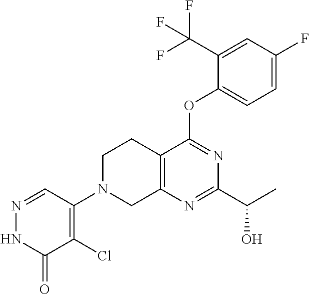 [SMILES: C[C@H](O)c1nc2c(c(Oc3ccc(F)cc3C(F)(F)F)n1)CCN(c1cn[nH]c(=O)c1Cl)C2] |
| 130 | 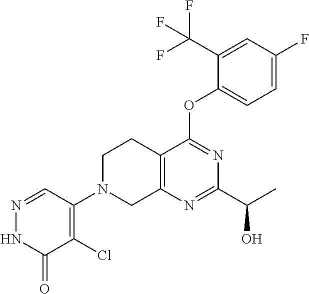 [SMILES: C[C@@H](O)c1nc2c(c(Oc3ccc(F)cc3C(F)(F)F)n1)CCN(c1cn[nH]c(=O)c1Cl)C2] |
| 131 | 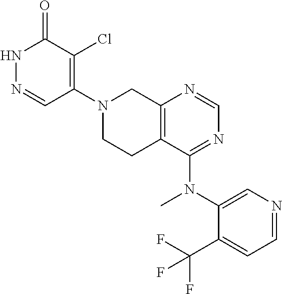 [SMILES: CN(c1cnccc1C(F)(F)F)c1ncnc2c1CCN(c1cn[nH]c(=O)c1Cl)C2] |
| 132 | 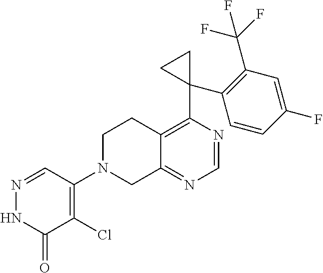 [SMILES: O=c1[nH]ncc(N2CCc3c(ncnc3C3(c4ccc(F)cc4C(F)(F)F)CC3)C2)c1Cl] |
| 133 | 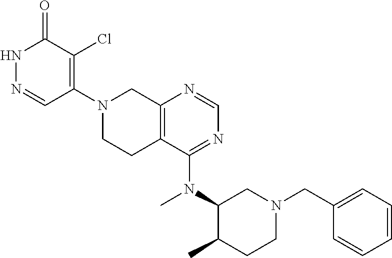 [SMILES: C[C@@H]1CCN(Cc2ccccc2)C[C@@H]1N(C)c1ncnc2c1CCN(c1cn[nH]c(=O)c1Cl)C2] |
| 133a | 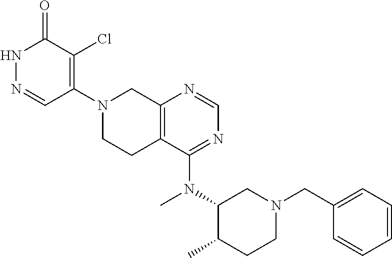 [SMILES: C[C@H]1CCN(Cc2ccccc2)C[C@H]1N(C)c1ncnc2c1CCN(c1cn[nH]c(=O)c1Cl)C2] |
| 134 | 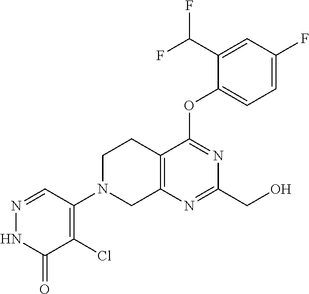 [SMILES: O=c1[nH]ncc(N2CCc3c(nc(CO)nc3Oc3ccc(F)cc3C(F)F)C2)c1Cl] |
| 135 | 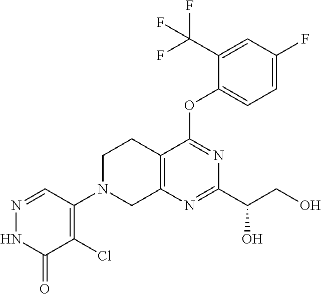 [SMILES: O=c1[nH]ncc(N2CCc3c(nc([C@@H](O)CO)nc3Oc3ccc(F)cc3C(F)(F)F)C2)c1Cl] |
| 136 | 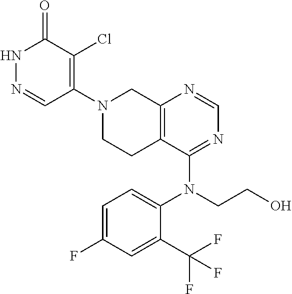 [SMILES: O=c1[nH]ncc(N2CCc3c(ncnc3N(CCO)c3ccc(F)cc3C(F)(F)F)C2)c1Cl] |
| 137 | 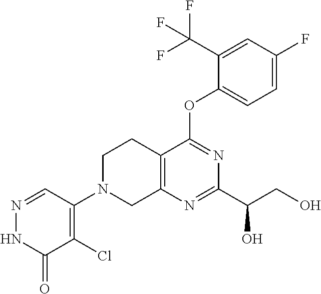 [SMILES: O=c1[nH]ncc(N2CCc3c(nc([C@H](O)CO)nc3Oc3ccc(F)cc3C(F)(F)F)C2)c1Cl] |
| 138 | 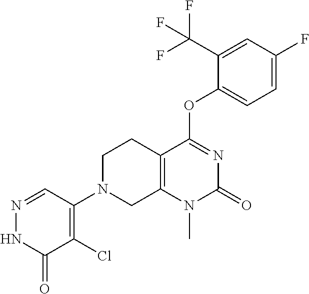 [SMILES: Cn1c2c(c(Oc3ccc(F)cc3C(F)(F)F)nc1=O)CCN(c1cn[nH]c(=O)c1Cl)C2] |
| 139 | 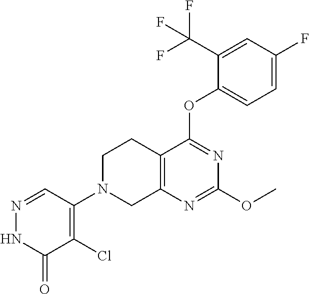 [SMILES: COc1nc2c(c(Oc3ccc(F)cc3C(F)(F)F)n1)CCN(c1cn[nH]c(=O)c1Cl)C2] |
| 140 | 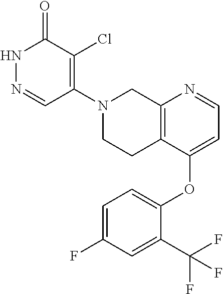 [SMILES: O=c1[nH]ncc(N2CCc3c(Oc4ccc(F)cc4C(F)(F)F)ccnc3C2)c1Cl] |

In some embodiments, the TRPC5 inhibitory compound is a compound disclosed in U.S. Provisional Patent Application No. 62/732,728, filed Sep. 18, 2018, or 62/780,553, filed Dec. 17, 2018, each of which is incorporated herein by reference in its entirety.

In some embodiments, the TRPC5 inhibitory compound is selected from any one of the following compounds, or a pharmaceutically acceptable salt thereof:

| Compound | Structure |
|---|---|
| 100 | 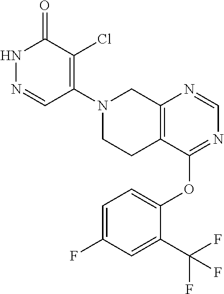 [SMILES: O=c1[nH]ncc(N2CCc3c(ncnc3Oc3ccc(F)cc3C(F)(F)F)C2)c1Cl] |
| 101 | 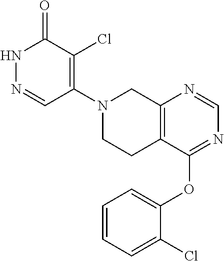 [SMILES: O=c1[nH]ncc(N2CCc3c(ncnc3Oc3ccccc3Cl)C2)c1Cl] |
| 102 | 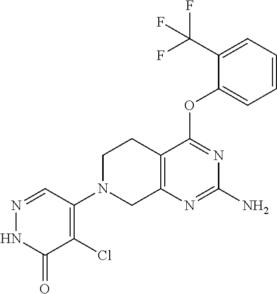 [SMILES: Nc1nc2c(c(Oc3ccccc3C(F)(F)F)n1)CCN(c1cn[nH]c(=O)c1Cl)C2] |
| 104 | 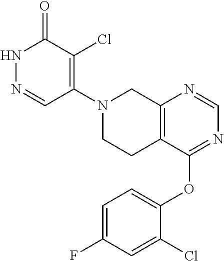 [SMILES: O=c1[nH]ncc(N2CCc3c(ncnc3Oc3ccc(F)cc3Cl)C2)c1Cl] |
| 105 | 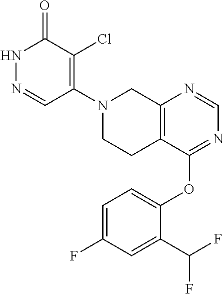 [SMILES: O=c1[nH]ncc(N2CCc3c(ncnc3Oc3ccc(F)cc3C(F)F)C2)c1Cl] |
| 114 | 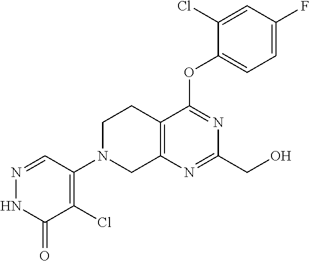 [SMILES: O=c1[nH]ncc(N2CCc3c(nc(CO)nc3Oc3ccc(F)cc3Cl)C2)c1Cl] |
| 116 | 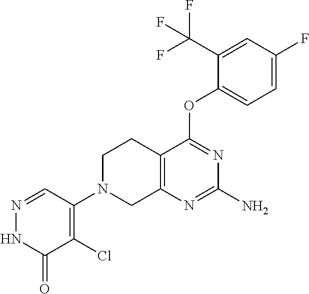 [SMILES: Nc1nc2c(c(Oc3ccc(F)cc3C(F)(F)F)n1)CCN(c1cn[nH]c(=O)c1Cl)C2] |
| 124 | 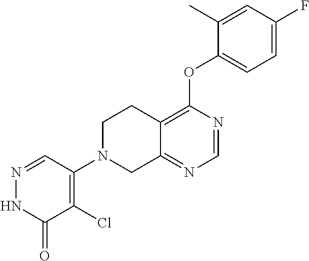 [SMILES: Cc1cc(F)ccc1Oc1ncnc2c1CCN(c1cn[nH]c(=O)c1Cl)C2] |
| 125 | 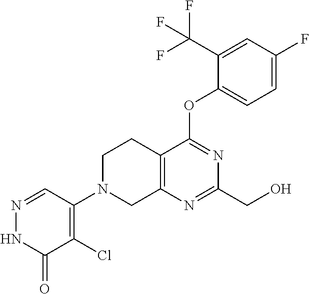 [SMILES: O=c1[nH]ncc(N2CCc3c(nc(CO)nc3Oc3ccc(F)cc3C(F)(F)F)C2)c1Cl] |
| 128 | 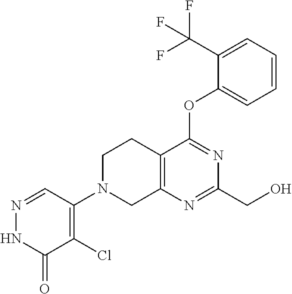 [SMILES: O=c1[nH]ncc(N2CCc3c(nc(CO)nc3Oc3ccccc3C(F)(F)F)C2)c1Cl] |
| 134 | 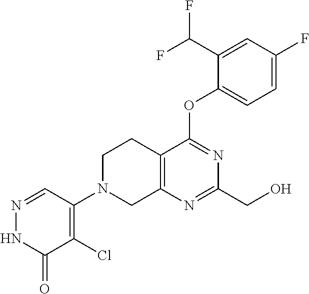 [SMILES: O=c1[nH]ncc(N2CCc3c(nc(CO)nc3Oc3ccc(F)cc3C(F)F)C2)c1Cl] |
| 135 | 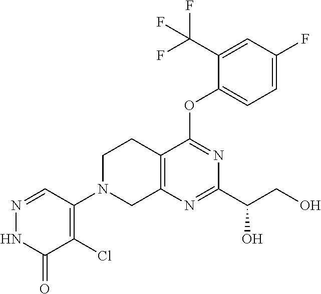 [SMILES: O=c1[nH]ncc(N2CCc3c(nc([C@@H](O)CO)nc3Oc3ccc(F)cc3C(F)(F)F)C2)c1Cl] |
| 137 | 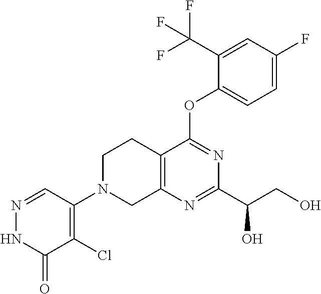 [SMILES: O=c1[nH]ncc(N2CCc3c(nc([C@H](O)CO)nc3Oc3ccc(F)cc3C(F)(F)F)C2)c1Cl] |

In some embodiments, the TRPC5 inhibitory compound is the following compound, or a pharmaceutically acceptable salt thereof:

| Compound | Structure |
|---|---|
| 100 | 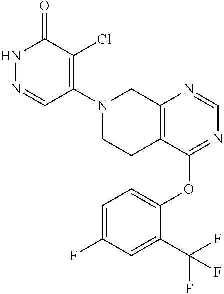 [SMILES: O=c1[nH]ncc(N2CCc3c(ncnc3Oc3ccc(F)cc3C(F)(F)F)C2)c1Cl] |

Second Therapeutic Agent

In one aspect, the present invention is directed to methods of treating kidney diseases comprising the step of co-administering to a subject in need thereof a TRPC5 inhibitory compound and a second therapeutic agent. In certain embodiments, the second therapeutic agent affects a biological pathway outside the TRPC5-Rac1 pathway; accordingly, a subject who receives such treatment can benefit from a combined effect of different therapeutic agents.

In certain embodiments, the second therapeutic agent is selected from an immunomodulator, a calcineurin inhibitor, a renin angiotensin aldosterone system inhibitor, an antiproliferative agent, a corticosteroid, an angiotensin converting enzyme inhibitor, an angiotensin receptor blocker, a sodium-glucose transport protein 2 inhibitor, a nuclear Factor-1 (erythroid-derived 2)-like 2 agonist, a chemokine receptor 2 inhibitor, a chemokine receptor 5 inhibitor, and an endothelin 1 receptor antagonist.

In some embodiments, the second therapeutic agent is additionally selected from an alkylating agent, an adrenocorticotropic hormone stimulant, a dual sodium-glucose transport protein 1/2 inhibitor, a beta blocker (such as metoprolol succinate, metoprolol tartrate, propranolol, carvedilol), a mineralocorticoid receptor antagonist (such as spironolactone, eplerenone, finerenone, esaxerenone, apararenone), a loop or thiazide diuretic (such as furosemide, bumetanide, torsemide, or Bendroflumethiazide), a calcium channel blocker (such as verapamil, diltiazem, amlodipine, or nifedipine), a statin (such as atorvastatin, pravastatin, fluvastatin, lovastatin, rosuvastatin, simvastatin, or pitavastatin), a short-intermediate or long-acting insulin (such as NPH insulin (Humulin R, Novolin R, biosimilars), Insulin Lispro (Humalog), Insulin glulisine), Insulin glargine (Basaglar, Lantus), Insulin Detemir (Levemir), Insulin degludec (Tresiba)), a dipeptidyl peptidase 4 inhibitor (such as sitagliptin, saxagliptin, linagliptin, vildagliptin), a glucagon-like peptide 1 receptor agonist (such as exenatide, liraglutide, dulaglutide, lixisenatide, albiglutide, semaglutide), a sulfonylurea (such as glimepiride, glipizide, glyburide, glibenclamide, chlorpropamide, tolazamide or tolbutamide), an apoptosis signal-regulating kinase-1 (such as selonsertib), a chymase inhibitor (fulacimstat (BAY1142524), a selective glycation inhibitor (such as GLY-230), a renin inhibitor (such as SCO-272), an interleukin-33 inhibitor (such as MEDI-3506), a farnesoid X receptor agonist (such as nidufexor (LMB763), a soluble guanylate cyclase stimulator (such as praliciguat, olinciguat, IW-6463, vericiguat, riociguat), a thromboxane receptor antagonist (such as SER150), a xanthine oxidase inhibitor (TMX-049), an erythropoietin receptor agonist (cibinetide (ARA-290), a cannabinoid receptor type 1 inverse agonist (such as nimacimab, GFB-024, CRB-4001), a NADPH oxidase inhibitor (such as APX-115), an anti-vascular endothelial growth factor B (such as CSL-346), an anti-fibrotic agent (such as FT011), a neprilysin inhibitor (such as TD-1439, TD-0714, sacubitril), a dual CD80/CD86 inhibitor (such as abatacept), a CD40 antagonist (such as bleselumab (ASKP1240), a cellular cholesterol and lipid blocker (VAR-200), a PDGFR antagonist (such as ANG 3070), a Slit guidance ligand 2 (such as PF-06730512), an APOL1 inhibitor (such as VX-147), an Nrl2 activator/NF-κB inhibitor (such as bardoxolone), a somatostatin receptor agonist (such as lanreotide), a PPAR gamma agonist (such as pioglitazone), a AMP activated protein kinase stimulator (such as metformin), a tyrosine kinase inhibitor (such as tesevatinib), a glucosylceramide synthase inhibitor (such as venglustat malate), an arginine vasopressin receptor 2 antagonist (such as lixivaptan), a xanthine oxidase inhibitor (such as oxypurinol), or vasopressin receptor 2 antagonist (such as tolvaptan).

In some embodiments, the immunomodulator is rituximab. Rituximab destroys both normal and malignant B cells that have CD20 on their surfaces and is therefore used to treat diseases which are characterized by having too many B cells, overactive B cells, or dysfunctional B cells; such disease include, but are not limited to, hematological cancers and autoimmune diseases.

In some embodiments, the immunomodulator is mycophenolate mofetil. Administration of mycophenolate mofetil can confer advantageous effects such as suppression of the immune system and preventing rejection in organ transplantation.

In some embodiments, the angiotensin converting enzyme inhibitor is captopril, zofenopril, enalapril, ramipril, quinapril, perindopril, lisinopril, benazepril, imidapril, trandolapril, or cilazapril. Angiotensin converting enzyme (ACE) inhibitors are used primarily for the treatment of hypertension and congestive heart failure. This group of drugs causes relaxation of blood vessels as well as a decrease in blood volume, which leads to lower blood pressure and decreased oxygen demand from the heart. They inhibit the angiotensin-converting enzyme, an important component of the renin-angiotensin system. They are also useful for treating other cardiovascular and kidney diseases including, but not limited to, acute myocardial infarction (heart attack), heart failure (left ventricular systolic dysfunction), and kidney complications of diabetes mellitus (diabetic nephropathy).

In some embodiments, the angiotensin receptor blocker is losartan, candesartan, valsartan, irbesartan, telmisartan, eprosartan, olmesartan, azilsartan, or fimasartan. Uses for angiotensin receptor blockers include, but are not limited to, treatment of hypertension (high blood pressure), diabetic nephropathy (kidney damage due to diabetes) and congestive heart failure.

In some embodiments, the renin angiotensin aldosterone system inhibitor is aliskiren. Inhibition of the renin angiotensin aldosterone system can confer such advantageous effects as reduction of blood pressure and improvements in intraglomerular hemodynamics. Renin, the first enzyme in the renin-angiotensin-aldosterone system, plays a role in blood pressure control. It cleaves angiotensinogen to angiotensin I, which is in turn converted by angiotensin-converting enzyme (ACE) to angiotensin II. Angiotensin II has both direct and indirect effects on blood pressure. It directly causes arterial smooth muscle to contract, leading to vasoconstriction and increased blood pressure. Angiotensin II also stimulates the production of aldosterone from the adrenal cortex, which causes the tubules of the kidneys to increase reabsorption of sodium, with water following, thereby increasing plasma volume, and thus blood pressure. Aliskiren binds to the S3bp binding site of renin, essential for its activity. Binding to this pocket prevents the conversion of angiotensinogen to angiotensin I. Aliskiren is also available as combination therapy with hydrochlorothiazide.

In some embodiments, the endothelin 1 receptor antagonist is ambrisentan, atrasentan, bosentan, macitentan, or sparsentan. Antagonism of the endothelin 1 receptor can confer such advantageous effects as reduction of blood pressure and improvements in intraglomerular hemodynamics. Macitentan, ambrisentan and bosentan are mainly used for the treatment of pulmonary arterial hypertension, which can have multifactorial mechanisms, which may include chronic kidney failure.

In some embodiments, the anti-proliferative agent is mycophenolate mofetil, mycophenolate sodium, or azathioprine. Administration of mycophenolate mofetil, mycophenolate sodium, or azathioprine can confer such advantageous effects as suppression of the immune system and preventing rejection in organ transplantation.

In some embodiments, the SGLT2 inhibitor is canagliflozin, dapagliflozin, empagliflozin, a combination of empagliflozin and linagliptin, a combination of empagliflozin and metformin, or a combination of dapagliflozin and metformin. Inhibition of SGLT2 can confer such advantageous effects as lowering of glucose and improvements in intraglomerular hemodynamics. SGLT2 inhibitors, also called gliflozins, are a class of medications that inhibit reabsorption of glucose in the kidney and therefore lower blood sugar. They act by inhibiting sodium-glucose transport protein 2 (SGLT2). SGLT2 inhibitors are used in the treatment of type II diabetes mellitus (T2DM). Apart from blood sugar control, gliflozins have been shown to provide significant cardiovascular benefit in T2DM patients. In studies on canagliflozin, the medication was found to enhance blood sugar control as well as reduce body weight and systolic and diastolic blood pressure. Sodium Glucose cotransporters (SGLTs) are proteins that occur primarily in the kidneys and play an important role in maintaining glucose balance in the blood. SGLT1 and SGLT2 are the two most know SGLTs of this family. SGLT2 is the major transport protein and promotes reabsorption from the glomerular filtration glucose back into circulation and is responsible for approximately 90% of the kidney's glucose reabsorption. SGLT2 is mainly expressed in the kidneys on the epithelial cells lining the first segment of the proximal convoluted tubule. By inhibiting SGLT2, gliflozins prevent the kidneys' reuptake of glucose from the glomerular filtrate and subsequently lower the glucose level in the blood and promote the excretion of glucose in the urine (glucosuria).

In some embodiments, the SGLT2 inhibitor also inhibits SGLT1. In some aspects of these embodiments, that SGLT1/2 inhibitor is sotagliflozin.

In some embodiments, the calcineurin inhibitor is cyclosporine A, voclosporin, or tacrolimus. Calcineurin (CaN) is a calcium and calmodulin dependent serine/threonine protein phosphatase (also known as protein phosphatase 3, and calcium-dependent serine-threonine phosphatase). It activates the T cells of the immune system and can be blocked by drugs including, but not limited to, ciclosporin, voclosporin, pimecrolimus and tacrolimus. Calcineurin activates nuclear factor of activated T cell cytoplasmic (NFATc), a transcription factor, by dephosphorylating it. The activated NFATc is then translocated into the nucleus, where it upregulates the expression of interleukin 2 (IL-2), which, in turn, stimulates the growth and differentiation of the T cell response. Calcineurin inhibitors such as tacrolimus are used to suppress the immune system in organ allotransplant recipients to prevent rejection of the transplanted tissue.

In some embodiments, the nuclear Factor-1 (erythroid-derived 2)-like 2 agonist is bardoxolone or CXA-10. Agonism of nuclear Factor-1 (erythroid-derived 2)-like 2 can confer such advantageous effects as anti-inflammatory effects. Nuclear factor (erythroid-derived 2)-like 2, also known as NFE2L2 or Nrf2, is a transcription factor that in humans is encoded by the NFE2L2 gene. Nrf2 is a basic leucine zipper (bZIP) protein that regulates the expression of antioxidant proteins that protect against oxidative damage triggered by injury and inflammation. Several drugs that stimulate the NFE2L2 pathway are being studied for treatment of diseases that are caused by oxidative stress. Heme oxygenase-1 (HMOX1, HO-1) is an enzyme that catalyzes the breakdown of heme into the antioxidant biliverdin, the anti-inflammatory agent carbon monoxide, and iron. HO-1 is a Nrf2 target gene that has been shown to protect from a variety of pathologies, including sepsis, hypertension, atherosclerosis, acute lung injury, kidney injury, and pain.

In some embodiments, the chemokine receptor 2 inhibitor is PF-04136309, ccx140, or propagemanium (DMX-200). Inhibition of chemokine receptor 2 can confer such advantageous effects as suppression of the immune system. Chemokine receptor 2 (CCR2)-mediated recruitment of monocytes and other inflammatory cells has been implicated in the etiology of diabetic nephropathy, and inhibition of CCR2 may decrease albuminuria and prevent kidney function decline in patients with diabetic nephropathy.

In some embodiments, the second therapeutic is an Nrl2 activator/NF-κB inhibitor (such as bardoxolone), a somatostatin receptor agonist (such as lanreotide), a PPAR gamma agonist (such as pioglitazone), a AMP activated protein kinase stimulator (such as metformin), a tyrosine kinase inhibitor (such as tesevatinib), a glucosylceramide synthase inhibitor (such as venglustat malate), an arginine vasopressin receptor 2 antagonist (such as lixivaptan), a xanthine oxidase inhibitor (such as oxypurinol), or vasopressin receptor 2 antagonist (such as tolvaptan). Each of these agents are either approved or in human clinical trials for the treatment of Polycystic Kidney Disease, and in particular, Autosomal Dominant Polycystic Kidney Disease.

In some embodiments, the second therapeutic agent is tacrolimus, cyclosporine A, rituximab, mycophenolate mofetil, a corticosteroid (such as prednisone), sparsentan, enalapril, or losartan. In some embodiments, the second therapeutic agent is voclosporin. In some embodiments, the second therapeutic agent is enalapril, losartan, or cyclosporine A. Corticosteroids are a class of steroid hormones that are produced in the adrenal cortex of vertebrates, as well as the synthetic analogues of these hormones. Two main classes of corticosteroids, glucocorticoids and mineralocorticoids, are involved in a wide range of physiological processes, including stress response, immune response, and regulation of inflammation, carbohydrate metabolism, protein catabolism, blood electrolyte levels, and behavior. Mineralocorticoids such as aldosterone are primarily involved in the regulation of electrolyte and water balance by modulating ion transport in the epithelial cells of the renal tubules of the kidney. Systemic corticosteroids are also used to treat diseases and conditions such as nephrotic syndrome, organ transplantation, adrenal insufficiency, and congenital adrenal hyperplasia.

In certain embodiments, the compounds of the invention may be racemic. In certain embodiments, the compounds of the invention may be enriched in one enantiomer. For example, a compound of the invention may have greater than 30% ee, 40% ee, 50% ee, 60% ee, 70% ee, 80% ee, 90% ee, or even 95% or greater ee.

The compounds of the invention have more than one stereocenter. Accordingly, the compounds of the invention may be enriched in one or more diastereomers. For example, a compound of the invention may have greater than 30% de, 40% de, 50% de, 60% de, 70% de, 80% de, 90% de, or even 95% or greater de. In certain embodiments, the compounds of the invention have substantially one isomeric configuration at one or more stereogenic centers, and have multiple isomeric configurations at the remaining stereogenic centers.

In certain embodiments, the enantiomeric excess of the stereocenter is at least 40% ee, 50% ee, 60% ee, 70% ee, 80% ee, 90% ee, 92% ee, 94% ee, 95% ee, 96% ee, 98% ee or greater ee.

As used herein, single bonds drawn without stereochemistry do not indicate the stereochemistry of the compound.

As used herein, hashed or bolded non-wedge bonds indicate relative, but not absolute, stereochemical configuration (e.g., do not distinguish between enantiomers of a given diastereomer).

As used herein, hashed or bolded wedge bonds indicate absolute stereochemical configuration.

In some embodiments, the invention relates to pharmaceutical composition comprising a compound of the invention and a pharmaceutically acceptable carrier. In certain embodiments, a therapeutic preparation or pharmaceutical composition of the compound of the invention may be enriched to provide predominantly one enantiomer of a compound. An enantiomerically enriched mixture may comprise, for example, at least 60 mol percent of one enantiomer, or more preferably at least 75, 90, 95, or even 99 mol percent. In certain embodiments, the compound enriched in one enantiomer is substantially free of the other enantiomer, wherein substantially free means that the substance in question makes up less than 10%, or less than 5%, or less than 4%, or less than 3%, or less than 2%, or less than 1% as compared to the amount of the other enantiomer, e.g., in the composition or compound mixture. For example, if a composition or compound mixture contains 98 grams of a first enantiomer and 2 grams of a second enantiomer, it would be said to contain 98 mol percent of the first enantiomer and only 2% of the second enantiomer.

In certain embodiments, a therapeutic preparation or pharmaceutical composition may be enriched to provide predominantly one diastereomer of the compound of the invention. A diastereomerically enriched mixture may comprise, for example, at least 60 mol percent of one diastereomer, or more preferably at least 75, 90, 95, or even 99 mol percent.

### Methods of Treatment

The non-selective Ca²⁺⁻ permeable Transient Receptor Potential (TRP) channels act as sensors that transduce extracellular cues to the intracellular environment in diverse cellular processes, including actin remodeling and cell migration (Greka et al., Nat Neurosci 6, 837-845, 2003; Ramsey et al., Annu Rev Physiol 68, 619-647, 2006; Montell, Pflugers Arch 451, 19-28, 2005; Clapham, Nature 426, 517-524, 2003). Dynamic rearrangement of the actin cytoskeleton relies on spatiotemporally regulated. Ca²⁺ influx (Zheng and Poo, Annu Rev Cell Dev Biol 23, 375-404, 2007); Brandman and Meyer, Science 322, 390-395, 2008); Collins and Meyer, Dev Cell 16, 160-161, 2009) and the small GTPases RhoA and Rac1 serve as key modulators of these changes (Etienne-Manneville and Hall, Nature 420, 629-635, 2002); Raftopoulou and Hall, Dev Biol. 265, 23-32, 2004). RhoA induces stress fiber and focal adhesion formation, while Rad, mediates lamellipodia formation (Etienne-Manneville and Hall, Nature 420, 629-635, 2002). The Transient Receptor Potential Cation Channel, subfamily C, member 5 (TRPC5) acts in concert with TRPC6 to regulate Ca2+ influx, actin remodeling, and cell motility in kidney podocytes and fibroblasts. TRPC5-mediated Ca^{2+.} influx increases Rac1 activity, whereas TRPC6-mediated Ca2+ influx promotes RhoA activity. Gene silencing of TRPC6 channels abolishes stress fibers and diminishes focal contacts, rendering a motile, migratory cell phenotype. In contrast, gene silencing of TRPC5 channels rescues stress fiber formation, rendering a contractile cell phenotype. The results described herein unveil a conserved signaling mechanism whereby TRPC5 and TRPC6 channels control a tightly regulated balance of cytoskeletal dynamics through differential coupling to Rac1 and RhoA.

Ca²⁺-dependent remodeling of the actin cytoskeleton is a dynamic process that drives cell migration (Wei et al., Nature 457, 901-905, 2009). RhoA and Rac1 act as switches responsible for cytoskeletal rearrangements in migrating cells (Etienne-Manneville and Hall, Nature 420, 629-635, 2002); Raftopoulou and Hall, Dev Biol 265, 23-32, 2004). Activation of Rad mediates a motile cell phenotype, whereas RhoA activity promotes a contractile phenotype (Etienne-Manneville and Hall, Nature 420, 629-635, 2002). Ca²⁺ plays a central role in small GTPase regulation (Aspenstrom et al., Biochem 377, 327-337, 2004). Spatially and temporally restricted flickers of Ca²⁺ are enriched near the leading edge of migrating cells (Wei et al., Nature 457, 901-905, 2009). Ca2+ microdomains have thus joined local bursts in Rac1 activity (Gardiner et al., Curr Biol 12, 2029-2034, 2002; Machacek et al., Nature 461, 99-103, 2009) as critical events at the leading edge. To date, the sources of Ca2+ influx responsible for GTPase regulation remain largely elusive. TRP (Transient Receptor Potential) channels generate time and space-limited Ca²⁺ signals linked to cell migration in fibroblasts and neuronal growth cones0. Specifically, TRPC5 channels are known regulators of neuronal growth cone guidance1 and their activity in neurons is dependent on PI3K and Rad activity (Bezzerides et al., Nat Cell Biol 6, 709-720, 2004).

Podocytes are neuronal-like cells that originate from the metanephric mesenchyme of the kidney glomerulus and are essential to the formation of the kidney filtration apparatus (Somlo and Mundel, Nat Genet. 24, 333-335, 2000; Fukasawa et al., J Am Soc Nephrol 20,1491-1503, 2009). Podocytes possess an exquisitely refined repertoire of cytoskeletal adaptations to environmental cues (Somlo and Mundel, Nat Genet 24, 333-335, 2000; Garg et al., Mol Cell Biol 27, 8698-8712, 2007; Verma et al., J Clin Invest 116, 1346-1359, 2006; Verma et al., J Biol Chem 278, 20716-20723, 2003; Barletta et al., J Biol Chem 278, 19266-19271, 2003; Holzman et al., Kidney Int 56, 1481-1491, 1999; Ahola et al., Am J Pathol 155, 907-913, 1999; Tryggvason and Wartiovaara, N Engl J Med 354, 1387-1401, 2006; Schnabel and Farquhar. J Cell Biol 111, 1255-1263, 1990; Kurihara et al., Proc Natl Acad Sci USA 89, 7075-7079, 1992). Early events of podocyte injury are characterized by dysregulation of the actin cytoskeleton (Faul et al, Trends Cell Biol 17, 428-437, 2007; Takeda et al., J Clin Invest 108, 289-301, 2001; Asanuma et al., Nat Cell Biol 8, 485-491, 2006) and Ca2+ homeostasis (Hunt et al., J Am Soc Nephrol 16, 1593-1602, 2005; Faul et al., Nat Med 14, 931-938, 2008). These changes are associated with the onset of proteinuria, the loss of albumin into the urinary space, and ultimately kidney failure (Tryggvason and Wartiovara, N Engl J Med 354, 1387-1401, 2006). The vasoactive hormone Angiotensin II induces Ca²⁺ influx in podocytes, and prolonged treatment results in loss of stress fibers (Hsu et al., J Mol Med 86, 1379-1394; 2008). While there is a recognized link between Ca2+ influx and cytoskeletal reorganization, the mechanisms by which the podocyte senses and transduces extracellular cues that modulate cell shape and motility remain elusive. TRP Canonical 6 (TRPC6) channel mutations have been linked to podocyte injury (Winn et at, Science 308, 1801-1804, 2005; Reiser et al., Nat Genet 37, 739-744, 2005; Moller et al., J Am Soc Nephrol 18, 29-36; 2007; Hsu et al., Biochim Biophys Acta 1772, 928-936, 2007); but little is known about the specific pathways that regulate this process. Moreover, TRPC6 shares close homology with six other members of the TRPC channel family (Ramsey et al., Annu Rev Physiol 68, 619-647, 2006; Clapham, Nature 426, 517-524, 2003). TRPC5 channels antagonize TRPC6 channel activity to control a tightly regulated balance of cytoskeletal dynamics through differential coupling to distinct small GTPases.

Proteinuria

Proteinuria is a pathological condition wherein protein is present in the urine. Albuminuria is a type of proteinuria. Microalbuminuria occurs when the kidney leaks small amounts of albumin into the urine. In a properly functioning body, albumin is not normally present in urine because it is retained in the bloodstream by the kidneys. Microalbuminuria is diagnosed either from a 24-hour urine collection (20 to 200 μg/min) or, more commonly, from elevated concentrations (30 to 300 mg/L) on at least two occasions. Microalbuminuria can be a forerunner of diabetic nephropathy. An albumin level above these values is called macroalbuminuria. Subjects with certain conditions, e.g., diabetic nephropathy, can progress from microalbuminuria to macroalbuminuria and reach a nephrotic range (>3.5 g/24 hours) as kidney disease reaches advanced stages.

Causes of Proteinuria

Proteinuria can be associated with a number of conditions, including focal segmental glomerulosclerosis, IgA nephropathy, diabetic nephropathy, lupus nephritis, membranoproliferative glomerulonephritis, progressive (crescentic) glomerulonephritis, and membranous glomerulonephritis.

A. Focal Segmental Glomerulosclerosis (FSGS)

Focal Segmental Glomerulosclerosis (FSGS) is a disease that attacks the kidney's filtering system (glomeruli) causing serious scarring. FSGS is one of the many causes of a disease known as Nephrotic Syndrome, which occurs when protein in the blood leaks into the urine (proteinuria). Primary FSGS, when no underlying cause is found, usually presents as nephrotic syndrome. Secondary FSGS, when an underlying cause is identified, usually presents with kidney failure and proteinuria. FSGS can be genetic; there are currently several known genetic causes of the hereditary forms of FSGS.

Very few treatments are available for patients with FSGS. Many patients are treated with steroid regimens, most of which have very harsh side effects. Some patients have shown to respond positively to immunosuppressive drugs as well as blood pressure drugs which have shown to lower the level of protein in the urine. To date, there is no commonly accepted effective treatment or cure and there are no FDA approved drugs to treat FSGS. Therefore, more effective methods to reduce or inhibit proteinuria are desirable.

B. IgA Nephropathy

IgA nephropathy (also known as IgA nephritis, IgAN, Berger's disease, and synpharyngitic glomerulonephritis) is a form of glomerulonephritis (inflammation of the glomeruli of the kidney). IgA nephropathy is the most common glomerulonephritis throughout the world. Primary IgA nephropathy is characterized by deposition of the IgA antibody in the glomerulus. There are other diseases associated with glomerular IgA deposits, the most common being Henoch-Schönlein purpura (HSP), which is considered by many to be a systemic form of IgA nephropathy. Henoch-Schönlein purpura presents with a characteristic purpuric skin rash, arthritis, and abdominal pain and occurs more commonly in young adults (16-35 yrs old). HSP is associated with a more benign prognosis than IgA nephropathy. In IgA nephropathy there is a slow progression to chronic renal failure in 25-30% of cases during a period of 20 years.

C. Diabetic Nephropathy

Diabetic nephropathy, also known as Kimmelstiel-Wilson syndrome and intercapillary glomerulonephritis, is a progressive kidney disease caused by angiopathy of capillaries in the kidney glomeruli. It is characterized by nephrotic syndrome and diffuse glomerulosclerosis. It is due to longstanding diabetes mellitus and is a prime cause for dialysis. The earliest detectable change in the course of diabetic nephropathy is a thickening in the glomerulus. At this stage, the kidney may start allowing more serum albumin than normal in the urine. As diabetic nephropathy progresses, increasing numbers of glomeruli are destroyed by nodular glomerulosclerosis and the amount of albumin excreted in the urine increases.

D. Lupus Nephritis

Lupus nephritis is a kidney disorder that is a complication of systemic lupus erythematosus. Lupus nephritis occurs when antibodies and complement build up in the kidneys, causing inflammation. It often causes proteinuria and may progress rapidly to renal failure. Nitrogen waste products build up in the bloodstream. Systemic lupus erythematosus causes various disorders of the internal structures of the kidney, including interstitial nephritis. Lupus nephritis affects approximately 3 out of 10,000 people.

Membranoproliferative Glomerulonephritis I/II/III

Membranoproliferative glomerulonephritis is a type of glomerulonephritis caused by deposits in the kidney glomerular mesangium and basement membrane thickening, activating complement and damaging the glomeruli. There are three types of membranoproliferative glomerulonephritis. Type I is caused by immune complexes depositing in the kidney and is believed to be associated with the classical complement pathway. Type II is similar to Type I, however, it is believed to be associated with the alternative complement pathway. Type fill is very rare and it is characterized by a mixture of subepithelial deposits and the typical pathological findings of Type I disease.

There are two major types of MPGN, which are based upon immunofluorescence microscopy: immune complex-mediated and complement-mediated. Hypocomplementemia is common in all types of MPGN. In immune complex-mediated MPGN, complement activation occurs via the classic pathway and is typically manifested by a normal or mildly decreased serum C₃ concentration and a low serum C4 concentration. In complement-mediated MPGN, there are usually low serum C3 and normal C4 levels due to activation of the alternate pathway. However, complement-mediated MPGN is not excluded by a normal serum C3 concentration, and it is not unusual to find a normal C3 concentration in adults with dense deposit disease (DDD) or C3 glomerulonephritis (C3GN).

C3 glomerulonephritis (C3GN) shows a glomerulonephritis on light microscopy (LM) bright C3 staining and the absence of C1.q, C4 and immunoglobulins (Ig) on immunofluorescence microscopy (IF), and mesangial and/or subendothelial electron dense deposits on electron microscopy (EM). Occasional intramembranous and subepithelial deposits are also frequently present. The term ‘C3 glomerulopathy’ is often used to include C3GN and Dense Deposit Disease (DDD), both of which result from dysregulation of the alternative pathway (AP) of complement. C3GN and DDD may be difficult to distinguish from each other on LM and IF studies. However, EM shows mesangial and/or subendothelial, intramembranous and subepithelial deposits in C3GN, while dense osmiophilic deposits are present along the glomerular basement membranes (GBM) and in the mesangium in DDD. Both C3GN and DDD are distinguished from immune-complex mediated glomerulonephritis by the lack of immunoglobulin staining on IF. (Sethi et al., Kidney Int. (2012) 82(4):465-473).

F. Progressive (Crescentic) Glomerulonephritis

Progressive (crescentic) glomerulonephritis (PG) is a syndrome of the kidney that, if left untreated, rapidly progresses into acute renal failure and death within months. In 50% of cases, PG is associated with an underlying disease such as Goodpasture's syndrome, systemic lupus erythematosus, or Wegener granulomatosis; the remaining cases are idiopathic. Regardless of the underlying cause, PG involves severe injury to the kidney's glomeruli, with many of the glomeruli containing characteristic crescent-shaped scars. Patients with PG have hematuria, proteinuria, and occasionally, hypertension and edema. The clinical picture is consistent with nephritic syndrome, although the degree of proteinuria may occasionally exceed 3 g/24 hours, a range associated with nephrotic syndrome. Untreated disease may progress to decreased urinary volume (oliguria), which is associated with poor kidney function.

G. Membranous Glomerulonephritis

Membranous glomerulonephritis (MGN) is a slowly progressive disease of the kidney affecting mostly patients between ages of 30 and 50 years, usually Caucasian. It can develop into nephrotic syndrome. MGN is caused by circulating immune complex. Current research indicates that the majority of the immune complexes are formed via binding of antibodies to antigens in situ to the glomerular basement membrane. The said antigens may be endogenous to the basement membrane, or deposited from systemic circulation.

H. Alport Syndrome

Alport syndrome is a genetic disorder affecting around 1 in 5,000-10,000 children, characterized by glomerulonephritis, end-stage kidney disease, and hearing loss. Alport syndrome can also affect the eyes, though the changes do not usually affect sight, except when changes to the lens occur in later life. Blood in urine is universal, Proteinuria is a feature as kidney disease progresses.

I. Hypertensive Kidney Disease

Hypertensive kidney disease (Hypertensive nephrosclerosis (HN or FINS) or hypertensive nephropathy (FIN)) is a medical condition referring to damage to the kidney due to chronic high blood pressure. HN can be divided into two types: benign and malignant. Benign nephrosclerosis is common in individuals over the age of 60 while malignant nephrosclerosis is uncommon and affects 1-5% of individuals with high blood pressure, that have diastolic blood pressure passing 130 mm Hg. Signs and symptoms of chronic kidney disease, including loss of appetite, nausea, vomiting, itching, sleepiness or confusion, weight loss, and an unpleasant taste in the mouth, may develop. Chronic high blood pressure causes damages to kidney tissue; this includes the small blood vessels, glomeruli, kidney tubules and interstitial tissues. The tissue hardens and thickens which is known as nephrosclerosis. The narrowing of the blood vessels means less blood is going to the tissue and so less oxygen is reaching the tissue resulting in tissue death (ischemia).

J. Nephrotic Syndrome

Nephrotic syndrome is a collection of symptoms due to kidney damage. This includes protein in the urine, low blood albumin levels, high blood lipids, and significant swelling. Other symptoms may include weight gain, feeling tired, and foamy urine. Complications may include blood clots, infections, and high blood pressure. Causes include a number of kidney diseases such as focal segmental glomerulosclerosis, membranous nephropathy, and minimal change disease, it may also occur as a complication of diabetes or lupus. The underlying mechanism typically involves damage to the glomeruli of the kidney. Diagnosis is typically based on urine testing and sometimes a kidney biopsy. It differs from nephritic syndrome in that there are no red blood cells in the urine. Nephrotic syndrome is characterized by large amounts of proteinuria (>3.5 g per 1.73 m2 body surface area per day, or >40 mg per square meter body surface area per hour in children), hypoalbuminemia (<2.5 g/dl), hyperlipidaemia, and edema that begins in the face. Lipiduria (lipids in urine) can also occur, but is not essential for the diagnosis of nephrotic syndrome. Hyponatremia also occur with a low fractional sodium excretion. Genetic forms of nephrotic syndrome are typically resistant to steroid and other immunosuppressive treatment. Goals of therapy are to control urinary protein loss and swelling, provide good nutrition to allow the child to grow, and prevent complications. Early and aggressive treatment are used to control the disorder.

K. Minimal Change Disease

Minimal change disease (also known as MCD, minimal change glomerulopathy, and nil disease, among others) is a disease affecting the kidneys which causes a nephrotic syndrome. The clinical signs of minimal change disease are proteinuria (abnormal excretion of proteins, mainly albumin, into the urine), edema (swelling of soft tissues as a consequence of water retention), weight gain, and hypoalbuminaemia (low serum albumin). These signs are referred to collectively as nephrotic syndrome. The first clinical sign of minimal change disease is usually edema with an associated increase in weight. The swelling may be mild but patients can present with edema in the lower half of the body, periorbital edema, swelling in the scrotal/labial area and anasarca in more severe cases. In older adults, patients may also present with acute kidney injury (20-25% of affected adults) and high blood pressure. Due to the disease process, patients with minimal change disease are also at risk of blood clots and infections.

L. Membranous Nephropathy

Membranous nephropathy refers to the deposition of immune complexes on the glomerular basement membrane (GBM) with GBM thickening. The cause is usually unknown (idiopathic), although secondary causes include drugs, infections, autoimmune disorders, and cancer. Manifestations include insidious onset of edema and heavy proteinuria with benign urinary sediment, normal renal function, and normal or elevated blood pressure. Membranous nephropathy is diagnosed by renal biopsy. Spontaneous remission is common. Treatment of patients at high risk of progression is usually with corticosteroids and cyclophosphamide or chlorambucil.

M. Postinfectious Glomerulonephritis

Acute proliferative glomerulonephritis is a disorder of the glomeruli (glomerulonephritis), or small blood vessels in the kidneys. It is a common complication of bacterial infections, typically skin infection by Streptococcus bacteria types 12, 4 and 1 (impetigo) but also after streptococcal pharyngitis, for which it is also known as postinfectious or poststreptococcal glomerulonephritis. It can be a risk factor for future albuminuria. In adults, the signs and symptoms of infection may still be present at the time when the kidney problems develop, and the terms infection-related glomerulonephritis or bacterial infection-related glomerulonephritis are also used. Acute glomerulonephritis resulted in 19,000 deaths in 2013 down from 24,000 deaths in 1990 worldwide. Acute proliferative glomerulonephritis (post-streptococcal glomerulonephritis) is caused by an infection with Streptococcus bacteria, usually three weeks after infection, usually of the pharynx or the skin, given the time required to raise antibodies and complement proteins. The infection causes blood vessels in the kidneys to develop inflammation, this hampers the renal organs ability to filter urine.[citation needed] Acute proliferative glomerulonephritis most commonly occurs in children.

N. Thin Basement Membrane Disease

Thin basement membrane disease (TBMD, also known as benign familial hematuria and thin basement membrane nephropathy or TBMN) is, along with IgA nephropathy, the most common cause of hematuria without other symptoms. The only abnormal finding in this disease is a thinning of the basement membrane of the glomeruli in the kidneys. Its importance lies in the fact that it has a benign prognosis, with patients maintaining a normal kidney function throughout their lives. Most patients with thin basement membrane disease are incidentally discovered to have microscopic hematuria on urinalysis. The blood pressure, kidney function, and the urinary protein excretion are usually normal. Mild proteinuria (less than 1.5 g/day) and hypertension are seen in a small minority of patients. Frank hematuria and loin pain should prompt a search for another cause, such as kidney stones or loin pain-hematuria syndrome. Also, there are no systemic manifestations, so presence of hearing impairment or visual impairment should prompt a search for hereditary nephritis such as Alport syndrome. Some individuals with TBMD are thought to be carriers for genes that cause Alport syndrome.

O. Mesangial Proliferative Glomerulonephritis

Mesangial proliferative glomerulonephritis is a form of glomerulonephritis associated primarily with the mesangium. There is some evidence that interleukin-10 may inhibit it in an animal model.[2] It is classified as type H lupus nephritis by the World Health Organization (WHO). Mesangial cells in the renal glomerulus use endocytosis to take up and degrade circulating immunoglobulin. This normal process stimulates mesangial cell proliferation and matrix deposition. Therefore, during times of elevated circulating immunoglobulin (i.e. lupus and IgA nephropathy) one would expect to see an increased number of mesangial cells and matrix in the glomerulus. This is characteristic of nephritic syndromes.

P. Amyloidosis (Primary)

Amyloidosis is a group of diseases in which abnormal protein, known as amyloid fibrils, builds up in tissue.[4] Symptoms depend on the type and are often variable.[2] They may include diarrhea, weight loss, feeling tired, enlargement of the tongue, bleeding, numbness, feeling faint with standing, swelling of the legs, or enlargement of the spleen.[2] There are about 30 different types of amyloidosis, each due to a specific protein misfolding.[5] Some are genetic while others are acquired.[3] They are grouped into localized and systemic forms.[2] The four most common types of systemic disease are light chain (AL), inflammation (AA), dialysis (Aβ2M), and hereditary and old age (ATTR). Primary amyloidosis refers to amyloidosis in which no associated clinical condition is identified.

Q. c1q Nephropathy

C1q nephropathy is a rare glomerular disease with characteristic mesangial Clq deposition noted on immunofluorescence microscopy. It is histologically defined and poorly understood. Light microscopic features are heterogeneous and comprise minimal change disease (MCD), focal segmental glomerulosclerosis (FSGS), and proliferative glomerulonephritis. Clinical presentation is also diverse, and ranges from asymptomatic hematuria or proteinuria to frank nephritic or nephrotic syndrome in both children and adults. Hypertension and renal insufficiency at the time of diagnosis are common findings. Optimal treatment is not clear and is usually guided by the underlying light microscopic lesion. Corticosteroids are the mainstay of treatment, with immunosuppressive agents reserved for steroid resistant cases. The presence of nephrotic syndrome and FSGS appear to predict adverse outcomes as opposed to favorable outcomes in those with MCD. (Devasahayam, et al., “Clq Nephropathy: The Unique Underrecognized Pathological Entity,” Analytical Cellular Pathology, vol. 2015, Article ID 490413, 5 pages, 2015. https://doi.org/10.1155/2015/490413.)

R. Anti-GBM Disease

Anti-glomerular basement membrane (GBM) disease, also known as Goodpasture's disease, is a rare condition that causes inflammation of the small blood vessels in the kidneys and lungs. The antiglomerular basement membrane (GBM) antibodies primarily attack the kidneys and lungs, although, generalized symptoms like malaise, weight loss, fatigue, fever, and chills are also common, as are joint aches and pains. 60 to 80% of those with the condition experience both lung and kidney involvement; 20-40% have kidney involvement alone, and less than 10% have lung involvement alone. Lung symptoms usually antedate kidney symptoms and usually include: coughing up blood, chest pain (in less than 50% of cases overall), cough, and shortness of breath. Kidney symptoms usually include blood in the urine, protein in the urine, unexplained swelling of limbs or face, high amounts of urea in the blood, and high blood pressure. GPS causes the abnormal production of anti-GBM antibodies, by the plasma cells of the blood. The anti-GBM antibodies attack the alveoli and glomeruli basement membranes. These antibodies bind their reactive epitopes to the basement membranes and activate the complement cascade, leading to the death of tagged cells. T cells are also implicated. It is generally considered a type II hypersensitivity reaction.

S. Polycystic Kidney Disease

Polycystic kidney disease (PKD) is a rare, progressive renal disease that is a major cause of chronic kidney disease. PKD accounts for 7%-10% of patients with end-stage renal disease (ESRD). Approximated half of all PKD patients progress to ESRD by fourth to sixth decade of life. PKD affects all ethnic groups and is typically slightly more progressive disease in men. There are two major categories of PKD—autosomal dominant PKD (ADPKD) and autosomal recessive PKD (ARPKD). The former is more common, while the latter is typically a pediatric condition that has a more severe, accelerated disease course. Renal cysts are the defining feature of PKD. Patients with PKD have increased risk of hypertension, CV events, aneurism, liver cysts, pyelonephritis, and pain.

Measurement of Urine Protein Levels

Protein levels in urine can be measured using methods known in the art. Until recently, an accurate protein measurement required a 24-hour urine collection. In a 24-hour collection, the patient urinates into a container, which is kept refrigerated between trips to the bathroom. The patient is instructed to begin collecting urine after the first trip to the bathroom in the morning. Every drop of urine for the rest of the day is to be collected in the container. The next morning, the patient adds the first urination after waking and the collection is complete.

More recently, researchers have found that a single urine sample can provide the needed information. In the newer technique, the amount of albumin in the urine sample is compared with the amount of creatinine, a waste product of normal muscle breakdown. The measurement is called a urine albumin-to-creatinine ratio (UACR). A urine sample containing more than 30 milligrams of albumin for each gram of creatinine (30 mg/g) is a warning that there may be a problem. If the laboratory test exceeds 30 mg/g, another UACR test should be performed 1 to 2 weeks later. If the second test also shows high levels of protein, the person has persistent proteinuria, a sign of declining kidney function, and should have additional tests to evaluate kidney function.

Tests that measure the amount of creatinine in the blood will also show whether a subject's kidneys are removing wastes efficiently. Too much creatinine in the blood is a sign that a person has kidney damage. A physician can use the creatinine measurement to estimate how efficiently the kidneys are filtering the blood. This calculation is called the estimated glomerular filtration rate, or eGFR. Chronic kidney disease is present when the eGFR is less than 60 milliliters per minute (mL/min).

TRPC5

TRPC is a family of transient receptor potential cation channels in animals. TRPC5 is subtype of the TRPC family of mammalian transient receptor potential ion channels. Three examples of TRPC5 are highlighted below in Table 1.

TABLE 1
The TRPC5 orthologs from three different species along
with their GenBank Ref Seq Accession Numbers.

| Species | Nucleic Acid | Amino Acid | GeneID |
|---|---|---|---|
| Homo sapiens | NM_012471.2 | NP_036603.1 | 7224 |
| Mus musculus | NM_009428.2 | NP_033454.1 | 22067 |
| Rattus norvegicus | NM_080898.2 | NP_543174.1 | 140933 |

Accordingly, in certain embodiments, the invention provides methods for treating, or the reducing risk of developing, a disease or condition selected from kidney disease, pulmonary arterial hypertension, anxiety, depression, cancer, diabetic retinopathy, or pain, comprising administering to a subject in need thereof a therapeutically effective amount of a compound of the invention (e.g., a compound of structural formula I) or a pharmaceutical composition comprising said compound.

In some embodiments, the disease is kidney disease, anxiety, depression, cancer, or diabetic retinopathy.

In some embodiments, the disease or condition is kidney disease selected from Focal Segmental Glomerulosclerosis (FSGS), Diabetic nephropathy, Alport syndrome, hypertensive kidney disease, nephrotic syndrome, steroid-resistant nephrotic syndrome, minimal change disease, membranous nephropathy, idiopathic membranous nephropathy, membranoproliferative glomerulonephritis (MPGN), immune complex-mediated MPGN, complement-mediated MPGN, Lupus nephritis, postinfectious glomerulonephritis, thin basement membrane disease, mesangial proliferative glomerulonephritis, amyloidosis (primary), c1q nephropathy, rapidly progressive GN, anti-GBM disease, C3 glomerulonephritis, hypertensive nephrosclerosis, or IgA nephropathy. In some embodiments, the kidney disease is proteinuric kidney disease. In some embodiments, the kidney disease is microalbuminuria or macroalbuminuria kidney disease.

In some embodiments, the disease or condition to be treated is pulmonary arterial hypertension.

In some embodiments, the disease or condition to be treated is pain selected from neuropathic pain and visceral pain.

In some embodiments, the disease or condition is cancer selected from chemoresistant breast carcinoma, adriamycin-resistant breast cancer, chemoresistant colorectal cancer, medulloblastoma, and tumor angiogenesis.

The invention also provides methods of treating, or the reducing risk of developing, anxiety, or depression, or cancer, comprising administering to a subject in need thereof a therapeutically effective amount of a compound of the invention (e.g., a compound of Formula I), or a pharmaceutical composition comprising said compound.

In some embodiments, the disease or condition to be treated is transplant-related FSGS, transplant-related nephrotic syndrome, transplant-related proteinuria, cholestatic liver disease, polycystic kidney disease, autosomal dominant polycystic kidney disease (ADPKD), obesity, insulin resistance, Type II diabetes, prediabetes, metabolic syndrome, non-alcoholic fatty liver disease (NAFLD), or non-alcoholic steatohepatitis (NASH).

Subjects to be Treated

In one aspect of the invention, a subject is selected on the basis that they have, or are at risk of developing, a kidney disease, pulmonary arterial hypertension, anxiety, depression, cancer, diabetic retinopathy, or pain. In another aspect, a subject is selected on the basis that they have, or are at risk of developing, kidney disease, anxiety, depression, cancer, or diabetic retinopathy. In another aspect of the invention, a subject is selected on the basis that they have, or are at risk of developing, pain, neuropathic pain, visceral pain, transplant-related FSGS, transplant-related nephrotic syndrome, transplant-related proteinuria, cholestatic liver disease, polycystic kidney disease, autosomal dominant polycystic kidney disease (ADPKD), obesity, insulin resistance, Type II diabetes, prediabetes, metabolic syndrome, non-alcoholic fatty liver disease (NAFLD), or non-alcoholic steatohepatitis (NASH).

Subjects that have, or are at risk of developing, proteinuria include those with diabetes, hypertension, or certain family backgrounds. In the United States, diabetes is the leading cause of end-stage renal disease (ESRD). In both type 1 and type 2 diabetes, albumin in the urine is one of the first signs of deteriorating kidney function. As kidney function declines, the amount of albumin in the urine increases. Another risk factor for developing proteinuria is hypertension. Proteinuria in a person with high blood pressure is an indicator of declining kidney function. If the hypertension is not controlled, the person can progress to full kidney failure. African Americans are more likely than Caucasians to have high blood pressure and to develop kidney problems from it, even when their blood pressure is only mildly elevated. Other groups at risk for proteinuria are American Indians, Hispanics/Latinos, Pacific Islander Americans, older adults, and overweight subjects.

In one aspect of the invention, a subject is selected on the basis that they have, or are at risk of developing proteinuria. A subject that has, or is at risk of developing, proteinuria is one having one or more symptoms of the condition. Symptoms of proteinuria are known to those of skill in the art and include, without limitation, large amounts of protein in the urine, which may cause it to look foamy in the toilet. Loss of large amounts of protein may result in edema, where swelling in the hands, feet, abdomen, or face may occur. These are signs of large protein loss and indicate that kidney disease has progressed. Laboratory testing is the only way to find out whether protein is in a subject's urine before extensive kidney damage occurs.

The methods are effective for a variety of subjects including mammals, e.g., humans and oilier animals, such as laboratory animals, e.g., mice, rats, rabbits, or monkeys, or domesticated and farm animals, e.g., cats, dogs, goats, sheep, pigs, cows, or horses. In some embodiments, the subject is a mammal. In some embodiments, the subject is a human.

### EXAMPLES

The invention is further described in the following examples, which do not limit the scope of the invention described in the claims.

### Example 1. Synthesis of Compound 100

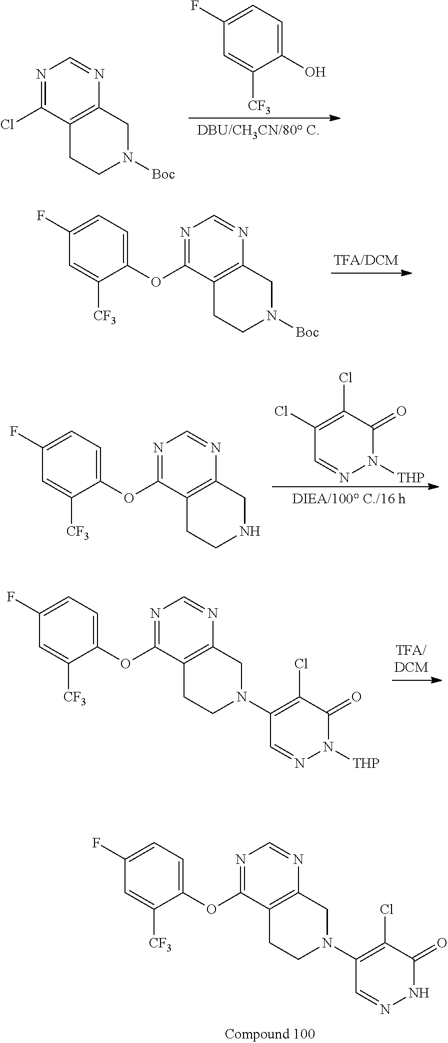

### tert-butyl 4-[4-fluoro-2-(trifluoromethyl)phenoxy]-5H,6H,7H,8H-pyrido[3,4-d]pyrimidine-7-carboxylate

To a stirred solution of tert-butyl 4-chloro-5H,6H,7H,8H-pyrido[3,4-d]pyrimidine-7-carboxylate (400 mg, 1.48 mmol, 1 equiv.) and 4-fluoro-2-(trifluoromethyl)phenol (400.6 mg, 2.22 mmol, 1.5 equiv.) in acetonitrile (10 mL) was added DBU (451.5 mg, 2.97 mmol, 2.00 equiv.) at room temperature. The resulting mixture was stirred for 2 h at 80° C. The reaction was monitored by LCMS. The mixture was allowed to cool down to room temperature. The resulting mixture was concentrated under reduced pressure. The resulting mixture was extracted with DCM (3×100 mL). The combined organic layers were washed with brine (3×100 mL), dried over anhydrous Na₂SO₄. After filtration, the filtrate was concentrated under reduced pressure. The residue was purified by Prep-TLC (PE/EtOAc 2:1) to afford tert-butyl 4-[4-fluoro-2-(trifluoromethyl)phenoxy]-5H,6H,7H,8H-pyrido[3,4-d]pyrimidine-7-carboxylate (110 mg, 17.94%) as a brown solid.

### 4-[4-fluoro-2-(trifluoromethyl)phenoxy]-5H,6H,7H,8H-pyrido[3,4-d]pyrimidine

To a stirred solution of tert-butyl 4-[4-fluoro-2-(trifluoromethyl)phenoxy]-5H,6H,7H,8H-pyrido[3,4-d]pyrimidine-7-carboxylate (110 mg, 0.27 mmol, 1 equiv.) in DCM (4 mL) was added TFA (1 mL, 13.46 mmol, 50.59 equiv.) at room temperature. The resulting mixture was stirred for 1 h at room temperature. The reaction was monitored by LCMS. The resulting mixture was concentrated under reduced pressure. The mixture was basified to pH 8 with saturated NaHCO₃(aq.). The resulting mixture was concentrated under reduced pressure. The residue was purified by Prep-TLC (DCM/MeOH 12:1) to afford 4-[4-fluoro-2-(trifluoromethyl)phenoxy]-5H,6H,7H,8H-pyrido[3,4-d]pyrimidine (50 mg, 59.98%) as a brown solid.

### 4-chloro-5-[4-[4-fluoro-2-(trifluoromethyl)phenoxy]-5H,6H,7H,8H-pyrido[3,4-d]pyrimidin-7-yl]-2-(oxan-2-yl)-2,3-dihydropyridazin-3-one

To a stirred solution of 4-[4-fluoro-2-(trifluoromethyl)phenoxy]-5H,6H,7H,8H-pyrido[3,4-d]pyrimidine (50 mg, 0.16 mmol, 1 equiv.) in DIEA (2 mL) was added 4,5-dichloro-2-(oxan-2-yl)-2,3-dihydropyridazin-3-one (47.5 mg, 0.19 mmol, 1.19 equiv.) at room temperature. The resulting mixture was stirred for 2 h at 100° C. The reaction was monitored by LCMS. The mixture was allowed to cool down to room temperature. The residue was purified by Prep-TLC (PE/EtOAc 2:1) to afford 4-chloro-5-[4-[4-fluoro-2-(trifluoromethyl)phenoxy]-5H,6H,7H,8H-pyrido[3,4-d]pyrimidin-7-yl]-2-(oxan-2-yl)-2,3-dihydropyridazin-3-one (40 mg, 47.65%) as a brown solid.

### 4-chloro-5-[4-[4-fluoro-2-(trifluoromethyl)phenoxy]-5H,6H,7H,8H-pyrido[3,4-d]pyrimidin-7-yl]-2,3-dihydropyridazin-3-one

To a stirred solution of 4-chloro-5-[4-[4-fluoro-2-(trifluoromethyl)phenoxy]-5H,6H,7H,8H-pyrido[3,4-d]pyrimidin-7-yl]-2-(oxan-2-yl)-2,3-dihydropyridazin-3-one (40 mg, 0.08 mmol, 1 equiv.) in DCM (4 mL) was added TFA (1 mL, 13.46 mmol, 177.00 equiv.) dropwise at room temperature. The resulting mixture was stirred for 1 h at room temperature. The reaction was monitored by LCMS. The resulting mixture was concentrated under reduced pressure. The mixture was basified to pH 8 with saturated NaHCO₃(aq.). The resulting mixture was concentrated under reduced pressure. The crude product (40 mg) was purified by Prep-HPLC with the following conditions (Column: XBridge Prep OBD C18 Column 30×150 mm Sum; Mobile Phase A:Water (10 MMOL/L NH4HCO3), Mobile Phase B: acetonitrile; Flow rate: 60 mL/min; Gradient: 18% B to 47% B in 7 min; 220 nm; Rt: 6.22 min) to afford 4-chloro-5-[4-[4-fluoro-2-(trifluoromethyl)phenoxy]-5H,6H,7H,8H-pyrido[3,4-d]pyrimidin-7-yl]-2,3-dihydropyridazin-3-one (8.6 mg, 25.59%) as a white solid.

### Example 2. Synthesis of Compound 140

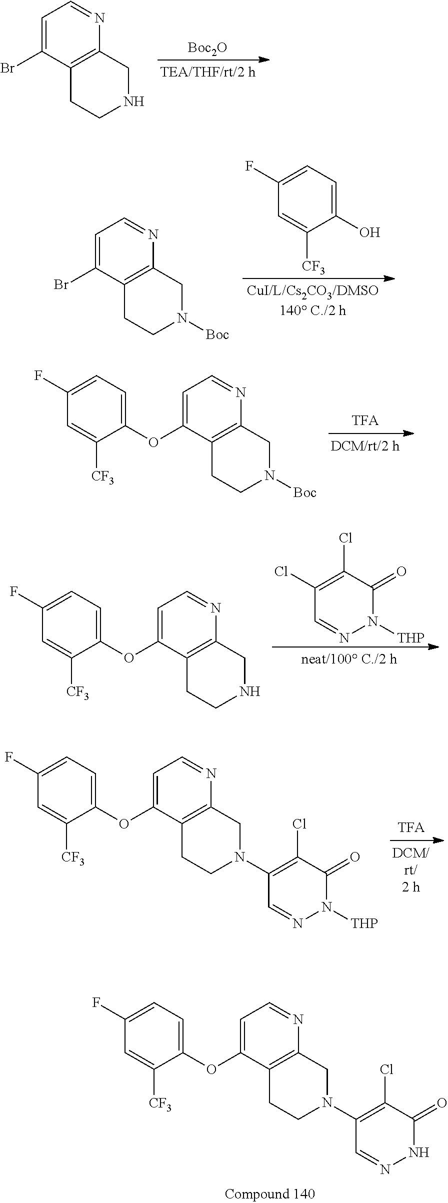

### Tert-butyl 4-bromo-5,6,7,8-tetrahydro-1,7-naphthyridine-7-carboxylate

To a solution of 4-bromo-5,6,7,8-tetrahydro-1,7-naphthyridine (250 mg, 1.173 mmol, 1 equiv.) in THF (10 mL, 123.430 mmol, 105.20 equiv.) were added Boc₂O (512.13 mg, 2.347 mmol, 2.00 equiv.) and TEA (474.90 mg, 4.693 mmol, 4 equiv.) at 25° C. The solution was stirred at 25° C. for 2 hours. The resulting mixture was concentrated under reduced pressure. The residue was purified by Prep-TLC (PE/EA 5/1) to afford tert-butyl 4-bromo-5,6,7,8-tetrahydro-1,7-naphthyridine-7-carboxylate (210 mg, 57.15%) as a light yellow oil.

### Tert-butyl 4-[4-fluoro-2-(trifluoromethyl)phenoxy]-5,6,7,8-tetrahydro-1,7-naphthyridine-7-carboxylate

To a solution of tert-butyl 4-bromo-5,6,7,8-tetrahydro-1,7-naphthyridine-7-carboxylate (210 mg, 0.671 mmol, 1 equiv.) and 4-fluoro-2-(trifluoromethyl)phenol (241.52 mg, 1.341 mmol, 2 equiv.) in DMSO (10 mL) were added Cs₂CO₃ (873.86 mg, 2.682 mmol, 4 equiv), 2-(dimethylamino)acetic acid (41.46 mg, 0.402 mmol, 0.6 equiv.) and CuI (76.62 mg, 0.402 mmol, 0.60 equiv). After stirring for 4 hours at 120° C. under a nitrogen atmosphere, the resulting mixture was concentrated under reduced pressure. The residue was purified by Prep-TLC, eluted with PE/EA (5/1) to afford tert-butyl 4-[4-fluoro-2-(trifluoromethyl)phenoxy]-5,6,7,8-tetrahydro-1,7-naphthyridine-7-carboxylate (100 mg, 36.17%) as a light yellow solid.

### 4-[4-fluoro-2-(trifluoromethyl)phenoxy]-5,6,7,8-tetrahydro-1,7-naphthyridine

To a solution of tert-butyl 4-[4-fluoro-2-(trifluoromethyl)phenoxy]-5,6,7,8-tetrahydro-1,7-naphthyridine-7-carboxylate (150 mg, 0.364 mmol, 1 equiv.) in DCM (10 mL, 157.300 mmol, 432.46 equiv.) was added TFA (414.75 mg, 3.637 mmol, 10 equiv.) at 25° C. The solution was stirred at 25° C. for 2 hours. The resulting mixture was concentrated under reduced pressure. The residue was used the next step.

### 4-chloro-5-[4-[4-fluoro-2-(trifluoromethyl)phenoxy]-5,6,7,8-tetrahydro-1,7-naphthyridin-7-yl]-2-(oxan-2-yl)-2,3-dihydropyridazin-3-one

A mixture of 4-[4-fluoro-2-(trifluoromethyl)phenoxy]-5,6,7,8-tetrahydro-1,7-naphthyridine (60 mg, 0.192 mmol, 1 equiv.) and 4,5-dichloro-2-(oxan-2-yl)-2,3-dihydropyridazin-3-one (47.86 mg, 0.192 mmol, 1.00 equiv.) in DIEA (49.67 mg, 0.384 mmol, 2 equiv.) was stirred for 2 hours at 100° C. under N₂ atmosphere. The residue was purified by Prep-TLC (PE/EA 1/1) to afford 4-chloro-5-[4-[4-fluoro-2-(trifluoromethyl)phenoxy]-5,6,7,8-tetrahydro-1,7-naphthyridin-7-yl]-2-(oxan-2-yl)-2,3-dihydropyridazin-3-one (100 mg, 99.15%) as a light yellow solid.

### 4-chloro-5-[4-[4-fluoro-2-(trifluoromethyl)phenoxy]-5,6,7,8-tetrahydro-1,7-naphthyridin-7-yl]-2,3-dihydropyridazin-3-one

To a solution of 4-chloro-5-[4-[4-fluoro-2-(trifluoromethyl)phenoxy]-5,6,7,8-tetrahydro-1,7-naphthyridin-7-yl]-2-(oxan-2-yl)-2,3-dihydropyridazin-3-one (100 mg, 0.191 mmol, 1 equiv.) in DCM (10 mL, 157.300 mmol, 825.67 equiv.) was added TFA (217.23 mg, 1.905 mmol, 10.00 equiv.) at 25° C. The solution was stirred at 25° C. for 2 hours. The crude product (150 mg) was purified by Prep-HPLC with the following conditions (Column: XBridge Shield RP18 OBD Column 30*150 mm, 5 um; Mobile Phase A:Water (10 mM NH₄HCO₃), Mobile Phase B: acetonitrile; Flow rate: 60 mL/min; Gradient: 20% B to 40% B in 7 min; 220 nm; Rt: 6.63 min) to afford 4-chloro-5-[4-[4-fluoro-2-(trifluoromethyl)phenoxy]-5,6,7,8-tetrahydro-1,7-naphthyridin-7-yl]-2,3-dihydropyridazin-3-one (42.9 mg, 51.09%) as a white solid.

### Example 3. Synthesis of Compound 120

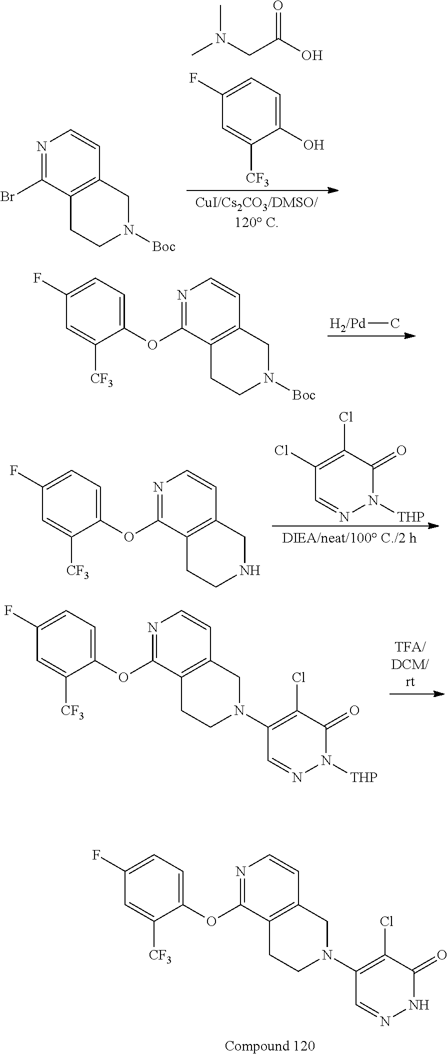

### 2-Benzyl-5-[4-fluoro-2-(trifluoromethyl)phenoxy]-1,2,3,4-tetrahydro-2,6-naphthyridine

To a stirred mixture of 2-benzyl-5-bromo-1,2,3,4-tetrahydro-2,6-naphthyridine (250 mg, 0.825 mmol, 1 equiv.) and 2-(dimethylamino)acetic acid (170.05 mg, 1.649 mmol, 2.00 equiv.) in DMSO (5 mL) were added 4-fluoro-2-(trifluoromethyl)phenol (89.10 mg, 0.495 mmol, 0.6 equiv.) and CuI (94.22 mg, 0.495 mmol, 0.6 equiv.) at room temperature. Then Cs₂CO₃ (1074.59 mg, 3.298 mmol, 4 equiv.) was added at room temperature. The final reaction mixture was irradiated with microwave radiation for 1 hours at 120° C. The reaction was monitored by LCMS. The mixture was allowed to cool down to room temperature. The crude product was purified by reverse phase flash with the following conditions (Column: XBridge Prep OBD C18 Column 30×150 mm Sum; Mobile Phase A:Water (10 mM NH₄HCO₃), Mobile Phase B: acetonitrile; Flow rate: 60 mL/min; Gradient: 18% B to 35% B in 8 min; 220 nm; Rt: 7.12 min) to afford 2-benzyl-5-[4-fluoro-2-(trifluoromethyl)phenoxy]-1,2,3,4-tetrahydro-2,6-naphthyridine (180 mg, 54.25%) as a brown solid.

### 5-[4-fluoro-2-(trifluoromethyl)phenoxy]-1,2,3,4-tetrahydro-2,6-naphthyridine

To a stirred solution of 2-benzyl-5-[4-fluoro-2-(trifluoromethyl)phenoxy]-1,2,3,4-tetrahydro-2,6-naphthyridine (180 mg) in MeOH (10 mL) was added Pd/C (20 mg) at room temperature under nitrogen atmosphere. The resulting mixture was stirred for 5 hours at room temperature under hydrogen atmosphere. The reaction was monitored by LCMS. The resulting mixture was concentrated under reduced pressure. The residue was purified by Prep-TLC (DCM/MeOH 12:1) to afford 5-[4-fluoro-2-(trifluoromethyl)phenoxy]-1,2,3,4-tetrahydro-2,6-naphthyridine (100 mg) as a brown solid.

### 4-chloro-5-[5-[4-fluoro-2-(trifluoromethyl)phenoxy]-1,2,3,4-tetrahydro-2,6-naphthyridin-2-yl]-2-(oxan-2-yl)-2,3-dihydropyridazin-3-one

To a stirred solution of 5-[4-fluoro-2-(trifluoromethyl)phenoxy]-1,2,3,4-tetrahydro-2,6-naphthyridine (100 mg, 0.320 mmol, 1 equiv.) in DIEA (0.1 mL) was added 4,5-dichloro-2-(oxan-2-yl)-2,3-dihydropyridazin-3-one (63.81 mg, 0.256 mmol, 0.8 equiv.) at room temperature. The resulting mixture was stirred for 1 hours at 90° C. The reaction was monitored by LCMS. The mixture was allowed to cool down to room temperature. The residue was purified by Prep-TLC (DCM/MeOH; 12:1) to afford 4-chloro-5-[5-[4-fluoro-2-(trifluoromethyl)phenoxy]-1,2,3,4-tetrahydro-2,6-naphthyridin-2-yl]-2-(oxan-2-yl)-2,3-dihydropyridazin-3-one (130 mg, 77.34%) as a white solid.

### 4-chloro-5-[5-[4-fluoro-2-(trifluoromethyl)phenoxy]-1,2,3,4-tetrahydro-2,6-naphthyridin-2-yl]-2,3-dihydropyridazin-3-one

To a stirred solution of 4-chloro-5-[5-[4-fluoro-2-(trifluoromethyl)phenoxy]-1,2,3,4-tetrahydro-2,6-naphthyridin-2-yl]-2-(oxan-2-yl)-2,3-dihydropyridazin-3-one (107 mg, 0.204 mmol, 1 equiv.) in DCM (4 mL) was added TFA (1 mL) at room temperature. The resulting mixture was stirred for 1 hours at room temperature. The reaction was monitored by LCMS. The mixture was basified to pH 7 with saturated NaHCO₃(aq.). The resulting mixture was concentrated under reduced pressure. The crude product (50 mg) was purified by Prep-HPLC with the following conditions (Column: XBridge Prep OBD C18 Column 30×150 mm Sum; Mobile Phase A:Water (10 mM NH₄HCO₃), Mobile Phase B: acetonitrile; Flow rate: 60 mL/min; Gradient: 30% B to 50% B in 8 min; 220 nm; Rt: 7.55 min) to afford 4-chloro-5-[5-[4-fluoro-2-(trifluoromethyl)phenoxy]-1,2,3,4-tetrahydro-2,6-naphthyridin-2-yl]-2,3-dihydropyridazin-3-one (60 mg, 66.78%) as a white solid.

### Example 4. Synthesis of Compound 118

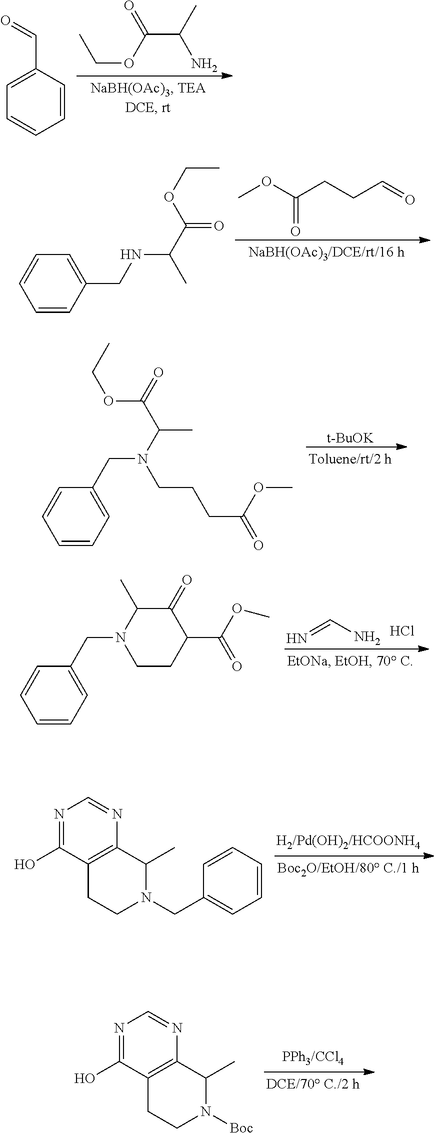

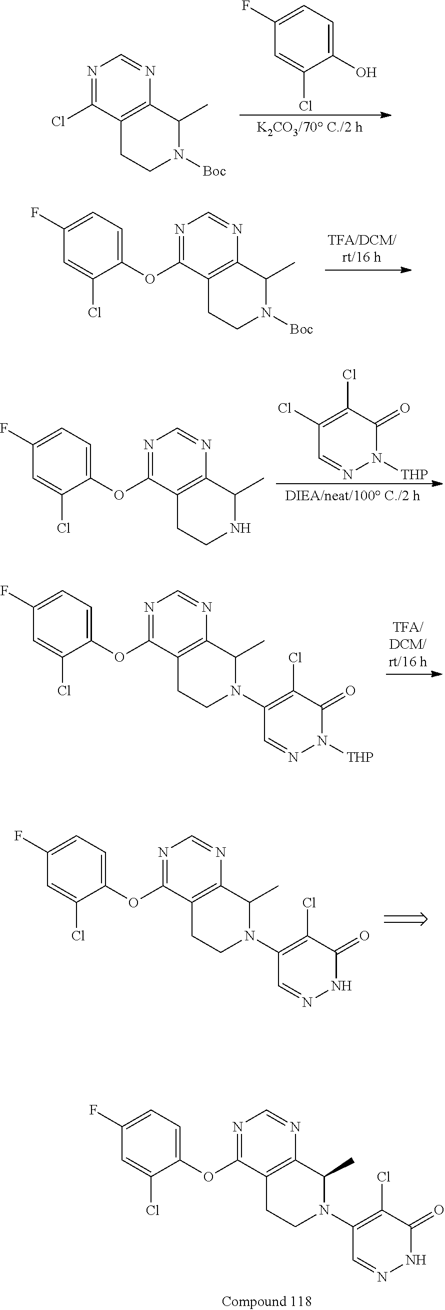

### Ethyl 2-(benzylamino)propanoate

To a stirred solution of benzaldehyde (8 g, 75.384 mmol, 1 equiv.) and TEA (7.63 g, 75.384 mmol, 1 equiv.) in DCE (100 mL, 1263.149 mmol, 16.76 equiv.) was added TEA (7.63 g, 75.384 mmol, 1 equiv.) and NaBH(OAc)₃ (31.95 g, 150.767 mmol, 2 equiv.) in portions at room temperature under nitrogen atmosphere. The mixture was stirred at rt overnight. Desired product could be detected by LCMS. The resulting mixture was extracted with DCM (2×150 mL). The combined organic layers were washed with brine (1×90 mL), dried over anhydrous Na₂SO₄. After filtration, the filtrate was concentrated under reduced pressure to afford ethyl 2-(benzylamino)propanoate (12 g, 76.80%) as colorless oil.

### Methyl 4-[benzyl(1-ethoxy-1-oxopropan-2-yl)amino]butanoate

To a stirred solution of ethyl 2-(benzylamino)propanoate (8 g, 38.596 mmol, 1 equiv.) and methyl 4-oxobutanoate (4.48 g, 38.596 mmol, 1.00 equiv.) in DCE (120 mL, 1515.779 mmol, 39.27 equiv.) was added TEA (3.91 g, 38.596 mmol, 1 equiv.) and NaBH(OAc)₃ (16.36 g, 77.193 mmol, 2 equiv.) in portions at room temperature under nitrogen atmosphere. The mixture was stirred at rt overnight. Desired product could be detected by LCMS. The resulting mixture was extracted with DCM (2×150 mL). The combined organic layers were washed with brine (1×90 mL), dried over anhydrous Na₂SO₄. After filtration, the filtrate was concentrated under reduced pressure to afford methyl 4-[benzyl(1-ethoxy-1-oxopropan-2-yl)amino]butanoate (10 g, 84.29%) as colorless oil.

### Methyl 1-benzyl-2-methyl-3-oxopiperidine-4-carboxylate

To a stirred solution of methyl 4-[benzyl(1-ethoxy-1-oxopropan-2-yl)amino]butanoate (8 g, 26.026 mmol, 1 equiv.) in Toluene (100 mL) was added t-BuOK (5.00 g, 52.051 mmol, 2 equiv.) in portions at room temperature under nitrogen atmosphere. The mixture was stirred at 80° C. for 2 hours. Desired product was detected by LCMS. The resulting mixture was concentrated under vacuum. The residue was purified by silica gel column chromatography, eluted with PE/EtOAc (5:1 to 2:1) to afford methyl 1-benzyl-2-methyl-3-oxopiperidine-4-carboxylate (6.5 g, 95.57%) as a white solid.

### 7-Benzyl-8-methyl-5H,6H,7H,8H-pyrido[3,4-d]pyrimidin-4-ol

To a stirred solution of methyl 1-benzyl-2-methyl-3-oxopiperidine-4-carboxylate (6 g, 22.960 mmol, 1 equiv.) in EtOH (80 mL, 1377.083 mmol, 59.98 equiv.) was added t-BuONa (4.41 g, 45.921 mmol, 2 equiv.) and methanimidamide hydrochloride (3.70 g, 45.921 mmol, 2.00 equiv.) in portions at room temperature under nitrogen atmosphere. The mixture was stirred at 80° C. for 2 h. Desired product could be detected by LCMS. The resulting mixture was concentrated under vacuum. The residue was purified by silica gel column chromatography, eluted with PE/EtOAc (3:1 to 2:1) to afford 7-benzyl-8-methyl-5H,6H,7H,8H-pyrido[3,4-d]pyrimidin-4-ol (5 g, 85.29%) as a white solid.

### Tert-Butyl 4-hydroxy-8-methyl-5H,6H,7H,8H-pyrido[3,4-d]pyrimidine-7-carboxylate

To a solution of 7-benzyl-8-methyl-5H,6H,7H,8H-pyrido[3,4-d]pyrimidin-4-ol (5 g, 19.583 mmol, 1 equiv.) in EtOH (60 mL, 1032.812 mmol, 52.74 equiv.) was added Boc₂O (8.55 g, 39.166 mmol, 2 equiv), CH₃COONa (1.81 g, 23.500 mmol, 1.2 equiv), Pd(OH)₂/C (275.01 mg, 1.958 mmol, 0.1 equiv.) under nitrogen atmosphere. The mixture was hydrogenated at room temperature for 2 h under hydrogen atmosphere, filtered through a Celite pad and concentrated under reduced pressure to afford tert-butyl 4-hydroxy-8-methyl-5H,6H,7H,8H-pyrido[3,4-d]pyrimidine-7-carboxylate (4.5 g, 86.61%) as white solid.

### Tert-butyl 4-chloro-8-methyl-5H,6H,7H,8H-pyrido[3,4-d]pyrimidine-7-carboxylate

To a stirred solution of tert-butyl 4-hydroxy-8-methyl-5H,6H,7H,8H-pyrido[3,4-d]pyrimidine-7-carboxylate (4.5 g, 16.961 mmol, 1 equiv.) and PPh₃ (6.67 g, 25.442 mmol, 1.5 equiv.) in DCE (60 mL, 0.606 mmol, 0.04 equiv.) was added CCl₄ (5.22 g, 33.922 mmol, 2 equiv.) in portions at room temperature under nitrogen atmosphere. The mixture was stirred at 70° C. for 2 hours. Desired product could be detected by LCMS. The resulting mixture was concentrated under vacuum. The residue was purified by silica gel column chromatography, eluted with PE/EtOAc (7:1) to afford tert-butyl 4-chloro-8-methyl-5H,6H,7H,8H-pyrido[3,4-d]pyrimidine-7-carboxylate (4 g, 83.11%) as a white solid.

### Tert-butyl 4-(2-chloro-4-fluorophenoxy)-8-methyl-5H,6H,7H,8H-pyrido[3,4-d]pyrimidine-7-carboxylate

To a stirred solution of tert-butyl 4-chloro-8-methyl-5H,6H,7H,8H-pyrido[3,4-d]pyrimidine-7-carboxylate (4 g, 14.096 mmol, 1 equiv.) and 2-chloro-4-fluorophenol (2.07 g, 14.096 mmol, 1 equiv.) in DMF (50 mL) was added K₂CO₃ (3.90 g, 28.193 mmol, 2 equiv.) in portions at room temperature under nitrogen atmosphere. The mixture was stirred at 70 for 1 h. Desired product could be detected by LCMS. The resulting mixture was concentrated under vacuum. The residue was purified by silica gel column chromatography, eluted with PE/EtOAc (1:1) to afford tert-butyl 4-(2-chloro-4-fluorophenoxy)-8-methyl-5H,6H,7H,8H-pyrido[3,4-d]pyrimidine-7-carboxylate (4 g, 72.05%) as a white solid.

### 4-(2-Chloro-4-fluorophenoxy)-8-methyl-5H,6H,7H,8H-pyrido[3,4-d]pyrimidine

To a stirred solution of tert-butyl 4-(2-chloro-4-fluorophenoxy)-8-methyl-5H,6H,7H,8H-pyrido[3,4-d]pyrimidine-7-carboxylate (4 g, 1 equiv.) in DCM (20 mL) was added TFA (4 mL) dropwise/in portions at room temperature under nitrogen atmosphere. The mixture was stirred at rt for 2 h. Desired product could be detected by LCMS. The resulting mixture was concentrated under reduced pressure to afford 4-(2-chloro-4-fluorophenoxy)-8-methyl-5H,6H,7H,8H-pyrido[3,4-d]pyrimidine (2.7 g, 90.51%) as off-white solid.

### 4-chloro-5-[4-(2-chloro-4-fluorophenoxy)-8-methyl-5H,6H,7H,8H-pyrido[3,4-d]pyrimidin-7-yl]-2-(oxan-2-yl)-2,3-dihydropyridazin-3-one

To a stirred solution of 4-(2-chloro-4-fluorophenoxy)-8-methyl-5H,6H,7H,8H-pyrido[3,4-d]pyrimidine (1 g, 3.404 mmol, 1 equiv.) in DIEA (1 mL) was added 4,5-dichloro-2-(oxan-2-yl)-2,3-dihydropyridazin-3-one (0.85 g, 3.404 mmol, 1 equiv.) in portions at room temperature under nitrogen atmosphere. The mixture was stirred at 100° C. overnight. The desired product could be detected by LCMS. The residue was purified by silica gel column chromatography, eluted with PE/EtOAc (1:1 to 1:2) to afford 4-chloro-5-[4-(2-chloro-4-fluorophenoxy)-8-methyl-5H,6H,7H,8H-pyrido[3,4-d]pyrimidin-7-yl]-2-(oxan-2-yl)-2,3-dihydropyridazin-3-one (1 g, 58.01%) as a white solid.

### 4-chloro-5-[4-(2-chloro-4-fluorophenoxy)-8-methyl-5H,6H,7H,8H-pyrido[3,4-d]pyrimidin-7-yl]-2,3-dihydropyridazin-3-one

To a stirred solution of 4-chloro-5-[4-(2-chloro-4-fluorophenoxy)-8-methyl-5H,6H,7H,8H-pyrido[3,4-d]pyrimidin-7-yl]-2-(oxan-2-yl)-2,3-dihydropyridazin-3-one (1 g, 1 equiv.) in DCM (10 mL) was added TFA (2 mL) dropwise at room temperature under nitrogen atmosphere. The mixture was stirred at rt for 1 h. Desired product could be detected by LCMS. The resulting mixture was concentrated under reduced pressure to afford 4-chloro-5-[4-(2-chloro-4-fluorophenoxy)-8-methyl-5H,6H,7H,8H-pyrido[3,4-d]pyrimidin-7-yl]-2,3-dihydropyridazin-3-one (600 mg, 71.95%) as white solid.

### 4-chloro-5-[(8R)-4-(2-chloro-4-fluorophenoxy)-8-methyl-5H,6H,7H,8H-pyrido[3,4-d]pyrimidin-7-yl]-2,3-dihydropyridazin-3-one

4-chloro-5-[4-(2-chloro-4-fluorophenoxy)-8-methyl-5H,6H,7H,8H-pyrido[3,4-d]pyrimidin-7-yl]-2,3-dihydropyridazin-3-one (250 mg, 1 equiv.) was separated by prep chiral—HPLC (Column: CHIRALPAK IG, 20*250 mm, 5 um; Mobile Phase A:Hex:DCM=3:1 (0.1% FA)—HPLC, Mobile Phase B: EtOH—HPLC; Flow rate: 20 mL/min; Gradient: 15 B to 15 B in 19 min; 220/254 nm; RT1:13.016; RT2:16.004) to afford 4-chloro-5-[(8R)-4-(2-chloro-4-fluorophenoxy)-8-methyl-5H,6H,7H,8H-pyrido[3,4-d]pyrimidin-7-yl]-2,3-dihydropyridazin-3-one (144 mg, 57.60%) as white solid.

### Example 5. Synthesis of Compound 103

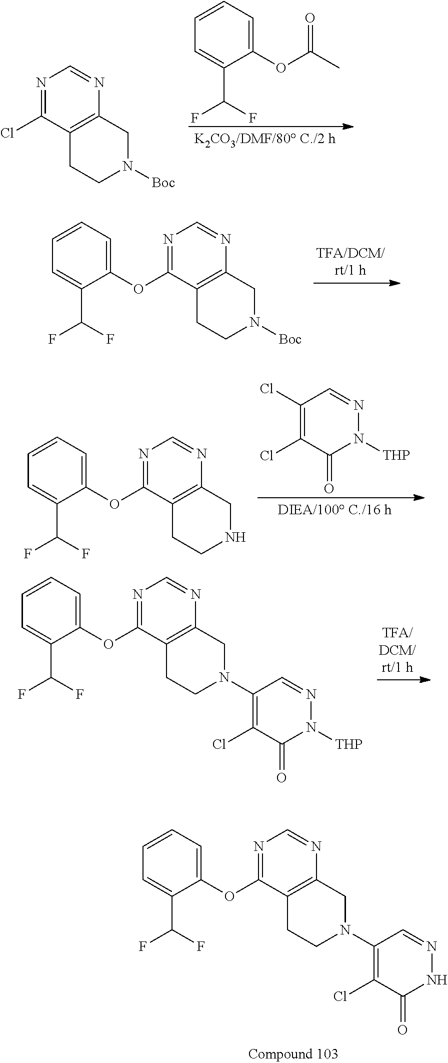

### Tert-Butyl 4-[2-(difluoromethyl)phenoxy]-5H,6H,7H,8H-pyrido[3,4-d]pyrimidine-7-carboxylate

To a stirred solution of tert-butyl 4-chloro-5H,6H,7H,8H-pyrido[3,4-d]pyrimidine-7-carboxylate (800 mg, 2.966 mmol, 1 equiv.) and 2-(difluoromethyl)phenyl acetate (1104.26 mg, 5.932 mmol, 2.00 equiv.) in DMF (20 mL) were added K₂CO₃ (1229.72 mg, 8.898 mmol, 3 equiv.) in portions at 80° C. under nitrogen atmosphere. The mixture was stirred for 2 hours. The reaction was monitored by LCMS. The reaction was quenched with Water at room temperature. The mixture was extracted with EtOAc (3×50 mL). The combined organic layers were washed with brine (3×100 mL), dried over anhydrous Na₂SO₄. After filtration, the filtrate was concentrated under reduced pressure. The residue was purified by reverse flash chromatography with the following conditions: column, C18 silica gel; mobile phase, MeOH in water, 10% to 50% gradient in 10 min; detector, UV 254 nm. to afford tert-butyl 4-[2-(difluoromethyl)phenoxy]-5H,6H,7H,8H-pyrido[3,4-d]pyrimidine-7-carboxylate (900 mg, 80.41%) as off-white solid.

### 4-[2-(Difluoromethyl)phenoxy]-5H,6H,7H,8H-pyrido[3,4-d]pyrimidine

To a stirred solution of tert-butyl 4-[2-(difluoromethyl)phenoxy]-5H,6H,7H,8H-pyrido[3,4-d]pyrimidine-7-carboxylate (900 mg, 2.385 mmol, 1 equiv.) in DCM was added 3,3,3-trifluoropropanoic acid (3 mL, 6.00 equiv.) dropwise at room temperature. The mixture was stirred for 1.5 hours. The reaction was monitored by TLC (PE/EtOAc 10:1). The residue was basified to pH 8 with saturated NaHCO₃(aq.). The mixture was concentrated under reduced pressure. The crude product (100 mg) was purified by Prep-HPLC with the following conditions to afford 4-[2-(difluoromethyl)phenoxy]-5H,6H,7H,8H-pyrido[3,4-d]pyrimidine (329 mg, 49.75%) as off-white solid.

### 4-Chloro-5-[4-[2-(difluoromethyl)phenoxy]-5H,6H,7H,8H-pyrido[3,4-d]pyrimidin-7-yl]-2-(oxan-2-yl)-2,3-dihydropyridazin-3-one

To a stirred solution of 4-[2-(difluoromethyl)phenoxy]-5H,6H,7H,8H-pyrido[3,4-d]pyrimidine (328 mg, 1.183 mmol, 1 equiv.) and 4,5-dichloro-2-(oxan-2-yl)-2,3-dihydropyridazin-3-one (169.05 mg, 0.679 mmol, 1.00 equiv.) were added DIEA (175.43 mg, 1.357 mmol, 2.00 equiv.) in portions at 70° C. The mixture was stirred for 2 hours at 70° C. The residue was purified by reverse flash chromatography with the following conditions: column, C18 silica gel; mobile phase, MeOH in water, 10% to 50% gradient in 10 min; detector, UV 254 nm. to afford 4-chloro-5-[4-[2-(difluoromethyl)phenoxy]-5H,6H,7H,8H-pyrido[3,4-d]pyrimidin-7-yl]-2-(oxan-2-yl)-2,3-dihydropyridazin-3-one (328 mg, 56.60%) as off-white solid.

### 4-Chloro-5-[4-[2-(difluoromethyl)phenoxy]-5H,6H,7H,8H-pyrido[3,4-d]pyrimidin-7-yl]-2,3-dihydropyridazin-3-one

To a stirred solution of 4-chloro-5-[4-[2-(difluoromethyl)phenoxy]-5H,6H,7H,8H-pyrido[3,4-d]pyrimidin-7-yl]-2-(oxan-2-yl)-2,3-dihydropyridazin-3-one (328 mg, 0.670 mmol, 1 equiv.) in DCM (10 mL) was added trifluoroacetic acid (3 mL) dropwise at room temperature. The mixture was concentrated under vacuum. The product was purified by Prep-HPLC to afford 4-chloro-5-[4-[2-(difluoromethyl)phenoxy]-5H,6H,7H,8H-pyrido[3,4-d]pyrimidin-7-yl]-2,3-dihydropyridazin-3-one (256.4 mg, 94.38%) as off-white solid.

### Example 6. Synthesis of Compound 117 and 117a

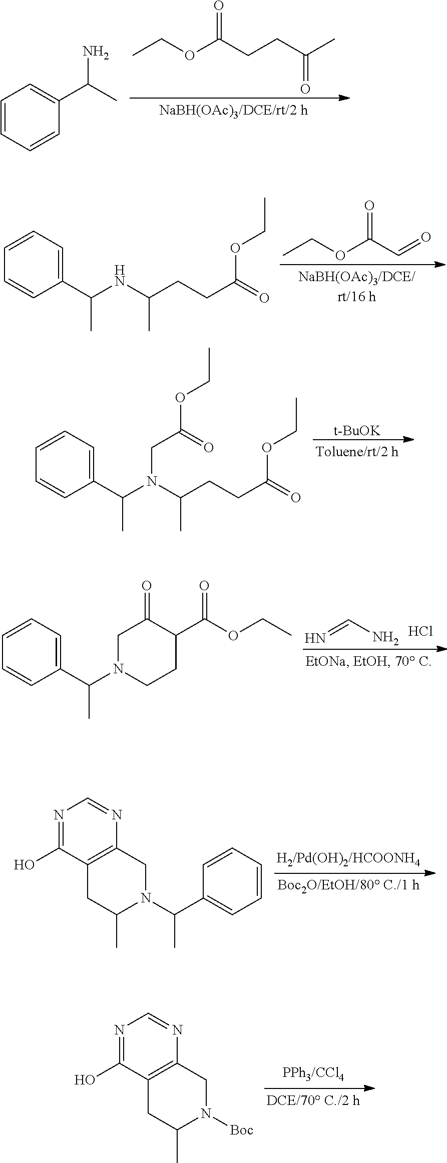

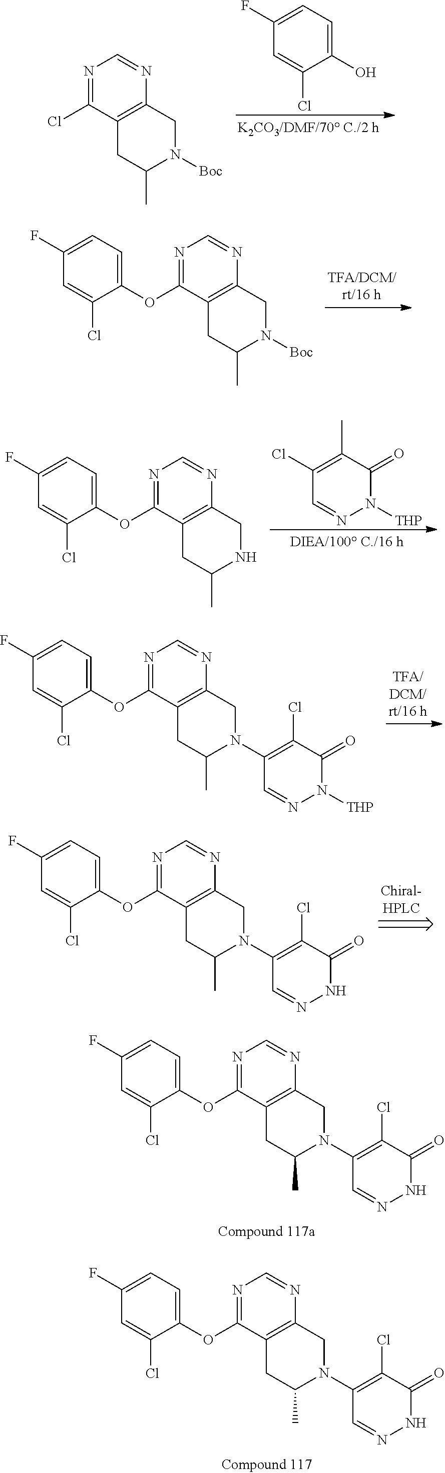

### Ethyl 4-[(1-phenylethyl)amino]pentanoate

To a stirred solution of 1-phenylethan-1-amine (25 g, 206.300 mmol, 1 equiv.) and ethyl 4-oxopentanoate (29.74 g, 206.300 mmol, 1 equiv.) in DCE (400 mL, 5052.598 mmol, 24.49 equiv.) was added NaBH(OAc)₃ (65.59 g, 309.449 mmol, 1.5 equiv.) in portions at 25° C. under nitrogen atmosphere. The solution was stirred at 25° C. for 2 hours. The reaction was quenched by the addition of H₂O (400 mL) at 0° C. The resulting mixture was extracted with DCM (3×200 mL). The combined organic layers were washed with saturated NaCl (aq.) (3×200 mL), dried over anhydrous Na₂SO₄. After filtration, the filtrate was concentrated under reduced pressure. The crude product was used to the next step.

### Ethyl 4-[(2-ethoxy-2-oxoethyl)(1-phenylethyl)amino]pentanoate

To a stirred solution of ethyl 4-[(1-phenylethyl)amino]pentanoate (49 g, 196.508 mmol, 1 equiv.) and ethyl 2-oxoacetate (40.12 g, 392.990 mmol, 2.00 equiv.) in DCE (500 mL, 6315.747 mmol, 32.14 equiv.) was added NaBH(OAc)₃ (62.47 g, 294.762 mmol, 1.5 equiv.) in portions at 25° C. under nitrogen atmosphere. The solution was stirred at 25° C. for 2 hours. The reaction was quenched by the addition of H₂O (400 mL) at 0° C. The resulting mixture was extracted with DCM (3×200 mL). The combined organic layers were washed with saturated NaCl (aq.) (3×200 mL), dried over anhydrous Na₂SO₄. After filtration, the filtrate was concentrated under reduced pressure. The crude product ethyl 4-[(2-ethoxy-2-oxoethyl)(1-phenylethyl)amino]pentanoate (57 g, 86.47%) was used to the next step.

### Ethyl 2-methyl-5-oxo-1-(1-phenylethyl)piperidine-4-carboxylate

To a solution of ethyl 4-[(2-ethoxy-2-oxoethyl)(1-phenylethyl)amino]pentanoate (57 g, 169.924 mmol, 1 equiv.) in Toluene (500 mL, 4699.452 mmol, 27.66 equiv.) was added t-BuOK (47.67 g, 424.810 mmol, 2.5 equiv.) in ports at 0° C. The mixture was stirred at 25° C. for 2 hours. The resulting mixture was concentrated under reduced pressure. The residue was purified by silica gel column chromatography, eluted with PE/EA (50/1 to 10/1) to afford ethyl 2-methyl-5-oxo-1-(1-phenylethyl)piperidine-4-carboxylate (29 g, 58.98%) as a yellow oil

### 7-(1-cyclohexylethyl)-6-methyl-decahydropyrido[3,4-d]pyrimidin-4-ol

To a solution of ethyl 2-methyl-5-oxo-1-(1-phenylethyl)piperidine-4-carboxylate (10 g, 34.557 mmol, 1 equiv.) and methanimidamide hydrochloride (4.17 g, 51.836 mmol, 1.50 equiv.) in EtOH (100 mL, 1721.353 mmol, 49.81 equiv.) was added EtONa (5.88 g, 86.393 mmol, 2.50 equiv.) in ports at 25° C. The mixture was stirred at 90° C. for 2 hours. The residue was purified by silica gel column chromatography, eluted with DCM/MeOH (20/1 to 10/1) to afford 7-(1-cyclohexylethyl)-6-methyl-decahydropyrido[3,4-d]pyrimidin-4-ol (3.4 g, 34.96%) as a yellow solid.

### Tert-Butyl 4-hydroxy-6-methyl-5H,6H,7H,8H-pyrido[3,4-d]pyrimidine-7-carboxylate

To a solution of 6-methyl-7-(1-phenylethyl)-5H,6H,7H,8H-pyrido[3,4-d]pyrimidin-4-ol (3.5 g, 12.994 mmol, 1 equiv), HCOONH₄ (4.10 g, 65.022 mmol, 5.00 equiv.) and Boc₂O (8.51 g, 38.983 mmol, 3 equiv.) in EtOH (50 mL, 860.677 mmol, 66.23 equiv.) was added Pd(OH)₂/C (0.36 g, 2.599 mmol, 0.2 equiv.) under nitrogen atmosphere. The mixture was hydrogenated at 70° C. for 2 hours under hydrogen atmosphere using a hydrogen balloon, filtered through a Celite pad and concentrated under reduced pressure. To afford tert-butyl 4-hydroxy-6-methyl-5H,6H,7H,8H-pyrido[3,4-d]pyrimidine-7-carboxylate (1.8 g, 52.21%) as a yellow solid.

### Tert-Butyl 4-chloro-6-methyl-5H,6H,7H,8H-pyrido[3,4-d]pyrimidine-7-carboxylate

To a solution of tert-butyl 4-hydroxy-6-methyl-5H,6H,7H,8H-pyrido[3,4-d]pyrimidine-7-carboxylate (1.8 g, 6.784 mmol, 1 equiv.) and PPh₃ (3.56 g, 13.569 mmol, 2 equiv.) in DCE (20 mL, 252.630 mmol, 37.24 equiv.) was added CCl₄ (3.13 g, 20.353 mmol, 3 equiv.) at 25° C. The mixture was stirred at 70° C. for 3 hours. The resulting mixture was concentrated under reduced pressure. The residue was purified by silica gel column chromatography, eluted with PE/EA (10/1 to 1/1) to afford tert-butyl 4-chloro-6-methyl-5H,6H,7H,8H-pyrido[3,4-d]pyrimidine-7-carboxylate (1.1 g, 57.14%) as a yellow solid.

### Tert-Butyl 4-(2-chloro-4-fluorophenoxy)-6-methyl-5H,6H,7H,8H-pyrido[3,4-d]pyrimidine-7-carboxylate

To a solution of tert-butyl 4-chloro-6-methyl-5H,6H,7H,8H-pyrido[3,4-d]pyrimidine-7-carboxylate (1.1 g, 3.877 mmol, 1 equiv.) and 2-chloro-4-fluorophenol (0.85 g, 5.800 mmol, 1.50 equiv.) in DMF (15 mL, 193.826 mmol, 50.00 equiv.) was added K₂CO₃ (1.07 g, 7.753 mmol, 2 equiv.) at 25° C. The mixture was stirred at 70° C. for 1 hour. The resulting mixture was concentrated under reduced pressure. The residue was purified by silica gel column chromatography, eluted with PE/EA (10/1 to 5/1) to afford tert-butyl 4-(2-chloro-4-fluorophenoxy)-6-methyl-5H,6H,7H,8H-pyrido[3,4-d]pyrimidine-7-carboxylate (1.2 g, 78.60%) as a yellow solid.

### 4-Chloro-5-[4-(2-chloro-4-fluorophenoxy)-6-methyl-5H,6H,7H,8H-pyrido[3,4-d]pyrimidin-7-yl]-2-(oxan-2-yl)-2,3-dihydropyridazin-3-one

A mixture of 4-(2-chloro-4-fluorophenoxy)-6-methyl-5H,6H,7H,8H-pyrido[3,4-d]pyrimidine (800 mg, 2.724 mmol, 1 equiv.) and 4,5-dichloro-2-(oxan-2-yl)-2,3-dihydropyridazin-3-one (678.42 mg, 2.724 mmol, 1.00 equiv.) in DIEA (704.01 mg, 5.447 mmol, 2 equiv.) was stirred for 16 hours at 100° C. under nitrogen atmosphere. The residue was purified by Prep-TLC (PE/EA 1/1) to afford 4-chloro-5-[4-(2-chloro-4-fluorophenoxy)-6-methyl-5H,6H,7H,8H-pyrido[3,4-d]pyrimidin-7-yl]-2-(oxan-2-yl)-2,3-dihydropyridazin-3-one (530 mg, 38.43%) as a light yellow solid.

### 4-chloro-5-[(6R)-4-(2-chloro-4-fluorophenoxy)-6-methyl-5H,6H,7H,8H-pyrido[3,4-d]pyrimidin-7-yl]-2,3-dihydropyridazin-3-one

To a solution of 4-chloro-5-[4-(2-chloro-4-fluorophenoxy)-6-methyl-5H,6H,7H,8H-pyrido[3,4-d]pyrimidin-7-yl]-2-(oxan-2-yl)-2,3-dihydropyridazin-3-one (530 mg, 1.047 mmol, 1 equiv.) in DCM (20 mL, 314.601 mmol, 300.57 equiv.) was added TFA (1193.47 mg, 10.467 mmol, 10 equiv.) at 25° C. The solution was stirred at 25° C. for 2 hours. The resulting mixture was concentrated under reduced pressure. The crude product (600 mg) was purified by Prep-HPLC with the following conditions (Column: XBridge Shield RP18 OBD Column 30*150 mm, 5 um; Mobile Phase A: Water (10 mM NH4HCO3), Mobile Phase B: acetonitrile; Flow rate: 60 mL/min; Gradient: 20% B to 40% B in 7 min; 220 nm; Rt: 6.63 min) to afford the racemate (200 mg). The residue (200 mg) was purified by Chiral-Prep-HPLC with the following conditions: Column: CHIRALPAK IE, 2*25 cm, 5 um; Mobile Phase A: MTBE (0.1% FA)-HPLC, Mobile Phase B: IPA—HPLC; Flow rate: 18 mL/min; Gradient: 20 B to 20 B in 15 min; 220/254 nm. Although the two isomers were separated by this technique, the absolute orientation was not determined. The compound designated as 4-chloro-5-[(6S)-4-(2-chloro-4-fluorophenoxy)-6-methyl-5H,6H,7H,8H-pyrido[3,4-d]pyrimidin-7-yl]-2,3-dihydropyridazin-3-one (60.9 mg, 13.78%) was obtained at 9.688 min as a white solid. The compound designated as 4-chloro-5-[(6R)-4-(2-chloro-4-fluorophenoxy)-6-methyl-5H,6H,7H,8H-pyrido[3,4-d]pyrimidin-7-yl]-2,3-dihydropyridazin-3-one (61.5 mg, 13.92%) was obtained at 11.813 min as a white solid.

### Example 7. Synthesis of Compound 134

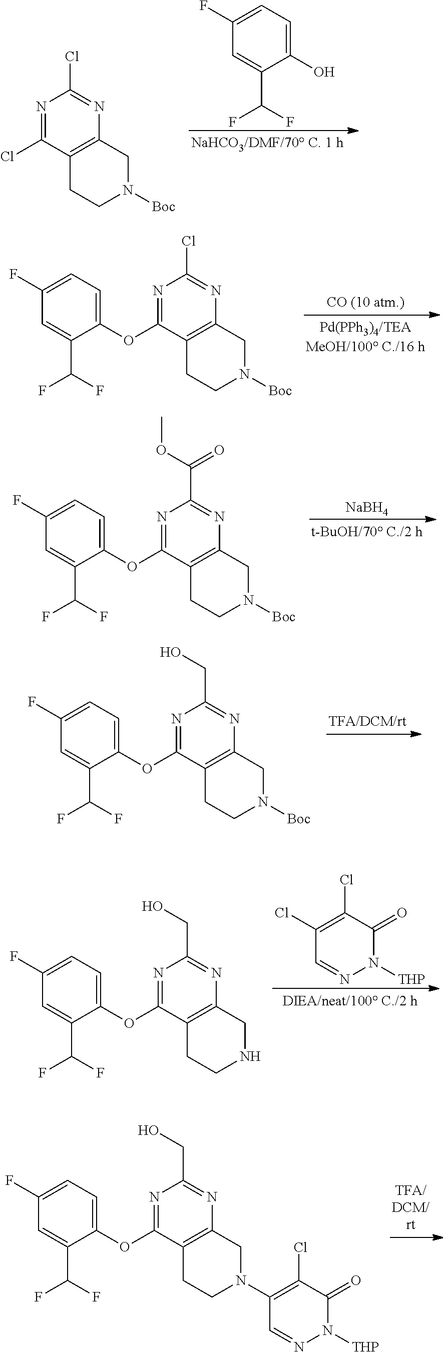

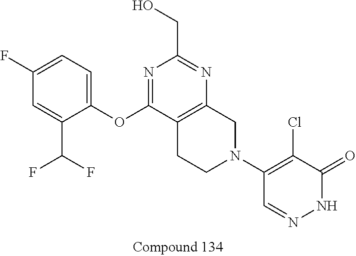 [SMILES: O=c1[nH]ncc(N2CCc3c(nc(CO)nc3Oc3ccc(F)cc3C(F)F)C2)c1Cl]

### Tert-butyl 2-chloro-4-[2-(difluoromethyl)-4-fluorophenoxy]-5H,6H,7H,8H-pyrido[3,4-d]pyrimidine-7-carboxylate

To a stirred solution of 2-(difluoromethyl)-4-fluorophenol (5.33 g, 32.879 mmol, 2.00 equiv.) and tert-butyl 2,4-dichloro-5H,6H,7H,8H-pyrido[3,4-d]pyrimidine-7-carboxylate (5 g, 16.438 mmol, 1 equiv.) in DMF (30 mL) was added NaHCO₃ (4.14 g, 49.282 mmol, 3.00 equiv.) at room temperature. The solution was stirred at 70° C. for 0.5 hours. The mixture was concentrated under reduced pressure. The residue was purified by reverse phase flash chromatography with the following conditions: Column: Spherical C18, 20-40 um, 330 g; Mobile Phase A: Water (plus 10 mM NH4HCO3); Mobile Phase B: acetonitrile; Flow rate: 80 mL/min; Gradient: 5%-5% B, 10 min, 70% B-95% B gradient in 100 min; Detector: 254 nm. The fractions containing the desired product were collected at 92% B and concentrated under reduced pressure to afford tert-butyl 2-chloro-4-[2-(difluoromethyl)-4-fluorophenoxy]-5H,6H,7H,8H-pyrido[3,4-d]pyrimidine-7-carboxylate (2.100 g) as off-white solid.

### 7-tert-Butyl 2-methyl 4-[2-(difluoromethyl)-4-fluorophenoxy]-5H,6H,7H,8H-pyrido[3,4-d]pyrimidine-2,7-dicarboxylate

To a solution of tert-butyl 2-chloro-4-[2-(difluoromethyl)-4-fluorophenoxy]-5H,6H,7H,8H-pyrido[3,4-d]pyrimidine-7-carboxylate (400 mg, 0.931 mmol, 1 equiv.) and TEA (188.34 mg, 1.861 mmol, 2 equiv.) in MeOH (15 mL, 370.484 mmol, 398.10 equiv.) was added Pd(PPh3)4 (107.54 mg, 0.093 mmol, 0.1 equiv.) in a pressure tank. The mixture was purged with nitrogen for 1 hours and then was pressurized to 10 atm with carbon monoxide at 100° C. for 16 hours. The reaction mixture was cooled to room temperature and filtered to remove insoluble solids. The residue was purified by reverse phase flash chromatography with the following conditions: Column: Spherical Cis, 20-40 um, 330 g; Mobile Phase A: Water (plus 10 mM NH₄HCO₃); Mobile Phase B: acetonitrile; Flow rate: 80 mL/min; Gradient: 5%-5% B, 10 min, 35% B-65% B gradient in 20 min; Detector: 254 nm. The fractions containing the desired product were collected at 62% B and concentrated under reduced pressure to afford 7-tert-butyl 2-methyl 4-[2-(difluoromethyl)-4-fluorophenoxy]-5H,6H,7H,8H-pyrido[3,4-d]pyrimidine-2,7-dicarboxylate (100 mg, 23.70%) as colorless oil.

### Tert-butyl 4-[2-(difluoromethyl)-4-fluorophenoxy]-2-(hydroxymethyl)-5H,6H,7H,8H-pyrido[3,4-d]pyrimidine-7-carboxylate

To a stirred solution of 7-tert-butyl 2-methyl 4-[2-(difluoromethyl)-4-fluorophenoxy]-5H,6H,7H,8H-pyrido[3,4-d]pyrimidine-2,7-dicarboxylate (100 mg, 0.221 mmol, 1 equiv.) in t-BuOH (6 mL, 63.139 mmol, 286.29 equiv.) was added NaBH₄ (16.69 mg, 0.441 mmol, 2 equiv.) at room temperature. The solution was stirred at 70° C. for 3 hours. To the mixture was added water (3 mL). The residue was purified by reverse phase flash chromatography with the following conditions: Column: Spherical C18, 20-40 um, 330 g; Mobile Phase A: Water (plus 10 mM NH₄HCO₃); Mobile Phase B: acetonitrile; Flow rate: 80 mL/min; Gradient: 5%-5% B, 10 min, 45% B-80% B gradient in 20 min; Detector: 254 nm. The fractions containing the desired product were collected at 74% B and concentrated under reduced pressure to afford tert-butyl 4-[2-(difluoromethyl)-4-fluorophenoxy]-2-(hydroxymethyl)-5H,6H,7H,8H-pyrido[3,4-d]pyrimidine-7-carboxylate (35 mg, 37.30%) as colorless oil.

### [4-[2-(difluoromethyl)-4-fluorophenoxy]-5H,6H,7H,8H-pyrido[3,4-d]pyrimidin-2-yl]methanol

To a stirred solution of tert-butyl 4-[2-(difluoromethyl)-4-fluorophenoxy]-2-(hydroxymethyl)-5H,6H,7H,8H-pyrido[3,4-d]pyrimidine-7-carboxylate (35 mg) in DCM (6 mg) was added TFA (1 mg) at room temperature. The solution was stirred at rt for 2 hours. The mixture was concentrated under reduced pressure. The residue was purified by reverse phase flash chromatography with the following conditions: Column: Spherical C18, 20-40 um, 330 g; Mobile Phase A: Water (plus 10 mM NH₄HCO₃); Mobile Phase B: acetonitrile; Flow rate: 80 mL/min; Gradient: 5%-5% B, 10 min, 25% B-55% B gradient in 20 min; Detector: 254 nm. The fractions containing the desired product were collected at 41% B and concentrated under reduced pressure to afford as [4-[2-(difluoromethyl)-4-fluorophenoxy]-5H,6H,7H,8H-pyrido[3,4-d]pyrimidin-2-yl]methanol (20 mg) as colorless oil.

### 4-chloro-5-[4-[2-(difluoromethyl)-4-fluorophenoxy]-2-(hydroxymethyl)-5H,6H,7H,8H-pyrido[3,4-d]pyrimidin-7-yl]-2-(oxan-2-yl)-2,3-dihydropyridazin-3-one

Into a 25 mL round-bottom flask were added [4-[2-(difluoromethyl)-4-fluorophenoxy]-5H,6H,7H,8H-pyrido[3,4-d]pyrimidin-2-yl]methanol (20 mg, 0.061 mmol, 1 equiv.) and 4,5-dichloro-2-(oxan-2-yl)-2,3-dihydropyridazin-3-one (15.31 mg, 0.061 mmol, 1 equiv.) at room temperature. To the mixture was added DIEA (15.89 mg, 0.123 mmol, 2 equiv.) at rt. The mixture was stirred at 90° C. for 2 hours. The residue was purified by reverse phase flash chromatography with the following conditions: Column: Spherical C18, 20-40 um, 330 g; Mobile Phase A: Water (plus 10 mM NH₄HCO₃); Mobile Phase B: acetonitrile; Flow rate: 80 mL/min; Gradient: 5%-5% B, 10 min, 35% B-70% B gradient in 20 min; Detector: 254 nm. The fractions containing the desired product were collected at 65% B and concentrated under reduced pressure to afford 4-chloro-5-[4-[2-(difluoromethyl)-4-fluorophenoxy]-2-(hydroxymethyl)-5H,6H,7H,8H-pyrido[3,4-d]pyrimidin-7-yl]-2-(oxan-2-yl)-2,3-dihydropyridazin-3-one (30 mg, 90.71%) as colorless oil.

### 4-chloro-5-[4-[2-(difluoromethyl)-4-fluorophenoxy]-2-(hydroxymethyl)-5H,6H,7H,8H-pyrido[3,4-d]pyrimidin-7-yl]-2,3-dihydropyridazin-3-one

To a stirred solution of 4-chloro-5-[4-[2-(difluoromethyl)-4-fluorophenoxy]-2-(hydroxymethyl)-5H,6H,7H,8H-pyrido[3,4-d]pyrimidin-7-yl]-2-(oxan-2-yl)-2,3-dihydropyridazin-3-one (30 mg) in DCM (5 mL) was added TFA (1 mL) at room temperature. The solution was stirred at rt for 2 hours. The mixture was concentrated under reduced pressure. The crude product (30 mg) was purified by Prep-HPLC with the following conditions (Column: XBridge Prep OBD C18 Column 30×150 mm Sum; Mobile Phase A: undefined, Mobile Phase B: undefined; Flow rate: 60 mL/min; Gradient: 20% B to 40% B in 8 min; 220 nm; Rt: 7.22 min) to afford 4-chloro-5-[4-[2-(difluoromethyl)-4-fluorophenoxy]-2-(hydroxymethyl)-5H,6H,7H,8H-pyrido[3,4-d]pyrimidin-7-yl]-2,3-dihydropyridazin-3-one (8.7 mg) as a white solid.

Compounds 128, 125, 114 were prepared by the methods and scheme described in this Example by using 2-trifluoromethylphenol, 4-fluoro-2-trifluoromethylphenol and 4-fluoro-2-chlorophenol respectively, in place of 2-(difluoromethyl)-4-fluorophenol in the first step of the synthesis.

### Example 8. Synthesis of Compound 112

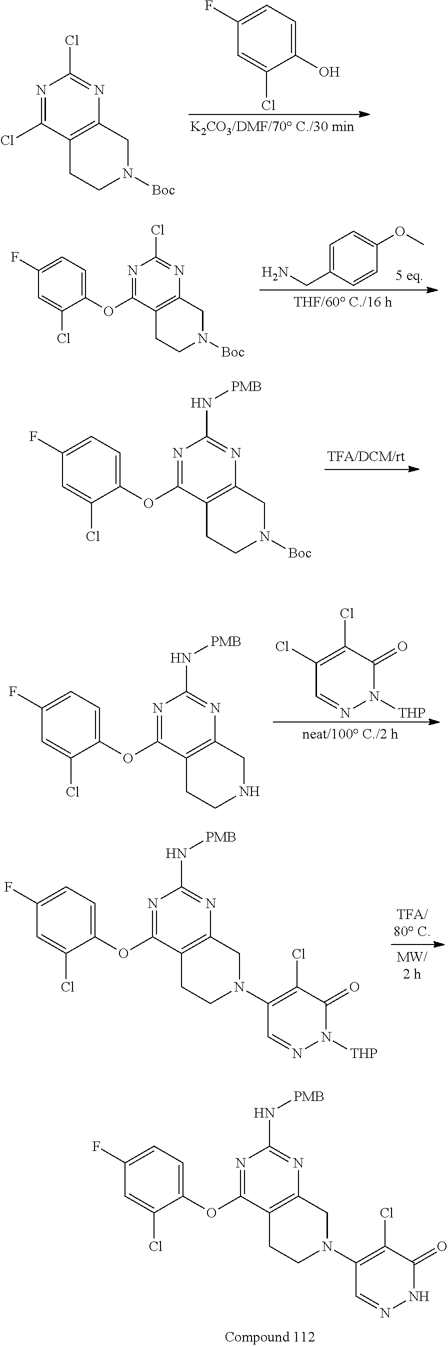

### Tert-butyl 2-chloro-4-(2-chloro-4-fluorophenoxy)-5H,6H,7H,8H-pyrido[3,4-d]pyrimidine-7-carboxylate

To a stirred mixture of tert-butyl 2,4-dichloro-5H,6H,7H,8H-pyrido[3,4-d]pyrimidine-7-carboxylate (800 mg, 2.630 mmol, 1 equiv.) and 2-chloro-4-fluorophenol (578.16 mg, 3.945 mmol, 1.50 equiv.) in DMF (15 mL) was added K₂CO₃ (726.99 mg, 5.260 mmol, 2.00 equiv.) in portions at rt under nitrogen atmosphere. The resulting mixture was stirred for 0.5 hours at 70° C. under nitrogen atmosphere. The reaction was monitored by LCMS. The mixture was allowed to cool down to rt. The resulting mixture was concentrated under reduced pressure. The resulting mixture was extracted with EtOAc (3×200 mL). The combined organic layers were washed with brine (2×100 mL), dried over anhydrous Na₂SO₄. After filtration, the filtrate was concentrated under reduced pressure. The residue was purified by silica gel column chromatography, eluted with PE/EtOAc (30/1 to 10/1) to afford tert-butyl 2-chloro-4-(2-chloro-4-fluorophenoxy)-5H,6H,7H,8H-pyrido[3,4-d]pyrimidine-7-carboxylate (1 g, 91.78%) as a yellow oil.

### Tert-Butyl 4-(2-chloro-4-fluorophenoxy)-2-[[(4-methoxyphenyl)methyl]amino]-5H,6H,7H,8H-pyrido[3,4-d]pyrimidine-7-carboxylate

To a stirred mixture of tert-butyl 2-chloro-4-(2-chloro-4-fluorophenoxy)-5H,6H,7H,8H-pyrido[3,4-d]pyrimidine-7-carboxylate (700 mg, 1.690 mmol, 1 equiv.) in THF (30 mL) was added 1-(4-methoxyphenyl)methanamine (1159.02 mg, 8.449 mmol, 5.00 equiv.) in portions at rt under nitrogen atmosphere. The resulting mixture was stirred for 16 hours at 60° C. under nitrogen atmosphere. The reaction was monitored by LCMS. The mixture was allowed to cool down to rt. The resulting mixture was concentrated under reduced pressure. The resulting mixture was extracted with EtOAc (3×200 mL). The combined organic layers were washed with brine (2×100 mL), dried over anhydrous Na₂SO₄. After filtration, the filtrate was concentrated under reduced pressure. The residue was purified by reverse flash chromatography with the following conditions (Column, C18 silica gel; mobile phase, acetonitrile in water, 60% to 95% gradient in 20 min; detector, UV 220 nm) to afford tert-butyl 4-(2-chloro-4-fluorophenoxy)-2-[[(4-methoxyphenyl)methyl]amino]-5H,6H,7H,8H-pyrido[3,4-d]pyrimidine-7-carboxylate (350 mg, 40.22%) as a yellow oil.

### 4-(2-chloro-4-fluorophenoxy)-N-[(4-methoxyphenyl)methyl]-5H,6H,7H,8H-pyrido[3,4-d]pyrimidin-2-amine

To a stirred solution of tert-butyl 4-(2-chloro-4-fluorophenoxy)-2-[[(4-methoxyphenyl)methyl]amino]-5H,6H,7H,8H-pyrido[3,4-d]pyrimidine-7-carboxylate (350 mg, 1 equiv.) in DCM (10 mL) was added TFA (1 mL) dropwise at rt. The reaction mixture was stirred for 2 hours at rt. The reaction was monitored by LCMS. The resulting mixture was concentrated under reduced pressure. The residue was basified to pH=8 with saturated NH₄HCO₃ (aq.). The resulting mixture was extracted with DCM (3×100 mL). The combined organic layers were washed with brine (1×100 mL), dried over anhydrous Na₂SO₄. After filtration, the filtrate was concentrated under reduced pressure. The residue was purified by Prep-HPLC with the following conditions (Column: XBridge Shield RP18 OBD Column, 5 um, 19*150 mm; Mobile Phase A: Water (10 mM NH₄HCO₃), Mobile Phase B: acetonitrile; Flow rate: 25 mL/min; Gradient: 2% B to 32% B in 1 min; 220/254 nm; Rt: 7.08 min) to afford 4-(2-chloro-4-fluorophenoxy)-N-[(4-methoxyphenyl)methyl]-5H,6H,7H,8H-pyrido[3,4-d]pyrimidin-2-amine (260 mg) as a yellow oil.

### 4-chloro-5-[4-(2-chloro-4-fluorophenoxy)-2-[[(4-methoxyphenyl)methyl]amino]-5H,6H,7H,8H-pyrido[3,4-d]pyrimidin-7-yl]-2-(oxan-2-yl)-2,3-dihydropyridazin-3-one

Into a 50 mL round-bottom flask were added 4-(2-chloro-4-fluorophenoxy)-N-[(4-methoxyphenyl)methyl]-5H,6H,7H,8H-pyrido[3,4-d]pyrimidin-2-amine (260 mg, 0.627 mmol, 1 equiv), 4,5-dichloro-2-(oxan-2-yl)-2,3-dihydropyridazin-3-one (156.11 mg, 0.627 mmol, 1.00 equiv.) and DIEA (242.99 mg, 1.880 mmol, 3.00 equiv.) at rt under nitrogen atmosphere. The resulting mixture was stirred for 2 hours at 90° C. under nitrogen atmosphere. The residue was purified by reverse flash chromatography with the following conditions (Column, C18 silica gel; mobile phase, acetonitrile in water, 50% to 85% gradient in 25 min; detector, UV 220 nm) to afford 4-chloro-5-[4-(2-chloro-4-fluorophenoxy)-2-[[(4-methoxyphenyl)methyl]amino]-5H,6H,7H,8H-pyrido[3,4-d]pyrimidin-7-yl]-2-(oxan-2-yl)-2,3-dihydropyridazin-3-one (350 mg, 89.00%) as a yellow solid.

### 5-[2-amino-4-(2-chloro-4-fluorophenoxy)-5H,6H,7H,8H-pyrido[3,4-d]pyrimidin-7-yl]-4-chloro-2,3-dihydropyridazin-3-one

To a stirred solution of 4-chloro-5-[4-(2-chloro-4-fluorophenoxy)-2-[[(4-methoxyphenyl)methyl]amino]-5H,6H,7H,8H-pyrido[3,4-d]pyrimidin-7-yl]-2-(oxan-2-yl)-2,3-dihydropyridazin-3-one (200 mg) in TFA (8 mL, 107.704 mmol, 328.23 equiv). The final reaction mixture was irradiated with microwave radiation for 2 hours at 80° C. The reaction was monitored by LCMS. The resulting mixture was concentrated under reduced pressure. The residue was basified to pH=8 with saturated NH₄HCO₃ (aq.). The resulting mixture was extracted with DCM (2×100 mL). The combined organic layers were washed with brine (1×100 mL), dried over anhydrous Na₂SO₄. After filtration, the filtrate was concentrated under reduced pressure. The residue was purified by Prep-HPLC with the following conditions (Column: XBridge Prep OBD C18 Column 30×150 mm Sum; Mobile Phase A: Water (10 mM NH4HCO3), Mobile Phase B: acetonitrile; Flow rate: 60 mL/min; Gradient: 25% B to 40% B in 8 min; 220 nm; Rt: 7.35 min) to afford 5-[2-amino-4-(2-chloro-4-fluorophenoxy)-5H,6H,7H,8H-pyrido[3,4-d]pyrimidin-7-yl]-4-chloro-2,3-dihydropyridazin-3-one (52.4 mg) as a yellow solid.

Compounds 113, 116, and 102 were prepared by the methods and scheme described in this Example by using 2-chlorophenol, 4-fluoro-2-trifluoromethylphenol, 2-trifluorophenol respectively, in place of 2-chloro-4-fluorophenol in the first step of the synthesis.

### Example 9. Synthesis of Compounds 129 and 130

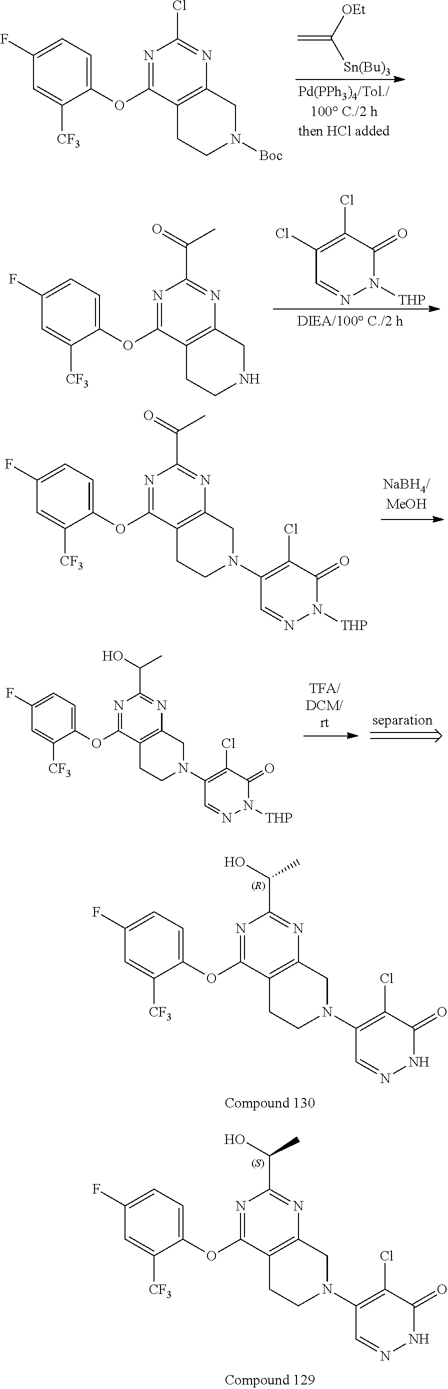

### 1-[4-[4-fluoro-2-(trifluoromethyl)phenoxy]-5H,6H,7H,8H-pyrido[3,4-d]pyrimidin-2-yl]ethan-1-one

To a mixture of tert-butyl 2-chloro-4-[4-fluoro-2-(trifluoromethyl)phenoxy]-5H,6H,7H,8H-pyrido[3,4-d]pyrimidine-7-carboxylate (600 mg, 1.340 mmol, 1 equiv.) and tributyl (1-ethoxyethenyl)stannane (967.80 mg, 2.680 mmol, 2.00 equiv.) in Toluene (10 mL) was added Pd(PPh₃)₄ (77.41 mg, 0.067 mmol, 0.05 equiv.) at room temperature under nitrogen atmosphere. The resulting mixture was stirred for 4 hours at 110° C. The reaction was monitored by LCMS. This resulted in tert-butyl 2-(1-ethoxyethenyl)-4-[4-fluoro-2-(trifluoromethyl)phenoxy]-5H,6H,7H,8H-pyrido[3,4-d]pyrimidine-7-carboxylate (700 mg, 108.06%) as a yellow oil. The crude resulting mixture was used in the next step directly without further purification.

To a stirred solution of tert-butyl 2-(1-ethoxyethenyl)-4-[4-fluoro-2-(trifluoromethyl)phenoxy]-5H,6H,7H,8H-pyrido[3,4-d]pyrimidine-7-carboxylate (1 g, 2.068 mmol, 1 equiv.) in DCM (5 mL) was added TFA (3.33 mL, 29.239 mmol, 21.70 equiv.) at room temperature. The resulting mixture was stirred for 2 hours at room temperature. The reaction was monitored by LCMS. The resulting mixture was concentrated under reduced pressure. The mixture/residue was basified to pH 8 with saturated NaHCO₃(aq.). The resulting mixture was concentrated under reduced pressure. The residue was purified by reverse phase flash chromatography with the following conditions: Column: Spherical C18, 20-40 um, 330 g; Mobile Phase A: Water (plus 5 mM NH₄HCO₃); Mobile Phase B: acetonitrile; Flow rate: 80 mL/min; Gradient: 5%-5% B, 10 min, 43% B-55% B gradient in 20 min; Detector: 220 nm. The fractions containing the desired product were collected at 50% B and concentrated under reduced pressure to afford 1-[4-[4-fluoro-2-(trifluoromethyl)phenoxy]-5H,6H,7H,8H-pyrido[3,4-d]pyrimidin-2-yl]ethan-1-one (750 mg, 102.06%) as a light yellow solid.

### 5-[2-Acetyl-4-[4-fluoro-2-(trifluoromethyl)phenoxy]-5H,6H,7H,8H-pyrido[3,4-d]pyrimidin-7-yl]-4-chloro-2-(oxan-2-yl)-2,3-dihydropyridazin-3-one

Into a 50 mL round-bottom flask were added 1-[4-[4-fluoro-2-(trifluoromethyl)phenoxy]-5H,6H,7H,8H-pyrido[3,4-d]pyrimidin-2-yl]ethan-1-one (750 mg, 2.111 mmol, 1 equiv.) and 4,5-dichloro-2-(oxan-2-yl)-2,3-dihydropyridazin-3-one (525.81 mg, 2.111 mmol, 1.00 equiv.) at room temperature. To the above mixture was added DIEA (818.47 mg, 6.333 mmol, 3.00 equiv). The resulting mixture was stirred for 2 hours at 100° C. The reaction was monitored by LCMS. The mixture was allowed to cool down to room temperature. The residue was purified by reverse phase flash chromatography with the following conditions: Column: Spherical C18, 20-40 um, 330 g; Mobile Phase A: Water (plus 5 mM NH₄HCO₃); Mobile Phase B: acetonitrile; Flow rate: 80 mL/min; Gradient: 5%-5% B, 10 min, 60% B-85% B gradient in 20 min; Detector: 220 nm. The fractions containing the desired product were collected at 80% B and concentrated under reduced pressure to afford 5-[2-acetyl-4-[4-fluoro-2-(trifluoromethyl)phenoxy]-5H,6H,7H,8H-pyrido[3,4-d]pyrimidin-7-yl]-4-chloro-2-(oxan-2-yl)-2,3-dihydropyridazin-3-one (230 mg, 19.18%) as a light yellow oil.

### 4-Chloro-5-[4-[4-fluoro-2-(trifluoromethyl)phenoxy]-2-(1-hydroxyethyl)-5H,6H,7H,8H-pyrido[3,4-d]pyrimidin-7-yl]-2-(oxan-2-yl)-2,3-dihydropyridazin-3-one

To a stirred solution of 5-[2-acetyl-4-[4-fluoro-2-(trifluoromethyl)phenoxy]-5H,6H,7H,8H-pyrido[3,4-d]pyrimidin-7-yl]-4-chloro-2-(oxan-2-yl)-2,3-dihydropyridazin-3-one (230 mg, 0.405 mmol, 1 equiv.) in MeOH (10 mL) was added NaBH₄ (30.64 mg, 0.810 mmol, 2.00 equiv.) in portions at 0° C. under nitrogen atmosphere. The resulting mixture was stirred for 2 hours at room temperature. The reaction was monitored by LCMS. The resulting mixture was concentrated under reduced pressure. The residue was purified by Prep-TLC (PE/EtOAc 1/1) to afford 4-chloro-5-[4-[4-fluoro-2-(trifluoromethyl)phenoxy]-2-(1-hydroxyethyl)-5H,6H,7H,8H-pyrido[3,4-d]pyrimidin-7-yl]-2-(oxan-2-yl)-2,3-dihydropyridazin-3-one (120 mg, 51.99%) as a light yellow oil.

### 4-Chloro-5-[4-[4-fluoro-2-(trifluoromethyl)phenoxy]-2-[(1S)-1-hydroxyethyl]-5H,6H,7H,8H-pyrido[3,4-d]pyrimidin-7-yl]-2,3-dihydropyridazin-3-one and 4-chloro-5-[4-[4-fluoro-2-(trifluoromethyl)phenoxy]-2-[(1R)-1-hydroxyethyl]-5H,6H,7H,8H-pyrido[3,4-d]pyrimidin-7-yl]-2,3-dihydropyridazin-3-one

To a stirred solution of 4-chloro-5-[4-[4-fluoro-2-(trifluoromethyl)phenoxy]-2-(1-hydroxyethyl)-5H,6H,7H,8H-pyrido[3,4-d]pyrimidin-7-yl]-2-(oxan-2-yl)-2,3-dihydropyridazin-3-one (120 mg, 0.211 mmol, 1 equiv.) in DCM (5 mL) was added TFA (2.00 mL, 17.541 mmol, 127.89 equiv.) dropwise at room temperature. The resulting mixture was stirred for 2 hours at room temperature. The reaction was monitored by LCMS. The residue was basified to pH 8 with saturated NaHCO₃(aq.). The resulting mixture was concentrated under reduced pressure. The residue was purified by reverse phase flash chromatography with the following conditions: Column: Spherical C18, 20-40 um, 330 g; Mobile Phase A: Water (plus 5 mM NH₄HCO₃); Mobile Phase B: acetonitrile; Flow rate: 80 mL/min; Gradient: 5%-5% B, 10 min, 40% B-80% B gradient in 25 min; Detector: 220 nm. The fractions containing the desired product were collected at 55% B and concentrated under reduced pressure. The crude product (50 mg) was purified by Chiral-Prep-HPLC with the following conditions (Column: CHIRALPAK IE, 2*25 cm, 5 um; Mobile Phase A: Hex (0.1% FA)—HPLC, Mobile Phase B: EtOH—HPLC; Flow rate: 16 mL/min; Gradient: 30 B to 30 B in 33 min; 220/254 nm; RT1:26.219; RT2:29.589). Although the two isomers were separated by this technique, the absolute orientation was not determined. The compound designated as 4-chloro-5-[4-[4-fluoro-2-(trifluoromethyl)phenoxy]-2-[(1S)-1-hydroxyethyl]-5H,6H,7H,8H-pyrido[3,4-d]pyrimidin-7-yl]-2,3-dihydropyridazin-3-one (27.1 mg) was obtained at 29.589 min as an off-white solid. The compound designated as 4-chloro-5-[4-[4-fluoro-2-(trifluoromethyl)phenoxy]-2-[(1R)-1-hydroxyethyl]-5H,6H,7H,8H-pyrido[3,4-d]pyrimidin-7-yl]-2,3-dihydropyridazin-3-one (22.6 mg) was obtained at 26.219 min as an off-white solid.

Compound 119 was prepared by the methods and scheme described in this Example using tert-butyl 2-chloro-4-[4-fluoro-2-chlorophenoxy]-5H,6H,7H,8H-pyrido[3,4-d]pyrimidine-7-carboxylate as the starting material.

Compounds 122 and 123 were prepared by the methods and scheme described in this Example using tert-butyl 2-chloro-4-[2-(trifluoromethyl)phenoxy]-5H,6H,7H,8H-pyrido[3,4-d]pyrimidine-7-carboxylate as the starting material. Again, the absolute orientation of these separated isomers was not determined and the designation as (S) or (R) was arbitrary.

### Example 10. Synthesis of Compound 115

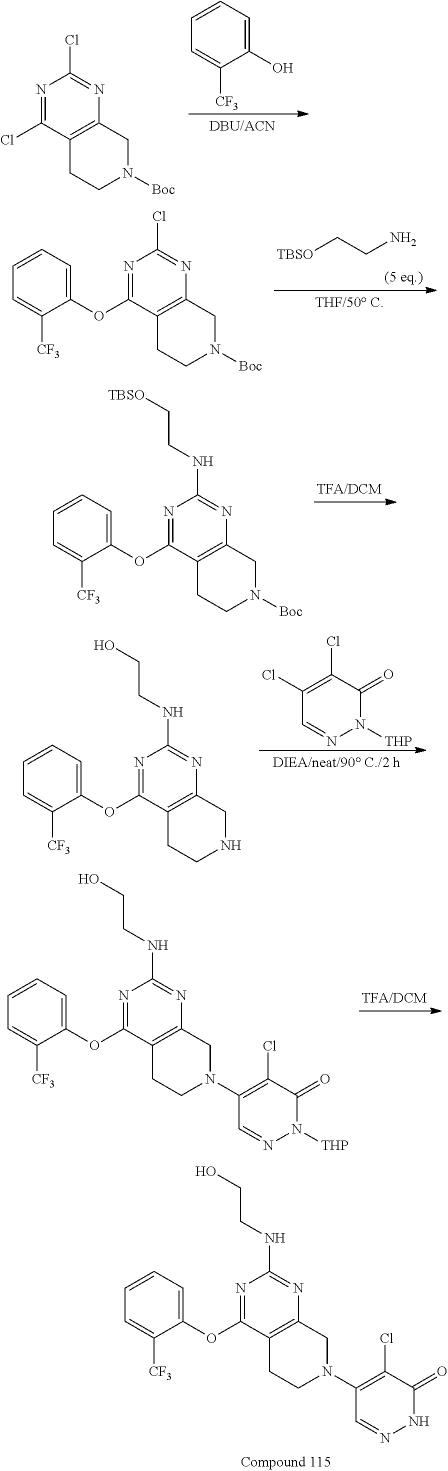

### tert-Butyl 2-chloro-4-[2-(trifluoromethyl)phenoxy]-5H,6H,7H,8H-pyrido[3,4-d]pyrimidine-7-carboxylate

To a stirred solution of tert-butyl 2,4-dichloro-5H,6H,7H,8H-pyrido[3,4-d]pyrimidine-7-carboxylate (2 g, 6.58 mmol, 1 equiv.) and 2-(trifluoromethyl)phenol (1.6 g, 9.86 mmol, 1.5 equiv.) in acetonitrile (20 mL) was added DBU (2.0 g, 13.15 mmol, 2 equiv.) at room temperature. The solution was stirred at rt for 4 hours. The mixture was concentrated under reduced pressure. The residue was purified by Prep-TLC (PE/EtOAc 10:1) to afford tert-butyl 2-chloro-4-[2-(trifluoromethyl)phenoxy]-5H,6H,7H,8H-pyrido[3,4-d]pyrimidine-7-carboxylate (700 mg, 24.77%) as colorless oil.

### tert-butyl 2-([2-[(tert-butyldimethylsilyl)oxy]ethyl]amino)-4-[2-(trifluoromethyl)phenoxy]-5H,6H,7H,8H-pyrido[3,4-d]pyrimidine-7-carboxylate

To a solution of tert-butyl 2-chloro-4-[2-(trifluoromethyl)phenoxy]-5H,6H,7H,8H-pyrido[3,4-d]pyrimidine-7-carboxylate (500 mg, 1.163 mmol, 1 equiv.) in THF (15 mL) was added (2-aminoethoxy)(tert-butyl)dimethylsilane (1019.89 mg, 5.816 mmol, 5.00 equiv.) at room temperature under nitrogen atmosphere. The resulting mixture was stirred for 16 h at 50° C. The reaction was monitored by LCMS. The resulting mixture was concentrated under reduced pressure. The residue was purified by Prep-TLC (PE/EtOAc 3/1) to afford tert-butyl 2-([2-[(tert-butyldimethylsilyl)oxy]ethyl]amino)-4-[2-(trifluoromethyl)phenoxy]-5H,6H,7H,8H-pyrido[3,4-d]pyrimidine-7-carboxylate (440 mg, 66.51%) as a light yellow oil.

### 2-([4-[2-(trifluoromethyl)phenoxy]-5H,6H,7H,8H-pyrido[3,4-d]pyrimidin-2-yl]amino)ethan-1-ol

To a stirred solution of tert-butyl 2-([2-[(tert-butyldimethylsilyl)oxy]ethyl]amino)-4-[2-(trifluoromethyl)phenoxy]-5H,6H,7H,8H-pyrido[3,4-d]pyrimidine-7-carboxylate (440 mg, 0.774 mmol, 1 equiv.) in DCM (10 mL) was added TFA (3 mL, 40.389 mmol, 52.20 equiv.) at room temperature. The resulting mixture was stirred for 2 h at room temperature. The reaction was monitored by LCMS. The resulting mixture was concentrated under reduced pressure. The residue was purified by reverse flash chromatography with the following conditions: column, C18 silica gel; mobile phase, ACN in water, 40% to 60% gradient in 15 min; detector, UV 254 nm to afford 2-([4-[2-(trifluoromethyl)phenoxy]-5H,6H,7H,8H-pyrido[3,4-d]pyrimidin-2-yl]amino)ethan-1-ol (220 mg) as light yellow oil.

### 4-chloro-5-[2-[(2-hydroxyethyl)amino]-4-[2-(trifluoromethyl)phenoxy]-5H,6H,7H,8H-pyrido[3,4-d]pyrimidin-7-yl]-2-(oxan-2-yl)-2,3-dihydropyridazin-3-one

Into a 50 mL round-bottom flask were added 2-([4-[2-(trifluoromethyl)phenoxy]-5H,6H,7H,8H-pyrido[3,4-d]pyrimidin-2-yl]amino)ethan-1-ol (220 mg, 0.621 mmol, 1 equiv.) and 4,5-dichloro-2-(oxan-2-yl)-2,3-dihydropyridazin-3-one (154.66 mg, 0.621 mmol, 1.00 equiv.) at room temperature. To the above mixture was added DIEA (240.74 mg, 1.863 mmol, 3.00 equiv). The resulting mixture was stirred for 2 h at 100 degrees C. The reaction was monitored by LCMS. The mixture was allowed to cool down to room temperature. The residue was purified by reverse phase flash chromatography with the following conditions: Column: Spherical C18, 20-40 um, 330 g; Mobile Phase A: Water (plus 5 mM NH₄HCO₃); Mobile Phase B: ACN; Flow rate: 80 mL/min; Gradient: 5%-5% B, 10 min, 45% B-60% B gradient in 20 min; Detector: 220 nm. The fractions containing the desired product were collected at 55% B and concentrated under reduced pressure to afford 4-chloro-5-[2-[(2-hydroxyethyl)amino]-4-[2-(trifluoromethyl)phenoxy]-5H,6H,7H,8H-pyrido[3,4-d]pyrimidin-7-yl]-2-(oxan-2-yl)-2,3-dihydropyridazin-3-one (210 mg, 59.66%) as a yellow solid.

### 4-chloro-5-[2-[(2-hydroxyethyl)amino]-4-[2-(trifluoromethyl)phenoxy]-5H,6H,7H,8H-pyrido[3,4-d]pyrimidin-7-yl]-2,3-dihydropyridazin-3-one

To a stirred solution of 4-chloro-5-[2-[(2-hydroxyethyl)amino]-4-[2-(trifluoromethyl)phenoxy]-5H,6H,7H,8H-pyrido[3,4-d]pyrimidin-7-yl]-2-(oxan-2-yl)-2,3-dihydropyridazin-3-one (200 mg, 0.353 mmol, 1 equiv.) in DCM (5 mL) was added TFA (2 mL) at room temperature. The resulting mixture was stirred for 1 h at room temperature. The reaction was monitored by LCMS. The mixture was basified to pH 8 with saturated NaHCO₃(aq.). The resulting mixture was concentrated under reduced pressure. The residue was purified by Prep-HPLC with the following conditions (Column: XBridge Prep OBD C18 Column 30×150 mm Sum; Mobile Phase A: undefined, Mobile Phase B: undefined; Flow rate: 60 mL/min; Gradient: 25% B to 50% B in 8 min; 220 nm; Rt: 7.67 min) to afford 4-chloro-5-[2-[(2-hydroxyethyl)amino]-4-[2-(trifluoromethyl)phenoxy]-5H,6H,7H,8H-pyrido[3,4-d]pyrimidin-7-yl]-2,3-dihydropyridazin-3-one (106.3 mg) as a white solid.

### Example 11. Synthesis of Compounds 138 and 139

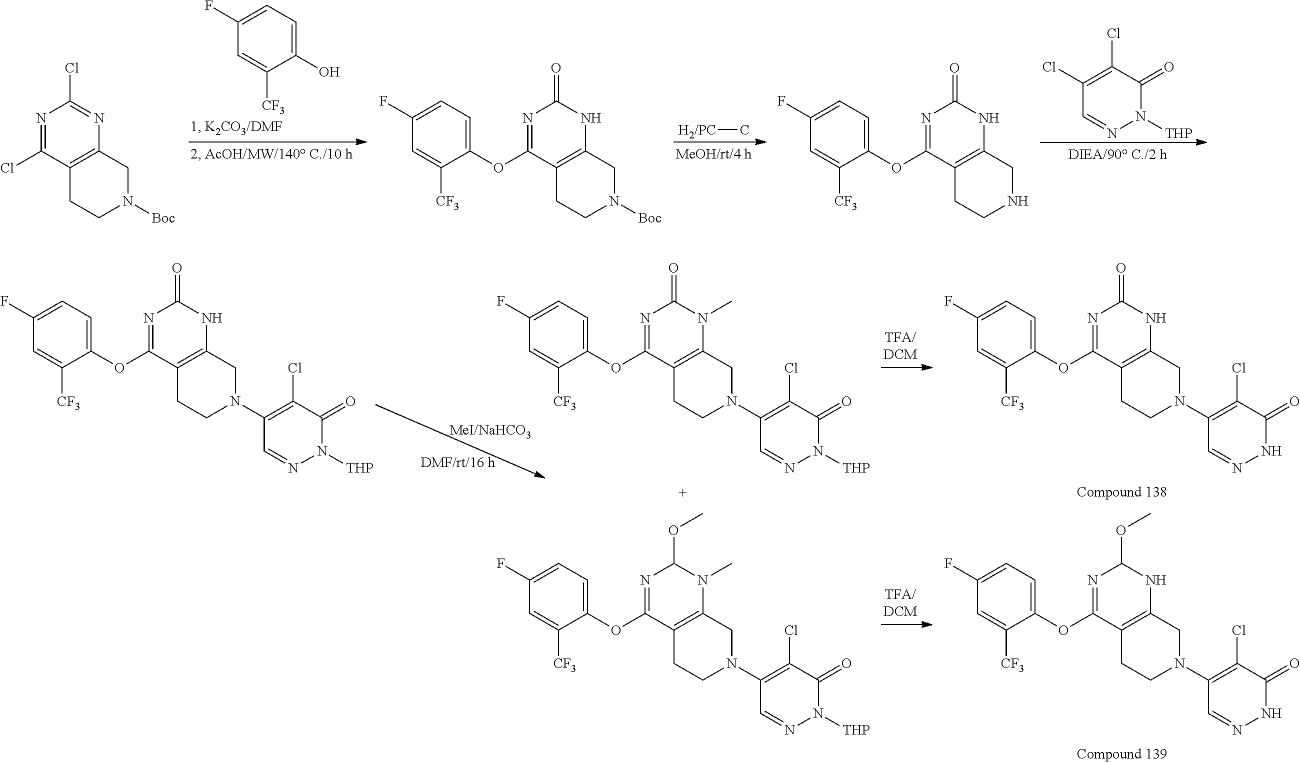

### 7-Benzyl-2-chloro-4-[4-fluoro-2-(trifluoromethyl)phenoxy]-5H,6H,7H,8H-pyrido[3,4-d]pyrimidine

To a stirred solution of 4-fluoro-2-(trifluoromethyl)phenol (1469.32 mg, 8.158 mmol, 1.20 equiv.) and 7-benzyl-2,4-dichloro-5H,6H,7H,8H-pyrido[3,4-d]pyrimidine (2000 mg, 6.799 mmol, 1 equiv.) in DMF (20 mL) was added K₂CO₃ (1879.20 mg, 13.597 mmol, 2 equiv.) at room temperature. The solution was stirred at 70° C. for 0.5 hours. The mixture was concentrated under reduced pressure. The residue was purified by reverse phase flash chromatography with the following conditions: Column: Spherical C18, 20-40 um, 330 g; Mobile Phase A: Water (plus 5 mM TFA); Mobile Phase B: acetonitrile; Flow rate: 80 mL/min; Gradient: 5%-5% B, 10 min, 70% B-95% B gradient in 20 min; Detector: 254 nm. The fractions containing the desired product were collected at 95% B and concentrated under reduced pressure to afford 7-benzyl-2-chloro-4-[4-fluoro-2-(trifluoromethyl)phenoxy]-5H,6H,7H,8H-pyrido[3,4-d]pyrimidine (2331 mg, 78.31%) as an off-white solid.

### 7-Benzyl-4-[4-fluoro-2-(trifluoromethyl)phenoxy]-1H,2H,5H,6H,7H,8H-pyrido[3,4-d]pyrimidin-2-one

A solution of 7-benzyl-2-chloro-4-[4-fluoro-2-(trifluoromethyl)phenoxy]-5H,6H,7H,8H-pyrido[3,4-d]pyrimidine (2 g, 4.568 mmol, 1 equiv.) in HAc (10 mL, 174.515 mmol, 38.20 equiv.) and H₂O (1 mL, 55.508 mmol, 12.15 equiv.) was stirred for 10 hours at 140° C. under N₂ atmosphere. The resulting mixture was concentrated under reduced pressure. The residue was purified by Prep-TLC (PE/EA 1/1) to afford 7-benzyl-4-[4-fluoro-2-(trifluoromethyl)phenoxy]-1H,2H,5H,6H,7H,8H-pyrido[3,4-d]pyrimidin-2-one (530 mg, 27.67%) as a light yellow solid.

### 4-[4-Fluoro-2-(trifluoromethyl)phenoxy]-1H,2H,5H,6H,7H,8H-pyrido[3,4-d]pyrimidin-2-one

To a solution of 7-benzyl-4-[4-fluoro-2-(trifluoromethyl)phenoxy]-1H,2H,5H,6H,7H,8H-pyrido[3,4-d]pyrimidin-2-one (530 mg, 1.264 mmol, 1 equiv.) in MeOH (10 mL, 246.989 mmol, 195.44 equiv.) was added Pd/C (268.98 mg, 2.528 mmol, 2 equiv.) under nitrogen atmosphere. The mixture was hydrogenated at room temperature for 4 hours under hydrogen atmosphere using a hydrogen balloon, filtered through a Celite pad and concentrated under reduced pressure. To afford 4-[4-fluoro-2-(trifluoromethyl)phenoxy]-1H,2H,5H,6H,7H,8H-pyrido[3,4-d]pyrimidin-2-one (430 mg, 103.34%) as a light yellow solid.

### 4-Chloro-5-[4-[4-fluoro-2-(trifluoromethyl)phenoxy]-2-oxo-1H,2H,5H,6H,7H,8H-pyrido[3,4-d]pyrimidin-7-yl]-2-(oxan-2-yl)-2,3-dihydropyridazin-3-one

A mixture of 4-[4-fluoro-2-(trifluoromethyl)phenoxy]-1H,2H,5H,6H,7H,8H-pyrido[3,4-d]pyrimidin-2-one (430 mg, 1.306 mmol, 1 equiv.) and 4,5-dichloro-2-(oxan-2-yl)-2,3-dihydropyridazin-3-one (357.84 mg, 1.437 mmol, 1.1 equiv.) in DIEA (337.58 mg, 2.612 mmol, 2.00 equiv.) was stirred for 2 hours at 100° C. under N2 atmosphere. The residue was purified by Prep-TLC (PE/EA 1/1) to afford 4-chloro-5-[4-[4-fluoro-2-(trifluoromethyl)phenoxy]-2-oxo-1H,2H,5H,6H,7H,8H-pyrido[3,4-d]pyrimidin-7-yl]-2-(oxan-2-yl)-2,3-dihydropyridazin-3-one (210 mg, 29.67%) as a light yellow solid.

### 4-Chloro-5-[4-[4-fluoro-2-(trifluoromethyl)phenoxy]-1-methyl-2-oxo-1H,2H,5H,6H,7H,8H-pyrido[3,4-d]pyrimidin-7-yl]-2-(oxan-2-yl)-2,3-dihydropyridazin-3-one and 4-chloro-5-[4-[4-fluoro-2-(trifluoromethyl)phenoxy]-2-methoxy-5H,6H,7H,8H-pyrido[3,4-d]pyrimidin-7-yl]-2-(oxan-2-yl)-2,3-dihydropyridazin-3-one

To a solution of 4-chloro-5-[4-[4-fluoro-2-(trifluoromethyl)phenoxy]-2-oxo-1H,2H,5H,6H,7H,8H-pyrido[3,4-d]pyrimidin-7-yl]-2-(oxan-2-yl)-2,3-dihydropyridazin-3-one (90 mg, 0.166 mmol, 1 equiv.) and NaHCO₃ (27.90 mg, 0.332 mmol, 2 equiv.) in DMF (10 mL, 129.218 mmol, 778.02 equiv.) was added CH₃I (47.15 mg, 0.332 mmol, 2.00 equiv.) dropwise at 0° C. under nitrogen atmosphere. The mixture was stirred at 25° C. for 16 hours. The resulting mixture was concentrated under reduced pressure. The residue was purified by Prep-TLC (PE/EA 0/1) to afford 4-chloro-5-[4-[4-fluoro-2-(trifluoromethyl)phenoxy]-1-methyl-2-oxo-1H,2H,5H,6H,7H,8H-pyrido[3,4-d]pyrimidin-7-yl]-2-(oxan-2-yl)-2,3-dihydropyridazin-3-one (60 mg, 64.99%) and 4-chloro-5-[4-[4-fluoro-2-(trifluoromethyl)phenoxy]-2-methoxy-5H,6H,7H,8H-pyrido[3,4-d]pyrimidin-7-yl]-2-(oxan-2-yl)-2,3-dihydropyridazin-3-one (15 mg) as a light yellow solid.

### 4-Chloro-5-[4-[4-fluoro-2-(trifluoromethyl)phenoxy]-1-methyl-2-oxo-1H,2H,5H,6H,7H,8H-pyrido[3,4-d]pyrimidin-7-yl]-2,3-dihydropyridazin-3-one

To a solution of 4-chloro-5-[4-[4-fluoro-2-(trifluoromethyl)phenoxy]-1-methyl-2-oxo-1H,2H,5H,6H,7H,8H-pyrido[3,4-d]pyrimidin-7-yl]-2-(oxan-2-yl)-2,3-dihydropyridazin-3-one (60 mg, 0.108 mmol, 1 equiv.) in DCM (10 mL, 157.300 mmol, 1457.41 equiv.) was added TFA (123.07 mg, 1.079 mmol, 10 equiv.) at 25° C. The resulting mixture was concentrated under reduced pressure. The crude product (100 mg) was purified by Prep-HPLC with the following conditions (Column: XBridge Shield RP18 OBD Column 30*150 mm, 5 um; Mobile Phase A: Water (10 mM NH₄HCO₃), Mobile Phase B: acetonitrile; Flow rate: 60 mL/min; Gradient: 20% B to 40% B in 7 min; 220 nm; Rt: 6.63 min) to afford 4-chloro-5-[4-[4-fluoro-2-(trifluoromethyl)phenoxy]-1-methyl-2-oxo-1H,2H,5H,6H,7H,8H-pyrido[3,4-d]pyrimidin-7-yl]-2,3-dihydropyridazin-3-one (29.3 mg, 57.54%) as a white solid.

### 4-Chloro-5-[4-[4-fluoro-2-(trifluoromethyl)phenoxy]-2-methoxy-5H,6H,7H,8H-pyrido[3,4-d]pyrimidin-7-yl]-2,3-dihydropyridazin-3-one

To a solution of 4-chloro-5-[4-[4-fluoro-2-(trifluoromethyl)phenoxy]-2-methoxy-5H,6H,7H,8H-pyrido[3,4-d]pyrimidin-7-yl]-2-(oxan-2-yl)-2,3-dihydropyridazin-3-one (15 mg, 0.027 mmol, 1 equiv.) in DCM (5 mL, 78.650 mmol, 2914.83 equiv.) was added TFA (30.77 mg, 0.270 mmol, 10 equiv.) at 25° C. The resulting mixture was concentrated under reduced pressure. The crude product (20 mg) was purified by Prep-HPLC with the following conditions (Column: XBridge Shield RP18 OBD Column 30*150 mm, 5 um; Mobile Phase A: Water (10 mM NH₄HCO₃), Mobile Phase B: acetonitrile; Flow rate: 60 mL/min; Gradient: 20% B to 40% B in 7 min; 220 nm; Rt: 6.63 min) to afford 4-chloro-5-[4-[4-fluoro-2-(trifluoromethyl)phenoxy]-2-methoxy-5H,6H,7H,8H-pyrido[3,4-d]pyrimidin-7-yl]-2,3-dihydropyridazin-3-one (7.5 mg, 58.91%) as a white solid.

### Example 12. Synthesis of Compound 110

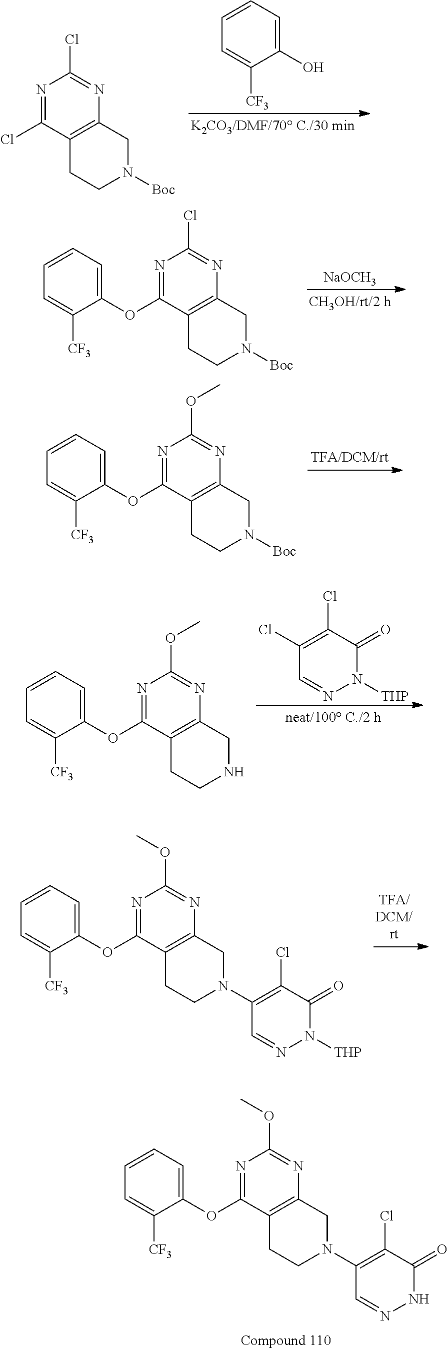

### Tert-Butyl 2-chloro-4-[2-(trifluoromethyl)phenoxy]-5H,6H,7H,8H-pyrido[3,4-d]pyrimidine-7-carboxylate

To a stirred solution of tert-butyl 2,4-dichloro-5H,6H,7H,8H-pyrido[3,4-d]pyrimidine-7-carboxylate (2 g, 6.58 mmol, 1 equiv.) and 2-(trifluoromethyl)phenol (1.6 g, 9.86 mmol, 1.5 equiv.) in acetonitrile (20 mL) was added DBU (2.0 g, 13.15 mmol, 2 equiv.) at room temperature. The solution was stirred at rt for 4 hours. The mixture was concentrated under reduced pressure. The residue was purified by Prep-TLC (PE/EtOAc 10:1) to afford tert-butyl 2-chloro-4-[2-(trifluoromethyl)phenoxy]-5H,6H,7H,8H-pyrido[3,4-d]pyrimidine-7-carboxylate (700 mg, 24.77%) as colorless oil.

### Tert-Butyl 2-methoxy-4-[2-(trifluoromethyl)phenoxy]-5H,6H,7H,8H-pyrido[3,4-d]pyrimidine-7-carboxylate

To a solution of tert-butyl 2-chloro-4-[2-(trifluoromethyl)phenoxy]-5H,6H,7H,8H-pyrido[3,4-d]pyrimidine-7-carboxylate (1 g, 2.327 mmol, 1 equiv.) in MeOH (20 mL, 493.978 mmol, 212.32 equiv.) was added NaOMe (0.25 g, 0.005 mmol, 2 equiv.) at 25° C. The mixture was stirred at 25° C. for 4 hours. The resulting mixture was concentrated under reduced pressure. The residue was purified by silica gel column chromatography, eluted with PE/EA (10/1 to 1/1) to afford tert-butyl 2-methoxy-4-[2-(trifluoromethyl)phenoxy]-5H,6H,7H,8H-pyrido[3,4-d]pyrimidine-7-carboxylate (100 mg, 10.10%) as a light yellow solid.

### 2-Methoxy-4-[2-(trifluoromethyl)phenoxy]-5H,6H,7H,8H-pyrido[3,4-d]pyrimidine

To a solution of tert-butyl 2-methoxy-4-[2-(trifluoromethyl)phenoxy]-5H,6H,7H,8H-pyrido[3,4-d]pyrimidine-7-carboxylate (100 mg, 0.235 mmol, 1 equiv.) in DCM (10 mL) was added TFA (268.03 mg, 2.351 mmol, 10 equiv.) at 25° C. The solution was stirred at 25° C. for 4 hours. The resulting mixture was concentrated under reduced pressure. The crude product (150 mg) was purified by Prep-HPLC with the following conditions (Column: XBridge Shield RP18 OBD Column 30*150 mm, 5 um; Mobile Phase A: Water (10 mM NH₄HCO₃), Mobile Phase B: acetonitrile; Flow rate: 60 mL/min; Gradient: 20% B to 40% B in 7 min; 220 nm; Rt: 6.63 min) to afford 2-methoxy-4-[2-(trifluoromethyl)phenoxy]-5H,6H,7H,8H-pyrido[3,4-d]pyrimidine (80 mg, 104.62%) as a light yellow solid.

### 4-Chloro-5-[2-methoxy-4-[2-(trifluoromethyl)phenoxy]-5H,6H,7H,8H-pyrido[3,4-d]pyrimidin-7-yl]-2-(oxan-2-yl)-2,3-dihydropyridazin-3-one

A solution of 2-methoxy-4-[2-(trifluoromethyl)phenoxy]-5H,6H,7H,8H-pyrido[3,4-d]pyrimidine (80 mg, 0.246 mmol, 1 equiv.) and 4,5-dichloro-2-(oxan-2-yl)-2,3-dihydropyridazin-3-one (61.26 mg, 0.246 mmol, 1 equiv.) in DIEA (63.57 mg, 0.492 mmol, 2.00 equiv.) was stirred for 2 hours at 100° C. under nitrogen atmosphere. The residue was purified by silica gel column chromatography, eluted with PE/EA (5/1 to 1/1) to afford 4-chloro-5-[2-methoxy-4-[2-(trifluoromethyl)phenoxy]-5H,6H,7H,8H-pyrido[3,4-d]pyrimidin-7-yl]-2-(oxan-2-yl)-2,3-dihydropyridazin-3-one (120 mg, 90.71%) as a light yellow solid.

### 4-Chloro-5-[2-methoxy-4-[2-(trifluoromethyl)phenoxy]-5H,6H,7H,8H-pyrido[3,4-d]pyrimidin-7-yl]-2,3-dihydropyridazin-3-one

To a solution of 4-chloro-5-[2-methoxy-4-[2-(trifluoromethyl)phenoxy]-5H,6H,7H,8H-pyrido[3,4-d]pyrimidin-7-yl]-2-(oxan-2-yl)-2,3-dihydropyridazin-3-one (120 mg, 0.223 mmol, 1 equiv.) in DCM (5 mL, 78.650 mmol, 352.56 equiv.) was added TFA (254.36 mg, 2.231 mmol, 10.00 equiv.) at 25° C. The resulting mixture was concentrated under reduced pressure. The crude product (150 mg) was purified by Prep-HPLC with the following conditions (Column: XBridge Shield RP18 OBD Column 30*150 mm, 5 um; Mobile Phase A: Water (10 mM NH₄HCO₃), Mobile Phase B: acetonitrile; Flow rate: 60 mL/min; Gradient: 20% B to 40% B in 7 min; 220 nm; Rt: 6.63 min) to afford 4-chloro-5-[2-methoxy-4-[2-(trifluoromethyl)phenoxy]-5H,6H,7H,8H-pyrido[3,4-d]pyrimidin-7-yl]-2,3-dihydropyridazin-3-one (24.1 mg, 23.81%) as a white solid.

### Example 13. Synthesis of Compound 108

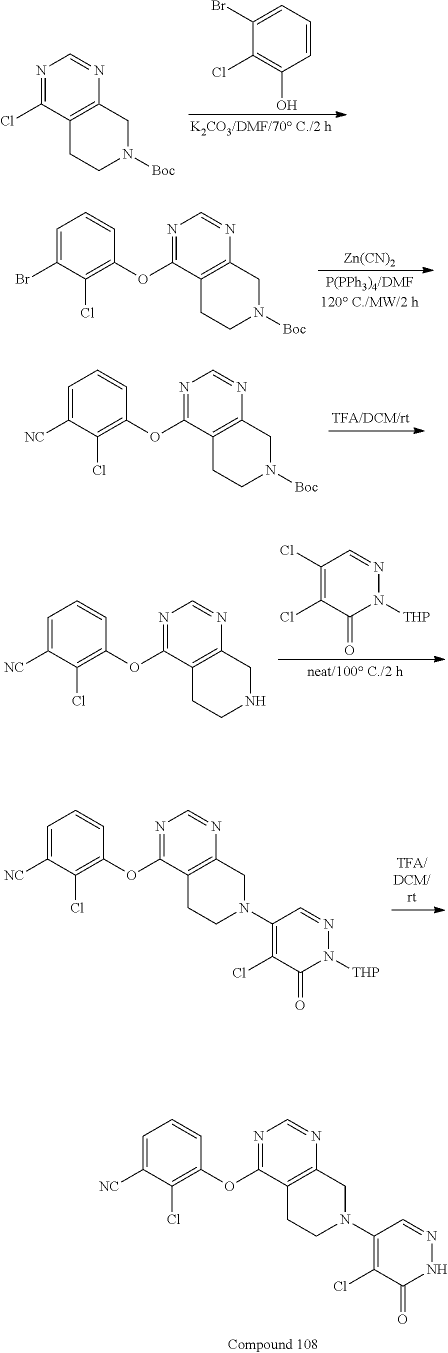

### Tert-Butyl 4-(3-bromo-2-chlorophenoxy)-5H,6H,7H,8H-pyrido[3,4-d]pyrimidine-7-carboxylate

To a stirred solution of tert-butyl 4-chloro-5H,6H,7H,8H-pyrido[3,4-d]pyrimidine-7-carboxylate (500 mg, 1.854 mmol, 1 equiv.) and 3-bromo-2-chlorophenol (461.46 mg, 2.224 mmol, 1.20 equiv.) in DMF (10 mL) was added K₂CO₃ (512.38 mg, 3.707 mmol, 2 equiv). The resulting mixture was stirred for 1 h at 70° C. The mixture was purified by reverse flash chromatography with the following conditions: Column: (spnerical C18, 20-40 um, 330 g; Mobile Phase A: Water (5 mM NH₄HCO₃), Mobile Phase B: acetonitrile; Flow rate: 80 mL/min; Gradient: 20% B to 60% B in 55 min; 254 nm). The fractions containing the desired product were collected at 40% B and concentrated under reduced pressure. This resulted in tert-butyl 4-(3-bromo-2-chlorophenoxy)-5H,6H,7H,8H-pyrido[3,4-d]pyrimidine-7-carboxylate (300 mg, 36.72%) as an off-white solid.

### Tert-Butyl 4-(2-chloro-3-cyanophenoxy)-5H,6H,7H,8H-pyrido[3,4-d]pyrimidine-7-carboxylate

To a stirred solution of tert-butyl 4-(3-bromo-2-chlorophenoxy)-5H,6H,7H,8H-pyrido[3,4-d]pyrimidine-7-carboxylate (450 mg, 1.021 mmol, 1 equiv.) and zinc dicarbonitrile (143.87 mg, 1.225 mmol, 1.20 equiv.) in DMF (5 mL) was added Pd(PPh₃)₄ (117.99 mg, 0.102 mmol, 0.1 equiv). The resulting mixture was stirred for 2 hours at 120° C. under nitrogen atmosphere. The residue was purified by reverse flash chromatography with the following conditions: Column: spnerical C18, 20-40 um, 180 g; Mobile Phase A: Water (5 mM NH₄HCO₃), Mobile Phase B: acetonitrile; Flow rate: 45 mL/min; Gradient: 10% B to 60% B in 55 min; 254 nm. The fractions containing the desired product were collected at 40% B and concentrated under reduced pressure. This resulted in tert-butyl 4-(2-chloro-3-cyanophenoxy)-5H,6H,7H,8H-pyrido[3,4-d]pyrimidine-7-carboxylate (280 mg, 70.89%) as a light yellow solid.

### 2-Chloro-3-[5H,6H,7H,8H-pyrido[3,4-d]pyrimidin-4-yloxy]benzonitrile

To a stirred solution of tert-butyl 4-(2-chloro-3-cyanophenoxy)-5H,6H,7H,8H-pyrido[3,4-d]pyrimidine-7-carboxylate (100 mg, 0.259 mmol, 1 equiv.) in DCM (3 mL) was added TFA (1 mL). The resulting mixture was stirred for 2 hours at room temperature under air atmosphere. The resulting mixture was concentrated under reduced pressure. The mixture was basified to pH 7 with saturated NH₄HCO₃ (aq.). The mixture was purified by reverse flash chromatography with the following conditions: Column: spnerical C18, 20-40 um, 180 g; Mobile Phase A: Water (5 mM NH4HCO3), Mobile Phase B: acetonitrile; Flow rate: 45 mL/min; Gradient: 30% B to 60% B in 30 min; 254 nm). The fractions containing the desired product were collected at 45% B and concentrated under reduced pressure. This resulted in 2-chloro-3-[5H,6H,7H,8H-pyrido[3,4-d]pyrimidin-4-yloxy]benzonitrile (60 mg, 80.95%) as a light yellow oil.

### 2-Chloro-3-([7-[5-chloro-1-(oxan-2-yl)-6-oxo-1,6-dihydropyridazin-4-yl]-5H,6H,7H,8H-pyrido[3,4-d]pyrimidin-4-yl]oxy)benzonitrile

To a stirred solution of tert-butyl 4-(2-chloro-3-cyanophenoxy)-5H,6H,7H,8H-pyrido[3,4-d]pyrimidine-7-carboxylate (60 mg, 0.155 mmol, 1 equiv.) and 4,5-dichloro-2-(oxan-2-yl)-2,3-dihydropyridazin-3-one (38.63 mg, 0.155 mmol, 1.00 equiv.) in DIEA (40.09 mg, 0.310 mmol, 2 equiv). The resulting mixture was stirred for hours at 100° C. under air atmosphere. The residue was purified by Prep-TLC (PE/EtOAc 1:1) to afford 2-chloro-3-([7-[5-chloro-1-(oxan-2-yl)-6-oxo-1,6-dihydropyridazin-4-yl]-5H,6H,7H,8H-pyrido[3,4-d]pyrimidin-4-yl]oxy)benzonitrile (50 mg, 64.56%) as a light yellow solid.

### 2-Chloro-3-[[7-(5-chloro-6-oxo-1,6-dihydropyridazin-4-yl)-5H,6H,7H,8H-pyrido[3,4-d]pyrimidin-4-yl]oxy]benzonitrile

To a stirred solution of 2-chloro-3-([7-[5-chloro-1-(oxan-2-yl)-6-oxo-1,6-dihydropyridazin-4-yl]-5H,6H,7H,8H-pyrido[3,4-d]pyrimidin-4-yl]oxy)benzonitrile (50 mg, 0.100 mmol, 1 equiv.) in DCM (3 mL) was added TFA (1 mL). The resulting mixture was stirred for 2 hours at room temperature. The resulting mixture was concentrated under reduced pressure. The mixture was basified to pH 7 with saturated NH₄CO₃ (aq.). The crude product was purified by Prep-HPLC with the following conditions (Column: XBridge Prep OBD C18 Column 30×150 mm Sum; Mobile Phase A: Water (10 mM NH4HCO3), Mobile Phase B: acetonitrile; Flow rate: 60 mL/min; Gradient: 20% B to 42% B in 8 min; 220 nm; Rt: 7.58 min) to afford 2-chloro-3-[[7-(5-chloro-6-oxo-1,6-dihydropyridazin-4-yl)-5H,6H,7H,8H-pyrido[3,4-d]pyrimidin-4-yl]oxy]benzonitrile (14.5 mg, 34.88%) as an off-white solid.

### Example 14. Synthesis of Compound 111

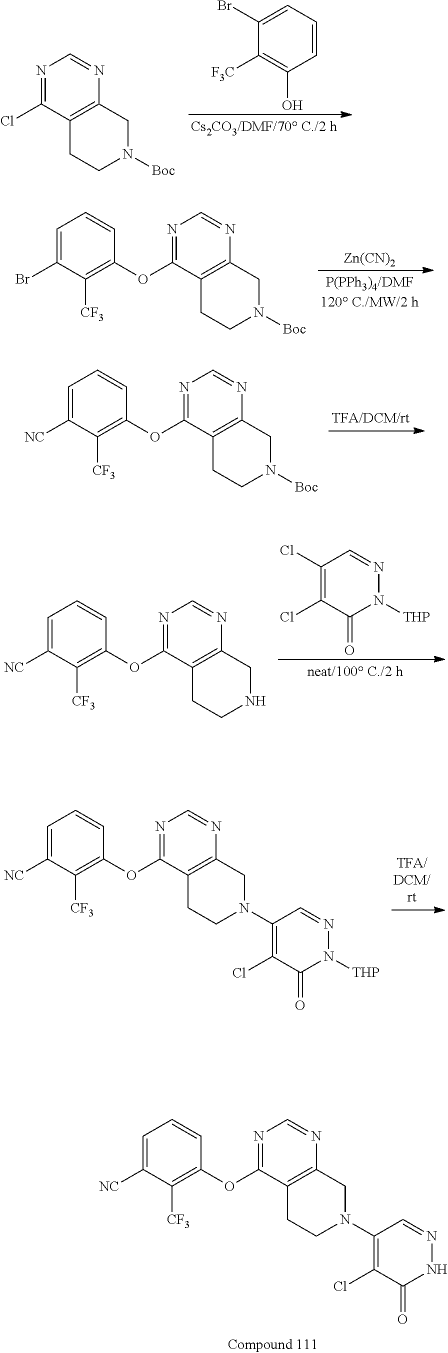

### Tert-Butyl 4-[3-bromo-2-(trifluoromethyl)phenoxy]-5H,6H,7H,8H-pyrido[3,4-d]pyrimidine-7-carboxylate

To a stirred mixture of tert-butyl 4-chloro-5H,6H,7H,8H-pyrido[3,4-d]pyrimidine-7-carboxylate (180 mg, 0.667 mmol, 1 equiv.) and 3-bromo-2-(trifluoromethyl)phenol (241.25 mg, 1.001 mmol, 1.50 equiv.) in DMF (10 mL) was added Cs₂CO₃ (434.86 mg, 1.335 mmol, 2.00 equiv.) in portions at rt under nitrogen atmosphere. The resulting mixture was stirred for 0.5 hours at 70° C. under nitrogen atmosphere. The reaction was monitored by LCMS. The mixture was allowed to cool down to rt. The resulting mixture was concentrated under reduced pressure. The resulting mixture was extracted with EtOAc (3×200 mL). The combined organic layers were washed with brine (2×100 mL), dried over anhydrous Na₂SO₄. After filtration, the filtrate was concentrated under reduced pressure. The residue was purified by reverse flash chromatography with the following conditions (Column, C18 silica gel; mobile phase, acetonitrile in water, 40% to 85% gradient in 30 min; detector, UV 220 nm) to afford tert-butyl 4-[3-bromo-2-(trifluoromethyl)phenoxy]-5H,6H,7H,8H-pyrido[3,4-d]pyrimidine-7-carboxylate (150 mg, 47.39%) as a yellow oil.

### Tert-Butyl 4-[3-cyano-2-(trifluoromethyl)phenoxy]-5H,6H,7H,8H-pyrido[3,4-d]pyrimidine-7-carboxylate

To a stirred mixture of tert-butyl 4-[3-bromo-2-(trifluoromethyl)phenoxy]-5H,6H,7H,8H-pyrido[3,4-d]pyrimidine-7-carboxylate (150 mg, 0.316 mmol, 1 equiv.) and Zn(CN)₂ (111.43 mg, 0.949 mmol, 3.00 equiv.) in DMF (8 mL) was added Pd(PPh₃)₄ (36.55 mg, 0.032 mmol, 0.1 equiv.) in portions at rt under nitrogen atmosphere. The final reaction mixture was irradiated with microwave radiation for 3 hours at 150° C. The reaction was monitored by LCMS. The residue was purified by reverse flash chromatography with the following conditions (Column, C18 silica gel; mobile phase, acetonitrile in water, 40% to 95% gradient in 30 min; detector, UV 220 nm) to afford tert-butyl4-[3-cyano-2-(trifluoromethyl)phenoxy]-5H,6H,7H,8H-pyrido[3,4-d]pyrimidine-7-carboxylate (70 mg, 52.65%) as a yellow oil.

### 3-[5H,6H,7H,8H-pyrido[3,4-d]pyrimidin-4-yloxy]-2-(trifluoromethyl)benzonitrile

To a stirred solution of tert-butyl 4-[3-cyano-2-(trifluoromethyl)phenoxy]-5H,6H,7H,8H-pyrido[3,4-d]pyrimidine-7-carboxylate (70 mg) in DCM (10 mL) was added TFA (1 mL) dropwise at rt. The reaction mixture was stirred for 2 hours at rt. The reaction was monitored by LCMS. The resulting mixture was concentrated under reduced pressure. The residue was basified to pH=8 with saturated NH₄HCO₃ (aq.). The resulting mixture was extracted with DCM (3×100 mL). The combined organic layers were washed with brine (1×100 mL), dried over anhydrous Na₂SO₄. After filtration, the filtrate was concentrated under reduced pressure. The residue was purified by reverse flash chromatography with the following conditions (Column, C18 silica gel; mobile phase, acetonitrile in water, 30% to 60% gradient in 20 min; detector, UV 220 nm) to afford 3-[5H,6H,7H,8H-pyrido[3,4-d]pyrimidin-4-yloxy]-2-(trifluoromethyl)benzonitrile (40 mg) as a yellow oil.

### 3-([7-[5-chloro-1-(oxan-2-yl)-6-oxo-1,6-dihydropyridazin-4-yl]-5H,6H,7H,8H-pyrido[3,4-d]pyrimidin-4-yl]oxy)-2-(trifluoromethyl)benzonitrile

Into a 25 mL round-bottom flask were added 3-[5H,6H,7H,8H-pyrido[3,4-d]pyrimidin-4-yloxy]-2-(trifluoromethyl)benzonitrile (40 mg, 0.125 mmol, 1 equiv), 4,5-dichloro-2-(oxan-2-yl)-2,3-dihydropyridazin-3-one (62.22 mg, 0.250 mmol, 2.00 equiv.) and DIEA (48.42 mg, 0.375 mmol, 3.00 equiv.) at rt under nitrogen atmosphere. The resulting mixture was stirred for 16 hours at 90° C. under nitrogen atmosphere. The residue was purified by Prep-TLC (PE/EtOAc=5/1) to afford 3-([7-[5-chloro-1-(oxan-2-yl)-6-oxo-1,6-dihydropyridazin-4-yl]-5H,6H,7H,8H-pyrido[3,4-d]pyrimidin-4-yl]oxy)-2-(trifluoromethyl)benzonitrile (50 mg, 75.12%) as a yellow oil.

### 3-[[7-(5-Chloro-6-oxo-1,6-dihydropyridazin-4-yl)-5H,6H,7H,8H-pyrido[3,4-d]pyrimidin-4-yl]oxy]-2-(trifluoromethyl)benzonitrile

To a stirred solution of 3-([7-[5-chloro-1-(oxan-2-yl)-6-oxo-1,6-dihydropyridazin-4-yl]-5H,6H,7H,8H-pyrido[3,4-d]pyrimidin-4-yl]oxy)-2-(trifluoromethyl)benzonitrile (50 mg) in DCM (10 mL) was added TFA (1 mL) dropwise at rt. The reaction mixture was stirred for 2 hours at rt. The reaction was monitored by LCMS. The resulting mixture was concentrated under reduced pressure. The residue was basified to pH=8 with saturated NH₄HCO₃ (aq.). The resulting mixture was extracted with DCM (3×100 mL). The combined organic layers were washed with brine (1×100 mL), dried over anhydrous Na₂SO₄. After filtration, the filtrate was concentrated under reduced pressure. The residue was purified by Prep-HPLC with the following conditions (Column: XBridge Prep OBD C18 Column 30×150 mm Sum; Mobile Phase A: Water (10 mM NH₄HCO₃), Mobile Phase B: acetonitrile; Flow rate: 60 mL/min; Gradient: 25% B to 45% B in 8 min; 220 nm; Rt: 7.07 min) to afford 3-[[7-(5-chloro-6-oxo-1,6-dihydropyridazin-4-yl)-5H,6H,7H,8H-pyrido[3,4-d]pyrimidin-4-yl]oxy]-2-(trifluoromethyl)benzonitrile (10.8 mg) as a white solid.

### Example 15. Synthesis of Compounds 126 and 126a

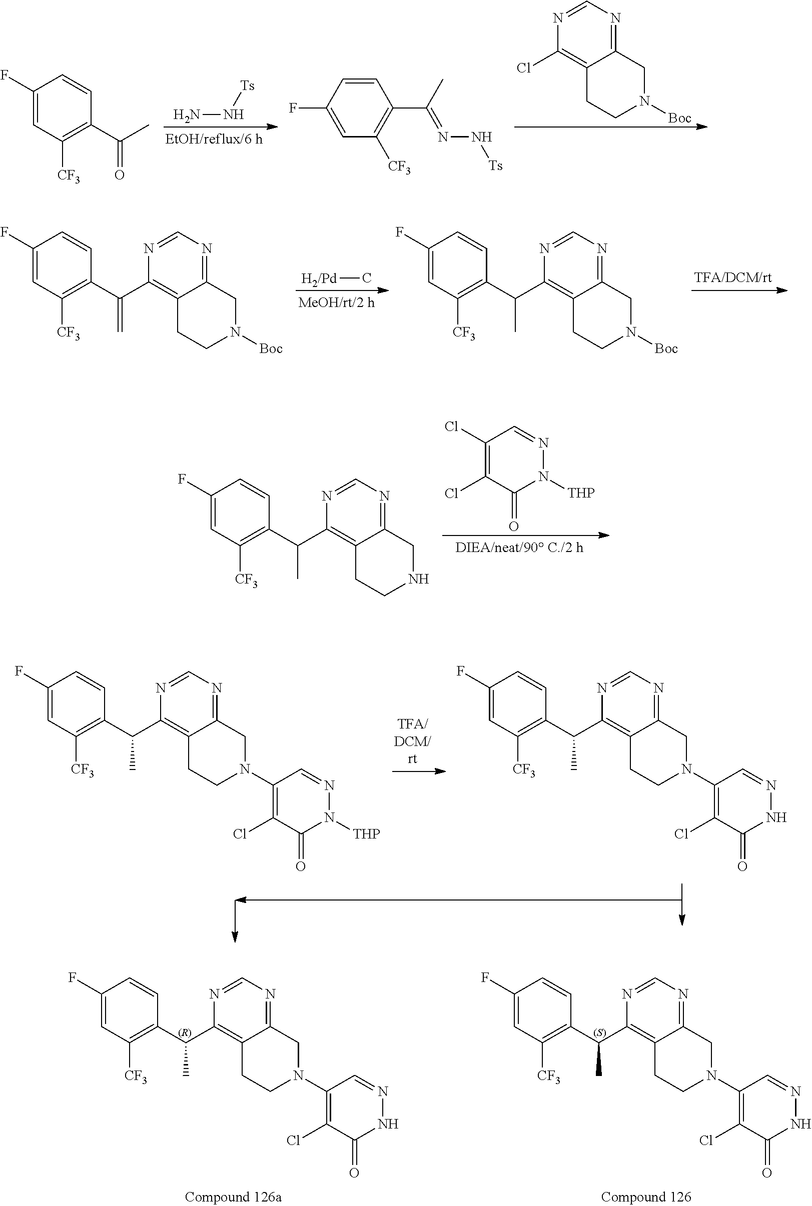

### N-[(1E)-1-[4-fluoro-2-(trifluoromethyl)phenyl]ethylidene]-4-methylbenzene-1-sulfonohydrazide

To a stirred solution of 1-[4-fluoro-2-(trifluoromethyl)phenyl]ethan-1-one (2 g, 9.702 mmol, 1 equiv.) in EtOH (40 mL) was added 4-methylbenzene-1-sulfonohydrazide (1.81 g, 9.719 mmol, 1.00 equiv.) in portions at rt under nitrogen atmosphere. The resulting mixture was stirred for 6 hours at 90° C. under nitrogen atmosphere. The reaction was monitored by LCMS. The mixture was allowed to cool down to rt. The resulting mixture was concentrated under vacuum. The residue was purified by reverse phase flash chromatography with the following conditions: Column: Spherical C18, 20-40 um, 330 g; Mobile Phase A: Water (plus 5 mM AcOH); Mobile Phase B: acetonitrile; Flow rate: 80 mL/min; Gradient: 5%-5% B, 10 min, 45% B-70% B gradient in 20 min; Detector: 220 nm. The fractions containing the desired product were collected at 60% B and concentrated under reduced pressure to afford N-[(1E)-1-[4-fluoro-2-(trifluoromethyl)phenyl]ethylidene]-4-methylbenzene-1-sulfonohydrazide (2.5 g, 68.83%) as a white solid.

### Tert-Butyl 4-[1-[4-fluoro-2-(trifluoromethyl)phenyl]ethenyl]-5H,6H,7H,8H-pyrido[3,4-d]pyrimidine-7-carboxylate

To a stirred mixture of tert-butyl 4-chloro-5H,6H,7H,8H-pyrido[3,4-d]pyrimidine-7-carboxylate (750 mg, 2.781 mmol, 1 equiv.) and N-[(1E)-1-[4-fluoro-2-(trifluoromethyl)phenyl]ethylidene]-4-methylbenzene-1-sulfonohydrazide (2081.80 mg, 5.561 mmol, 2.00 equiv.) in 1,4-dioxane (20 mL) were added Pd(acetonitrile)₂Cl₂ (72.14 mg, 0.278 mmol, 0.10 equiv), Dppf (307.18 mg, 0.556 mmol, 0.2 equiv.) and t-BuOLi (489.71 mg, 6.117 mmol, 2.20 equiv.) in portions at rt under nitrogen atmosphere. The final reaction mixture was irradiated with microwave radiation for 2 hours at 100° C. The reaction was monitored by LCMS. The mixture was allowed to cool down to rt. The resulting mixture was filtered, the filter cake was washed with EtOAc (2×50 mL). The filtrate was concentrated under reduced pressure. The residue was purified by reverse phase flash chromatography with the following conditions: Column: Spherical C18, 20-40 um, 330 g; Mobile Phase A: Water (plus 5 mM AcOH); Mobile Phase B: acetonitrile; Flow rate: 80 mL/min; Gradient: 5%-5% B, 10 min, 50% B-90% B gradient in 30 min; Detector: 220 nm. The fractions containing the desired product were collected at 85% B and concentrated under reduced pressure to afford tert-butyl 4-[1-[4-fluoro-2-(trifluoromethyl)phenyl]ethenyl]-5H,6H,7H,8H-pyrido[3,4-d]pyrimidine-7-carboxylate (800 mg, 67.95%) as a brown oil.

### Tert-Butyl 4-[1-[4-fluoro-2-(trifluoromethyl)phenyl]ethyl]-5H,6H,7H,8H-pyrido[3,4-d]pyrimidine-7-carboxylate

To a solution of tert-butyl 4-[1-[4-fluoro-2-(trifluoromethyl)phenyl]ethenyl]-5H,6H,7H,8H-pyrido[3,4-d]pyrimidine-7-carboxylate (150 mg) in 30 mL MeOH was added Pd/C (10%, 30 mg) under nitrogen atmosphere in a 100 mL round-bottom flask. The mixture was hydrogenated at room temperature for 4 hours under hydrogen atmosphere using a hydrogen balloon, filtered through a celite pad and concentrated under reduced pressure. This resulted in tert-butyl 4-[1-[4-fluoro-2-(trifluoromethyl)phenyl]ethyl]-5H,6H,7H,8H-pyrido[3,4-d]pyrimidine-7-carboxylate (150 mg) as a yellow oil.

### 4-[1-[4-Fluoro-2-(trifluoromethyl)phenyl]ethyl]-5H,6H,7H,8H-pyrido[3,4-d]pyrimidine

To a stirred solution of tert-butyl 4-[1-[4-fluoro-2-(trifluoromethyl)phenyl]ethyl]-5H,6H,7H,8H-pyrido[3,4-d]pyrimidine-7-carboxylate (150 mg) in DCM (10 mL) was added TFA (1 mL) dropwise at rt. The reaction mixture was stirred for 2 hours at rt. The reaction was monitored by LCMS. The resulting mixture was concentrated under reduced pressure. The residue was basified to pH=8 with saturated NH₄HCO₃ (aq.). The resulting mixture was extracted with DCM (3×50 mL). The combined organic layers were washed with brine (1×30 mL), dried over anhydrous Na₂SO₄. After filtration, the filtrate was concentrated under reduced pressure. The residue was purified by reverse phase flash chromatography with the following conditions: Column: Spherical C18, 20-40 um, 120 g; Mobile Phase A: Water (plus 5 mM AcOH); Mobile Phase B: acetonitrile; Flow rate: 45 mL/min; Gradient: 5%-5% B, 10 min, 40% B-58% B gradient in 15 min; Detector: 254 nm. The fractions containing the desired product were collected at 53% B and concentrated under reduced pressure to afford 4-[1-[4-fluoro-2-(trifluoromethyl)phenyl]ethyl]-5H,6H,7H,8H-pyrido[3,4-d]pyrimidine (100 mg) as a yellow oil.

### 4-chloro-5-(4-[1-[4-fluoro-2-(trifluoromethyl)phenyl]ethyl]-5H,6H,7H,8H-pyrido[3,4-d]pyrimidin-7-yl)-2-(oxan-2-yl)-2,3-dihydropyridazin-3-one

Into a 50 mL round-bottom flask were added 4-[1-[4-fluoro-2-(trifluoromethyl)phenyl]ethyl]-5H,6H,7H,8H-pyrido[3,4-d]pyrimidine (100 mg, 0.307 mmol, 1 equiv), 4,5-dichloro-2-(oxan-2-yl)-2,3-dihydropyridazin-3-one (91.88 mg, 0.369 mmol, 1.20 equiv.) and DIEA (119.19 mg, 0.922 mmol, 3.00 equiv.) at rt under nitrogen atmosphere. The resulting mixture was stirred for 2 hours at 90° C. under nitrogen atmosphere. The reaction was monitored by LCMS. The mixture was allowed to cool down to rt. The residue was purified by reverse phase flash chromatography with the following conditions: Column: Spherical C18, 20-40 um, 120 g; Mobile Phase A: Water (plus 5 mM AcOH); Mobile Phase B: acetonitrile; Flow rate: 45 mL/min; Gradient: 5%-5% B, 10 min, 40% B-60% B gradient in 15 min; Detector: 220 nm. The fractions containing the desired product were collected at 53% B and concentrated under reduced pressure to afford 4-chloro-5-(4-[1-[4-fluoro-2-(trifluoromethyl)phenyl]ethyl]-5H,6H,7H,8H-pyrido[3,4-d]pyrimidin-7-yl)-2-(oxan-2-yl)-2,3-dihydropyridazin-3-one (120 mg, 72.57%) as a yellow oil.

### 4-Chloro-5-[4-[(1S)-1-[4-fluoro-2-(trifluoromethyl)phenyl]ethyl]-5H,6H,7H,8H-pyrido[3,4-d]pyrimidin-7-yl]-2,3-dihydropyridazin-3-one and 4-chloro-5-[4-[(1R)-1-[4-fluoro-2-(trifluoromethyl)phenyl]ethyl]-5H,6H,7H,8H-pyrido[3,4-d]pyrimidin-7-yl]-2,3-dihydropyridazin-3-one

To a stirred solution of 4-chloro-5-(4-[1-[4-fluoro-2-(trifluoromethyl)phenyl]ethyl]-5H,6H,7H,8H-pyrido[3,4-d]pyrimidin-7-yl)-2-(oxan-2-yl)-2,3-dihydropyridazin-3-one (200 mg) in DCM (10 mL) was added TFA (1 mL) dropwise at rt. The reaction mixture was stirred for 4 hours at rt. The reaction was monitored by LCMS. The resulting mixture was concentrated under reduced pressure. The residue was basified to pH=8 with saturated NH₄HCO₃ (aq.). The resulting mixture was extracted with DCM (3×100 mL). The combined organic layers were washed with brine (1×100 mL), dried over anhydrous Na₂SO₄. After filtration, the filtrate was concentrated under reduced pressure. The residue was purified by Chiral-Prep-HPLC with the following conditions (Column: XBridge Prep Phenyl OBD Column 19×150 mm Sum 13 nm; Mobile Phase A: Mobile Phase B: Flow rate: 60 mL/min; Gradient: 20% B to 37% B in 8 min; 220 nm; Rt: 7.97 min). Although the two isomers were separated by this technique, the absolute orientation was not determined. The compound designated as 4-chloro-5-[4-[(1S)-1-[4-fluoro-2-(trifluoromethyl)phenyl]ethyl]-5H,6H,7H,8H-pyrido[3,4-d]pyrimidin-7-yl]-2,3-dihydropyridazin-3-one (11.8 mg) was obtained at 1.819 min as an off-white solid. The compound designated as 4-chloro-5-[4-[(1R)-1-[4-fluoro-2-(trifluoromethyl)phenyl]ethyl]-5H,6H,7H,8H-pyrido[3,4-d]pyrimidin-7-yl]-2,3-dihydropyridazin-3-one (13.5 mg) was obtained at 2.470 min as a white solid.

### Example 16. Synthesis of Compound 133

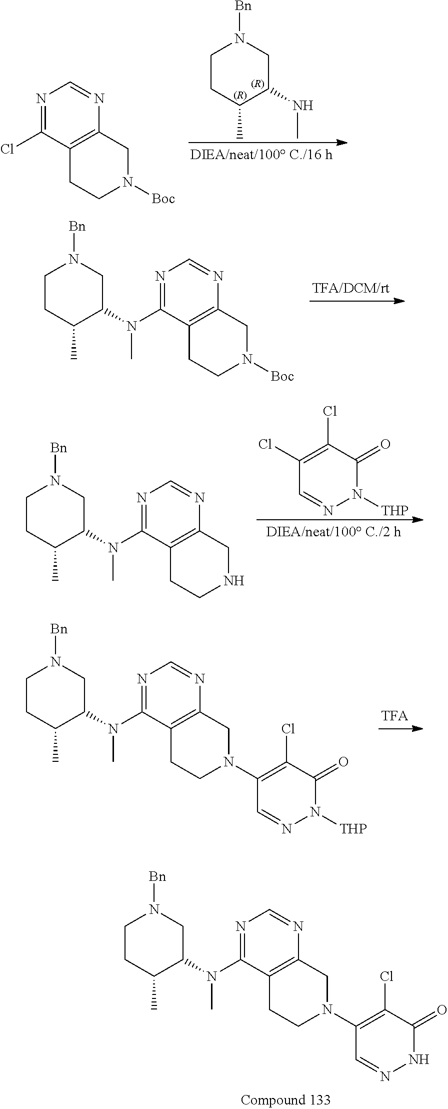

### tert-Butyl 4-[methyl[(3R,4R)-4-methylpiperidin-3-yl]amino]-5H,6H,7H,8H-pyrido[3,4-d]pyrimidine-7-carboxylate

Into a 25 mL round-bottom flask were added (3R,4R)-1-benzyl-N,4-dimethylpiperidin-3-amine (2.43 g, 0.011 mmol, 1.50 equiv.) and tert-butyl 4-chloro-5H,6H,7H,8H-pyrido[3,4-d]pyrimidine-7-carboxylate (2 g, 0.007 mmol, 1 equiv.) at room temperature. To the mixture was added DIEA (1.92 g, 0.015 mmol, 2.00 equiv.) at rt. The mixture was stirred at 100° C. for 2 hours. The residue was purified by Prep-TLC (PE/EtOAc 1:1) to afford tert-butyl 4-[methyl[(3R,4R)-4-methylpiperidin-3-yl]amino]-5H,6H,7H,8H-pyrido[3,4-d]pyrimidine-7-carboxylate (670 mg, 25.00%) as an off-white solid.

### (3R,4R)-1-Benzyl-N,4-dimethyl-N-[5H,6H,7H,8H-pyrido[3,4-d]pyrimidin-4-yl]piperidin-3-amine

To a stirred solution of tert-butyl 4-[[(3R,4R)-1-benzyl-4-methylpiperidin-3-yl](methyl)amino]-5H,6H,7H,8H-pyrido[3,4-d]pyrimidine-7-carboxylate (413 mg, 0.914 mmol, 1 equiv.) in DCM (10 mL) was added trifluoroacetic acid (3 mL, 0.026 mmol, 6.00 equiv.) dropwise at 0° C. The mixture was stirred for 2 hours at room temperature. The reaction was monitored by LCMS. The solution was concentrated under reduced pressure. The crude product (362 mg) was purified by Prep-HPLC with the following conditions (Column: XBridge Shield RP18 OBD Column, 5 um, 19*150 mm; Mobile Phase A: Water (10 mM NH₄HCO₃), Mobile Phase B: acetonitrile; Flow rate: 80 mL/min; Gradient: 30% B to 80% B in 25 min; 220 nm; Rt: 21.65 min) to afford (3R,4R)-1-benzyl-N,4-dimethyl-N-[5H,6H,7H,8H-pyrido[3,4-d]pyrimidin-4-yl]piperidin-3-amine (250 mg, 77.77%) as red oil.

### 5-(4-[[(3R,4R)-1-Benzyl-4-methylpiperidin-3-yl](methyl)amino]-5H,6H,7H,8H-pyrido[3,4-d]pyrimidin-7-yl)-4-chloro-2-(oxan-2-yl)-2,3-dihydropyridazin-3-one

Into a 25 mL round-bottom flask were added (3R,4R)-1-benzyl-N,4-dimethyl-N-[5H,6H,7H,8H-pyrido[3,4-d]pyrimidin-4-yl]piperidin-3-amine (263 mg, 0.748 mmol, 1 equiv.) and 4,5-dichloro-2-(oxan-2-yl)-2,3-dihydropyridazin-3-one (186.38 mg, 0.748 mmol, 1.00 equiv.) at room temperature. To the mixture was added DIEA (193.41 mg, 1.261 mmol, 2 equiv.) at rt. The mixture was stirred for 2 hours at 100° C. The residue was purified by reverse phase flash chromatography with the following conditions: Column: Spherical C18, 20-40 um, 330 g; Mobile Phase A: Water (plus 5 mM NH₄HCO₃); Mobile Phase B: acetonitrile; Flow rate: 80 mL/min; Gradient: 5%-5% B, 10 min, 45% B-95% B gradient in 30 min; Detector: 254 nm. The fractions containing the desired product were collected at 85% B and concentrated under reduced pressure to afford 5-(4-[[(3R,4R)-1-benzyl-4-methylpiperidin-3-yl](methyl)amino]-5H,6H,7H,8H-pyrido[3,4-d]pyrimidin-7-yl)-4-chloro-2-(oxan-2-yl)-2,3-dihydropyridazin-3-one (245 mg, 58.04%) as an off-white solid.

### 5-(4-[[(3R,4R)-1-Benzyl-4-methylpiperidin-3-yl](methyl)amino]-5H,6H,7H,8H-pyrido[3,4-d]pyrimidin-7-yl)-4-chloro-2,3-dihydropyridazin-3-one

To a stirred solution of 5-(4-[[(3R,4R)-1-benzyl-4-methylpiperidin-3-yl](methyl)amino]-5H,6H,7H,8H-pyrido[3,4-d]pyrimidin-7-yl)-4-chloro-2-(oxan-2-yl)-2,3-dihydropyridazin-3-one (88 mg, 1 equiv.) in DCM (10 mL) was added trifluoroacetic acid (3 mL, 0.026 mmol, 6.00 equiv.) dropwise at 0° C. The mixture was stirred for 2 hours at room temperature. The solution was concentrated under reduced pressure. The residue was purified by reverse phase flash chromatography with the following conditions: Column: Spherical C18, 20-40 um, 330 g; Mobile Phase A: Water (plus 5 mM TFA); Mobile Phase B: acetonitrile; Flow rate: 80 mL/min; Gradient: 5%-5% B, 10 min, 33% B-95% B gradient in 30 min; Detector: 254 nm. The fractions containing the desired product were collected at 90% B and concentrated under reduced pressure to afford 5-(4-[[(3R,4R)-1-benzyl-4-methylpiperidin-3-yl](methyl)amino]-5H,6H,7H,8H-pyrido[3,4-d]pyrimidin-7-yl)-4-chloro-2,3-dihydropyridazin-3-one (33.5 mg, 44.74%) as an off-white solid.

Compound 133a was prepared by the methods and scheme described in this example by using (3S,4S)-1-benzyl-N,4-dimethylpiperidin-3-amine in place of (3R,4R)-1-benzyl-N,4-dimethylpiperidin-3-amine.

### Example 17. Synthesis of Compound 136

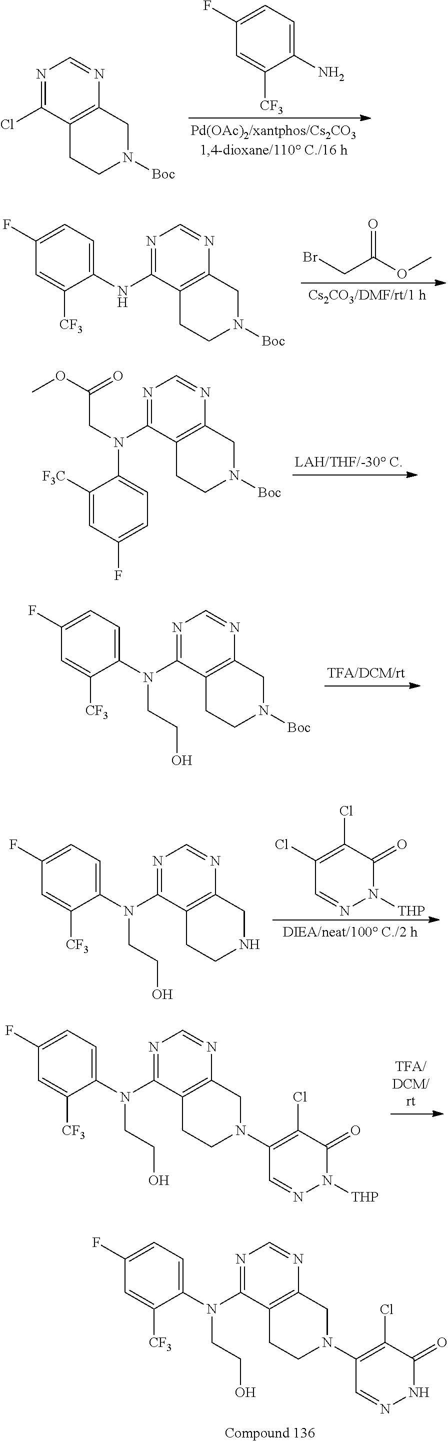

### Tert-Butyl 4-[[4-fluoro-2-(trifluoromethyl)phenyl]amino]-5H,6H,7H,8H-pyrido[3,4-d]pyrimidine-7-carboxylate

A mixture of 4-fluoro-2-(trifluoromethyl)aniline (6.64 g, 37.074 mmol, 2 equiv), tert-butyl 4-chloro-5H,6H,7H,8H-pyrido[3,4-d]pyrimidine-7-carboxylate (5 g, 18.537 mmol, 1 equiv), Pd(AcO)2 (0.83 g, 3.707 mmol, 0.2 equiv), XantPhos (4.29 g, 7.415 mmol, 0.4 equiv.) and Cs2CO3 (12.08 g, 37.074 mmol, 2 equiv.) in 1,4-dioxane (80 mL) was stirred at 110° C. for 16 hours. The reaction mixture was filtered and the filtrate was concentrated to give the crude product which was purified by silica gel column chromatography, eluted with PE:EA (20:1 to 1:2) to afford tert-butyl4-[[4-fluoro-2-(trifluoromethyl)phenyl]amino]-5H,6H,7H,8H-pyrido[3,4-d]pyrimidine-7-carboxylate (5.6 g, 73.26%) as a white solid.

### Tert-Butyl 4-[[4-fluoro-2-(trifluoromethyl)phenyl](2-methoxy-2-oxoethyl)amino]-5H,6H,7H,8H-pyrido[3,4-d]pyrimidine-7-carboxylate

To a stirred mixture of tert-butyl4-[[4-fluoro-2-(trifluoromethyl)phenyl]amino]-5H,6H,7H,8H-pyrido[3,4-d]pyrimidine-7-carboxylate (3 g, 7.275 mmol, 1 equiv.) and methyl 2-bromoacetate (2.23 g, 14.578 mmol, 2.00 equiv.) in DMF (30 mL) was added Cs₂CO₃ (4.74 g, 14.548 mmol, 2.00 equiv.) in portions at rt under nitrogen atmosphere. The resulting mixture was stirred for 2 hours at rt. The reaction was monitored by LCMS. The resulting mixture was extracted with EtOAc (3×400 mL). The combined organic layers were washed with brine (2×200 mL), dried over anhydrous Na₂SO₄. After filtration, the filtrate was concentrated under reduced pressure. The residue was purified by reverse phase flash chromatography with the following conditions: Column: Spherical C18, 20-40 um, 330 g; Mobile Phase A: Water (plus 5 mM TFA); Mobile Phase B: acetonitrile; Flow rate: 80 mL/min; Gradient: 5%-5% B, 10 min, 55% B-85% B gradient in 30 min; Detector: 220 nm. The fractions containing the desired product were collected at 79% B and concentrated under reduced pressure to afford tert-butyl 4-[[4-fluoro-2-(trifluoromethyl)phenyl](2-methoxy-2-oxoethyl)amino]-5H,6H,7H,8H-pyrido[3,4-d]pyrimidine-7-carboxylate (500 mg, 14.19%) as a yellow solid.

### Tert-Butyl 4-[[4-fluoro-2-(trifluoromethyl)phenyl](2-hydroxyethyl)amino]-5H,6H,7H,8H-pyrido[3,4-d]pyrimidine-7-carboxylate

To a stirred solution of tert-butyl 4-[[4-fluoro-2-(trifluoromethyl)phenyl](2-methoxy-2-oxoethyl)amino]-5H,6H,7H,8H-pyrido[3,4-d]pyrimidine-7-carboxylate (500 mg, 1.032 mmol, 1 equiv.) in THF (50 mL) was added LiAlH₄ (78.34 mg, 2.064 mmol, 2.00 equiv.) in portions at −30° C. under nitrogen atmosphere. The reaction mixture was stirred for 16 hours at rt. The reaction was monitored by LCMS. The reaction was quenched by the addition of Water (1 mL) at −30° C. The precipitated solids were collected by filtration and washed with MeOH (3×30 mL). The resulting mixture was concentrated under vacuum. The residue was purified by Prep-TLC (PE/EA=1/1) to afford tert-butyl 4-[[4-fluoro-2-(trifluoromethyl)phenyl](2-hydroxyethyl)amino]-5H,6H,7H,8H-pyrido[3,4-d]pyrimidine-7-carboxylate (100 mg, 21.23%) as a yellow oil.

### 4-[1-[4-Fluoro-2-(trifluoromethyl)phenyl]ethyl]-5H,6H,7H,8H-pyrido[3,4-d]pyrimidine

To a stirred solution of tert-butyl 4-[1-[4-fluoro-2-(trifluoromethyl)phenyl]ethyl]-5H,6H,7H,8H-pyrido[3,4-d]pyrimidine-7-carboxylate (150 mg) in DCM (10 mL) was added TFA (1 mL) dropwise at rt. The reaction mixture was stirred for 2 hours at rt. The reaction was monitored by LCMS. The resulting mixture was concentrated under reduced pressure. The residue was basified to pH=8 with saturated NH₄HCO₃ (aq.). The resulting mixture was extracted with DCM (3×50 mL). The combined organic layers were washed with brine (1×30 mL), dried over anhydrous Na₂SO₄. After filtration, the filtrate was concentrated under reduced pressure. The residue was purified by reverse phase flash chromatography with the following conditions: Column: Spherical C18, 20-40 um, 120 g; Mobile Phase A: Water (plus 5 mM NH₄HCO₃); Mobile Phase B: acetonitrile; Flow rate: 80 mL/min; Gradient: 5%-5% B, 10 min, 40% B-58% B gradient in 15 min; Detector: 220 nm. The fractions containing the desired product were collected at 53% B and concentrated under reduced pressure to afford 4-[1-[4-fluoro-2-(trifluoromethyl)phenyl]ethyl]-5H,6H,7H,8H-pyrido[3,4-d]pyrimidine (100 mg) as a yellow oil.

### 4-Chloro-5-(4-[[4-fluoro-2-(trifluoromethyl)phenyl](2-hydroxyethyl)amino]-5H,6H,7H,8H-pyrido[3,4-d]pyrimidin-7-yl)-2-(oxan-2-yl)-2,3-dihydropyridazin-3-one

Into a 50 mL round-bottom flask were added 2-[[4-fluoro-2-(trifluoromethyl)phenyl]([5H,6H,7H,8H-pyrido[3,4-d]pyrimidin-4-yl])amino]ethan-1-ol (40 mg, 0.112 mmol, 1 equiv), 4,5-dichloro-2-(oxan-2-yl)-2,3-dihydropyridazin-3-one (55.92 mg, 0.224 mmol, 2.00 equiv.) and DIEA (43.53 mg, 0.337 mmol, 3.00 equiv.) at rt under nitrogen atmosphere. The resulting mixture was stirred for 2 hours at 90° C. under nitrogen atmosphere. The reaction was monitored by LCMS. The mixture was allowed to cool down to rt. The residue was purified by reverse phase flash chromatography with the following conditions: Column: Spherical C18, 20-40 um, 120 g; Mobile Phase A: Water (plus 5 mM NH₄HCO₃); Mobile Phase B: acetonitrile; Flow rate: 45 mL/min; Gradient: 5%-5% B, 10 min, 40% B-60% B gradient in 15 min; Detector: 220 nm. The fractions containing the desired product were collected at 55% B and concentrated under reduced pressure to afford 4-chloro-5-(4-[[4-fluoro-2-(trifluoromethyl)phenyl](2-hydroxyethyl)amino]-5H,6H,7H,8H-pyrido[3,4-d]pyrimidin-7-yl)-2-(oxan-2-yl)-2,3-dihydropyridazin-3-one (50 mg, 78.28%) as a yellow oil.

### 4-Chloro-5-(4-[[4-fluoro-2-(trifluoromethyl)phenyl](2-hydroxyethyl)amino]-5H,6H,7H,8H-pyrido[3,4-d]pyrimidin-7-yl)-2,3-dihydropyridazin-3-one

To a stirred solution of 4-chloro-5-(4-[[4-fluoro-2-(trifluoromethyl)phenyl](2-hydroxyethyl)amino]-5H,6H,7H,8H-pyrido[3,4-d]pyrimidin-7-yl)-2-(oxan-2-yl)-2,3-dihydropyridazin-3-one (50 mg) in DCM (10 mL) was added TFA (1 mL) dropwise at rt. The reaction mixture was stirred for 2 hours at rt. The reaction was monitored by LCMS. The resulting mixture was concentrated under reduced pressure. The residue was basified to pH=8 with saturated NH₄HCO₃ (aq.). The resulting mixture was extracted with EtOAc (2×50 mL). The combined organic layers were washed with brine (1×100 mL), dried over anhydrous Na₂SO₄. After filtration, the filtrate was concentrated under reduced pressure. The residue was purified by Prep-HPLC with the following conditions (Column: XBridge Prep OBD C18 Column 30×150 mm Sum; Mobile Phase A: undefined, Mobile Phase B: undefined; Flow rate: 60 mL/min; Gradient: 30% B to 45% B in 8 min; 220 nm; Rt: 7.6 min) to afford 4-chloro-5-(4-[[4-fluoro-2-(trifluoromethyl)phenyl](2-hydroxyethyl)amino]-5H,6H,7H,8H-pyrido[3,4-d]pyrimidin-7-yl)-2,3-dihydropyridazin-3-one (6.2 mg) as a white solid.

### Example 18. Synthesis of Compound 132

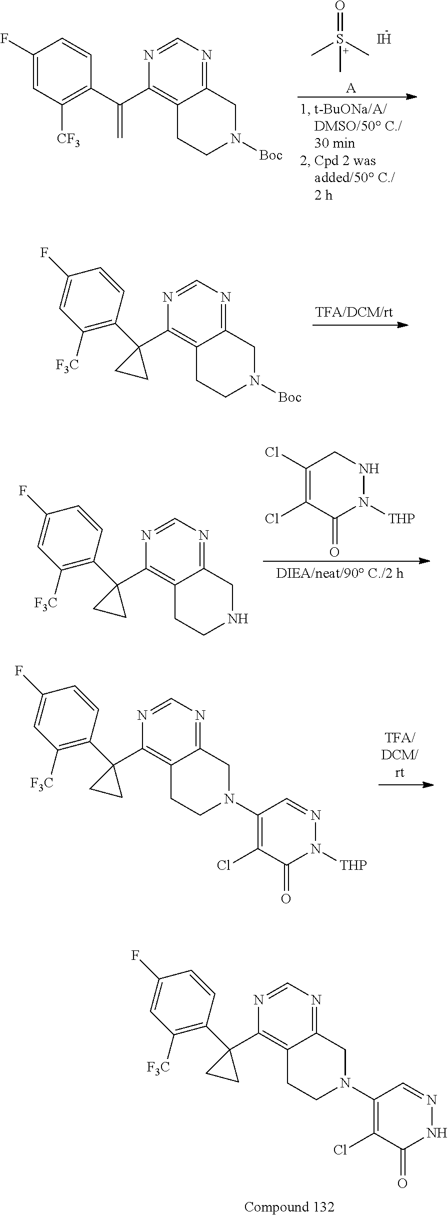

### Tert-Butyl 4-[1-[4-fluoro-2-(trifluoromethyl)phenyl]cyclopropyl]-5H,6H,7H,8H-pyrido[3,4-d]pyrimidine-7-carboxylate

To a stirred solution of t-BuONa (226.97 mg, 2.362 mmol, 2.00 equiv.) in DMSO (20 mL) was added Me3SiI (472.57 mg, 2.362 mmol, 2.00 equiv.) in portions at 40° C. under nitrogen atmosphere. The resulting mixture was stirred for 0.5 hours at 40° C. under nitrogen atmosphere. Then tert-butyl 4-[1-[4-fluoro-2-(trifluoromethyl)phenyl]ethenyl]-5H,6H,7H,8H-pyrido[3,4-d]pyrimidine-7-carboxylate (500 mg, 1.181 mmol, 1 equiv.) in DMSO (5 mL) was dropwise at rt under nitrogen atmosphere. The resulting mixture was stirred for 1 hours at rt under nitrogen atmosphere. The reaction was monitored by LCMS. The mixture was allowed to cool down to rt. The resulting mixture was extracted with EtOAc (3×100 mL). The combined organic layers were washed with brine (2×100 mL), dried over anhydrous Na₂SO₄. After filtration, the filtrate was concentrated under reduced pressure. The residue was purified by reverse phase flash chromatography with the following conditions: Column: Spherical C18, 20-40 um, 330 g; Mobile Phase A: Water (plus 5 mM NH₄HCO₃); Mobile Phase B: acetonitrile; Flow rate: 80 mL/min; Gradient: 5%-5% B, 10 min, 55% B-80% B gradient in 25 min; Detector: 220 nm. The fractions containing the desired product were collected at 73% B and concentrated under reduced pressure to afford tert-butyl 4-[1-[4-fluoro-2-(trifluoromethyl)phenyl]cyclopropyl]-5H,6H,7H,8H-pyrido[3,4-d]pyrimidine-7-carboxylate (240 mg, 46.46%) as a yellow oil.

### 4-[1-[4-Fluoro-2-(trifluoromethyl)phenyl]cyclopropyl]-5H,6H,7H,8H-pyrido[3,4-d]pyrimidine

To a stirred solution of tert-butyl 4-[1-[4-fluoro-2-(trifluoromethyl)phenyl]cyclopropyl]-5H,6H,7H,8H-pyrido[3,4-d]pyrimidine-7-carboxylate (240 mg, 0.549 mmol, 1 equiv.) in DCM (10 mL) was added TFA (1 mL, 13.463 mmol, 24.54 equiv.) dropwise at rt. The reaction mixture was stirred for 2 hours at rt. The reaction was monitored by LCMS. The resulting mixture was concentrated under reduced pressure. The residue was basified to pH=8 with saturated NH₄HCO₃ (aq.). The resulting mixture was extracted with EtOAc (2×100 mL). The combined organic layers were washed with brine (1×100 mL), dried over anhydrous Na₂SO₄. After filtration, the filtrate was concentrated under reduced pressure. The residue was purified by reverse phase flash chromatography with the following conditions: Column: Spherical C18, 20-40 um, 120 g; Mobile Phase A: Water (plus 5 mM AcOH); Mobile Phase B: acetonitrile; Flow rate: 45 mL/min; Gradient: 5%-5% B, 10 min, 33% B-45% B gradient in 20 min; Detector: 254 nm. The fractions containing the desired product were collected at 40% B and concentrated under reduced pressure to afford 4-[1-[4-fluoro-2-(trifluoromethyl)phenyl]cyclopropyl]-5H,6H,7H,8H-pyrido[3,4-d]pyrimidine (150 mg, 81.05%) as a yellow oil.

### 4-Chloro-5-(4-[1-[4-fluoro-2-(trifluoromethyl)phenyl]cyclopropyl]-5H,6H,7H,8H-pyrido[3,4-d]pyrimidin-7-yl)-2-(oxan-2-yl)-2,3-dihydropyridazin-3-one

Into a 50 mL round-bottom flask were added 4-[1-[4-fluoro-2-(trifluoromethyl)phenyl]cyclopropyl]-5H,6H,7H,8H-pyrido[3,4-d]pyrimidine (150 mg, 0.445 mmol, 1 equiv), 4,5-dichloro-2-(oxan-2-yl)-1,2,3,6-tetrahydropyridazin-3-one (134.00 mg, 0.534 mmol, 1.20 equiv.) and DIEA (172.42 mg, 1.334 mmol, 3.00 equiv.) at rt under nitrogen atmosphere. The resulting mixture was stirred for 2 hours at 90° C. under nitrogen atmosphere. The reaction was monitored by LCMS. The mixture was allowed to cool down to rt. The residue was purified by reverse phase flash chromatography with the following conditions: Column: Spherical C18, 20-40 um, 330 g; Mobile Phase A: Water (plus 5 mM NH₄HCO₃); Mobile Phase B: acetonitrile; Flow rate: 80 mL/min; Gradient: 5%-5% B, 10 min, 40% B-60% B gradient in 15 min; Detector: 220 nm. The fractions containing the desired product were collected at 54% B and concentrated under reduced pressure to afford 4-chloro-5-(4-[1-[4-fluoro-2-(trifluoromethyl)phenyl]cyclopropyl]-5H,6H,7H,8H-pyrido[3,4-d]pyrimidin-7-yl)-2-(oxan-2-yl)-2,3-dihydropyridazin-3-one (200 mg, 81.78%) as a yellow oil.

### 4-Chloro-5-(4-[1-[4-fluoro-2-(trifluoromethyl)phenyl]cyclopropyl]-5H,6H,7H,8H-pyrido[3,4-d]pyrimidin-7-yl)-2,3-dihydropyridazin-3-one

To a stirred solution of 4-chloro-5-(4-[1-[4-fluoro-2-(trifluoromethyl)phenyl]cyclopropyl]-5H,6H,7H,8H-pyrido[3,4-d]pyrimidin-7-yl)-2-(oxan-2-yl)-2,3-dihydropyridazin-3-one (200 mg) in DCM (10 mL) was added TFA (2 mL) dropwise at rt. The reaction mixture was stirred for 2 hours at rt. The reaction was monitored by LCMS. The resulting mixture was concentrated under reduced pressure. The residue was basified to pH=8 with saturated NH₄HCO₃ (aq.). The resulting mixture was extracted with EtOAc (2×100 mL). The combined organic layers were washed with brine (1×100 mL), dried over anhydrous Na₂SO₄. After filtration, the filtrate was concentrated under reduced pressure. The residue was purified by Prep-HPLC with the following conditions (Column: XBridge Prep OBD C18 Column 30×150 mm Sum; Mobile Phase A: undefined, Mobile Phase B: undefined; Flow rate: 60 mL/min; Gradient: 30% B to 55% B in 8 min; 220 nm; Rt: 7.232 min) to afford 4-chloro-5-(4-[1-[4-fluoro-2-(trifluoromethyl)phenyl]cyclopropyl]-5H,6H,7H,8H-pyrido[3,4-d]pyrimidin-7-yl)-2,3-dihydropyridazin-3-one (39.2 mg) as an off-white solid.

### Example 19. Synthesis of Compound 109

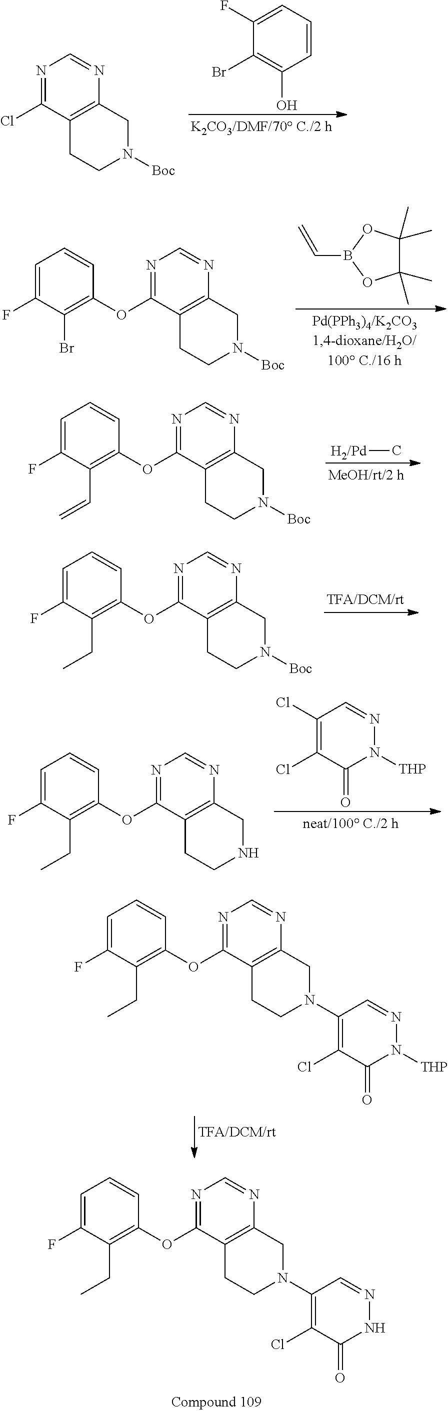

### Tert-Butyl 4-(2-bromo-3-fluorophenoxy)-5H,6H,7H,8H-pyrido[3,4-d]pyrimidine-7-carboxylate

To a stirred solution of tert-butyl 4-chloro-5H,6H,7H,8H-pyrido[3,4-d]pyrimidine-7-carboxylate (500 mg, 1.854 mmol, 1 equiv.) and 2-bromo-3-fluorophenol (424.87 mg, 2.224 mmol, 1.20 equiv.) in DMF (10 mL) was added K₂CO₃ (512.38 mg, 3.707 mmol, 2 equiv). The resulting mixture was stirred for 0.5 hours at 70° C. The mixture was allowed to cool down to room temperature. The reaction was quenched with Water at room temperature. The resulting mixture was extracted with EtOAc (2×100 mL). The combined organic layers were washed with brine (2×100 mL), dried over anhydrous Na₂SO₄. After filtration, the filtrate was concentrated under reduced pressure. The residue was purified by silica gel column chromatography, eluted with PE/EtOAc (5:1) to afford tert-butyl4-(2-bromo-3-fluorophenoxy)-5H,6H,7H,8H-pyrido[3,4-d]pyrimidine-7-carboxylate (500 mg, 63.58%) as a white solid.

### Tert-Butyl 4-(2-ethenyl-3-fluorophenoxy)-5H,6H,7H,8H-pyrido[3,4-d]pyrimidine-7-carboxylate

To a solution of tert-butyl 4-(2-bromo-3-fluorophenoxy)-5H,6H,7H,8H-pyrido[3,4-d]pyrimidine-7-carboxylate (500 mg, 1.178 mmol, 1 equiv.) and pentamethyl-1,3,2-dioxaborolane (334.72 mg, 2.357 mmol, 2.00 equiv.) in H₂O (2 mL) and 1,4-dioxane (16 mL) were added K₂CO₃ (325.75 mg, 2.357 mmol, 2 equiv.) and Pd(PPh₃)₄ (68.09 mg, 0.059 mmol, 0.05 equiv). After stirring for overnight at 90° C. under a nitrogen atmosphere, the resulting mixture was concentrated under reduced pressure. The residue was purified by silica gel column chromatography, eluted with PE/EtOAc (5:1) to afford tert-butyl4-(2-ethenyl-3-fluorophenoxy)-5H,6H,7H,8H-pyrido[3,4-d]pyrimidine-7-carboxylate (250 mg, 57.12%) as a yellow oil.

### Tert-Butyl 4-(2-ethyl-3-fluorophenoxy)-5H,6H,7H,8H-pyrido[3,4-d]pyrimidine-7-carboxylate

To a stirred solution of tert-butyl4-(2-ethenyl-3-fluorophenoxy)-5H,6H,7H,8H-pyrido[3,4-d]pyrimidine-7-carboxylate (250 mg, 0.673 mmol, 1 equiv.) in MeOH (10 mL) was added Pd/C (100 mg, 0.940 mmol, 1.40 equiv). The resulting mixture was stirred for 2 hours at RT under hydrogen atmosphere. The resulting mixture was filtered, the filter cake was washed with MeOH (2×10 mL). The filtrate was concentrated under reduced pressure. The resulting mixture was concentrated under reduced pressure. This resulted in tert-butyl 4-(2-ethyl-3-fluorophenoxy)-5H,6H,7H,8H-pyrido[3,4-d]pyrimidine-7-carboxylate (210 mg, 0.08%) as a black oil.

### 4-(2-Ethyl-3-fluorophenoxy)-5H,6H,7H,8H-pyrido[3,4-d]pyrimidine

To a stirred solution of tert-butyl 4-(2-ethyl-3-fluorophenoxy)-5H,6H,7H,8H-pyrido[3,4-d]pyrimidine-7-carboxylate (210 mg, 0.562 mmol, 1 equiv.) in DCM (3 mL) was added TFA (1 mL). The resulting mixture was stirred for 2 hours at room temperature under air atmosphere. The resulting mixture was concentrated under reduced pressure. The mixture was basified to pH 8 with saturated NH₄HCO₃ (aq.). The mixture was purified by reverse flash chromatography with the following conditions: Column: spnerical C18, 20-40 um, 180 g; Mobile Phase A: Water (5 mM NH₄HCO₃), Mobile Phase B: acetonitrile; Flow rate: 45 mL/min; Gradient: 25% B to 60% B in 40 min; 254 nm). The fractions containing the desired product were collected at 40% B and concentrated under reduced pressure. This resulted in 4-(2-ethyl-3-fluorophenoxy)-5H,6H,7H,8H-pyrido[3,4-d]pyrimidine (120 mg, 78.07%) as a light yellow oil.

### 4-Chloro-5-[4-(2-ethyl-3-fluorophenoxy)-5H,6H,7H,8H-pyrido[3,4-d]pyrimidin-7-yl]-2-(oxan-2-yl)-2,3-dihydropyridazin-3-one

To a stirred solution of 4-(2-ethyl-3-fluorophenoxy)-5H,6H,7H,8H-pyrido[3,4-d]pyrimidine (120 mg, 0.439 mmol, 1 equiv.) and 4,5-dichloro-2-(oxan-2-yl)-2,3-dihydropyridazin-3-one (109.37 mg, 0.439 mmol, 1.00 equiv.) in DIEA (113.49 mg, 0.878 mmol, 2 equiv). The resulting mixture was stirred for 2 hours at 100° C. under air atmosphere. The residue was purified by Prep-TLC (PE/EtOAc 1:1) to afford 4-chloro-5-[4-(2-ethyl-3-fluorophenoxy)-5H,6H,7H,8H-pyrido[3,4-d]pyrimidin-7-yl]-2-(oxan-2-yl)-2,3-dihydropyridazin-3-one (100 mg, 46.87%) as a light yellow solid.

### 4-Chloro-5-[4-(2-ethyl-3-fluorophenoxy)-5H,6H,7H,8H-pyrido[3,4-d]pyrimidin-7-yl]-2,3-dihydropyridazin-3-one

To a stirred solution of 4-chloro-5-[4-(2-ethyl-3-fluorophenoxy)-5H,6H,7H,8H-pyrido[3,4-d]pyrimidin-7-yl]-2-(oxan-2-yl)-2,3-dihydropyridazin-3-one (100 mg, 0.206 mmol, 1 equiv.) in DCM (3 mL) was added TFA (1 mL). The resulting mixture was stirred for 2 hours at room temperature. The resulting mixture was concentrated under reduced pressure. The mixture was basified to pH 7 with saturated NH₄HCO₃ (aq.). The crude product was purified by Prep-HPLC with the following conditions (Column: XBridge Prep OBD C18 Column 30×150 mm Sum; Mobile Phase A: Water (10 mM NH₄HCO₃), Mobile Phase B: acetonitrile; Flow rate: 60 mL/min; Gradient: 30% B to 50% B in 8 min; 220 nm; Rt: 7.27 min) to afford 4-chloro-5-[4-(2-ethyl-3-fluorophenoxy)-5H,6H,7H,8H-pyrido[3,4-d]pyrimidin-7-yl]-2,3-dihydropyridazin-3-one (41.6 mg, 50.31%) as a white solid.

### Example 20. Synthesis of Compound 127

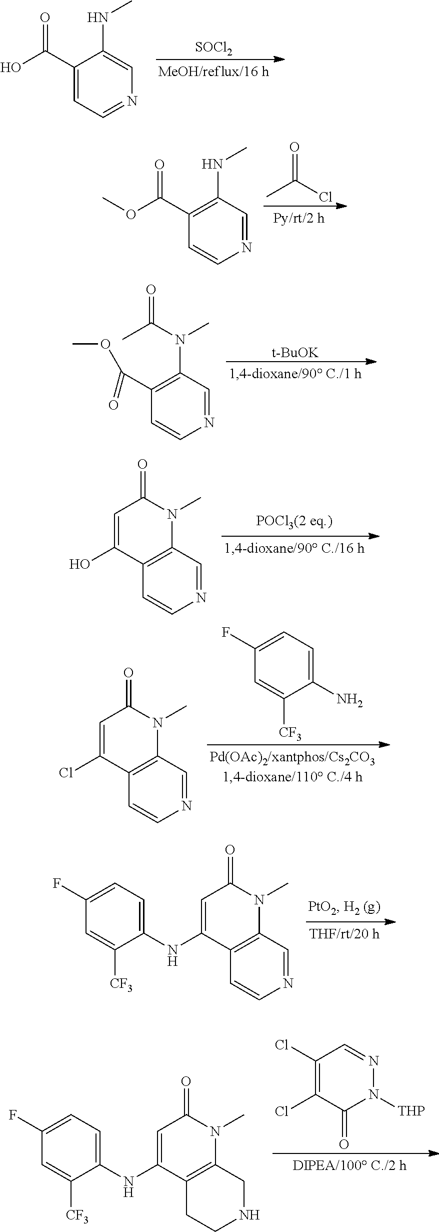

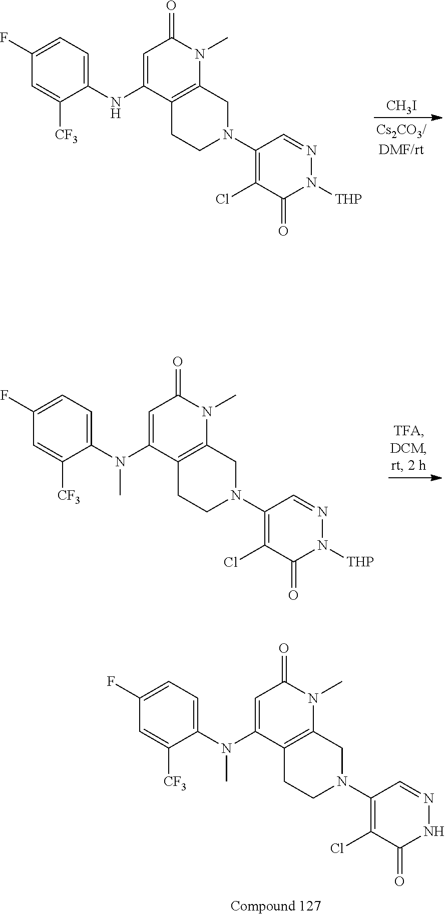

### Methyl 3-(methylamino)pyridine-4-carboxylate

To a stirred solution of 3-(methylamino)pyridine-4-carboxylic acid (11 g, 72.296 mmol, 1 equiv.) in MeOH (500 mL, 12349.455 mmol, 170.82 equiv.) was added SOCl₂ (43.01 g, 361.478 mmol, 5 equiv.) dropwise at 0° C. The resulting mixture was stirred for 30 hours at 70° C. The reaction was monitored by LCMS. The mixture was allowed to cool down to room temperature. The resulting mixture was concentrated under reduced pressure. The residue was dissolved in ethyl acetate (50 mL). The mixture basified to pH 8 with saturated NaHCO₃(aq.). The resulting mixture was extracted with EtOAc (2×20 mL). The combined organic layers were washed with brine (1×30 mL), dried over anhydrous Na₂SO₄. After filtration, the filtrate was concentrated under reduced pressure to afford methyl 3-(methylamino)pyridine-4-carboxylate (9 g, crude) as a yellow solid.

### Methyl 3-(N-methylacetamido)pyridine-4-carboxylate

To a stirred solution of methyl 3-(methylamino)pyridine-4-carboxylate (9 g, 54.158 mmol, 1 equiv.) in DCM (100 mL) were added Pyridine (21.42 g, 270.791 mmol, 5 equiv.) and acetyl chloride (6.38 g, 81.237 mmol, 1.5 equiv.) dropwise at room temperature. The resulting mixture was stirred for 2 hours at room temperature. The reaction was monitored by LCMS. The solution was basified to pH 8 with saturated NaHCO₃(aq.). The resulting mixture was concentrated under reduced pressure. The residue was purified by reverse phase flash with the following conditions (Column: C18 Column 330 g; Mobile Phase A: Water (10 mM NH₄HCO₃), Mobile Phase B: acetonitrile; Flow rate: 80 mL/min; Gradient: 10% B to 30% B in 25 min; 254/220 nm) to afford methyl 3-(N-methylacetamido)pyridine-4-carboxylate (8 g, 70.94%) as a brown liquid.

### 4-Hydroxy-1-methyl-1,2-dihydro-1,7-naphthyridin-2-one

To a stirred solution of methyl 3-(N-methylacetamido)pyridine-4-carboxylate (6 g, 28.816 mmol, 1 equiv.) in dry 1,4-dioxane (100 mL) was added t-BuOK (6.47 g, 57.632 mmol, 2 equiv.) at room temperature under nitrogen atmosphere. The resulting mixture was stirred for 1 hours at 90° C. under nitrogen atmosphere. The reaction was monitored by LCMS. The mixture was allowed to cool down to room temperature. The resulting mixture was concentrated under vacuum. The residue was purified by silica gel column chromatography, eluted with DCM/MeOH (10:1) to afford 4-hydroxy-1-methyl-1,2-dihydro-1,7-naphthyridin-2-one (4.5 g, 88.64%) as a orange solid.

### 4-Chloro-1-methyl-1,2-dihydro-1,7-naphthyridin-2-one

To a stirred solution of 4-hydroxy-1-methyl-1,2-dihydro-1,7-naphthyridin-2-one (4.5 g, 25.543 mmol, 1 equiv.) in dry 1,4-dioxane (100 mL) was added POCl₃ (3.92 g, 25.543 mmol, 1 equiv.) dropwise at room temperature. The resulting mixture was stirred for 16 hours at 90° C. The mixture was allowed to cool down to room temperature. The resulting mixture was concentrated under reduced pressure. The residue was purified by silica gel column chromatography, eluted with DCM/MeOH (10:1) to afford 4-chloro-1-methyl-1,2-dihydro-1,7-naphthyridin-2-one (2 g, 40.23%) as a red solid.

### 4-[[4-Fluoro-2-(trifluoromethyl)phenyl]amino]-1-methyl-1,2-dihydro-1,7-naphthyridin-2-one

To a stirred solution of 4-chloro-1-methyl-1,2-dihydro-1,7-naphthyridin-2-one (0.8 g, 4.111 mmol, 1 equiv.) in dry 1,4-dioxane (15 mL) were added Cs₂CO₃ (2.68 g, 8.221 mmol, 2 equiv), 4-fluoro-2-(trifluoromethyl)aniline (1.47 g, 8.221 mmol, 2.00 equiv), XantPhos (0.95 g, 1.644 mmol, 0.4 equiv.) and Pd(AcO)₂ (0.18 g, 0.822 mmol, 0.2 equiv.) at room temperature under nitrogen atmosphere. The final reaction mixture was irradiated with microwave radiation for 4 hours at 110° C. The reaction was monitored by LCMS. The resulting mixture was extracted with EtOAc (3×50 mL). The combined organic layers were washed with brine (1×100 mL), dried over anhydrous Na₂SO₄. After filtration, the filtrate was concentrated under reduced pressure. The residue was purified by reverse phase flash with the following conditions (Column: C18 Column 330 g; Mobile Phase A: Water (10 mM AcOH), Mobile Phase B: acetonitrile; Flow rate: 50 mL/min; Gradient: 20% B to 40% B in 40 min; 254/220 nm) to afford 4-[[4-fluoro-2-(trifluoromethyl)phenyl]amino]-1-methyl-1,2-dihydro-1,7-naphthyridin-2-one (1.1 g, 79.34%) as an off-white solid.

### 4-[[4-Fluoro-2-(trifluoromethyl)phenyl]amino]-1-methyl-1,2,5,6,7,8-hexahydro-1,7-naphthyridin-2-one

To a stirred solution of 4-[[4-fluoro-2-(trifluoromethyl)phenyl]amino]-1-methyl-1,2-dihydro-1,7-naphthyridin-2-one (1 g, 2.965 mmol, 1 equiv.) in THF (20 mL) was added PtO₂ (67.33 mg, 0.296 mmol, 0.10 equiv.) at room temperature under nitrogen atmosphere. The resulting mixture was stirred for 16 hours at room temperature under hydrogen atmosphere. The reaction was monitored by LCMS. The resulting mixture was filtered, the filter cake was washed with EtOAc (3×20 mL). The filtrate was concentrated under reduced pressure. The resulting mixture was concentrated under reduced pressure. The residue was purified by reverse phase flash with the following conditions (Column: C18 Column 330 g; Mobile Phase A: Water (10 mM AcOH), Mobile Phase B: acetonitrile; Flow rate: 80 mL/min; Gradient: 5% B to 20% B in 40 min; 254/220 nm) The fractions containing the desired product were collected at 16% B and concentrated under reduced pressure to afford 4-[[4-fluoro-2-(trifluoromethyl)phenyl]amino]-1-methyl-1,2,5,6,7,8-hexahydro-1,7-naphthyridin-2-one (750 mg, 74.11%) as an off-white solid.

### 7-[5-chloro-1-(oxan-2-yl)-6-oxo-1,6-dihydropyridazin-4-yl]-4-[[4-fluoro-2-(trifluoromethyl)phenyl]amino]-1-methyl-1,2,5,6,7,8-hexahydro-1,7-naphthyridin-2-one

To a stirred mixture of 4-[[4-fluoro-2-(trifluoromethyl)phenyl]amino]-1-methyl-1,2,5,6,7,8-hexahydro-1,7-naphthyridin-2-one (750 mg, 2.197 mmol, 1 equiv.) and 4,5-dichloro-2-(oxan-2-yl)-2,3-dihydropyridazin-3-one (1.09 g, 4.395 mmol, 2 equiv.) was added DIPEA (568.00 mg, 4.395 mmol, 2 equiv.) at room temperature. The resulting mixture was stirred for 2 hours at 100° C. The reaction was monitored by LCMS. The residue was dissolved in DMF (10 mL). The solution was purified by reverse phase flash with the following conditions (Column: C18 Column 330 g; Mobile Phase A: Water (10 mM FA), Mobile Phase B: acetonitrile; Flow rate: 80 mL/min; Gradient: 30% B to 50% B in 40 min; 254/220 nm). The fractions containing the desired product were collected at 44% B and concentrated under reduced pressure to afford 7-[5-chloro-1-(oxan-2-yl)-6-oxo-1,6-dihydropyridazin-4-yl]-4-[[4-fluoro-2-(trifluoromethyl)phenyl]amino]-1-methyl-1,2,5,6,7,8-hexahydro-1,7-naphthyridin-2-one (1 g, 82.15%) as a yellow oil.

### 7-[5-Chloro-1-(oxan-2-yl)-6-oxo-1,6-dihydropyridazin-4-yl]-4-[[4-fluoro-2-(trifluoromethyl)phenyl](methyl)amino]-1-methyl-1,2,5,6,7,8-hexahydro-1,7-naphthyridin-2-one

To a stirred solution of 7-[5-chloro-1-(oxan-2-yl)-6-oxo-1,6-dihydropyridazin-4-yl]-4-[[4-fluoro-2-(trifluoromethyl)phenyl]amino]-1-methyl-1,2,5,6,7,8-hexahydro-1,7-naphthyridin-2-one (800 mg, 1.444 mmol, 1 equiv.) in IMF (20 mL) were added Cs₂CO₃ (0.94 g, 2.888 mmol, 2 equiv.) and MeI (614.96 mg, 4.333 mmol, 3 equiv.) at room temperature. The resulting mixture was stirred for 16 hours at room temperature. The reaction was monitored by LCMS. The mixture was purified by reverse phase flash with the following conditions (Column: C18 Column 120 g; Mobile Phase A: Water (10 mM AcOH), Mobile Phase B: acetonitrile; Flow rate: 60 mL/min; Gradient: 40% B to 60% B in 40 min; 254/220 nm). The fractions containing the desired product were collected at 49% B and concentrated under reduced pressure to afford 7-[5-chloro-1-(oxan-2-yl)-6-oxo-1,6-dihydropyridazin-4-yl]-4-[[4-fluoro-2-(trifluoromethyl)phenyl](methyl)amino]-1-methyl-1,2,5,6,7,8-hexahydro-1,7-naphthyridin-2-one (80 mg, 9.75%) as a yellow oil.

### 7-(5-Chloro-6-oxo-1,6-dihydropyridazin-4-yl)-4-[[4-fluoro-2-(trifluoromethyl)phenyl](methyl)amino]-1-methyl-1,2,5,6,7,8-hexahydro-1,7-naphthyridin-2-one

To a stirred solution of 7-[5-chloro-1-(oxan-2-yl)-6-oxo-1,6-dihydropyridazin-4-yl]-4-[[4-fluoro-2-(trifluoromethyl)phenyl](methyl)amino]-1-methyl-1,2,5,6,7,8-hexahydro-1,7-naphthyridin-2-one (80 mg, 0.141 mmol, 1 equiv.) in DCM (4.5 mL) was added TFA (0.5 mL, 6.732 mmol, 31.07 equiv.) dropwise at room temperature. The resulting mixture was stirred for 2 hours at room temperature. The reaction was monitored by LCMS. The resulting mixture was concentrated under reduced pressure. The residue was basified to pH 8 with saturated NaHCO₃(aq.). The solution was purified by reverse phase flash to afford 7-(5-chloro-6-oxo-1,6-dihydropyridazin-4-yl)-4-[[4-fluoro-2-(trifluoromethyl)phenyl](methyl)amino]-1-methyl-1,2,5,6,7,8-hexahydro-1,7-naphthyridin-2-one (40 mg, 58.69%) as a white solid.

### Example 21. Synthesis of Compounds 135 and 137

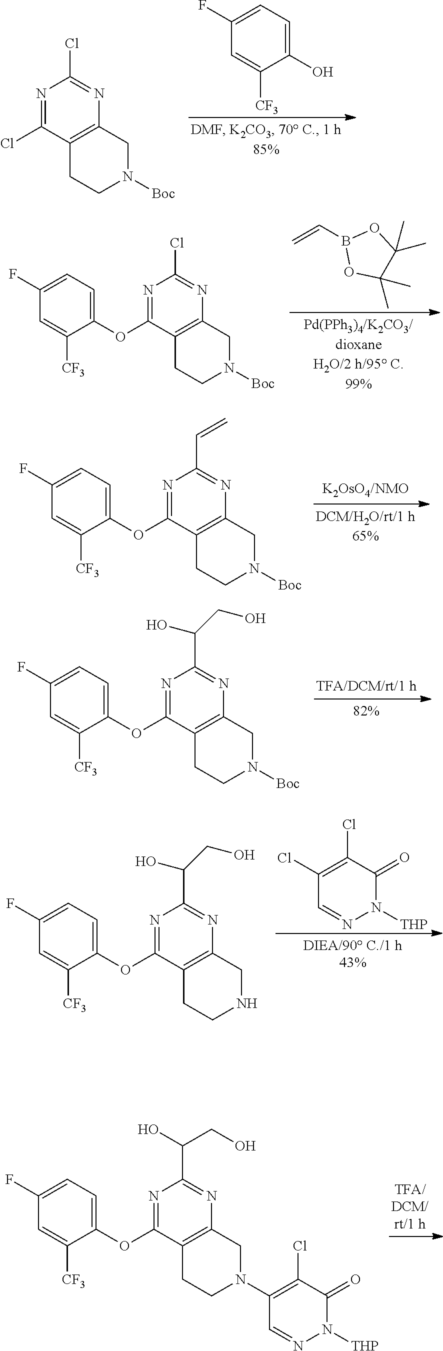

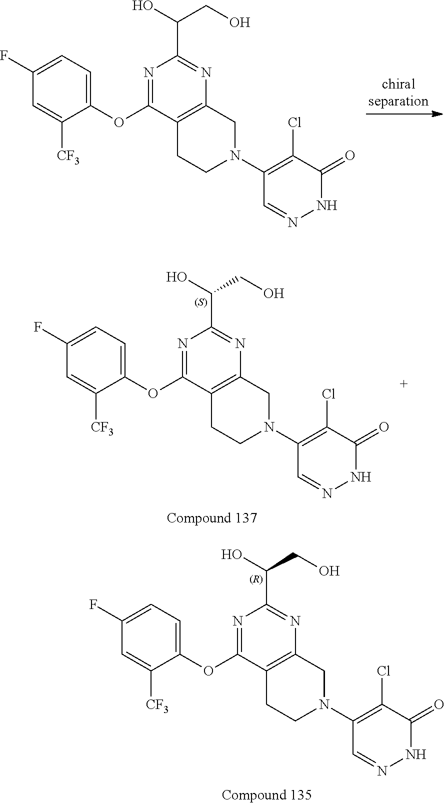

### Tert-butyl 2-chloro-4-(4-fluoro-2-(trifluoromethyl)phenoxy)-5,8-dihydropyrido[3,4-d]pyrimidine-7(6H)-carboxylate

To a solution of tert-butyl2,4-dichloro-5H,6H,7H,8H-pyrido[3,4-d]pyrimidine-7-carboxylate (5 g, 16.44 mmol) in DMF (50 mL) were added 4-fluoro-2-(trifluoromethyl)phenol (4.44 g, 24.66 mmol) and added K₂CO₃ (3.41 g, 24.66 mmol) at room temperature. The resulting mixture was stirred for 1 hours at 70° C. After cooling to room temperature. A filtration was performed and the filtrate was concentrated under reduced pressure. The residue was purified by reverse phase flash chromatography with the following conditions (Column: Spherical C18, 20˜40 um, 330 g; Mobile Phase A: Water (plus 5 mM NH₄HCO₃; Mobile Phase B: acetonitrile; Flow rate: 80 mL/min; Gradient: 5% in 10 min, 35% B to 45% B in 10 min; Detector: 254 nm/220 nm. The fractions containing desired product were collected at 44% B and concentrated under reduced pressure to afford tert-butyl 2-chloro-4-[4-fluoro-2-(trifluoromethyl)phenoxy]-5H,6H,7H,8H-pyrido[3,4-d]pyrimidine-7-carboxylate (6.2 g, 85%) as a white solid.

### Tert-butyl 4-(4-fluoro-2-(trifluoromethyl)phenoxy)-2-vinyl-5,8-dihydropyrido[3,4-d]pyrimidine-7(6H)-carboxylate

To a solution of tert-butyl 2-chloro-4-[4-fluoro-2-(trifluoromethyl)phenoxy]-5H,6H,7H,8H-pyrido[3,4-d]pyrimidine-7-carboxylate (500 mg, 1.12 mmol) in dioxane (10 mL) were added 2-ethenyl-4,4,5,5-tetramethyl-1,3,2-dioxaborolane (344 mg, 2.23 mmol) and H₂O (0.5 mL, 27.75 mmol) K₂CO₃ (309 mg, 2.23 mmol) and Pd(PPh₃)₄ (129 mg, 0.11 mmol). After stirring for 2 hours at 95° C. under a nitrogen atmosphere, the resulting mixture was concentrated under reduced pressure. The residue was purified by Prep-TLC, eluted with 17% ethyl acetate in petroleum ether to afford tert-butyl 2-ethenyl-4-[4-fluoro-2-(trifluoromethyl)phenoxy]-5H,6H,7H,8H-pyrido[3,4-d]pyrimidine-7-carboxylate (490 mg, 99%) as a light yellow solid.

### Tert-butyl 2-(1,2-dihydroxyethyl)-4-(4-fluoro-2-(trifluoromethyl)phenoxy)-5,8-dihydropyrido[3,4-d]pyrimidine-7(6H)-carboxylate

To a solution of tert-butyl 2-ethenyl-4-[4-fluoro-2-(trifluoromethyl)phenoxy]-5H,6H,7H,8H-pyrido[3,4-d]pyrimidine-7-carboxylate (400 mg, 0.91 mmol) in DCM (20 mL) were added 4-hydroxy-4-methylmorpholin-4-ium (323 mg, 2.73 mmol) and K₂OsO₄.2H₂O (34 mg, 0.091 mmol) at room temperature. After stirring for additional 1 hour, the resulting mixture was concentrated under reduced pressure and the residue was purified by reverse phase flash chromatography with the following conditions: Column: Spherical C18, 20˜40 um, 120 g; Mobile Phase A: Water (plus 5 mM NH₄HCO₃; Mobile Phase B: acetonitrile; Flow rate: 45 mL/min; Gradient: 5% B in 10 min, 45% B to 65% B in 15 min; Detector: 254 nm and 220 nm. The fractions containing desired product were collected at 64% B and concentrated under reduced pressure to afford tert-butyl 2-(1,2-dihydroxyethyl)-4-[4-fluoro-2-(trifluoromethyl)phenoxy]-5H,6H,7H,8H-pyrido[3,4-d]pyrimidine-7-carboxylate (280 mg, 65%) as a white solid.

### 1-(4-(4-fluoro-2-(trifluoromethyl)phenoxy)-5,6,7,8-tetrahydropyrido[3,4-d]pyrimidin-2-yl)ethane-1,2-diol

To a stirred solution of tert-butyl 2-(1,2-dihydroxyethyl)-4-[4-fluoro-2-(trifluoromethyl)phenoxy]-5H,6H,7H,8H-pyrido[3,4-d]pyrimidine-7-carboxylate (280 mg, 0.59 mmol) in DCM (4 mL) was added TFA (1 mL) at room temperature. The resulting mixture was stirred for 1 hours at room temperature. The resulting mixture was concentrated under vacuum. The residue was dissolved into DCM (50 mL) and washed with saturated aqueous NaHCO₃ (20 mL) the organic layer was separated out and dried over anhydrous sodium sulfate. After filtration, the filtrate was concentrated under reduced pressure and the residue was purified by Prep-TLC with 8% methanol in dichloromethane to afford 1-[4-[4-fluoro-2-(trifluoromethyl)phenoxy]-5H,6H,7H,8H-pyrido[3,4-d]pyrimidin-2-yl]ethane-1,2-diol (180 mg, 82%) as a brown solid.

### 4-chloro-5-(2-(1,2-dihydroxyethyl)-4-(4-fluoro-2-(trifluoromethyl)phenoxy)-5,8-dihydropyrido[3,4-d]pyrimidin-7(6H)-yl)-2-(tetrahydro-2H-pyran-2-yl)pyridazin-3(2H)-one

To a stirred solution of 2-[5H,6H,7H,8H-pyrido[3,4-d]pyrimidin-4-yloxy]benzaldehyde (180 mg, 0.71 mmol) in DIEA (0.5 mL) was added 4,5-dichloro-2-(oxan-2-yl)-2,3-dihydropyridazin-3-one (176 mg, 0.71 mmol) at room temperature. The resulting mixture was stirred for 1 hours at 90° C. After cooling to ambient temperature, the resulting mixture was concentrated under reduced pressure. The residue was purified by Prep-TLC, eluted with 8% methanol in dichloromethane to afford 4-chloro-5-(2-(1,2-dihydroxyethyl)-4-(4-fluoro-2-(trifluoromethyl)phenoxy)-5,8-dihydropyrido[3,4-d]pyrimidin-7(6H)-yl)-2-(tetrahydro-2H-pyran-2-yl)pyridazin-3(2H)-one (140 mg, 43%) as a brown solid.

### (S)-4-chloro-5-(2-(1,2-dihydroxyethyl)-4-(4-fluoro-2-(trifluoromethyl)phenoxy)-5,8-dihydropyrido[3,4-d]pyrimidin-7(6H)-yl)pyridazin-3(2H)-one and (R)-4-chloro-5-(2-(1,2-dihydroxyethyl)-4-(4-fluoro-2-(trifluoromethyl)phenoxy)-5,8-dihydropyrido[3,4-d]pyrimidin-7(6H)-yl)pyridazin-3(2H)-one

To a solution of 4-chloro-5-[2-(1,2-dihydroxyethyl)-4-[4-fluoro-2-(trifluoromethyl)phenoxy]-5H,6H,7H,8H-pyrido[3,4-d]pyrimidin-7-yl]-2-(oxan-2-yl)-2,3-dihydropyridazin-3-one (150 mg, 0.27 mmol) in DCM (4 mL) was added TFA (1 mL) at room temperature. The resulting mixture was stirred for 1 hours at room temperature. The resulting mixture was concentrated under reduced pressure. The residue was purified by reverse phase flash chromatography with the following conditions: Column: Spherical C18, 20˜40 um, 120 g; Mobile Phase A: Water (plus 5 mM NH₄HCO₃); Mobile Phase B: acetonitrile; Flow rate: 45 mL/min; Gradient: 5% B in 10 min, 45% B to 65% B in 15 min; Detector: 254 nm and 220 nm. The fractions containing desired product were collected at 64% B and concentrated under reduced pressure to afford the racemic product (130 mg) which was separated by Prep-Chiral-HPLC with the following conditions: Column: XBridge Prep OBD C18 Column 30×150 mm, 5 um; Mobile Phase A: Hexane, Mobile Phase B: EtOH; Flow rate: 20 mL/min; Gradient: 35% B in 10 min; Detector: 254/220 nm). Although the two isomers were separated by this technique, the absolute orientation was not determined. The fractions containing desired product were collected and concentrated under reduced pressure to afford the product: The compound designated as (S)-4-chloro-5-(2-(1,2-dihydroxyethyl)-4-(4-fluoro-2-(trifluoromethyl)phenoxy)-5,8-dihydropyrido[3,4-d]pyrimidin-7(6H)-yl)pyridazin-3(2H)-one: retention time (4.97 min) (49.5 mg, 39%) as a white solid and The compound designated as (R)-4-chloro-5-(2-(1,2-dihydroxyethyl)-4-(4-fluoro-2-(trifluoromethyl)phenoxy)-5,8-dihydropyrido[3,4-d]pyrimidin-7(6H)-yl)pyridazin-3(2H)-one: retention time (8.05 min) (45.7 mg, 36%) as a white solid.

### Example 22. Synthesis of Compound 131

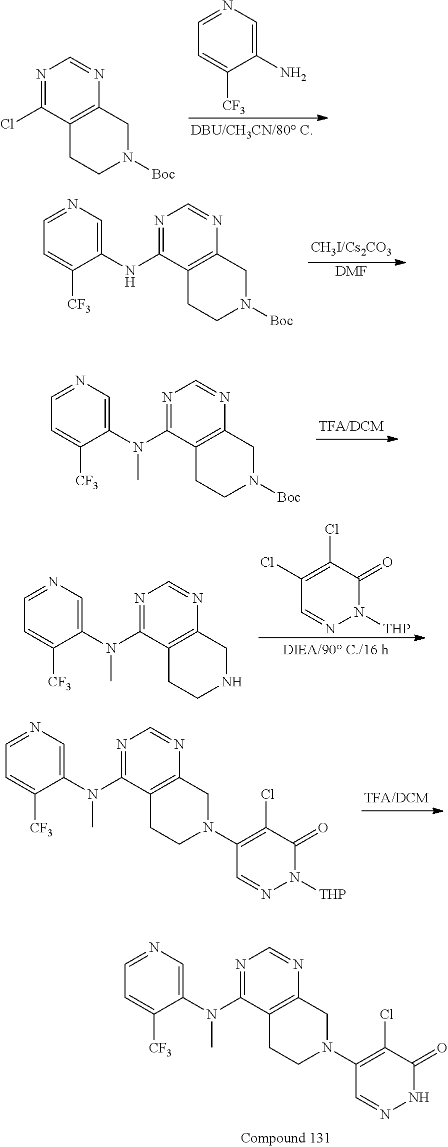

### Tert-butyl 4-[[4-(trifluoromethyl)pyridin-3-yl]amino]-5H,6H,7H,8H-pyrido[3,4-d]pyrimidine-7-carboxylate

To a stirred mixture of tert-butyl 4-chloro-5H,6H,7H,8H-pyrido[3,4-d]pyrimidine-7-carboxylate (500 mg, 1.854 mmol, 1 equiv.) and 4-(trifluoromethyl)pyridin-3-amine (601.03 mg, 3.707 mmol, 2.0 equiv.) in 1,4-dioxane (5 mL) were added Pd(AcO)₂ (83.24 mg, 0.371 mmol, 0.2 equiv.) and Cs₂CO₃ (1207.95 mg, 3.707 mmol, 2.0 equiv.) and XantPhos (429.04 mg, 0.741 mmol, 0.4 equiv.) at room temperature under nitrogen atmosphere. The resulting mixture was stirred for 2 h at 110 degrees C. under nitrogen atmosphere. The reaction was monitored by LCMS. The mixture was allowed to cool down to room temperature. The resulting mixture was filtered, the filter cake was washed with DCM (3×2 mL). The filtrate was concentrated under reduced pressure. The resulting mixture was concentrated under reduced pressure. The crude product was purified by reverse phase flash with the following conditions (Column: C18,120 g; Mobile Phase A: Water/0.05% NH4HCO3, Mobile Phase B: ACN; Flow rate: 45 mL/min; Gradient: 45% B to 65% B in 15 min; Detector, 254 nm and 220 nm, the desired product were collected at 64% B) to afford tert-butyl 4-[[4-(trifluoromethyl)pyridin-3-yl]amino]-5H,6H,7H,8H-pyrido[3,4-d]pyrimidine-7-carboxylate (600 mg, 81.86%) as a white solid.

### Tert-butyl 4-[methyl[4-(trifluoromethyl)pyridin-3-yl]amino]-5H,6H,7H,8H-pyrido[3,4-d]pyrimidine-7-carboxylate

To a stirred mixture of tert-butyl4-[[4-(trifluoromethyl)pyridin-3-yl]amino]-5H,6H,7H,8H-pyrido[3,4-d]pyrimidine-7-carboxylate (1.32 g, 3.339 mmol, 1 equiv.) and Cs₂CO₃ (2.18 g, 6.677 mmol, 2.0 equiv.) in DMF (10 mL) was added CH3I (0.95 g, 6.677 mmol, 2.0 equiv.) at 0° C. under nitrogen atmosphere. The resulting mixture was stirred for 2 h at room temperature under nitrogen atmosphere. The reaction was monitored by LCMS. The crude product was purified by reverse phase flash with the following conditions (Column: C18,120 g; Mobile Phase A: Water/0.05% NH₄HCO₃, Mobile Phase B: ACN; Flow rate: 45 mL/min; Gradient: 45% B to 65% B in 15 min; Detector, 254 nm and 220 nm, the desired product were collected at 64% B) to afford tert-butyl4-[methyl[4-(trifluoromethyl)pyridin-3-yl]amino]-5H,6H,7H,8H-pyrido[3,4-d]pyrimidine-7-carboxylate (400 mg, 29.26%) as a brown solid.

### N-methyl-N-[5H,6H,7H,8H-pyrido[3,4-d]pyrimidin-4-yl]-4-(trifluoromethyl)pyridin-3-amine

To a stirred solution of tert-butyl 4-[methyl[4-(trifluoromethyl)pyridin-3-yl]amino]-5H,6H,7H,8H-pyrido[3,4-d]pyrimidine-7-carboxylate (220 mg, 0.537 mmol, 1 equiv.) in DCM (4 mL) was added TFA (1 mL) at room temperature under nitrogen atmosphere. The resulting mixture was stirred for 1 h at room temperature. The reaction was monitored by LCMS. The resulting mixture was concentrated under reduced pressure. The mixture was basified to pH 8 with saturated NaHCO₃(aq.). The resulting mixture was concentrated under reduced pressure. The residue was purified by Prep-TLC (DCM/MeOH 12:1) to afford N-methyl-N-[5H,6H,7H,8H-pyrido[3,4-d]pyrimidin-4-yl]-4-(trifluoromethyl)pyridin-3-amine (130 mg, 78.22%) as a brown solid.

### 4-chloro-5-(4-[methyl[4-(trifluoromethyl)pyridin-3-yl]amino]-5H,6H,7H,8H-pyrido[3,4-d]pyrimidin-7-yl)-2-(oxan-2-yl)-2,3-dihydropyridazin-3-one

To a stirred solution of N-methyl-N-[5H,6H,7H,8H-pyrido[3,4-d]pyrimidin-4-yl]-4-(trifluoromethyl)pyridin-3-amine (130 mg, 0.420 mmol, 1 equiv.) in DIEA (0.5 mg) was added 4,5-dichloro-2-(oxan-2-yl)-2,3-dihydropyridazin-3-one (104.69 mg, 0.420 mmol, 1.0 equiv.) at room temperature. The resulting mixture was stirred for 1 h at 90° C. The reaction was monitored by LCMS. The mixture was allowed to cool down to room temperature. The resulting mixture was concentrated under reduced pressure. The residue was purified by Prep-TLC (DCM/MeOH 12:1) to afford 4-chloro-5-(4-[methyl[4-(trifluoromethyl)pyridin-3-yl]amino]-5H,6H,7H,8H-pyrido[3,4-d]pyrimidin-7-yl)-2-(oxan-2-yl)-2,3-dihydropyridazin-3-one (100 mg, 45.58%) as a brown solid.

### 4-chloro-5-(4-[methyl[4-(trifluoromethyl)pyridin-3-yl]amino]-5H,6H,7H,8H-pyrido[3,4-d]pyrimidin-7-yl)-2,3-dihydropyridazin-3-one

To a stirred solution of 4-chloro-5-(4-[methyl[4-(trifluoromethyl)pyridin-3-yl]amino]-5H,6H,7H,8H-pyrido[3,4-d]pyrimidin-7-yl)-2-(oxan-2-yl)-2,3-dihydropyridazin-3-one (100 mg, 0.192 mmol, 1 equiv.) in DCM (4 mL) was added TFA (1 mL) at room temperature. The resulting mixture was stirred for 1 h at room temperature. The reaction was monitored by LCMS. The resulting mixture was concentrated under reduced pressure. The mixture was basified to pH 8 with saturated NaHCO₃(aq.). The resulting mixture was concentrated under reduced pressure. The crude product (100 mg) was purified by Prep-HPLC with the following conditions (Column: XBridge Prep Phenyl OBD Column 19×150 mm Sum 13 nm; Mobile phase A: water, 5 mM NH₄HCO₃, Mobile phase B: Acetonitrile; Flow rate: 60 mL/min; Gradient: 35% B to 55% B in 8 min; 220 nm; Rt: 7.13 min) to afford 4-chloro-5-(4-[methyl[4-(trifluoromethyl)pyridin-3-yl]amino]-5H,6H,7H,8H-pyrido[3,4-d]pyrimidin-7-yl)-2,3-dihydropyridazin-3-one (52 mg, 61.99%) as a white solid.

### Example 23. Synthesis of Intermediates

### A. 2-(Difluoromethyl)-4-fluorophenyl acetate

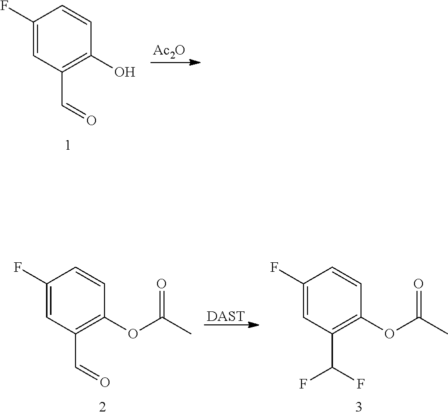

### 4-Fluoro-2-formylphenyl acetate

To a solution of 5-fluoro-2-hydroxybenzaldehyde (10 g, 71.371 mmol, 1 equiv.) in Pyridine (100 mL, 1242.353 mmol, 17.41 equiv.) was added acetyl acetate (14.57 g, 0.143 mmol, 2 equiv.) at 25° C. The solution was stirred at 25° C. for 30 min. The resulting solution was concentrated under reduced pressure. The residue was purified by silica gel column chromatography, eluted with PE/EA (100/1 to 20/1) to afford 4-fluoro-2-formylphenyl acetate (12 g, 92.31%) as a light yellow oil.

### 2-(Difluoromethyl)-4-fluorophenyl acetate

To a solution of 4-fluoro-2-formylphenyl acetate (12 g, 65.880 mmol, 1 equiv.) in DCM (200 mL, 3146.009 mmol, 47.75 equiv.) was added DAST (21.24 g, 131.760 mmol, 2 equiv.) at 0° C. The solution was stirred at 25° C. for 4 hours. The resulting solution was quenched with water (100 mL). The resulting mixture was extracted with DCM (100 mL×2). The combined organic layers were washed with saturated NaCl aq. (100 mL×2) dried over anhydrous Na₂SO₄. After filtration, the filtrate was concentrated under reduced pressure. The residue was purified by silica gel column chromatography, eluted with PE/EA (10/1 to 5/1) to afford 2-(difluoromethyl)-4-fluorophenyl acetate (10 g, 74.35%) as a light yellow oil.

### B. 2-(Difluoromethyl)-4-fluorophenol

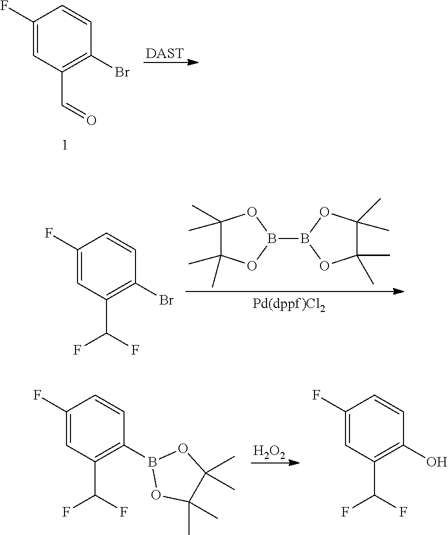

### 1-Bromo-2-(difluoromethyl)-4-fluorobenzene

To a stirred solution of 2-bromo-5-fluorobenzaldehyde (10 g, 49.26 mmol, 1 equiv.) in DCM (60 mL) was added DAST (15.9 g, 98.52 mmol, 2 equiv). The resulting mixture was stirred for 2 hours at −10° C. The reaction was quenched with Water at −10° C. The resulting mixture was extracted with EtOAc (4×30 mL). The combined organic layers were washed with brine (2×40 mL), dried over anhydrous Na₂SO₄. After filtration, the filtrate was concentrated under reduced pressure. The residue was purified by silica gel column chromatography, eluted with PE/EtOAc (6:1) to afford 1-bromo-2-(difluoromethyl)-4-fluorobenzene (8 g, 72.18%) as a light yellow oil.

### 2-[2-(Difluoromethyl)-4-fluorophenyl]-4,4,5,5-tetramethyl-1,3,2-dioxaborolane

To a solution of 1-bromo-2-(difluoromethyl)-4-fluorobenzene (31 g, 137.773 mmol, 1 equiv.) and BPD (52.48 g, 206.664 mmol, 1.50 equiv.) in 1,4-dioxane (300 mL, 3541.225 mmol, 25.70 equiv.) were added AcOK (27.04 g, 275.546 mmol, 2 equiv.) and Pd(dppf)Cl₂.CH₂Cl₂ (5.63 g, 6.889 mmol, 0.05 equiv.) at 25° C. under nitrogen atmosphere. The mixture was stirred at 90° C. for 2 hours. The resulting mixture was concentrated under reduced pressure. The residue was purified by silica gel column chromatography, eluted with PE/EA (10/1) to afford 2-[2-(difluoromethyl)-4-fluorophenyl]-4,4,5,5-tetramethyl-1,3,2-dioxaborolane (30 g, 80.03%) as a light yellow oil. The reaction was monitored by TLC. The crude was used the next step directly.

### 2-(Difluoromethyl)-4-fluorophenol

To a solution of 2-[2-(difluoromethyl)-4-fluorophenyl]-4,4,5,5-tetramethyl-1,3,2-dioxaborolane (50 g, 183.776 mmol, 1 equiv.) in MeOH (300 mL, 7409.673 mmol, 40.32 equiv.) and H₂O (100 mL, 5550.837 mmol, 30.20 equiv.) was added H₂O₂ (30%) (50 mL, 2146.131 mmol, 11.68 equiv.) dropwise at 0° C. The solution was stirred at 25° C. for 3 hours. The resulting solution was concentrated under reduced pressure. The residue was diluted with EA (500 mL), The organic layer was washed with 3×200 mL of saturated NaCl (aq.). Combined organic layers was dried with anhydrous Na₂SO₄, concentrated under reduced pressure to afford 2-(difluoromethyl)-4-fluorophenol (25 g, 83.91%) as a light yellow oil.

### Example 24. Synthesis of Compound MS

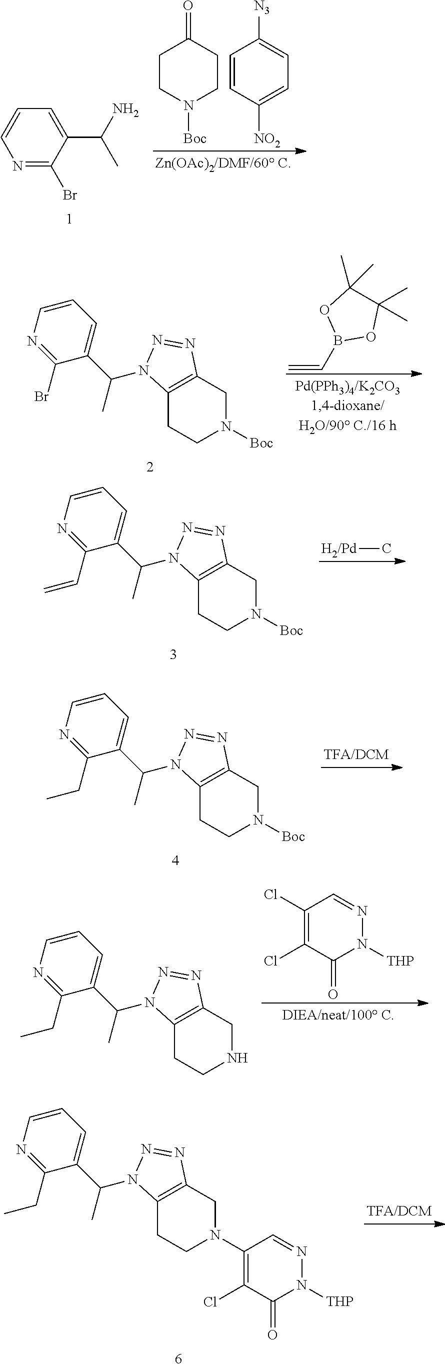

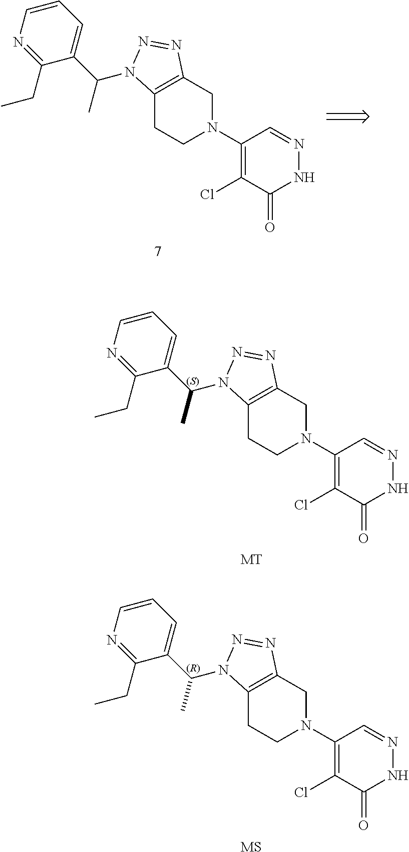

### tert-Butyl 1-[1-(2-bromopyridin-3-yl)ethyl]-1H,4H,5H,6H,7H-[1,2,3]triazolo[4,5-c]pyridine-5-carboxylate

To a stirred mixture of 1-(2-bromopyridin-3-yl)ethan-1-amine (1009.1 mg, 5.02 mmol, 2.00 equiv.) and tert-butyl 4-oxopiperidine-1-carboxylate (500 mg, 2.51 mmol, 1 equiv.) in DMF (10 mL) were added 1-azido-4-nitrobenzene (576.6 mg, 3.51 mmol, 1.40 equiv.) and Zn(OAc)₂ (460.5 mg, 2.51 mmol, 1.00 equiv.) in portions at rt under nitrogen atmosphere. The resulting mixture was stirred for 16 h at 60 degrees Celsius under nitrogen atmosphere. The reaction was monitored by LCMS. The mixture was allowed to cool down to rt. The residue was purified by reverse phase flash with the following conditions (Column: XBridge Shield RP18 OBD Column, 20-40 um, 19*150 mm; Mobile Phase A: Water (10 MMOL/L NH₄HCO₃), Mobile Phase B: ACN; Flow rate: 80 mL/min; Gradient: 40% B to 80% B in 30 min; 220 nm; Rt: 7.08 min) to afford tert-butyl 1-[1-(2-bromopyridin-3-yl)ethyl]-1H,4H,5H,6H,7H-[1,2,3]triazolo[4,5-c]pyridine-5-carboxylate (800 mg, 78.08%) as a yellow oil.

### tert-butyl 1-[1-(2-ethenylpyridin-3-yl)ethyl]-1H,4H,5H,6H,7H-[1,2,3]triazolo[4,5-c]pyridine-5-carboxylate

To a stirred mixture of tert-butyl 1-[1-(2-bromopyridin-3-yl)ethyl]-1H,4H,5H,6H,7H-[1,2,3]triazolo[4,5-c]pyridine-5-carboxylate (800 mg, 1.96 mmol, 1 equiv.) and 2-ethenyl-4,4,5,5-tetramethyl-1,3,2-dioxaborolane (301.8 mg, 1.96 mmol, 1.00 equiv.) in dioxane (30 mL) and H2O (6 mL) were added Pd(PPh3)4 (226.4 mg, 0.20 mmol, 0.10 equiv.) and K2CO3 (812.4 mg, 5.88 mmol, 3.00 equiv.) in portions at rt under nitrogen atmosphere. The resulting mixture was stirred for 16 h at 90 degrees Celsius under nitrogen atmosphere. The reaction was monitored by LCMS. The mixture was allowed to cool down to rt. The resulting mixture was concentrated under reduced pressure. The residue was purified by silica gel column chromatography, eluted with PE/EtOAc (30/1 to 5/1) to afford tert-butyl 1-[1-(2-ethenylpyridin-3-yl)ethyl]-1H,4H,5H,6H,7H-[1,2,3]triazolo[4,5-c]pyridine-5-carboxylate (600 mg, 86.15%) as a yellow oil.

### tert-Butyl 1-[1-(2-ethylpyridin-3-yl)ethyl]-1H,4H,5H,6H,7H-[1,2,3]triazolo[4,5-c]pyridine-5-carboxylate

To a solution of tert-butyl 1-[1-(2-ethenylpyridin-3-yl)ethyl]-1H,4H,5H,6H,7H-[1,2,3]triazolo[4,5-c]pyridine-5-carboxylate (300 mg, 0.84 mmol, 1 equiv.) in 20 mL MeOH was added Pd/C (10%, 0.02 g) under nitrogen atmosphere in a 50 mL round-bottom flask. The mixture was hydrogenated at room temperature for 2 h under hydrogen atmosphere using a hydrogen balloon, filtered through a celite pad and concentrated under reduced pressure. This resulted in tert-butyl 1-[1-(2-ethylpyridin-3-yl)ethyl]-1H,4H,5H,6H,7H-[1,2,3]triazolo[4,5-c]pyridine-5-carboxylate (260 mg, 86.18%) as a yellow oil.

### 2-Ethyl-3-(1-[1H,4H,5H,6H,7H-[1,2,3]triazolo[4,5-c]pyridin-1-yl]ethyl)pyridine

To a stirred solution of tert-butyl 1-[1-(2-ethylpyridin-3-yl)ethyl]-1H,4H,5H,6H,7H-[1,2,3]triazolo[4,5-c]pyridine-5-carboxylate (260 mg, 0.73 mmol, 1 equiv.) in DCM (10 mL) was added TFA (1 mL, 13.46 mmol, 18.51 equiv.) dropwise at rt. The reaction mixture was stirred for 16 h at rt. The reaction was monitored by LCMS. The resulting mixture was concentrated under reduced pressure. The residue was basified to pH=8 with saturated NH₄HCO₃ (aq.). The resulting mixture was extracted with CH₂C₁₂ (3×100 mL). The combined organic layers were washed with brine (1×100 mL), dried over anhydrous Na₂SO₄. After filtration, the filtrate was concentrated under reduced pressure. The residue was purified by Prep-TLC (PE/EtOAc 1/1) to afford 2-ethyl-3-(1-[1H,4H,5H,6H,7H-[1,2,3]triazolo[4,5-c]pyridin-1-yl]ethyl)pyridine (150 mg, 80.14%) as a yellow oil.

### 4-Chloro-5-[1-[1-(2-ethylpyridin-3-yl)ethyl]-1H,4H,5H,6H,7H-[1,2,3]triazolo[4,5-c]pyridin-5-yl]-2-(oxan-2-yl)-2,3-dihydropyridazin-3-one

Into a 25 mL round-bottom flask were added 2-ethyl-3-(1-[1H,4H,5H,6H,7H-[1,2,3]triazolo[4,5-c]pyridin-1-yl]ethyl)pyridine (150 mg, 0.58 mmol, 1 equiv.), 4,5-dichloro-2-(oxan-2-yl)-2,3-dihydropyridazin-3-one (290.4 mg, 1.17 mmol, 2.00 equiv.) and DIEA (150.7 mg, 1.17 mmol, 2.00 equiv.) at rt under nitrogen atmosphere. The resulting mixture was stirred for 16 h at 90 degrees Celsius under nitrogen atmosphere. The residue was purified by Prep-TLC (PE/EtOAc=1/1) to afford 4-chloro-5-[1-[1-(2-ethylpyridin-3-yl)ethyl]-1H,4H,5H,6H,7H-[1,2,3]triazolo[4,5-c]pyridin-5-yl]-2-(oxan-2-yl)-2,3-dihydropyridazin-3-one (145 mg, 52.93%) as a yellow oil.

### 4-chloro-5-[1-[(1R)-1-(2-ethylpyridin-3-yl)ethyl]-1H,4H,5H,6H,7H-[1,2,3]triazolo[4,5-c]pyridin-5-yl]-2,3-dihydropyridazin-3-one and 4-chloro-5-[1-[(1S)-1-(2-ethylpyridin-3-yl)ethyl]-1H,4H,5H,6H,7H-[1,2,3]triazolo[4,5-c]pyridin-5-yl]-2,3-dihydropyridazin-3-one

To a stirred solution of 4-chloro-5-[1-[1-(2-ethylpyridin-3-yl)ethyl]-1H,4H,5H,6H,7H-[1,2,3]triazolo[4,5-c]pyridin-5-yl]-2-(oxan-2-yl)-2,3-dihydropyridazin-3-one (145 mg, 0.31 mmol, 1 equiv.) in DCM (10 mL) was added TFA (1 mL, 13.46 mmol, 43.64 equiv.) dropwise at rt. The reaction mixture was stirred for 4 h at rt. The reaction was monitored by LCMS. The resulting mixture was concentrated under reduced pressure. The residue was basified to pH=8 with saturated NH4HCO3 (aq.). The resulting mixture was extracted with CH2Cl2 (3×100 mL). The combined organic layers were washed with brine (1×100 mL), dried over anhydrous Na2SO4. After filtration, the filtrate was concentrated under reduced pressure. The residue (75 mg) was purified by Chiral-Prep-HPLC with the following conditions (Column: CHIRALPAK IE, 2*25 cm, 5 um; Mobile Phase: (Hex/DCM=3/1)/EtOH=80/20; Flow rate: 20 mL/min; Gradient: 20 B to 20 B in 20 min; 220/254 nm; RT1:12.678; RT2:16.738). 4-chloro-5-[1-[(1R)-1-(2-ethylpyridin-3-yl)ethyl]-1H,4H,5H,6H,7H-[1,2,3]triazolo[4,5-c]pyridin-5-yl]-2,3-dihydropyridazin-3-one (16.8 mg, 14.11%) was obtained at 1.380 min as a white solid. 4-chloro-5-[1-[(1S)-1-(2-ethylpyridin-3-yl)ethyl]-1H,4H,5H,6H,7H-[1,2,3]triazolo[4,5-c]pyridin-5-yl]-2,3-dihydropyridazin-3-one (19.8 mg) was obtained at 1.832 min as a white solid (E01224-021).

### Example 25. Synthesis of MX

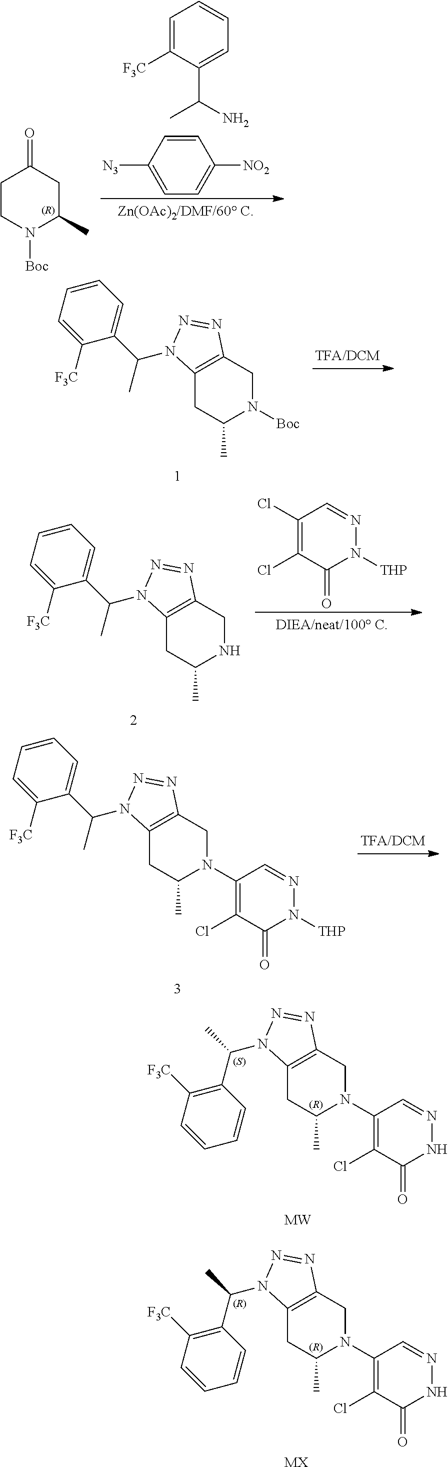

### tert-Butyl (4R)-4-methyl-1-[1-[2-(trifluoromethyl)phenyl]ethyl]-1H,4H,5H,6H,7H-[1,2,3]triazolo[4,5-c]pyridine-5-carboxylate

To a stirred solution of tert-butyl (2R)-2-methyl-4-oxopiperidine-1-carboxylate (1 g, 4.69 mmol, 1 equiv.) and 1-[2-(trifluoromethyl)phenyl]ethan-1-amine (0.9 g, 4.76 mmol, 1.01 equiv.) in DMF (20 mL) were added 1-azido-4-nitrobenzene (1.1 g, 6.56 mmol, 1.4 equiv.) and Zn(OAc)₂ (0.9 g, 4.69 mmol, 1 equiv.). The resulting mixture was stirred for overnight at 60 degrees C. The residue was purified by reverse flash chromatography with the following conditions: column, C18 silica gel; mobile phase, MeCN in water, 20% to 60% gradient in 40 min; detector, UV 254 nm. This resulted in tert-butyl (4R)-4-methyl-1-[1-[2-(trifluoromethyl)phenyl]ethyl]-1H,4H,5H,6H,7H-[1,2,3]triazolo[4,5-c]pyridine-5-carboxylate (1.5 g, 77.94%) as a off-white solid.

### (4R)-4-Methyl-1-[1-[2-(trifluoromethyl)phenyl]ethyl]-1H,4H,5H,6H,7H-[1,2,3]triazolo[4,5-c]pyridine

To a stirred solution of tert-butyl (4R)-4-methyl-1-[1-[2-(trifluoromethyl)phenyl]ethyl]-1H,4H,5H,6H,7H-[1,2,3]triazolo[4,5-c]pyridine-5-carboxylate (1.5 g, 3.65 mmol, 1 equiv.) in DCM (9 mL) was added TFA (3 mL). The resulting mixture was stirred for 2 h at room temperature. The mixture was basified to pH 8 with saturated NH₄HCO₃ (aq.). The solution was concentrated under reduced pressure. The residue was purified by reverse flash chromatography with the following conditions: column, C18 silica gel; mobile phase, MeCN in water, 10% to 50% gradient in 30 min; detector, UV 254 nm. This resulted in (4R)-4-methyl-1-[1-[2-(trifluoromethyl)phenyl]ethyl]-1H,4H,5H,6H,7H-[1,2,3]triazolo[4,5-c]pyridine (1 g, 88.17%) as a yellow solid.

### 4-Chloro-5-[(6R)-6-methyl-1-[1-[2-(trifluoromethyl)phenyl]ethyl]-1H,4H,5H,6H,7H-[1,2,3]triazolo[4,5-c]pyridin-5-yl]-2-(oxan-2-yl)-2,3-dihydropyridazin-3-one

To a stirred solution of (6R)-6-methyl-1-[1-[2-(trifluoromethyl)phenyl]ethyl]-1H,4H,5H,6H,7H-[1,2,3]triazolo[4,5-c]pyridine (200 mg, 0.64 mmol, 1 equiv.), 4,5-dichloro-2-(oxan-2-yl)-2,3-dihydropyridazin-3-one (192.6 mg, 0.77 mmol, 1.2 equiv.) and DIEA (249.9 mg, 1.93 mmol, 3 equiv.). The resulting mixture was stirred for overnight at 100 degrees C. The residue was purified by reverse flash chromatography with the following conditions: column, C18 silica gel; mobile phase, MeCN in water, 20% to 60% gradient in 40 min; detector, UV 254 nm. This resulted in 4-chloro-5-[(6R)-6-methyl-1-[1-[2-(trifluoromethyl)phenyl]ethyl]-1H,4H,5H,6H,7H-[1,2,3]triazolo[4,5-c]pyridin-5-yl]-2-(oxan-2-yl)-2,3-dihydropyridazin-3-one (250 mg, 74.17%) as a yellow solid.

### 4-chloro-5-[(6R)-6-methyl-1-[(1R)-1-[2-(trifluoromethyl)phenyl]ethyl]-1H,4H,5H,6H,7H-[1,2,3]triazolo[4,5-c]pyridin-5-yl]-2,3-dihydropyridazin-3-one and 4-chloro-5-[(6R)-6-methyl-1-[(1S)-1-[2-(trifluoromethyl)phenyl]ethyl]-1H,4H,5H,6H,7H-[1,2,3]triazolo[4,5-c]pyridin-5-yl]-2,3-dihydropyridazin-3-one

To a stirred solution of 4-chloro-5-[(6R)-6-methyl-1-[1-[2-(trifluoromethyl)phenyl]ethyl]-1H,4H,5H,6H,7H-[1,2,3]triazolo[4,5-c]pyridin-5-yl]-2-(oxan-2-yl)-2,3-dihydropyridazin-3-one (240 mg, 0.46 mmol, 1 equiv.) in DCM (6 mL) was added TFA (2 mL). The resulting mixture was stirred for 2 h at room temperature. The resulting mixture was concentrated under reduced pressure. The crude product was purified by Prep-HPLC with the following conditions (Column: XBridge Prep OBD C18 Column 30×150 mm Sum; Mobile Phase A: Water (10 MMOL/L NH₄HCO₃), Mobile Phase B: ACN; Flow rate: 60 mL/min; Gradient: 27% B to 50% B in 7 min; 220 nm; Rt: 6.38 min) to afford 4-chloro-5-[(6R)-6-methyl-1-[(1R)-1-[2-(trifluoromethyl)phenyl]ethyl]-1H,4H,5H,6H,7H-[1,2,3]triazolo[4,5-c]pyridin-5-yl]-2,3-dihydropyridazin-3-one (22.4 mg, 11.12%) as a yellow solid and 4-chloro-5-[(6R)-6-methyl-1-[(1S)-1-[2-(trifluoromethyl)phenyl]ethyl]-1H,4H,5H,6H,7H-[1,2,3]triazolo[4,5-c]pyridin-5-yl]-2,3-dihydropyridazin-3-one (58.5 mg, 29.05%) as a off-white solid.

### Example 26. Synthesis of Compound LY

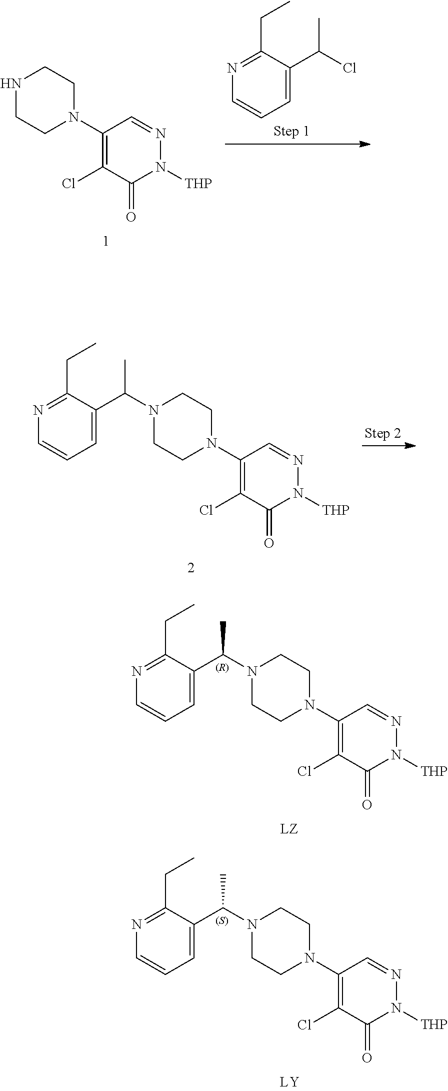

3-(1-chloroethyl)-2-ethylpyridine was prepared by the methods and scheme described for 3-(1-chloropropyl)-2-ethylpyridine by using the corresponding pyridine.

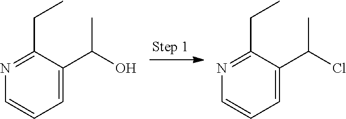

### 3-(1-chloropropyl)-2-ethylpyridine

To a stirred solution of 1-(2-ethylpyridin-3-yl)propan-1-ol (300 mg, 1.82 mmol, 1 equiv.) in DCM (20 mL) was added SOCl₂ (432.0 mg, 3.63 mmol, 2.00 equiv.) dropwise at 0 degrees C. under nitrogen atmosphere. The resulting mixture was stirred for 16 h at under nitrogen atmosphere. The reaction was monitored by LCMS. The resulting mixture was concentrated under vacuum. This resulted in 3-(1-chloropropyl)-2-ethylpyridine (350 mg, 104.95%) as a yellow oil.

### Step 1

### 4-chloro-5-[4-[1-(2-ethylpyridin-3-yl)ethyl]piperazin-1-yl]-2-(oxan-2-yl)-2,3-dihydropyridazin-3-one

To a stirred mixture of 4-chloro-2-(oxan-2-yl)-5-(piperazin-1-yl)-2,3-dihydropyridazin-3-one (100 mg, 0.33 mmol, 1 equiv.) and 3-(1-chloroethyl)-2-ethylpyridine (68.1 mg, 0.40 mmol, 1.20 equiv.) in ACN (10 mL) were added K2CO3 (92.5 mg, 0.67 mmol, 2.0 equiv.) and KI (111.1 mg, 0.67 mmol, 2.00 equiv.) in portions at rt under nitrogen atmosphere. The resulting mixture was stirred for 16 h at 70 degrees C. under nitrogen atmosphere. The reaction was monitored by LCMS. The resulting mixture was filtered, the filter cake was washed with ACN (2×30 mL). The filtrate was concentrated under reduced pressure. The residue was purified by Prep-TLC (CH2Cl2/MeOH 20/1) to afford 4-chloro-5-[4-[1-(2-ethylpyridin-3-yl)ethyl]piperazin-1-yl]-2-(oxan-2-yl)-2,3-dihydropyridazin-3-one (120 mg, 83.00%) as a yellow oil.

### Step 2

### LY and LZ

### 4-chloro-5-[4-[(1S)-1-(2-ethylpyridin-3-yl)ethyl]piperazin-1-yl]-2,3-dihydropyridazin-3-one & 4-chloro-5-[4-[(1R)-1-(2-ethylpyridin-3-yl)ethyl]piperazin-1-yl]-2,3-dihydropyridazin-3-one

To a stirred solution of 4-chloro-5-[4-[1-(2-ethylpyridin-3-yl)ethyl]piperazin-1-yl]-2-(oxan-2-yl)-2,3-dihydropyridazin-3-one (120 mg, 0.28 mmol, 1 equiv.) in DCM (10 mL) was added TFA (1 mL, 13.46 mmol, 48.46 equiv.) dropwise at rt. The reaction mixture was stirred for 4 h at rt. The reaction was monitored by LCMS. The resulting mixture was concentrated under reduced pressure. The residue was basified to pH=8 with saturated NH₄HCO₃ (aq.). The resulting mixture was extracted with CH2Cl2 (3×100 mL). The combined organic layers were washed with brine (1×50 mL), dried over anhydrous Na₂SO₄. After filtration, the filtrate was concentrated under reduced pressure. The residue (70 mg) was purified by Chiral-Prep-HPLC with the following conditions (Column: CHIRALPAK IE, 2*25 cm, 5 um; Mobile Phase: MTBE/EtOH=80/20; Flow rate: 20 mL/min; Gradient: 20 B to 20 B in 20 min; 220/254 nm; RT1:12.678; RT2:16.738). 4-chloro-5-[4-[(1S)-1-(2-ethylpyridin-3-yl)ethyl]piperazin-1-yl]-2,3-dihydropyridazin-3-one (9.5 mg, 9.83%) was obtained at 2.544 min as a light yellow solid. 4-chloro-5-[4-[(1R)-1-(2-ethylpyridin-3-yl)ethyl]piperazin-1-yl]-2,3-dihydropyridazin-3-one (14.2 mg) was obtained at 2.984 min as a light yellow solid.

### Example 27. Synthesis of LW

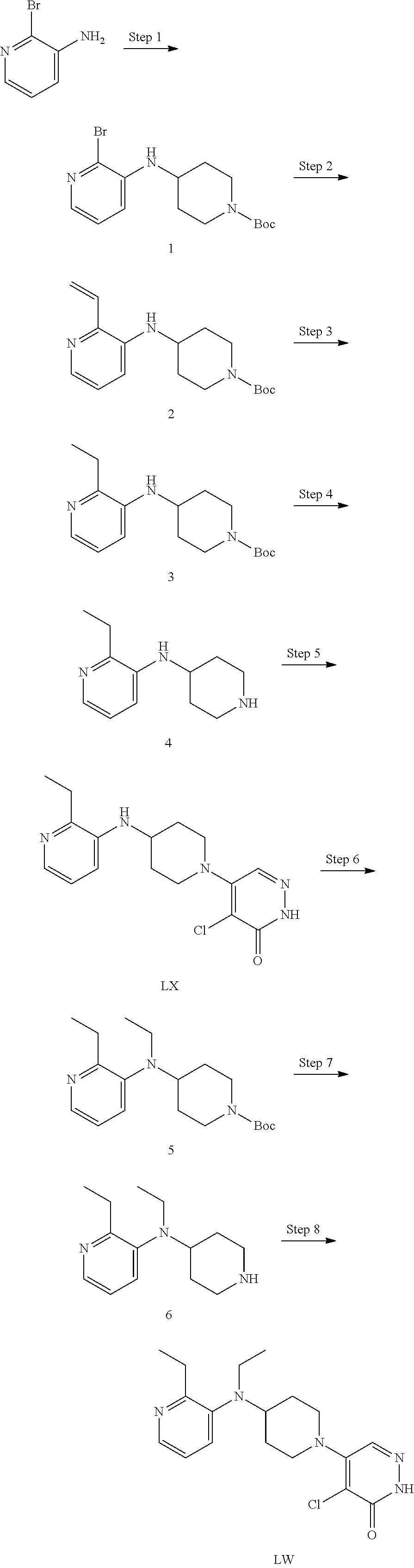

### Step 1

### tert-butyl 4-[(2-bromopyridin-3-yl)amino]piperidine-1-carboxylate

To a stirred solution of 2-bromopyridin-3-amine (600 mg, 3.468 mmol, 1 equiv.) and tert-butyl 4-oxopiperidine-1-carboxylate (690.99 mg, 3.468 mmol, 1 equiv.) in DCM (20 mL) was added AcOH (208.26 mg, 3.468 mmol, 1 equiv.) dropwise/in portions at 0 degrees C. under nitrogen atmosphere. The mixture was stirred at rt for 2 h. NaBH(OAc)₃ (1470.00 mg, 6.936 mmol, 2.00 equiv.) was added to the mixture at 0 degrees C. The mixture was stirred at rt overnight. Desired product could be detected by LCMS. The reaction was quenched by the addition of Water (40 mL) at 0 degrees C. The aqueous layer was extracted with CH2Cl2 (2×30 mL). The organic layer was concentrated under reduced pressure to afford tert-butyl 4-[(2-bromopyridin-3-yl)amino]piperidine-1-carboxylate (800 mg, 64.75%) as yellow solid.

### Step 2

### tert-butyl 4-[(2-ethenylpyridin-3-yl)amino]piperidine-1-carboxylate

To a solution of 2-ethenyl-4,4,5,5-tetramethyl-1,3,2-dioxaborolane (691.71 mg, 4.491 mmol, 2 equiv.) and tert-butyl 4-[(2-bromopyridin-3-yl)amino]piperidine-1-carboxylate (800 mg, 2.246 mmol, 1 equiv.) in 1,4-dioxane (10 mL) and H₂O (2 mL) were added K2CO3 (931.03 mg, 6.737 mmol, 3 equiv.) and Pd(PPh3)4 (259.48 mg, 0.225 mmol, 0.1 equiv.). After stirring for overnight at 80 degrees C. under a nitrogen atmosphere, the resulting mixture was concentrated under reduced pressure. The residue was purified by silica gel column chromatography, eluted with PE/EtOAc (5:1 to 3:1) to afford tert-butyl 4-[(2-ethenylpyridin-3-yl)amino]piperidine-1-carboxylate (600 mg, 88.07%) as a yellow solid.

### Step 3

### tert-butyl 4-[(2-ethylpyridin-3-yl)amino]piperidine-1-carboxylate

To a solution of tert-butyl4-[(2-ethenylpyridin-3-yl)amino]piperidine-1-carboxylate (600 mg, 1.978 mmol, 1 equiv.) in 30 mL MeOH was added Pd/C (10%, 21.05 mg) under nitrogen atmosphere in a 250 mL round-bottom flask. The mixture was hydrogenated at room temperature for 3 h under hydrogen atmosphere using a hydrogen balloon, filtered through a Celite pad and concentrated under reduced pressure to afford tert-butyl 4-[(2-ethylpyridin-3-yl)amino]piperidine-1-carboxylate (590 mg, 97.68%) as yellow solid.

### Step 4

### 2-ethyl-N-(piperidin-4-yl)pyridin-3-amine

To a stirred solution of tert-butyl 4-[(2-ethylpyridin-3-yl)amino]piperidine-1-carboxylate (590 mg, 1 equiv.) in DCM (15 mL) was added TFA (3 mL) dropwise at 0 degrees C. under nitrogen atmosphere. The mixture was stirred at rt for 1 h. Desired product could be detected by LCMS. The resulting mixture was concentrated under reduced pressure to afford 2-ethyl-N-(piperidin-4-yl)pyridin-3-amine (390 mg, 98.34%) as white solid.

### Step 5

### Compound LX

### 4-chloro-5-[4-[(2-ethylpyridin-3-yl)amino]piperidin-1-yl]-2,3-dihydropyridazin-3-one

To a stirred solution of 2-ethyl-N-(piperidin-4-yl)pyridin-3-amine (100 mg, 0.487 mmol, 1 equiv.) and 4,5-dichloro-2,3-dihydropyridazin-3-one (80.35 mg, 0.487 mmol, 1.00 equiv.) in DMA (8 mL) was added DIEA (125.90 mg, 0.974 mmol, 2 equiv.) dropwise at room temperature under nitrogen atmosphere. The mixture was stirred at 100 degrees C. overnight. Desired product could be detected by LCMS. The resulting mixture was concentrated under reduced pressure. The crude product (60 mg) was purified by Prep-HPLC with the following conditions (Column: XBridge Prep OBD C18 Column 30×150 mm Sum; Mobile Phase A: Water (10 MMOL/L NH4HCO3), Mobile Phase B: ACN; Flow rate: 60 mL/min; Gradient: 18% B to 30% B in 6.5 min; 220 nm; Rt: 5.37 8.55 min) to afford 4-chloro-5-[4-[(2-ethylpyridin-3-yl)amino]piperidin-1-yl]-2,3-dihydropyridazin-3-one (20 mg) as a white solid and 5-chloro-4-[4-[(2-ethylpyridin-3-yl)amino]piperidin-1-yl]-2,3-dihydropyridazin-3-one (7 mg) as a white solid.

### Step 6

### tert-butyl 4-[ethyl(2-ethylpyridin-3-yl)amino]piperidine-1-carboxylate

To a stirred solution of tert-butyl 4-[(2-ethylpyridin-3-yl)amino]piperidine-1-carboxylate (150 mg, 0.491 mmol, 1 equiv.) and acetaldehyde (32.45 mg, 0.737 mmol, 1.5 equiv.) in DCM (10 mL) was added AcOH (29.49 mg, 0.491 mmol, 1 equiv.) dropwise at 0 degrees C. under nitrogen atmosphere. The mixture was stirred at rt for 2 h. NaBH3CN (92.59 mg, 1.473 mmol, 3 equiv.) was added to the mixture at 0 degrees C. The mixture was stirred at rt overnight. Desired product could be detected by LCMS. The reaction was quenched by the addition of Water (40 mL) at 0 degrees C. The aqueous layer was extracted with CH2Cl2 (2×30 mL). The organic layer was concentrated under reduced pressure to afford tert-butyl 4-[ethyl(2-ethylpyridin-3-yl)amino]piperidine-1-carboxylate (150 mg, 91.59%) as white solid.

### Step 8

### N,2-diethyl-N-(piperidin-4-yl)pyridin-3-amine

To a stirred solution of tert-butyl 4-[ethyl(2-ethylpyridin-3-yl)amino]piperidine-1-carboxylate (150 mg, 1 equiv.) in DCM (10 mL) was added TFA (2 mL) dropwise at 0 degrees C. under nitrogen atmosphere. The mixture was stirred at rt for 2 h. Desired product could be detected by LCMS. The resulting mixture was concentrated under reduced pressure to afford N,2-diethyl-N-(piperidin-4-yl)pyridin-3-amine (100 mg, 95.27%) as yellow solid.

### Step 8

### Compound LW

### 4-chloro-5-[4-[ethyl(2-ethylpyridin-3-yl)amino]piperidin-1-yl]-2,3-dihydropyridazin-3-one

To a stirred solution of N,2-diethyl-N-(piperidin-4-yl)pyridin-3-amine (60 mg, 0.26 mmol, 1 equiv.) and 4,5-dichloro-2,3-dihydropyridazin-3-one (42.4 mg, 0.26 mmol, 1.00 equiv.) in DMA (5 mL, 53.78 mmol, 209.15 equiv.) was added DIEA (66.5 mg, 0.51 mmol, 2 equiv.) at room temperature under nitrogen atmosphere. The mixture was stirred at 100 degrees C. overnight. Desired product could be detected by LCMS. The mixture was concentrated under reduced pressure. The crude product (50 mg) was purified by Prep-HPLC with the following conditions (Column: XBridge Prep OBD C18 Column 30×150 mm Sum; Mobile Phase A: Water (10 MMOL/L NH4HCO3), Mobile Phase B: ACN; Flow rate: 60 mL/min; Gradient: 25% B to 40% B in 8 min; 220 nm; Rt: 7.58 min) to afford 4-chloro-5-[4-[ethyl(2-ethylpyridin-3-yl)amino]piperidin-1-yl]-2,3-dihydropyridazin-3-one (24.3 mg) as a white solid.

### Example 28. Synthesis of OM

Compound OM

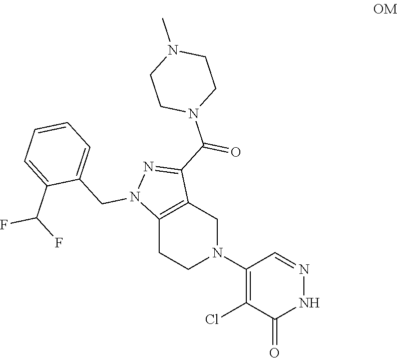 [SMILES: CN1CCN(C(=O)c2nn(Cc3ccccc3C(F)F)c3c2CN(c2cn[nH]c(=O)c2Cl)CC3)CC1]

was prepared by the methods and scheme described for compound OK by using the corresponding amine.

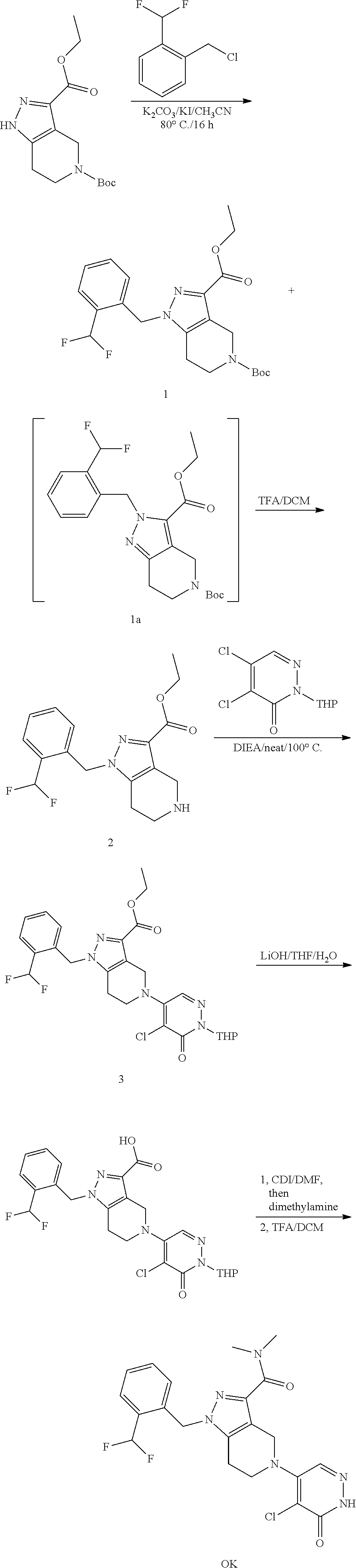

### Preparation of OK

### 5-tert-Butyl 3-ethyl 2-[[2-(difluoromethyl)phenyl]methyl]-2H,4H,5H,6H,7H-pyrazolo[4,3-c]pyridine-3,5-dicarboxylate

To a stirred solution of 1-(chloromethyl)-2-(difluoromethyl)benzene (800 mg, 4.530 mmol, 1 equiv.) and 5-tert-butyl 3-ethyl 1H,4H,5H,6H,7H-pyrazolo[4,3-c]pyridine-3,5-dicarboxylate (1337.96 mg, 4.530 mmol, 1.00 equiv.) in MeCN (15 mL) was added KI (752.04 mg, 4.530 mmol, 1 equiv.) and K2CO3 (1252.22 mg, 9.061 mmol, 2 equiv.) at room temperature under nitrogen atmosphere. The mixture was stirred at 80 degrees Celsius overnight. Desired product could be detected by LCMS. The resulting mixture was concentrated under reduced pressure. The residue was purified by silica gel column chromatography, eluted with PE/EtOAc (5:1 to 3:1) to afford 5-tert-butyl 3-ethyl 1-[[2-(difluoromethyl)phenyl]methyl]-1H,4H,5H,6H,7H-pyrazolo[4,3-c]pyridine-3,5-dicarboxylate (500 mg, 25.34%) as a yellow solid and 5-tert-butyl 3-ethyl 2-[[2-(difluoromethyl)phenyl]methyl]-2H,4H,5H,6H,7H-pyrazolo[4,3-c]pyridine-3,5-dicarboxylate (200 mg, 10.14%) as a yellow solid.

### Ethyl 1-[[2-(difluoromethyl)phenyl]methyl]-1H,4H,5H,6H,7H-pyrazolo[4,3-c]pyridine-3-carboxylate

To a stirred solution of 5-tert-butyl 3-ethyl 1-[[2-(difluoromethyl)phenyl]methyl]-1H,4H,5H,6H,7H-pyrazolo[4,3-c]pyridine-3,5-dicarboxylate (500 mg, 1.148 mmol, 1 equiv.) in DCM (10 mL, 157.300 mmol, 137.00 equiv.) was added TFA (2 mL, 26.926 mmol, 23.45 equiv.) dropwise at room temperature under nitrogen atmosphere. The mixture was stirred at rt for 2 h. Desired product could be detected by LCMS. The resulting mixture was concentrated under reduced pressure to afford ethyl 1-[[2-(difluoromethyl)phenyl]methyl]-1H,4H,5H,6H,7H-pyrazolo[4,3-c]pyridine-3-carboxylate (380 mg, 98.69%) as yellow solid.

### Ethyl 5-[5-chloro-1-(oxan-2-yl)-6-oxo-1,6-dihydropyridazin-4-yl]-1-[[2-(difluoromethyl)phenyl]methyl]-1H,4H,5H,6H,7H-pyrazolo[4,3-c]pyridine-3-carboxylate

To a stirred solution of ethyl 1-[[2-(difluoromethyl)phenyl]methyl]-1H,4H,5H,6H,7H-pyrazolo[4,3-c]pyridine-3-carboxylate (380 mg, 1.133 mmol, 1 equiv.) in DIEA (292.90 mg, 2.266 mmol, 2 equiv.) was added 4,5-dichloro-2-(oxan-2-yl)-2,3-dihydropyridazin-3-one (282.25 mg, 1.133 mmol, 1.00 equiv.) in portions at room temperature under nitrogen atmosphere. The mixture was stirred at 100 degrees Celsius overnight. Desired product could be detected by LCMS. The residue was purified by silica gel column chromatography, eluted with PE/EtOAc (5:1 to 3:1) to afford ethyl 5-[5-chloro-1-(oxan-2-yl)-6-oxo-1,6-dihydropyridazin-4-yl]-1-[[2-(difluoromethyl)phenyl]methyl]-1H,4H,5H,6H,7H-pyrazolo[4,3-c]pyridine-3-carboxylate (500 mg, 80.52%) as a yellow solid.

### 5-[5-chloro-1-(oxan-2-yl)-6-oxo-1,6-dihydropyridazin-4-yl]-1-[[2-(difluoromethyl)phenyl]methyl]-1H,4H,5H,6H,7H-pyrazolo[4,3-c]pyridine-3-carboxylic acid

To a stirred solution of ethyl 5-[5-chloro-1-(oxan-2-yl)-6-oxo-1,6-dihydropyridazin-4-yl]-1-[[2-(difluoromethyl)phenyl]methyl]-1H,4H,5H,6H,7H-pyrazolo[4,3-c]pyridine-3-carboxylate (460 mg, 0.839 mmol, 1 equiv.) in THF (5 mL) and H₂O (5 mL) was added LiOH (100.51 mg, 4.197 mmol, 5 equiv.) in portions at room temperature under nitrogen atmosphere. The mixture was stirred at 50 degrees Celsius overnight. Desired product could be detected by LCMS. The resulting mixture was concentrated under reduced pressure. The residue was purified by reverse phase flash with the following conditions (Column: XBridge Prep OBD C18 Column 30×150 mm Sum; Mobile Phase A: Water (10 MMOL/L NH4HCO3), Mobile Phase B: ACN; Flow rate: 60 mL/min; Gradient: 27% B to 55% B in 8 min; 220 nm; Rt: 7.82 min) to afford 5-[5-chloro-1-(oxan-2-yl)-6-oxo-1,6-dihydropyridazin-4-yl]-1-[[2-(difluoromethyl)phenyl]methyl]-1H,4H,5H,6H,7H-pyrazolo[4,3-c]pyridine-3-carboxylic acid (430 mg, 98.52%) as a colorless oil.

### 5-[5-Chloro-1-(oxan-2-yl)-6-oxo-1,6-dihydropyridazin-4-yl]-1-[[2-(difluoromethyl)phenyl]methyl]-N,N-dimethyl-1H,4H,5H,6H,7H-pyrazolo[4,3-c]pyridine-3-carboxamide

To a stirred solution of 5-[5-chloro-1-(oxan-2-yl)-6-oxo-1,6-dihydropyridazin-4-yl]-1-[[2-(difluoromethyl)phenyl]methyl]-1H,4H,5H,6H,7H-pyrazolo[4,3-c]pyridine-3-carboxylic acid (80 mg, 0.15 mmol, 1 equiv.) in DMF (5 mL) was added CDI (37.4 mg, 0.23 mmol, 1.5 equiv.) in portions at room temperature under nitrogen atmosphere. The mixture was stirred at 50 degrees Celsius for 2 h. dimethylamine (13.9 mg, 0.31 mmol, 2.00 equiv.) was added to the mixture. The mixture was stirred at 50 degrees Celsius overnight. Desired product could be detected by LCMS. The resulting mixture was concentrated under vacuum to afford 5-[5-chloro-1-(oxan-2-yl)-6-oxo-1,6-dihydropyridazin-4-yl]-1-[[2-(difluoromethyl)phenyl]methyl]-N,N-dimethyl-1H,4H,5H,6H,7H-pyrazolo[4,3-c]pyridine-3-carboxamide (60 mg, 71.29%) as yellow solid.

### 5-(5-Chloro-6-oxo-1,6-dihydropyridazin-4-yl)-1-[[2-(difluoromethyl)phenyl]methyl]-N,N-dimethyl-1H,4H,5H,6H,7H-pyrazolo[4,3-c]pyridine-3-carboxamide

To a stirred solution of 5-[5-chloro-1-(oxan-2-yl)-6-oxo-1,6-dihydropyridazin-4-yl]-1-[[2-(difluoromethyl)phenyl]methyl]-N,N-dimethyl-1H,4H,5H,6H,7H-pyrazolo[4,3-c]pyridine-3-carboxamide (50 mg, 1 equiv.) in DCM (10 mL) was added TFA (2 mL) dropwise at room temperature under nitrogen atmosphere. The mixture was stirred at rt for 1 h. Desired product could be detected by LCMS. The resulting mixture was concentrated under reduced pressure. The crude product (30 mg) was purified by Prep-HPLC with the following conditions (Column: XBridge Prep OBD C18 Column 30×150 mm Sum; Mobile Phase A: Water (10 MMOL/L NH4HCO3), Mobile Phase B: ACN; Flow rate: 60 mL/min; Gradient: 23% B to 45% B in 7 min; 220 nm; Rt: 6.47 min) to afford 5-(5-chloro-6-oxo-1,6-dihydropyridazin-4-yl)-1-[[2-(difluoromethyl)phenyl]methyl]-N,N-dimethyl-1H,4H,5H,6H,7H-pyrazolo[4,3-c]pyridine-3-carboxamide (25 mg) as a white solid.

### Example 29. Synthesis of Compound OU

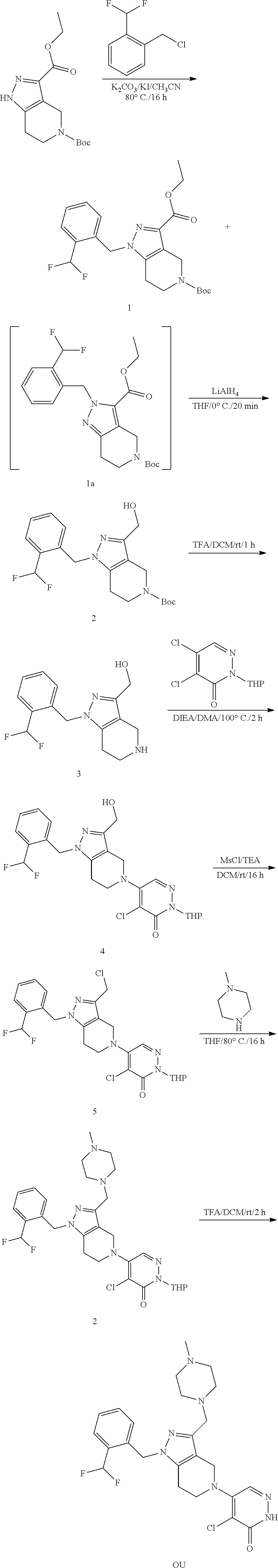

### Preparation of OU

### 5-tert-Butyl 3-ethyl 2-[[2-(difluoromethyl)phenyl]methyl]-2H,4H,5H,6H,7H-pyrazolo[4,3-c]pyridine-3,5-dicarboxylate

To a stirred solution of 1-(chloromethyl)-2-(difluoromethyl)benzene (800 mg, 4.530 mmol, 1 equiv.) and 5-tert-butyl3-ethyl 1H,4H,5H,6H,7H-pyrazolo[4,3-c]pyridine-3,5-dicarboxylate (1337.96 mg, 4.530 mmol, 1.00 equiv.) in MeCN (15 mL) was added KI (752.04 mg, 4.530 mmol, 1 equiv.) and K2CO3 (1252.22 mg, 9.061 mmol, 2 equiv.) at room temperature under nitrogen atmosphere. The mixture was stirred at 80 degrees Celsius overnight. Desired product could be detected by LCMS. The resulting mixture was concentrated under reduced pressure. The residue was purified by silica gel column chromatography, eluted with PE/EtOAc (5:1 to 3:1) to afford 5-tert-butyl 3-ethyl 1-[[2-(difluoromethyl)phenyl]methyl]-1H,4H,5H,6H,7H-pyrazolo[4,3-c]pyridine-3,5-dicarboxylate (500 mg, 25.34%) as a yellow solid and 5-tert-butyl 3-ethyl 2-[[2-(difluoromethyl)phenyl]methyl]-2H,4H,5H,6H,7H-pyrazolo[4,3-c]pyridine-3,5-dicarboxylate (200 mg, 10.14%) as a yellow solid.

### tert-Butyl 1-[[2-(difluoromethyl)phenyl]methyl]-3-(hydroxymethyl)-1H,4H,5H,6H,7H-pyrazolo[4,3-c]pyridine-5-carboxylate

To a stirred solution of 5-tert-butyl 3-ethyl 1-[[2-(difluoromethyl)phenyl]methyl]-1H,4H,5H,6H,7H-pyrazolo[4,3-c]pyridine-3,5-dicarboxylate (600 mg, 1.378 mmol, 1 equiv.) in THF (10 mL, 123.430 mmol, 89.58 equiv.) was added LiAlH4 (62.75 mg, 1.653 mmol, 1.2 equiv.) in portions at 0 degrees Celsius under nitrogen atmosphere. The mixture was stirred at rt for 1 h. Desired product could be detected by LCMS. The reaction was quenched by the addition of Water (5 mL) at 0 degrees C. The mixture was concentrated and purified by silica gel column chromatography (PE:EA=2:1) to afford tert-butyl 1-[[2-(difluoromethyl)phenyl]methyl]-3-(hydroxymethyl)-1H,4H,5H,6H,7H-pyrazolo[4,3-c]pyridine-5-carboxylate (500 mg, 92.24%) as white solid.

### (1-[[2-(Difluoromethyl)phenyl]methyl]-1H,4H,5H,6H,7H-pyrazolo[4,3-c]pyridin-3-yl)methanol

To a stirred solution of tert-butyl 1-[[2-(difluoromethyl)phenyl]methyl]-3-(hydroxymethyl)-1H,4H,5H,6H,7H-pyrazolo[4,3-c]pyridine-5-carboxylate (500 mg, 1.271 mmol, 1 equiv.) in DCM (10 mL, 157.300 mmol, 123.78 equiv.) was added TFA (2 mL, 26.926 mmol, 21.19 equiv.) dropwise at 0 degrees Celsius under nitrogen atmosphere. The mixture was stirred at rt for 2 h. Desired product could be detected by LCMS. The mixture was concentrated to afford (1-[[2-(difluoromethyl)phenyl]methyl]-1H,4H,5H,6H,7H-pyrazolo[4,3-c]pyridin-3-yl)methanol (370 mg, 99.26%) as yellow solid.

### 4-Chloro-5-(1-[[2-(difluoromethyl)phenyl]methyl]-3-(hydroxymethyl)-1H,4H,5H,6H,7H-pyrazolo[4,3-c]pyridin-5-yl)-2-(oxan-2-yl)-2,3-dihydropyridazin-3-one

To a stirred solution of (1-[[2-(difluoromethyl)phenyl]methyl]-1H,4H,5H,6H,7H-pyrazolo[4,3-c]pyridin-3-yl)methanol (370 mg, 1.261 mmol, 1 equiv.) and 4,5-dichloro-2-(oxan-2-yl)-2,3-dihydropyridazin-3-one (471.31 mg, 1.892 mmol, 1.5 equiv.) in DMA (1 mL) was added DIEA (326.06 mg, 2.523 mmol, 2 equiv.) dropwise at room temperature under nitrogen atmosphere. The mixture was stirred at 100 degrees Celsius overnight. Desired product could be detected by LCMS. The resulting mixture was concentrated under reduced pressure. The residue was purified by silica gel column chromatography, eluted with PE/EtOAc (6:1 to 3:1) to afford 4-chloro-5-(1-[[2-(difluoromethyl)phenyl]methyl]-3-(hydroxymethyl)-1H,4H,5H,6H,7H-pyrazolo[4,3-c]pyridin-5-yl)-2-(oxan-2-yl)-2,3-dihydropyridazin-3-one (420 mg, 65.81%) as a white solid.

### 4-Chloro-5-[3-(chloromethyl)-1-[[2-(difluoromethyl)phenyl]methyl]-1H,4H,5H,6H,7H-pyrazolo[4,3-c]pyridin-5-yl]-2-(oxan-2-yl)-2,3-dihydropyridazin-3-one

To a stirred solution of 4-chloro-5-(1-[[2-(difluoromethyl)phenyl]methyl]-3-(hydroxymethyl)-1H,4H,5H,6H,7H-pyrazolo[4,3-c]pyridin-5-yl)-2-(oxan-2-yl)-2,3-dihydropyridazin-3-one (400 mg, 0.791 mmol, 1 equiv.) and TEA (160.00 mg, 1.581 mmol, 2 equiv.) in DCM (8 mL, 125.840 mmol, 159.17 equiv.) was added MsCl (108.68 mg, 0.949 mmol, 1.2 equiv.) dropwise at 0 degrees Celsius under nitrogen atmosphere. The mixture was stirred at rt overnight. Desired product could be detected by LCMS. The resulting mixture was concentrated under reduced pressure. The residue was purified by silica gel column chromatography, eluted with PE/EtOAc (5:1) to afford 4-chloro-5-[3-(chloromethyl)-1-[[2-(difluoromethyl)phenyl]methyl]-1H,4H,5H,6H,7H-pyrazolo[4,3-c]pyridin-5-yl]-2-(oxan-2-yl)-2,3-dihydropyridazin-3-one (400 mg, 96.48%) as a yellow solid.

### 4-Chloro-5-(1-[[2-(difluoromethyl)phenyl]methyl]-3-[(4-methylpiperazin-1-yl)methyl]-1H,4H,5H,6H,7H-pyrazolo[4,3-c]pyridin-5-yl)-2-(oxan-2-yl)-2,3-dihydropyridazin-3-one

To a stirred solution of 4-chloro-5-[3-(chloromethyl)-1-[[2-(difluoromethyl)phenyl]methyl]-1H,4H,5H,6H,7H-pyrazolo[4,3-c]pyridin-5-yl]-2-(oxan-2-yl)-2,3-dihydropyridazin-3-one (60 mg, 0.103 mmol, 1 equiv.) in MeCN (10 mL) was added 1-methylpiperazine (51.45 mg, 0.514 mmol, 5 equiv.) dropwise at room temperature under nitrogen atmosphere. The mixture was stirred at 80 degrees Celsius overnight. Desired product could be detected by LCMS. The resulting mixture was concentrated under reduced pressure. The residue was purified by silica gel column chromatography, eluted with PE/EtOAc (5:1 to 1:1) to afford 4-chloro-5-(1-[[2-(difluoromethyl)phenyl]methyl]-3-[(4-methylpiperazin-1-yl)methyl]-1H,4H,5H,6H,7H-pyrazolo[4,3-c]pyridin-5-yl)-2-(oxan-2-yl)-2,3-dihydropyridazin-3-one (60 mg, 99.31%) as a white solid.

### 4-Chloro-5-(1-[[2-(difluoromethyl)phenyl]methyl]-3-[(4-methylpiperazin-1-yl)methyl]-1H,4H,5H,6H,7H-pyrazolo[4,3-c]pyridin-5-yl)-2,3-dihydropyridazin-3-one

To a stirred solution of 4-chloro-5-(1-[[2-(difluoromethyl)phenyl]methyl]-3-[(4-methylpiperazin-1-yl)methyl]-1H,4H,5H,6H,7H-pyrazolo[4,3-c]pyridin-5-yl)-2-(oxan-2-yl)-2,3-dihydropyridazin-3-one (60 mg) in DCM (10 mL) were added TFA (2 mL) dropwise at 0 degrees Celsius under nitrogen atmosphere. The mixture was stirred at rt for 2 h. Desired product could be detected by LCMS. The reaction was quenched by the addition of sat. NaHCO₃(aq.) (5 mL) at room temperature. The resulting mixture was concentrated under reduced pressure. The crude product (mg) was purified by Prep-HPLC with the following conditions (Column: XBridge Prep OBD C18 Column 30×150 mm Sum; Mobile Phase A: Water (10 MMOL/L NH4HCO3), Mobile Phase B: ACN; Flow rate: 60 mL/min; Gradient: 20% B to 35% B in 8 min; 220 nm; Rt: 7.25 min) to afford 4-chloro-5-(1-[[2-(difluoromethyl)phenyl]methyl]-3-[(4-methylpiperazin-1-yl)methyl]-1H,4H,5H,6H,7H-pyrazolo[4,3-c]pyridin-5-yl)-2,3-dihydropyridazin-3-one (30 mg) as a white solid.

### Example 30. Synthesis of Compound OP

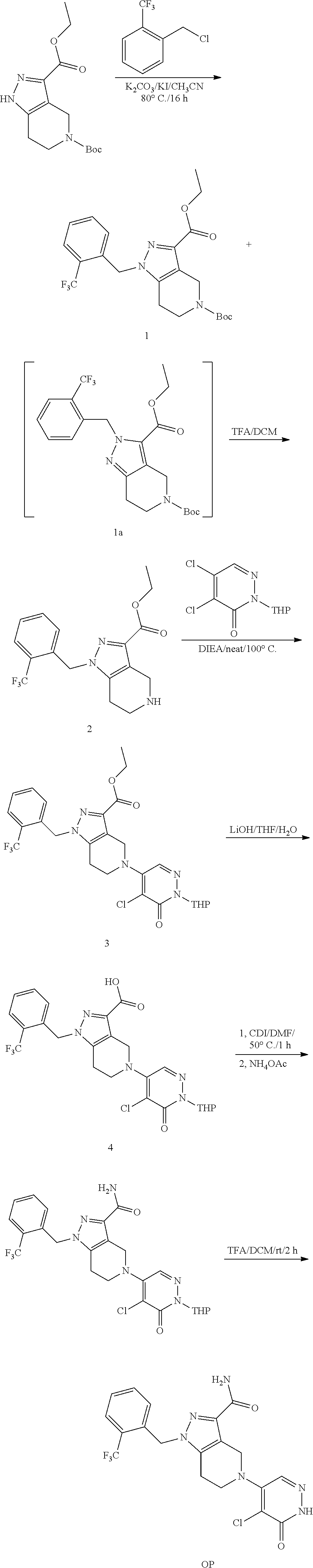

### Preparation of OP

### 2-tert-Butyl 7-ethyl 5-[[2-(trifluoromethyl)phenyl]methyl]-1H,2H,3H,4H,5H-cyclopenta[c]pyridine-2,7-dicarboxylate

To a stirred solution of 5-tert-butyl 3-ethyl 1H,4H,5H,6H,7H-pyrazolo[4,3-c]pyridine-3,5-dicarboxylate (1.5 g, 5.079 mmol, 1 equiv.) and 1-(bromomethyl)-2-(trifluoromethyl)benzene (1.46 g, 6.095 mmol, 1.2 equiv.) in ACN (20 mL, 380.494 mmol) were added K2CO3 (1.40 g, 10.158 mmol, 2 equiv.) and KI (0.84 g, 5.079 mmol, 1 equiv.) in portions at room temperature under nitrogen atmosphere. The resulting mixture was stirred for 2 h at 80 degrees Celsius under nitrogen atmosphere. The reaction was monitored by LCMS. The resulting mixture was concentrated under reduced pressure. The resulting mixture was extracted with EtOAc (50 mL). The combined organic layers were washed with brine (3×100 mL), dried over anhydrous Na2SO4. After filtration, the filtrate was concentrated under reduced pressure. The resulting mixture was used in the next step (E00692-127) directly without further purification.

### Ethyl 1-[[2-(trifluoromethyl)phenyl]methyl]-1H,4H,5H,6H,7H-pyrazolo[4,3-c]pyridine-3-carboxylate

To a stirred solution of 2-tert-butyl 7-ethyl 5-[[2-(trifluoromethyl)phenyl]methyl]-1H,2H,3H,4H,5H-cyclopenta[c]pyridine-2,7-dicarboxylate (1 g, 2.215 mmol, 1 equiv.) in DCM (10 mL) was added TFA (3 mL) in portions at room temperature under nitrogen atmosphere. The resulting mixture was stirred for 2 h at room temperature under nitrogen atmosphere. The reaction was monitored by LCMS. The resulting mixture was concentrated under reduced pressure. The resulting mixture was used in the next step (E00692-129) directly without further purification.

### Ethyl 5-[5-chloro-1-(oxan-2-yl)-6-oxo-1,6-dihydropyridazin-4-yl]-1-[[2-(trifluoromethyl)phenyl]methyl]-1H,4H,5H,6H,7H-pyrazolo[4,3-c]pyridine-3-carboxylate

To a stirred solution of ethyl 1-[[2-(trifluoromethyl)phenyl]methyl]-1H,4H,5H,6H,7H-pyrazolo[4,3-c]pyridine-3-carboxylate (750 mg, 2.123 mmol, 1 equiv.) and 4,5-dichloro-2-(oxan-2-yl)-2,3-dihydropyridazin-3-one (528.71 mg, 2.123 mmol, 1 equiv.) was added DIEA (5 mL) in portions at room temperature under nitrogen atmosphere. The resulting mixture was stirred for overnight at 100 degrees Celsius under nitrogen atmosphere as a neat reaction. The reaction was monitored by LCMS. The mixture was allowed to cool down to room temperature. The resulting mixture was extracted with EtOAc (30 mL). The combined organic layers were washed with brine (3×50 mL), dried over anhydrous Na2SO4. After filtration, the filtrate was concentrated under reduced pressure. The residue was purified by Prep-TLC (PE/EtOAc 1:1) to afford ethyl 5-[5-chloro-1-(oxan-2-yl)-6-oxo-1,6-dihydropyridazin-4-yl]-1-[[2-(trifluoromethyl)phenyl]methyl]-1H,4H,5H,6H,7H-pyrazolo[4,3-c]pyridine-3-carboxylate (1 g, 83.24%) as a light yellow oil.

### 5-[5-chloro-1-(oxan-2-yl)-6-oxo-1,6-dihydropyridazin-4-yl]-1-[[2-(trifluoromethyl)phenyl]methyl]-1H,4H,5H,6H,7H-pyrazolo[4,3-c]pyridine-3-carboxylic acid

To a stirred solution of ethyl 5-[5-chloro-1-(oxan-2-yl)-6-oxo-1,6-dihydropyridazin-4-yl]-1-[[2-(trifluoromethyl)phenyl]methyl]-1H,4H,5H,6H,7H-pyrazolo[4,3-c]pyridine-3-carboxylate (1 g, 1.767 mmol, 1 equiv.) in THF (5 mL) and H₂O (5 mL) was added LiOH (0.21 g, 0.009 mmol, 5 equiv.) in portions at room temperature under nitrogen atmosphere. The resulting mixture was stirred for 3 h at 50 degrees Celsius under nitrogen atmosphere. The reaction was monitored by LCMS. The mixture was acidified to pH 6 with HCl (aq.). The resulting mixture was extracted with EtOAc (30 mL). The combined organic layers were washed with brine (3×10 mL), dried over anhydrous Na2SO4. After filtration, the filtrate was concentrated under reduced pressure. The residue was purified by silica gel column chromatography, eluted with CH2Cl2/MeOH (50:1 to 5:1) to afford 5-[5-chloro-1-(oxan-2-yl)-6-oxo-1,6-dihydropyridazin-4-yl]-1-[[2-(trifluoromethyl)phenyl]methyl]-1H,4H,5H,6H,7H-pyrazolo[4,3-c]pyridine-3-carboxylic acid (700 mg, 73.65%) as a light yellow oil.

### 5-[5-Chloro-1-(oxan-2-yl)-6-oxo-1,6-dihydropyridazin-4-yl]-1-[[2-(trifluoromethyl)phenyl]methyl]-1H,4H,5H,6H,7H-pyrazolo[4,3-c]pyridine-3-carboxamide

To a stirred solution of 5-[5-chloro-1-(oxan-2-yl)-6-oxo-1,6-dihydropyridazin-4-yl]-1-[[2-(trifluoromethyl)phenyl]methyl]-1H,4H,5H,6H,7H-pyrazolo[4,3-c]pyridine-3-carboxylic acid (700 mg, 1.301 mmol, 1 equiv.) in DMF (10 mL) was added CDI (316.51 mg, 1.952 mmol, 1.5 equiv.) in portions at room temperature under nitrogen atmosphere. The resulting mixture was stirred for 1 h at 50 degrees Celsius under nitrogen atmosphere. To the above mixture was added NH4OAc (300.92 mg, 3.904 mmol, 3 equiv.) in portions over 5 min at 50 degrees C. The resulting mixture was stirred for additional 2 h at 50 degrees C. The reaction was monitored by LCMS. The mixture was allowed to cool down to room temperature. The resulting mixture was extracted with EtOAc (30 mL). The combined organic layers were washed with brine (3×50 mL), dried over anhydrous Na2SO4. After filtration, the filtrate was concentrated under reduced pressure. The residue was purified by silica gel column chromatography, eluted with PE/EtOAc (20:1 to 5:1) to afford 5-[5-chloro-1-(oxan-2-yl)-6-oxo-1,6-dihydropyridazin-4-yl]-1-[[2-(trifluoromethyl)phenyl]methyl]-1H,4H,5H,6H,7H-pyrazolo[4,3-c]pyridine-3-carboxamide (40 mg, 5.72%) as a light yellow oil.

### 5-(5-Chloro-6-oxo-1,6-dihydropyridazin-4-yl)-1-[[2-(trifluoromethyl)phenyl]methyl]-1H,4H,5H,6H,7H-pyrazolo[4,3-c]pyridine-3-carboxamide

To a stirred solution of 5-[5-chloro-1-(oxan-2-yl)-6-oxo-1,6-dihydropyridazin-4-yl]-1-[[2-(trifluoromethyl)phenyl]methyl]-1H,4H,5H,6H,7H-pyrazolo[4,3-c]pyridine-3-carboxamide (40 mg, 0.074 mmol, 1 equiv.) in DCM (10 mL) was added TFA (3 mL, 40.389 mmol, 542.16 equiv.) at room temperature. The resulting mixture was stirred for 2 h at room temperature. The reaction was monitored by LCMS. The resulting mixture was concentrated under reduced pressure. The mixture was basified to pH 8 with saturated NaHCO₃(aq.). The crude product (30 mg) was purified by Prep-HPLC with the following conditions (Column: XBridge Shield RP18 OBD Column, 5 um, 19*150 mm; Mobile Phase A: Water (10 MMOL/L NH4HCO3), Mobile Phase B: ACN; Flow rate: 20 mL/min; Gradient: 24% B to 45% B in 7 min; 220/254 nm; Rt: 6.45 min) to afford 5-(5-chloro-6-oxo-1,6-dihydropyridazin-4-yl)-1-[[2-(trifluoromethyl)phenyl]methyl]-1H,4H,5H,6H,7H-pyrazolo[4,3-c]pyridine-3-carboxamide (12.5 mg, 37.06%) as a white solid.

### Example 31. Synthesis of Compound NL

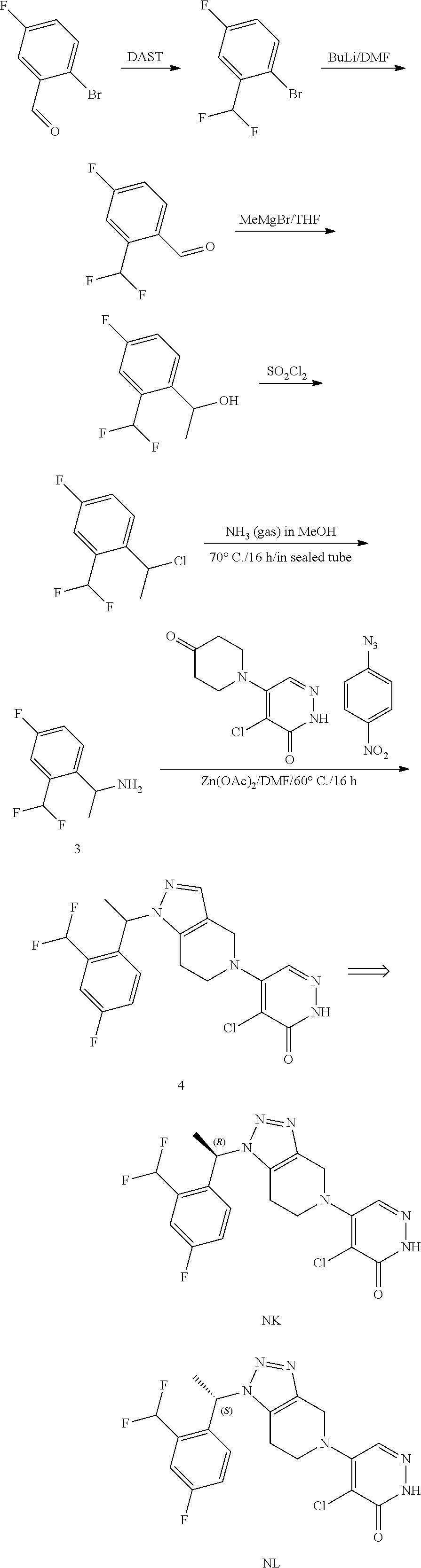

### Preparation of NK and NL

### 1-Bromo-2-(difluoromethyl)-4-fluorobenzene

To a stirred solution of 2-bromo-5-fluorobenzaldehyde (10 g, 49.26 mmol, 1 equiv.) in DCM (10 mL) was added DAST (15.9 g, 98.64 mmol, 2.00 equiv.). The resulting mixture was stirred for 2 h at −10 degrees C. The resulting mixture was concentrated under reduced pressure. The residue was purified by silica gel column chromatography, eluted with PE/EtOAc (5:1) to afford 1-bromo-2-(difluoromethyl)-4-fluorobenzene (8.2 g, 73.98%) as a light yellow oil.

### 2-(Difluoromethyl)-4-fluorobenzaldehyde

A solution of 1-bromo-2-(difluoromethyl)-4-fluorobenzene (8 g, 35.55 mmol, 1 equiv.) and n-BuLi (2.7 g, 42.15 mmol, 1.19 equiv.) in THF (150 mL) was stirred for 2 h at −78 degrees C. To the above mixture was added DMF (3.9 g, 53.33 mmol, 1.5 equiv.). The resulting mixture was stirred for 1 h at −78 degrees C. The reaction was quenched by the addition of Water (50 mL) at −70 degrees C. The solution was extracted with EtOAc (3×50 mL). The combined organic layers were washed with brine (2×30 mL), dried over anhydrous Na2SO4. After filtration, the filtrate was concentrated under reduced pressure. The residue was purified by silica gel column chromatography, eluted with PE/EtOAc (5:1) to afford 2-(difluoromethyl)-4-fluorobenzaldehyde (3 g, 48.46%) as a light yellow oil.

### 1-[2-(Difluoromethyl)-4-fluorophenyl]ethan-1-ol

To a stirred solution of 2-(difluoromethyl)-4-fluorobenzaldehyde (3 g, 17.23 mmol, 1 equiv.) in THF (30 mL, 416.06 mmol, 10 equiv.) was added CH₃MgBr (25.84 mL, 25.84 mmol, 1.5 equiv.) dropwise at −30 degrees Celsius under nitrogen atmosphere. The resulting mixture was stirred for 2 h at −10 degrees Celsius under nitrogen atmosphere. The reaction was quenched with sat. NH4Cl(aq.) at 0 degrees C. The mixture was extracted with EtOAc (3×300 mL). The combined organic layers were washed with brine (3×300 mL), dried over anhydrous Na2SO4. After filtration, the filtrate was concentrated under reduced pressure. The residue was purified by silica gel column chromatography, eluted with PE/EtOAc (100:1 to 50:1) to afford 1-[2-(difluoromethyl)-4-fluorophenyl]ethan-1-ol (2.68 g, 81.80%) as red oil.

### 1-(1-Chloroethyl)-2-(difluoromethyl)-4-fluorobenzene

To a stirred solution/mixture of 1-[2-(difluoromethyl)-4-fluorophenyl]ethan-1-ol (2.68 g, 14.09 mmol, 1 equiv.) in DCM (30 mL, 140.93 mmol, 10 equiv.) was added SO2Cl2 (6.7 g, 49.64 mmol, 3.52 equiv.) dropwise in portions at 0 degrees Celsius under air atmosphere. The resulting mixture was stirred for 2 h at 20 degrees C. The resulting oil was dried under vacuum. to afford 1-(1-chloroethyl)-2-(difluoromethyl)-4-fluorobenzene (2.36 g, 80.27%) as red oil.

### 1-[2-(difluoromethyl)-4-fluorophenyl]ethan-1-amine

To a stirred solution of 1-(1-chloroethyl)-2-(difluoromethyl)-4-fluorobenzene (300 mg, 1.44 mmol, 1 equiv.) in MeOH with NH3(g) at rt under nitrogen atmosphere. The resulting mixture was stirred for 20 h at 70 degrees Celsius under nitrogen atmosphere. The reaction was monitored by LCMS. The mixture was allowed to cool down to rt. The resulting mixture was concentrated under reduced pressure. This resulted in 1-[2-(difluoromethyl)-4-fluorophenyl]ethan-1-amine (130 mg, 47.78%) as a yellow oil. The resulting mixture was used in the next step directly without further purification

### 4-chloro-5-[1-[(1S)-1-[2-(difluoromethyl)-4-fluorophenyl]ethyl]-1H,4H,5H,6H,7H-[1,2,3]triazolo[4,5-c]pyridin-5-yl]-2,3-dihydropyridazin-3-one and 4-chloro-5-[1-[(1R)-1-[2-(difluoromethyl)-4-fluorophenyl]ethyl]-1H,4H,5H,6H,7H-[1,2,3]triazolo[4,5-c]pyridin-5-yl]-2,3-dihydropyridazin-3-one

To a stirred mixture of 1-[2-(difluoromethyl)-4-fluorophenyl]ethan-1-amine (130.0 mg, 0.69 mmol, 2.00 equiv.) and 4-chloro-5-(4-oxopiperidin-1-yl)-2,3-dihydropyridazin-3-one (78.2 mg, 0.34 mmol, 1 equiv.) in DMF (10 mL) were added 1-azido-4-nitrobenzene (78.9 mg, 0.48 mmol, 1.40 equiv.) and Zn(OAc)2 (63.0 mg, 0.34 mmol, 1.00 equiv.) in portions at rt under nitrogen atmosphere. The resulting mixture was stirred for 16 h at 60 degrees Celsius under nitrogen atmosphere. The reaction was monitored by LCMS. The mixture was allowed to cool down to rt. The residue was purified by reverse phase flash with the following conditions (Column: XBridge Shield RP18 OBD Column, 20-40 um, 19*150 mm; Mobile Phase A: Water (10 MMOL/L NH4HCO3), Mobile Phase B: ACN; Flow rate: 80 mL/min; Gradient: 30% B to 70% B in 30 min; 220 nm; Rt: 7.08 min) to afford mixture product. The residue (100 mg) was purified by Chiral-Prep-HPLC with the following conditions: Column, CHIRALPAK IF-3, 0.46*5 cm; 3 um; Mobile phase: MtBE (0.1% DEA):EtOH=80:20; Detector: UV-254 nm. 4-chloro-5-[1-[(1S)-1-[2-(difluoromethyl)-4-fluorophenyl]ethyl]-1H,4H,5H,6H,7H-[1,2,3]triazolo[4,5-c]pyridin-5-yl]-2,3-dihydropyridazin-3-one (19.0 mg) was obtained at 3.835 min as a off-white solid. 4-chloro-5-[1-[(1R)-1-[2-(difluoromethyl)-4-fluorophenyl]ethyl]-1H,4H,5H,6H,7H-[1,2,3]triazolo[4,5-c]pyridin-5-yl]-2,3-dihydropyridazin-3-one (33.8 mg) was obtained at 3.185 min as a off-white solid.

### Example 32. Synthesis of Compound QM

Compound QM

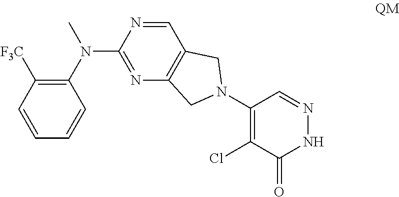 [SMILES: CN(c1ncc2c(n1)CN(c1cn[nH]c(=O)c1Cl)C2)c1ccccc1C(F)(F)F]

was prepared by the methods and scheme described for QL by using the corresponding aniline.

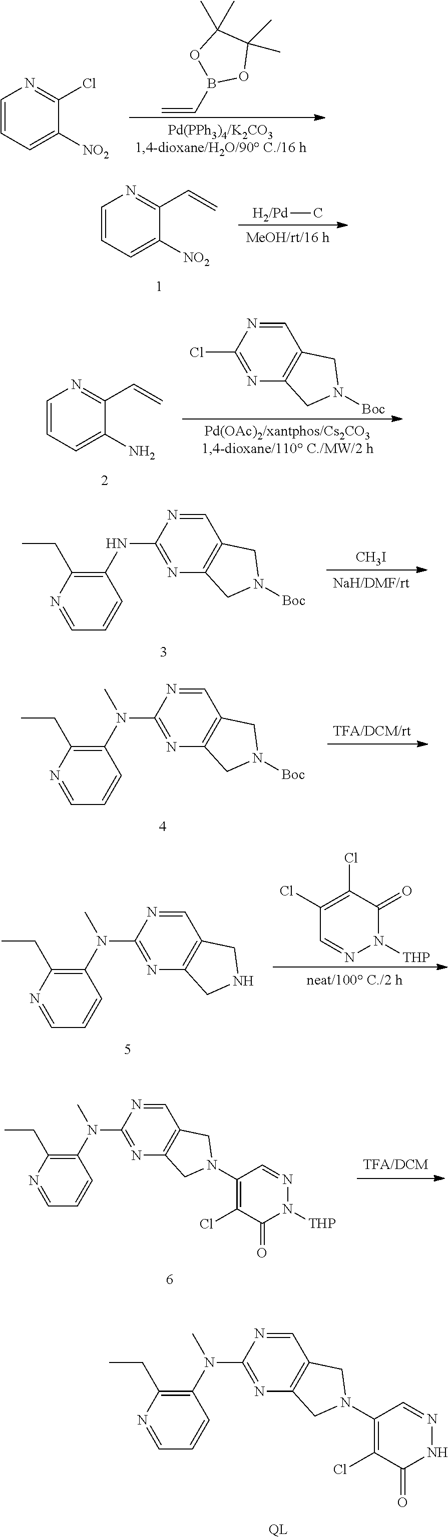

### Preparation of QL

### 2-Ethenyl-3-nitropyridine

To a stirred mixture of 2-chloro-3-nitropyridine (2 g, 12.615 mmol, 1 equiv.) and Na2CO3 (2.67 g, 25.230 mmol, 2.0 equiv.) in 1,4-dioxane (20 mL) and H₂O (1 mL) were added Pd(PPh3)4 (0.73 g, 0.631 mmol, 0.05 equiv.) and 2-ethenyl-4,4,5,5-tetramethyl-1,3,2-dioxaborolane (1.94 g, 12.615 mmol, 1.00 equiv.) at 0 degrees Celsius under nitrogen atmosphere. The resulting mixture was stirred for 3 h at room temperature under nitrogen atmosphere. The reaction was monitored by LCMS. The resulting mixture was concentrated under reduced pressure. The residue was purified by silica gel column chromatography, eluted with PE/EtOAc (10:1 to 5:1) to afford 2-ethenyl-3-nitropyridine (1.1 g, 58.08%) as a brown solid.

### 2-Ethylpyridin-3-amine

To a stirred solution of 2-ethenyl-3-nitropyridine (1.1 g, 7.327 mmol, 1 equiv.) in MeOH (10 mL) was added Pd/C (100 mg, 0.266 mmol, 0.04 equiv.) at room temperature under hydrogen atmosphere. The resulting mixture was stirred for 16 h at room temperature under hydrogen atmosphere. The reaction was monitored by LCMS. The resulting mixture was filtered, the filter cake was washed with MeOH (2×10 mL). The filtrate was concentrated under reduced pressure. The residue product was purified by reverse phase flash with the following conditions (Column: XBridge Prep OBD C18 Column 30×150 mm Sum; Mobile Phase A: Water (10 MMOL/L NH4HCO3), Mobile Phase B: ACN; Flow rate: 60 mL/min; Gradient: 20% B to 40% B in 11 min; 220 nm; Rt: 11.77 min) to afford 2-ethylpyridin-3-amine (620 mg, 69.27%) as a white solid.

### tert-Butyl 2-[(2-ethylpyridin-3-yl)amino]-5H,6H,7H-pyrrolo[3,4-d]pyrimidine-6-carboxylate

To a stirred mixture of tert-butyl2-chloro-5H,6H,7H-pyrrolo[3,4-d]pyrimidine-6-carboxylate (200 mg, 0.782 mmol, 1 equiv.) and 2-ethyl-3-nitropyridine (238.02 mg, 1.564 mmol, 2.0 equiv.) in 1,4-dioxane (20 mL) were added Cs2CO3 (509.69 mg, 1.564 mmol, 2.0 equiv.) and Pd(AcO)2 (35.12 mg, 0.156 mmol, 0.2 equiv.) at room temperature under nitrogen atmosphere. Then XantPhos (181.03 mg, 0.313 mmol, 0.4 equiv.) was added at room temperature under nitrogen atmosphere. The final reaction mixture was irradiated with microwave radiation for 2 h at 110 degrees C. The reaction was monitored by LCMS. The mixture was allowed to cool down to room temperature. The resulting mixture was filtered, the filter cake was washed with CH2Cl2 (2×10 mL). The filtrate was concentrated under reduced pressure. The crude product was purified by reverse phase flash with the following conditions (Column: XBridge Prep OBD C18 Column 30×150 mm Sum; Mobile Phase A: Water (10 MMOL/L NH4HCO3), Mobile Phase B: ACN; Flow rate: 60 mL/min; Gradient: 20% B to 40% B in 11 min; 220 nm; Rt: 11.77 min) to afford tert-butyl2-[(2-ethylpyridin-3-yl)amino]-5H,6H,7H-pyrrolo[3,4-d]pyrimidine-6-carboxylate (250 mg, 93.62%) as a brown solid.

### tert-Butyl 2-[(2-ethylpyridin-3-yl)(methyl)amino]-5H,6H,7H-pyrrolo[3,4-d]pyrimidine-6-carboxylate

To a stirred solution of tert-butyl 2-[(2-ethylpyridin-3-yl)amino]-5H,6H,7H-pyrrolo[3,4-d]pyrimidine-6-carboxylate (300 mg, 0.879 mmol, 1 equiv.) in DMF (10 mL) was added NaH (42.17 mg, 1.757 mmol, 2.0 equiv.) at 0 degrees Celsius under nitrogen atmosphere. The resulting mixture was stirred for 1 h at degrees Celsius under nitrogen atmosphere. Then CH₃I (249.44 mg, 1.757 mmol, 2.00 equiv.) was added at 0 degrees Celsius under nitrogen atmosphere. The resulting mixture was stirred for 1 h at room temperature under nitrogen atmosphere. The reaction was monitored by LCMS. The resulting mixture was diluted with water (2 mL). The crude product was purified by reverse phase flash with the following conditions (Column: XBridge Prep OBD C18 Column 30×150 mm Sum; Mobile Phase A: Water (10 MMOL/L NH4HCO3), Mobile Phase B: ACN; Flow rate: 60 mL/min; Gradient: 20% B to 40% B in 11 min; 220 nm; Rt: 11.77 min) to afford tert-butyl 2-[(2-ethylpyridin-3-yl)(methyl)amino]-5H,6H,7H-pyrrolo[3,4-d]pyrimidine-6-carboxylate (250 mg, 80.04%) as a brown solid.

### N-(2-ethylpyridin-3-yl)-N-methyl-6,7-dihydro-5H-pyrrolo[3,4-d]pyrimidin-2-amine

To a stirred solution of tert-butyl 2-[(2-ethylpyridin-3-yl)(methyl)amino]-5H,6H,7H-pyrrolo[3,4-d]pyrimidine-6-carboxylate (200 mg, 0.586 mmol, 1 equiv.) in DCM (4 mL) was added TFA (1 mL, 13.463 mmol, 22.98 equiv.) at room temperature. The resulting mixture was stirred for 1 h at room temperature. The reaction was monitored by LCMS. The resulting mixture was concentrated under reduced pressure. The mixture was basified to pH 8 with saturated NaHCO₃(aq.). The crude product was purified by reverse phase flash with the following conditions (Column: XBridge Prep OBD C18 Column 30×150 mm Sum; Mobile Phase A: Water (10 MMOL/L NH4HCO3), Mobile Phase B: ACN; Flow rate: 60 mL/min; Gradient: 18% B to 35% B in 8 min; 220 nm; Rt: 7.12 min) to afford N-(2-ethylpyridin-3-yl)-N-methyl-6,7-dihydro-5H-pyrrolo[3,4-d]pyrimidin-2-amine (120 mg, 84.89%) as a brown solid.

### 4-Chloro-5-[2-[(2-ethylpyridin-3-yl)(methyl)amino]-5H,6H,7H-pyrrolo[3,4-d]pyrimidin-6-yl]-2-(oxan-2-yl)-2,3-dihydropyridazin-3-one

To a stirred solution of N-(2-ethylpyridin-3-yl)-N-methyl-6,7-dihydro-5H-pyrrolo[3,4-d]pyrimidin-2-amine (120 mg, 0.497 mmol, 1 equiv.) in DIEA (0.1 mL) was added 4,5-dichloro-2-(oxan-2-yl)-2,3-dihydropyridazin-3-one (99.10 mg, 0.398 mmol, 0.80 equiv.) at room temperature. The resulting mixture was stirred for 1 h at 90 degrees C. The reaction was monitored by LCMS. The mixture was allowed to cool down to room temperature. The resulting mixture was concentrated under reduced pressure. The residue was purified by Prep-TLC (CH2Cl₂/MeOH 12:1) to afford 4-chloro-5-[2-[(2-ethylpyridin-3-yl)(methyl)amino]-5H,6H,7H-pyrrolo[3,4-d]pyrimidin-6-yl]-2-(oxan-2-yl)-2,3-dihydropyridazin-3-one (100 mg, 42.97%) as a brown solid.

### 4-Chloro-5-[2-[(2-ethylpyridin-3-yl)(methyl)amino]-5H,6H,7H-pyrrolo[3,4-d]pyrimidin-6-yl]-2,3-dihydropyridazin-3-one

To a stirred solution of 4-chloro-5-[2-[(2-ethylpyridin-3-yl)(methyl)amino]-5H,6H,7H-pyrrolo[3,4-d]pyrimidin-6-yl]-2-(oxan-2-yl)-2,3-dihydropyridazin-3-one (100 mg, 0.214 mmol, 1 equiv.) in DCM (4 mL) was added TFA (1 mL) at room temperature. The resulting mixture was stirred for 1 h at room temperature. The reaction was monitored by LCMS. The resulting mixture was concentrated under reduced pressure. The mixture was basified to pH 8 with saturated NaHCO₃(aq.). The crude product (80 mg) was purified by Prep-HPLC with the following conditions (Column: XBridge Prep OBD C18 Column 30×150 mm Sum; Mobile Phase A: Water (10 MMOL/L NH4HCO3), Mobile Phase B: ACN; Flow rate: 60 mL/min; Gradient: 15% B to 35% B in 8 min; 220 nm; Rt: 6.65 min) to afford 4-chloro-5-[2-[(2-ethylpyridin-3-yl)(methyl)amino]-5H,6H,7H-pyrrolo[3,4-d]pyrimidin-6-yl]-2,3-dihydropyridazin-3-one (67.4 mg, 82.17%) as a white solid.

### Example 33. Synthesis of Compounds MD and ME

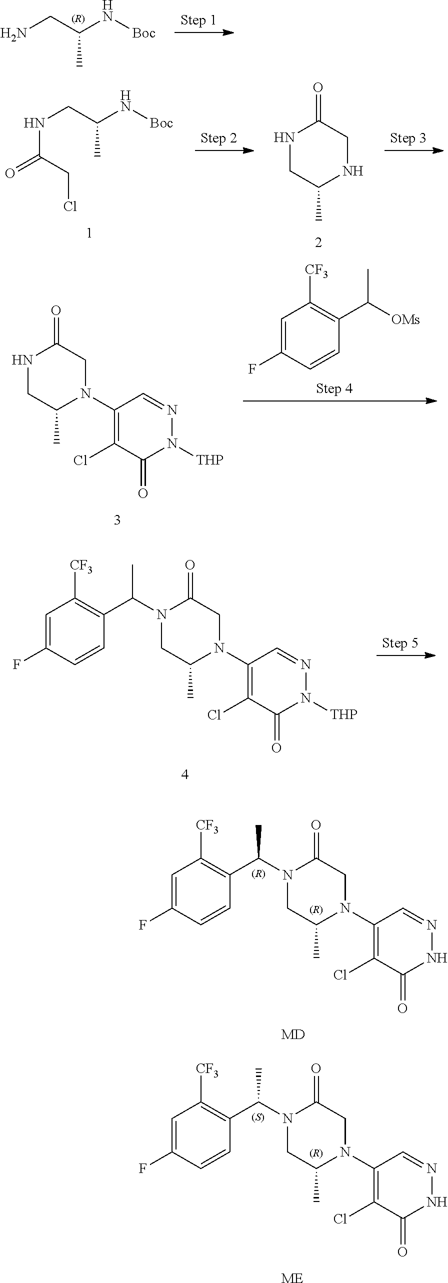

### Step 1

### tert-butyl N-[(2R)-1-(2-chloroacetamido)propan-2-yl]carbamate

To a stirred solution of tert-butyl N-[(2R)-1-aminopropan-2-yl]carbamate (3 g, 17.217 mmol, 1 equiv.) in EA (50 mL) was added the solution of Na2CO3 (3649.65 mg, 34.434 mmol, 2 equiv.) in H2O (10 mL) at room temperature. Then the solution of 2-chloroacetyl chloride (3.89 g, 34.434 mmol, 2 equiv.) in EA (10 mL) was added dropwise at 0 degrees C. The resulting mixture was stirred for 2 h at room temperature. The reaction was monitored by LCMS. The reaction was quenched with Water at room temperature. The resulting mixture was extracted with EtOAc (3×100 mL). The combined organic layers were washed with brine (1×100 mL), dried over anhydrous Na2SO4. After filtration, the filtrate was concentrated under reduced pressure. This resulted in tert-butyl N-[(2R)-1-(2-chloroacetamido)propan-2-yl]carbamate (4.5 g, crude) as a white solid.

### Step 2

### (5R)-5-methylpiperazin-2-one

To a stirred solution of tert-butyl N-[(2R)-1-(2-chloroacetamido)propan-2-yl]carbamate (4.5 g, 17.948 mmol, 1 equiv.) in DCM (30 mL) was added the solution of TFA (10 mL, 134.630 mmol, 7.50 equiv.) in DCM (10 mL) dropwise at 0 degrees C. The resulting mixture was stirred for 2 h at room temperature. The reaction was monitored by LCMS. The resulting mixture was concentrated under reduced pressure. To the above mixture was added K2CO3 (4.96 g, 35.897 mmol, 2 equiv.) and KI (2.98 g, 17.948 mmol, 1 equiv.) at room temperature. The resulting mixture was stirred for additional 16 h at 80 degrees C. The reaction was monitored by LCMS. The resulting mixture was concentrated under reduced pressure. The residue was purified by silica gel column chromatography, eluted with CH2Cl2/MeOH (20:1 to 10:1) to afford (5R)-5-methylpiperazin-2-one (2.5 g, crude) as a yellow oil.

### Step 3

### 4-chloro-5-[(2R)-2-methyl-5-oxopiperazin-1-yl]-2-(oxan-2-yl)-2,3-dihydropyridazin-3-one

To a stirred solution of (5R)-5-methylpiperazin-2-one (2.5 g, 21.901 mmol, 1 equiv.) in DIEA (2 mL) was added 4,5-dichloro-2-(oxan-2-yl)-2,3-dihydropyridazin-3-one (5.46 g, 21.901 mmol, 1 equiv.) at room temperature. The resulting mixture was stirred for 16 h at 100 degrees C. The reaction was monitored by LCMS. The mixture was allowed to cool down to room temperature. The residue was purified by reverse phase flash with the following conditions (Column: C18,330 g; Mobile Phase A: Water/0.05% NH4HCO3, Mobile Phase B: ACN; Flow rate: 80 mL/min; Gradient: 20% B to 30% B in 10 min; Detector, 220 nm; Monitor, 254 nm) to afford 4-chloro-5-[(2R)-2-methyl-5-oxopiperazin-1-yl]-2-(oxan-2-yl)-2,3-dihydropyridazin-3-one (600 mg, 8.38%) as a yellow solid.

### Step 4

### 4-chloro-5-[(2R)-4-[1-[4-fluoro-2-(trifluoromethyl)phenyl]ethyl]-2-methyl-5-oxopiperazin-1-yl]-2-(oxan-2-yl)-2,3-dihydropyridazin-3-one

To a stirred mixture of 4-chloro-5-[(2R)-2-methyl-5-oxopiperazin-1-yl]-2-(oxan-2-yl)-2,3-dihydropyridazin-3-one (500 mg, 1.530 mmol, 1 equiv.) and 1-[4-fluoro-2-(trifluoromethyl)phenyl]ethyl methanesulfonate (656.96 mg, 2.295 mmol, 1.5 equiv.) in ACN (20 mL) was added t-BuONa (220.57 mg, 2.295 mmol, 1.5 equiv.) at room temperature under nitrogen atmosphere. The final reaction mixture was irradiated with microwave radiation for 3 h at 110 degrees C. The reaction was monitored by LCMS. The mixture was allowed to cool down to room temperature. The resulting mixture was concentrated under reduced pressure. The residue was purified by reverse phase flash with the following conditions (Column: C18,330 g; Mobile Phase A: Water/0.05% NH4HCO3, Mobile Phase B: ACN; Flow rate: 80 mL/min; Gradient: 55% B to 75% B in 15 min; Detector, 220 nm; Monitor, 254 nm) to afford 4-chloro-5-[(2R)-4-[1-[4-fluoro-2-(trifluoromethyl)phenyl]ethyl]-2-methyl-5-oxopiperazin-1-yl]-2-(oxan-2-yl)-2,3-dihydropyridazin-3-one (120 mg, 15.17%) as a yellow solid.

### Step 5

### MD and ME

### 4-chloro-5-[(2R)-4-[(1S)-1-[4-fluoro-2-(trifluoromethyl)phenyl]ethyl]-2-methyl-5-oxopiperazin-1-yl]-2,3-dihydropyridazin-3-one & 4-chloro-5-[(2R)-4-[(1R)-1-[4-fluoro-2-(trifluoromethyl)phenyl]ethyl]-2-methyl-5-oxopiperazin-1-yl]-2,3-dihydropyridazin-3-one

To a stirred solution of 4-chloro-5-[(2R)-4-[1-[4-fluoro-2-(trifluoromethyl)phenyl]ethyl]-2-methyl-5-oxopiperazin-1-yl]-2-(oxan-2-yl)-2,3-dihydropyridazin-3-one (120 mg, 0.232 mmol, 1 equiv.) in DCM (8 mL) was added TFA (2 mL, 26.926 mmol, 115.99 equiv.) at room temperature. The resulting mixture was stirred for 1 h at room temperature. The reaction was monitored by LCMS. The resulting mixture was concentrated under reduced pressure. The residue was basified to pH 8 with saturated NaHCO₃(aq.). The resulting mixture was concentrated under reduced pressure. The residue was purified by reverse phase flash with the following conditions (Column: XBridge Prep C18 OBD Column 19×150 mm Sum; Mobile Phase A: Water (10 MMOL/L NH4HCO3), Mobile Phase B: ACN; Flow rate: 60 mL/min; Gradient: 22% B to 51% B in 7 min; 254/220 nm; Rt: 6.4 min) to afford 4-chloro-5-[(2R)-4-[(1S)-1-[4-fluoro-2-(trifluoromethyl)phenyl]ethyl]-2-methyl-5-oxopiperazin-1-yl]-2,3-dihydropyridazin-3-one (16.3 mg, 16.22%) as a white solid and 4-chloro-5-[(2R)-4-[(1R)-1-[4-fluoro-2-(trifluoromethyl)phenyl]ethyl]-2-methyl-5-oxopiperazin-1-yl]-2,3-dihydropyridazin-3-one (18.6 mg, 18.51%) as a white solid.

### Example 34. Synthesis of Compound MF

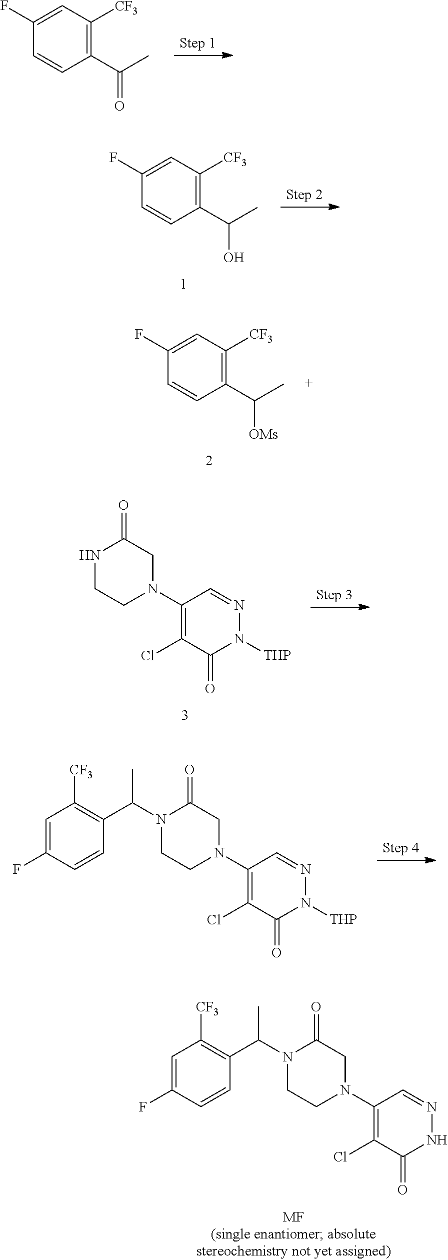

### Step 1

### 1-[4-fluoro-2-(trifluoromethyl)phenyl]ethan-1-ol

To a stirred solution of 4-fluoro-2-(trifluoromethyl)benzaldehyde (3 g, 15.616 mmol, 1 equiv.) in THF (50 mL) was added MeMgBr in Et2O (3 mol/L, 30 ml) dropwise at −30 degrees C. under nitrogen atmosphere. The resulting mixture was stirred for 2 h at room temperature under nitrogen atmosphere. The reaction was monitored by TLC. The reaction was quenched with sat. NH4Cl(aq.) at 0 degrees C. The resulting mixture was extracted with EtOAc (50 mL). The combined organic layers were washed with brine (3×50 mL), dried over anhydrous Na2SO4. After filtration, the filtrate was concentrated under reduced pressure. The resulting mixture was used in the next step directly without further purification.

### Step 2

### 1-[4-fluoro-2-(trifluoromethyl)phenyl]ethyl methanesulfonate

To a stirred solution of 1-[4-fluoro-2-(trifluoromethyl)phenyl]ethan-1-ol (3 g, 14.412 mmol, 1 equiv.) and Et3N (2.92 g, 28.825 mmol, 2 equiv.) in DCM (60 mL) was added MSCl (2.48 g, 21.618 mmol, 1.5 equiv.) dropwise at 0 degrees C. under nitrogen atmosphere. The resulting mixture was stirred for 2 h at room temperature under nitrogen atmosphere. The reaction was monitored by TLC. The reaction was quenched by the addition of sat. NH4Cl (aq.) (50 mL) at 0 degrees C. The resulting mixture was extracted with EtOAc (50 mL). The combined organic layers were washed with brine (3×100 mL), dried over anhydrous Na2SO4. After filtration, the filtrate was concentrated under reduced pressure to afford 1-[4-fluoro-2-(trifluoromethyl)phenyl]ethyl methanesulfonate (1.6 g, 38.78%) as a light yellow oil.

### Step 3

### 4-chloro-5-(4-[1-[4-fluoro-2-(trifluoromethyl)phenyl]ethyl]-3-oxopiperazin-1-yl)-2-(oxan-2-yl)-2,3-dihydropyridazin-3-one

To a stirred solution of 4-chloro-2-(oxan-2-yl)-5-(3-oxopiperazin-1-yl)-2,3-dihydropyridazin-3-one (800 mg, 2.558 mmol, 1 equiv.) and 1-[4-fluoro-2-(trifluoromethyl)phenyl]ethyl methanesulfonate (878.63 mg, 3.070 mmol, 1.2 equiv.) in ACN (8 mL) was added sodium 2,2-dimethylpropan-1-olate (563.43 mg, 5.116 mmol, 2 equiv.) in portions at room temperature under nitrogen atmosphere. The final reaction mixture was irradiated with microwave radiation for 3 h at 110 degrees C. The reaction was monitored by LCMS. The mixture was allowed to cool down to room temperature. The resulting mixture was extracted with EtOAc (20 mL). The combined organic layers were washed with brine (3×10 mL), dried over anhydrous Na2SO4. After filtration, the filtrate was concentrated under reduced pressure. The resulting mixture was used in the next step directly without further purification.

### Step 4

### Compound MF

### 4-chloro-5-[4-[(1R)-1-[4-fluoro-2-(trifluoromethyl)phenyl]ethyl]-3-oxopiperazin-1-yl]-2,3-dihydropyridazin-3-one & 4-chloro-5-[4-[(1S)-1-[4-fluoro-2-(trifluoromethyl)phenyl]ethyl]-3-oxopiperazin-1-yl]-2,3-dihydropyridazin-3-one

To a stirred solution of 4-chloro-5-(4-[1-[4-fluoro-2-(trifluoromethyl)phenyl]ethyl]-3-oxopiperazin-1-yl)-2-(oxan-2-yl)-2,3-dihydropyridazin-3-one (110 mg, 0.219 mmol, 1 equiv.) in DCM (10 mL) was added TFA (3 mL) in portions at room temperature under nitrogen atmosphere. The resulting mixture was stirred for 3 h at room temperature under nitrogen atmosphere. The reaction was monitored by LCMS. The resulting mixture was concentrated under vacuum. The residue was basified to pH 8 with saturated NaHCO₃(aq.). The resulting mixture was extracted with EtOAc (20 mL). The combined organic layers were washed with brine (3×20 mL), dried over anhydrous Na2SO4. After filtration, the filtrate was concentrated under reduced pressure. The crude product (50 mg) was purified by CHIRAL-HPLC with the following conditions (Column: XBridge Prep OBD C18 Column 30×150 mm Sum; Mobile Phase A: Water (10 MMOL/L NH4HCO3), Mobile Phase B: ACN; Flow rate: 60 mL/min; Gradient: 20% B to 32% B in 16 min; 220 nm; Rt: 14.27 min) to afford 4-chloro-5-[4-[(1R)-1-[4-fluoro-2-(trifluoromethyl)phenyl]ethyl]-3-oxopiperazin-1-yl]-2,3-dihydropyridazin-3-one (6.0 mg, 6.55%) as a white solid and 4-chloro-5-[4-[(1S)-1-[4-fluoro-2-(trifluoromethyl)phenyl]ethyl]-3-oxopiperazin-1-yl]-2,3-dihydropyridazin-3-one (6.2 mg, 6.77%) as a white solid.

### Example 35. Synthesis of Compound PW

Compound PW

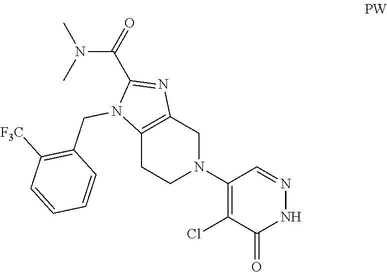 [SMILES: CN(C)C(=O)c1nc2c(n1Cc1ccccc1C(F)(F)F)CCN(c1cn[nH]c(=O)c1Cl)C2]

was prepared by the methods and scheme described for PU by using the corresponding amine.

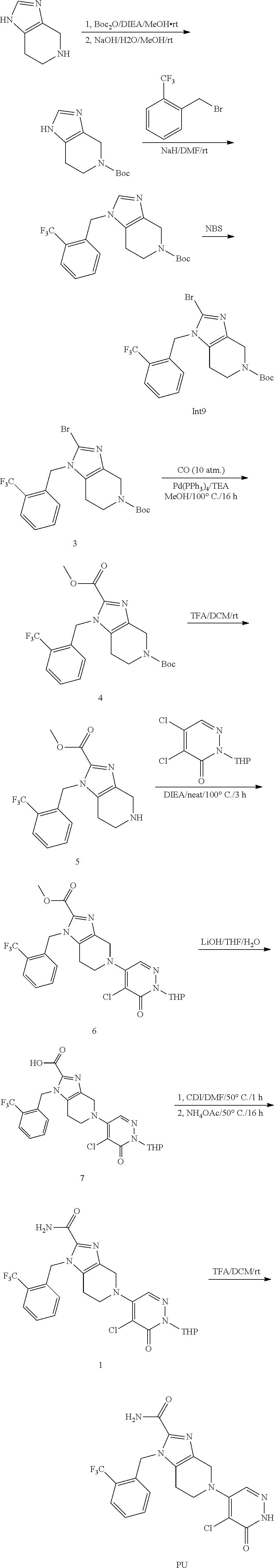

### Preparation of PU

### Preparation of Intermediate 9 (Int9)

### 1,5-di-tert-butyl 1H,4H,5H,6H,7H-imidazo[4,5-c]pyridine-1,5-dicarboxylate

To a stirred solution of 1H,4H,5H,6H,7H-imidazo[4,5-c]pyridine dihydrochloride (22 g, 112.20 mmol, 1 equiv.) in MeOH (300 mL) was added di-tert-butyl decarbonate (61.2 g, 280.50 mmol, 2.5 equiv.) and ethylbis(propan-2-yl)amine (50.8 g, 392.70 mmol, 3.5 equiv.) dropwise at 0 degree Celsius under nitrogen atmosphere. The solution was stirred at room temperature overnight. Desired product could be detected by LCMS. The mixture was concentrated under reduced pressure. The residue was purified by silica gel column chromatography, eluted with PE/EtOAc (5:1 to 2:1) to afford 1,5-di-tert-butyl1H,4H,5H,6H,7H-imidazo[4,5-c]pyridine-1,5-dicarboxylate (30 g, 82.68%) as white solid.

To a stirred solution of 1,5-di-tert-butyl 1H,4H,5H,6H,7H-imidazo[4,5-c]pyridine-1,5-dicarboxylate (7 g, 1 equiv.) in MeOH (80 mL) and H₂O (17 mL) was added NaOH (1.7 g, 43.29 mmol, 2.00 equiv.) in portions at room temperature under nitrogen atmosphere. The mixture was stirred at room temperature for 2 h. Desired product could be detected by LCMS. The mixture was basified to pH 8 with citric acid. The resulting mixture was extracted with CH2Cl2 (3×100 mL). The combined organic layers were washed with brine (1×100 mL), dried over anhydrous Na2SO4. After filtration, the filtrate was concentrated under reduced pressure to afford tert-butyl 1H,4H,5H,6H,7H-imidazo[4,5-c]pyridine-5-carboxylate (4.1 g, 84.84%) as an off-white semi-solid.

### tert-butyl 1-[[2-(trifluoromethyl)phenyl]methyl]-1H,4H,5H,6H,7H-imidazo[4,5-c]pyridine-5-carboxylate

To a stirred solution of tert-butyl1H,4H,5H,6H,7H-imidazo[4,5-c]pyridine-5-carboxylate (93.4 mg, 0.42 mmol, 1 equiv.) in DMF (8 mL) was added NaH (25.1 mg, 0.63 mmol, 1.5 equiv., 60%) in portions at 0 degree Celsius under nitrogen atmosphere. The mixture was stirred at room temperature for 1 h. To the mixture was added 1-(bromomethyl)-2-(trifluoromethyl)benzene (100 mg, 0.42 mmol, 1 equiv.) at 0 degree Celsius. The mixture was stirred at room temperature for 1 h. Desired product could be detected by LCMS. It was a pilot reaction, no work up was performed.

### tert-butyl 2-bromo-1-[[2-(trifluoromethyl)phenyl]methyl]-1H,4H,5H,6H,7H-imidazo[4,5-c]pyridine-5-carboxylate

To a stirred solution of tert-butyl 1-[[2-(trifluoromethyl)phenyl]methyl]-1H,4H,5H,6H,7H-imidazo[4,5-c]pyridine-5-carboxylate (1 g, 2.62 mmol, 1 equiv.) in DMF (15 mL) was added NBS (0.5 g, 2.81 mmol, 1.07 equiv.) in portions at 0 degree Celsius under nitrogen atmosphere. The mixture was stirred at room temperature for 1 h. Desired product could be detected by LCMS. The resulting mixture was concentrated under reduced pressure. The residue was purified by silica gel column chromatography, eluted with PE/EtOAc (5:1 to 1:1) to afford tert-butyl 2-bromo-1-[[2-(trifluoromethyl)phenyl]methyl]-1H,4H,5H,6H,7H-imidazo[4,5-c]pyridine-5-carboxylate (800 mg, 66.29%) as colorless oil.

### 5-tert-Butyl 2-methyl 1-[[2-(trifluoromethyl)phenyl]methyl]-1H,4H,5H,6H,7H-imidazo[4,5-c]pyridine-2,5-dicarboxylate

To a stirred mixture of tert-butyl2-bromo-1-[[2-(trifluoromethyl)phenyl]methyl]-1H,4H,5H,6H,7H-imidazo[4,5-c]pyridine-5-carboxylate (1 g, 2.173 mmol, 1 equiv.) and TEA (0.44 g, 4.348 mmol, 2.00 equiv.) in MeOH (100 mL) was added Pd(PPh3)4 (0.25 g, 0.217 mmol, 0.1 equiv.) at room temperature under nitrogen atmosphere. The resulting mixture was stirred for 1 h at 100 degrees Celsius under nitrogen atmosphere. The reaction was monitored by LCMS. The mixture was allowed to cool down to room temperature. The resulting mixture was concentrated under reduced pressure. The residue was purified by silica gel column chromatography, eluted with PE/EtOAc (10:1 to 1:1) to afford 5-tert-butyl 2-methyl 1-[[2-(trifluoromethyl)phenyl]methyl]-1H,4H,5H,6H,7H-imidazo[4,5-c]pyridine-2,5-dicarboxylate (800 mg, 83.80%) as a brown solid.

### Methyl 1-(2-(trifluoromethyl)benzyl)-4,5,6,7-tetrahydro-1H-imidazo[4,5-c]pyridine-2-carboxylate

To a stirred solution of 5-tert-butyl 2-methyl 1-[[2-(trifluoromethyl)phenyl]methyl]-1H,4H,5H,6H,7H-imidazo[4,5-c]pyridine-2,5-dicarboxylate (350 mg, 0.71 mmol, 1 equiv.) in DCM (12 mL) was added TFA (2 mL, 26.93 mmol, 37.81 equiv.) at room temperature. The solution was stirred at rt for 2 h. The residue was purified by reverse phase flash to afford methyl 1-(2-(trifluoromethyl)benzyl)-4,5,6,7-tetrahydro-1H-imidazo[4,5-c]pyridine-2-carboxylate (220 mg, 78.94%) as colorless oil.

### Methyl 5-[5-chloro-1-(oxan-2-yl)-6-oxo-1,6-dihydropyridazin-4-yl]-1-[[2-(trifluoromethyl)phenyl]methyl]-1H,4H,5H,6H,7H-imidazo[4,5-c]pyridine-2-carboxylate

To a stirred solution of methyl 1-[[2-(trifluoromethyl)phenyl]methyl]-1H,4H,5H,6H,7H-imidazo[4,5-c]pyridine-2-carboxylate (500 mg, 1.474 mmol, 1 equiv.) in DIEA (2 mL) was added 4,5-dichloro-2-(oxan-2-yl)-2,3-dihydropyridazin-3-one (734.09 mg, 2.947 mmol, 2.00 equiv.) at room temperature. The resulting mixture was stirred for 2 h at 90 degrees C. The reaction was monitored by LCMS. The mixture was allowed to cool down to room temperature. The residue was purified by silica gel column chromatography, eluted with PE/EtOAc (5:1 to 1:1) to afford methyl 5-[5-chloro-1-(oxan-2-yl)-6-oxo-1,6-dihydropyridazin-4-yl]-1-[[2-(trifluoromethyl)phenyl]methyl]-1H,4H,5H,6H,7H-imidazo[4,5-c]pyridine-2-carboxylate (600 mg, 73.77%) as a brown solid.

### 5-[5-chloro-1-(oxan-2-yl)-6-oxo-1,6-dihydropyridazin-4-yl]-1-[[2-(trifluoromethyl)phenyl]methyl]-1H,4H,5H,6H,7H-imidazo[4,5-c]pyridine-2-carboxylic acid

To a stirred solution of methyl 5-[5-chloro-1-(oxan-2-yl)-6-oxo-1,6-dihydropyridazin-4-yl]-1-[[2-(trifluoromethyl)phenyl]methyl]-1H,4H,5H,6H,7H-imidazo[4,5-c]pyridine-2-carboxylate (600 mg, 1.087 mmol, 1 equiv.) in THF (10 mL) and H₂O (10 mL) was added LiOH (260.33 mg, 10.871 mmol, 10.00 equiv.) at room temperature. The resulting mixture was stirred for 16 h at 45 degrees C. The reaction was monitored by LCMS. The mixture was allowed to cool down to room temperature. The resulting mixture was concentrated under reduced pressure. The crude product was purified by reverse phase flash with the following conditions (Column: XBridge Prep OBD C18 Column 30×150 mm Sum; Mobile Phase A: Water (10 MMOL/L NH4HCO3), Mobile Phase B: ACN; Flow rate: 60 mL/min; Gradient: 25% B to 45% B in 8 min; 220 nm; Rt: 7.48 min) to afford 5-[5-chloro-1-(oxan-2-yl)-6-oxo-1,6-dihydropyridazin-4-yl]-1-[[2-(trifluoromethyl)phenyl]methyl]-1H,4H,5H,6H,7H-imidazo[4,5-c]pyridine-2-carboxylic acid (560 mg, 95.77%) as a brown solid.

### 5-[5-chloro-1-(oxan-2-yl)-6-oxo-1,6-dihydropyridazin-4-yl]-1-[[2-(trifluoromethyl)phenyl]methyl]-1H,4H,5H,6H,7H-imidazo[4,5-c]pyridine-2-carboxamide

To a stirred mixture of 5-[5-chloro-1-(oxan-2-yl)-6-oxo-1,6-dihydropyridazin-4-yl]-1-[[2-(trifluoromethyl)phenyl]methyl]-1H,4H,5H,6H,7H-imidazo[4,5-c]pyridine-2-carboxylic acid (80 mg, 0.149 mmol, 1 equiv.) in DMF (10 mL) was added CDI (36.17 mg, 0.223 mmol, 1.5 equiv.) at room temperature under nitrogen atmosphere. The resulting mixture was stirred for 2 h at 45 degrees C. The reaction was monitored by LCMS. Then NH4OAc (22.93 mg, 0.297 mmol, 2.0 equiv.) was added at 45 degrees C. The resulting mixture was stirred for 16 h at 45 degrees C. The reaction was monitored by LCMS. The mixture was allowed to cool down to room temperature. The resulting mixture was concentrated under reduced pressure. The crude product was purified by reverse phase flash with the following conditions (Column: XBridge Prep OBD C18 Column 30×150 mm Sum; Mobile Phase A: Water (10 MMOL/L NH4HCO3), Mobile Phase B: ACN; Flow rate: 60 mL/min; Gradient: 25% B to 48% B in 8 min; 220 nm; Rt: 7.78 min) to afford 5-[5-chloro-1-(oxan-2-yl)-6-oxo-1,6-dihydropyridazin-4-yl]-1-[[2-(trifluoromethyl)phenyl]methyl]-1H,4H,5H,6H,7H-imidazo[4,5-c]pyridine-2-carboxamide (30 mg, 37.57%) as a brown solid.

### 5-(5-chloro-6-oxo-1,6-dihydropyridazin-4-yl)-1-[[2-(trifluoromethyl)phenyl]methyl]-1H,4H,5H,6H,7H-imidazo[4,5-c]pyridine-2-carboxamide

To a stirred solution of 5-[5-chloro-1-(oxan-2-yl)-6-oxo-1,6-dihydropyridazin-4-yl]-1-[[2-(trifluoromethyl)phenyl]methyl]-1H,4H,5H,6H,7H-imidazo[4,5-c]pyridine-2-carboxamide (30 mg, 0.056 mmol, 1 equiv.) in DCM (4 mL) was added TFA (1 mL) at room temperature. The resulting mixture was stirred for 1 h at room temperature. The reaction was monitored by LCMS. The resulting mixture was concentrated under reduced pressure. The mixture was basified to pH 7 with saturated NaHCO₃(aq.). The crude product (20 mg) was purified by Prep-HPLC with the following conditions (Column: XBridge Prep OBD C18 Column 30×150 mm Sum; Mobile Phase A: Water (10 MMOL/L NH4HCO3), Mobile Phase B: ACN; Flow rate: 60 mL/min; Gradient: 25% B to 48% B in 8 min; 220 nm; Rt: 7.78 min) to afford 5-(5-chloro-6-oxo-1,6-dihydropyridazin-4-yl)-1-[[2-(trifluoromethyl)phenyl]methyl]-1H,4H,5H,6H,7H-imidazo[4,5-c]pyridine-2-carboxamide (10.7 mg, 42.29%) as a white solid.

### Example 36. Synthesis of Compound PR

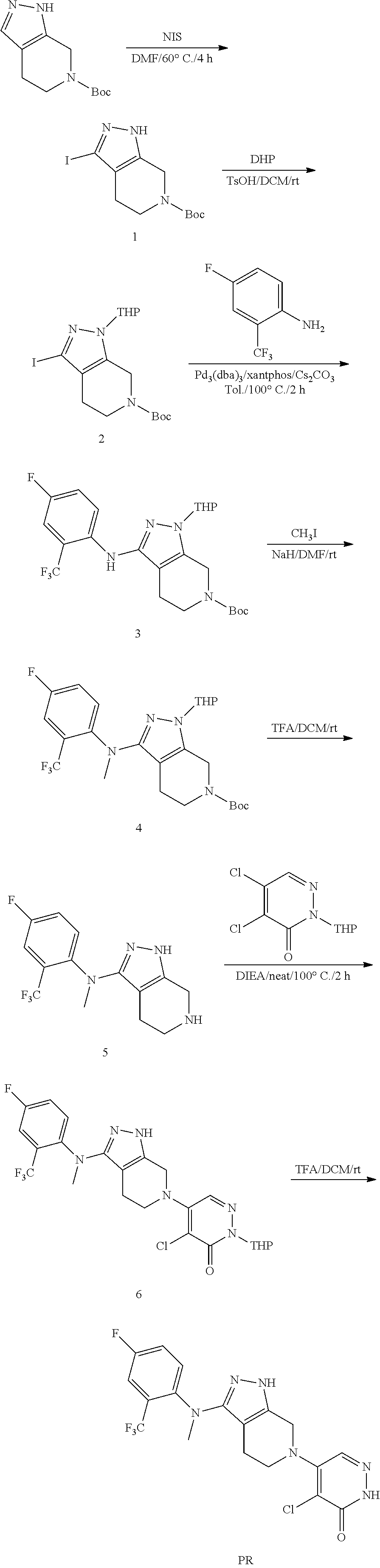

### Preparation of PR

### tert-Butyl 3-iodo-1H,4H,5H,6H,7H-pyrazolo[4,3-c]pyridine-5-carboxylate

To a stirred solution of tert-butyl1H,4H,5H,6H,7H-pyrazolo[3,4-c]pyridine-6-carboxylate (1.0 g, 4.479 mmol, 1 equiv.) in DMF (20 mL) was added MS (1209.18 mg, 5.375 mmol, 1.20 equiv.) in portions at rt under nitrogen atmosphere. The resulting mixture was stirred for 4 h at 60 degrees Celsius under nitrogen atmosphere. The reaction was monitored by LCMS. The mixture was allowed to cool down to rt. The resulting mixture was extracted with EtOAc (3×200 mL). The combined organic layers were washed with brine (2×100 mL), dried over anhydrous Na2SO4. After filtration, the filtrate was concentrated under reduced pressure. The residue was purified by reverse phase flash chromatography with the following conditions: Column: Spherical C18, 20-40 um, 330 g; Mobile Phase A: Water (plus 5 mM NH4NO3); Mobile Phase B: ACN; Flow rate: 80 mL/min; Gradient: 5%-5% B, 10 min, 45% B-60% B gradient in 20 min; Detector: 220 nm. The fractions containing the desired product were collected at 55% B and concentrated under reduced pressure to afford tert-butyl 3-iodo-1H,4H,5H,6H,7H-pyrazolo[3,4-c]pyridine-6-carboxylate (1.1 g, 83.13%) as a yellow solid.

### tert-Butyl 3-iodo-1-(oxan-2-yl)-1H,4H,5H,6H,7H-pyrazolo[3,4-c]pyridine-6-carboxylate

To a stirred mixture of tert-butyl3-iodo-1H,4H,5H,6H,7H-pyrazolo[3,4-c]pyridine-6-carboxylate (1.1 g, 3.150 mmol, 1 equiv.) and 3,4-dihydro-2H-pyran (1.32 g, 15.692 mmol, 4.98 equiv.) in DCM (20 mL) was added TsOH (54.25 mg, 0.315 mmol, 0.10 equiv.) in portions at 0 degrees Celsius under nitrogen atmosphere. The resulting mixture was stirred for 2 h at rt under nitrogen atmosphere. The reaction was monitored by LCMS. The resulting mixture was concentrated under reduced pressure. The resulting mixture was extracted with EtOAc (3×500 mL). The combined organic layers were washed with brine (2×300 mL), dried over anhydrous Na2SO4. After filtration, the filtrate was concentrated under reduced pressure. The residue was purified by reverse phase flash chromatography with the following conditions: Column: Spherical C18, 20-40 um, 330 g; Mobile Phase A: Water (plus 5 mM NH4NO3); Mobile Phase B: ACN; Flow rate: 80 mL/min; Gradient: 5%-5% B, 10 min, 55% B-70% B gradient in 20 min; Detector: 220 nm. The fractions containing the desired product were collected at 65% B and concentrated under reduced pressure to afford tert-butyl3-iodo-1-(oxan-2-yl)-1H,4H,5H,6H,7H-pyrazolo[3,4-c]pyridine-6-carboxylate (1.3 g) as a yellow oil.

### tert-Butyl 3-[[4-fluoro-2-(trifluoromethyl)phenyl]amino]-1-(oxan-2-yl)-1H,4H,5H,6H,7H-pyrazolo[3,4-c]pyridine-6-carboxylate

To a stirred mixture of tert-butyl3-iodo-1-(oxan-2-yl)-1H,4H,5H,6H,7H-pyrazolo[3,4-c]pyridine-6-carboxylate (1.0 g, 2.308 mmol, 1 equiv.) and 4-fluoro-2-(trifluoromethyl)aniline (0.62 g, 3.461 mmol, 1.50 equiv.) in Toluene (40 mL) were added XantPhos (534.16 mg, 0.923 mmol, 0.4 equiv.), Pd2(dba)3 (211.34 mg, 0.231 mmol, 0.1 equiv.) and Cs2CO3 (1503.93 mg, 4.616 mmol, 2.00 equiv.) in portions at rt under nitrogen atmosphere. The resulting mixture was stirred for 2 h at 100 degrees Celsius under nitrogen atmosphere. The reaction was monitored by LCMS. The mixture was allowed to cool down to rt. The resulting mixture was extracted with EtOAc (3×300 mL). The combined organic layers were washed with brine (2×200 mL), dried over anhydrous Na2SO4. After filtration, the filtrate was concentrated under reduced pressure. The residue was purified by reverse phase flash chromatography with the following conditions: Column: Spherical C18, 20-40 um, 330 g; Mobile Phase A: Water (plus 5 mM NH4NO3); Mobile Phase B: ACN; Flow rate: 80 mL/min; Gradient: 5%-5% B, 10 min, 45% B-90% B gradient in 30 min; Detector: 220 nm. The fractions containing the desired product were collected at 85% B and concentrated under reduced pressure to afford tert-butyl 3-[[4-fluoro-2-(trifluoromethyl)phenyl]amino]-1-(oxan-2-yl)-1H,4H,5H,6H,7H-pyrazolo[3,4-c]pyridine-6-carboxylate (150 mg, 13.41%) as a yellow oil.

### tert-Butyl 3-[[4-fluoro-2-(trifluoromethyl)phenyl](methyl)amino]-1-(oxan-2-yl)-1H,4H,5H,6H,7H-pyrazolo[3,4-c]pyridine-6-carboxylate

To a stirred solution of tert-butyl3-[[4-fluoro-2-(trifluoromethyl)phenyl]amino]-1-(oxan-2-yl)-1H,4H,5H,6H,7H-pyrazolo[3,4-c]pyridine-6-carboxylate (100 mg, 0.206 mmol, 1 equiv.) in DMF (10 mL) was added NaH (9.91 mg, 0.248 mmol, 1.20 equiv, 60%) at rt under nitrogen atmosphere. The reaction was stirred for 0.5 h at rt. Then CH3I (43.94 mg, 0.310 mmol, 1.5 equiv.) was added. The reaction mixture was stirred for 2 h at rt. The reaction was monitored by LCMS. The resulting mixture was extracted with EtOAc (3×200 mL). The combined organic layers were washed with brine (2×100 mL), dried over anhydrous Na2SO4. After filtration, the filtrate was concentrated under reduced pressure. The residue was purified by reverse phase flash chromatography with the following conditions: Column: Spherical C18, 20-40 um, 120 g; Mobile Phase A: Water (plus 5 mM NH4NO3); Mobile Phase B: ACN; Flow rate: 40 mL/min; Gradient: 5%-5% B, 10 min, 40% B-60% B gradient in 15 min; Detector: 220 nm. The fractions containing the desired product were collected at 50% B and concentrated under reduced pressure to afford tert-butyl 3-[[4-fluoro-2-(trifluoromethyl)phenyl](methyl)amino]-1-(oxan-2-yl)-1H,4H,5H,6H,7H-pyrazolo[3,4-c]pyridine-6-carboxylate (100 mg, 97.19%) as a yellow solid.

### N-[4-fluoro-2-(trifluoromethyl)phenyl]-N-methyl-1H,4H,5H,6H,7H-pyrazolo[3,4-c]pyridin-3-amine

To a stirred solution of tert-butyl 3-[[4-fluoro-2-(trifluoromethyl)phenyl](methyl)amino]-1-(oxan-2-yl)-1H,4H,5H,6H,7H-pyrazolo[3,4-c]pyridine-6-carboxylate (100 mg) in DCM (10 mL) was added TFA (1 mL) dropwise at rt. The reaction mixture was stirred for 2 h at rt. The reaction was monitored by LCMS. The resulting mixture was concentrated under reduced pressure. The residue was basified to pH=8 with saturated NH4HCO3 (aq.). The resulting mixture was extracted with CH2Cl2 (2×50 mL). The combined organic layers were washed with brine (1×50 mL), dried over anhydrous Na2SO4. After filtration, the filtrate was concentrated under reduced pressure. The residue was purified by reverse phase flash chromatography with the following conditions: Column: Spherical C18, 20-40 um, 120 g; Mobile Phase A: Water (plus 5 mM NH4NO3); Mobile Phase B: ACN; Flow rate: 80 mL/min; Gradient: 5%-5% B, 10 min, 35% B-55% B gradient in 20 min; Detector: 220 nm. The fractions containing the desired product were collected at 45% B and concentrated under reduced pressure to afford N-[4-fluoro-2-(trifluoromethyl)phenyl]-N-methyl-1H,4H,5H,6H,7H-pyrazolo[3,4-c]pyridin-3-amine (50 mg) as a yellow oil.

### 4-Chloro-5-(3-[[4-fluoro-2-(trifluoromethyl)phenyl](methyl)amino]-1H,4H,5H,6H,7H-pyrazolo[3,4-c]pyridin-6-yl)-2-(oxan-2-yl)-2,3-dihydropyridazin-3-one

Into a 25 mL round-bottom flask were added N-[4-fluoro-2-(trifluoromethyl)phenyl]-N-methyl-1H,4H,5H,6H,7H-pyrazolo[3,4-c]pyridin-3-amine (50 mg, 0.159 mmol, 1 equiv.), 4,5-dichloro-2-(oxan-2-yl)-2,3-dihydropyridazin-3-one (79.26 mg, 0.318 mmol, 2.00 equiv.) and DIEA (61.68 mg, 0.477 mmol, 3.00 equiv.) at rt under nitrogen atmosphere. The resulting mixture was stirred for 2 h at 90 degrees Celsius under nitrogen atmosphere. The reaction was monitored by LCMS. The mixture was allowed to cool down to rt. The residue was purified by reverse phase flash chromatography with the following conditions: Column: Spherical C18, 20-40 um, 120 g; Mobile Phase A: Water (plus 5 mM NH4NO3); Mobile Phase B: ACN; Flow rate: 40 mL/min; Gradient: 5%-5% B, 10 min, 50% B-65% B gradient in 20 min; Detector: 220 nm. The fractions containing the desired product were collected at 55% B and concentrated under reduced pressure to afford 4-chloro-5-(3-[[4-fluoro-2-(trifluoromethyl)phenyl](methyl)amino]-1H,4H,5H,6H,7H-pyrazolo[3,4-c]pyridin-6-yl)-2-(oxan-2-yl)-2,3-dihydropyridazin-3-one (80 mg) as a yellow oil.

### 4-Chloro-5-(3-[[4-fluoro-2-(trifluoromethyl)phenyl](methyl)amino]-1H,4H,5H,6H,7H-pyrazolo[3,4-c]pyridin-6-yl)-2,3-dihydropyridazin-3-one

To a stirred solution of 4-chloro-5-(3-[[4-fluoro-2-(trifluoromethyl)phenyl](methyl)amino]-1H,4H,5H,6H,7H-pyrazolo[3,4-c]pyridin-6-yl)-2-(oxan-2-yl)-2,3-dihydropyridazin-3-one (80 mg, 0.152 mmol, 1 equiv.) in DCM (10 mL) was added TFA (1 mL, 13.463 mmol, 88.67 equiv.) dropwies at rt. The reaction mixture was stirred for 2 h at rt. The reaction was monitored by LCMS. The resulting mixture was concentrated under reduced pressure. The residue was basified to pH=8 with saturated NH4HCO3 (aq.). The resulting mixture was extracted with EtOAc (2×100 mL). The combined organic layers were washed with brine (1×100 mL), dried over anhydrous Na2SO4. After filtration, the filtrate was concentrated under reduced pressure. The residue was purified by Prep-HPLC with the following conditions (Column: Sunfire Prep C18 OBD Column, 10 um, 19*250 mm; Mobile Phase A: Water (0.1% FA), Mobile Phase B: ACN; Flow rate: 25 mL/min; Gradient: 35% B to 55% B in 7 min; 254 nm; Rt: 6.5 min) to afford 4-chloro-5-(3-[[4-fluoro-2-(trifluoromethyl)phenyl](methyl)amino]-1H,4H,5H,6H,7H-pyrazolo[3,4-c]pyridin-6-yl)-2,3-dihydropyridazin-3-one (10.4 mg) as a white solid.

### Example 37. Synthesis of JX

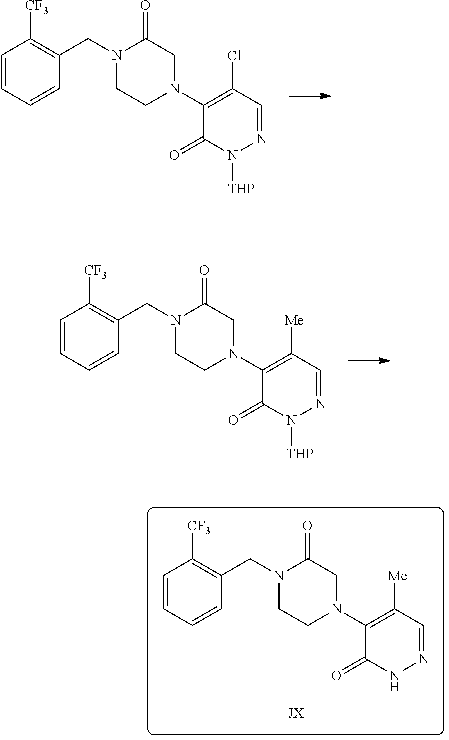

### 5-methyl-2-(oxan-2-yl)-4-(3-oxo-4-[[2-(trifluoromethyl)phenyl]methyl]piperazin-1-yl)-2,3-dihydropyridazin-3-one

To a solution of 5-chloro-2-(oxan-2-yl)-4-(3-oxo-4-[[2-(trifluoromethyl)phenyl]methyl]piperazin-1-yl)-2,3-dihydropyridazin-3-one (120 mg, 0.25 mmol, 1 equiv.) and methylboronic acid (45.8 mg, 760 mmol, 3 equiv.) in 1,4-dioxane (5 mL) and H2O (1 mL) were added K2CO3 (70.4 mg, 0.51 mmol, 2 equiv) and Pd(PPh3)4 (29.4 mg, 0.03 mmol, 0.1 equiv). The final reaction mixture was irradiated with microwave radiation for 3 h at 130 degrees Celsius under nitrogen atmosphere, the resulting mixture was concentrated under reduced pressure. The residue was purified by Prep-TLC (PE:EA=1:1) to afford 5-methyl-2-(oxan-2-yl)-4-(3-oxo-4-[[2-(trifluoromethyl)phenyl]methyl]piperazin-1-yl)-2,3-dihydropyridazin-3-one (I00 mg, 87.11%) as a white solid.

### Compound JX

### 5-methyl-4-(3-oxo-4-[[2-(trifluoromethyl)phenyl]methyl]piperazin-1-yl)-2,3-dihydropyridazin-3-one

To a stirred solution of 5-methyl-2-(oxan-2-yl)-4-(3-oxo-4-[[2-(trifluoromethyl)phenyl]methyl]piperazin-1-yl)-2,3-dihydropyridazin-3-one (80 mg) in DCM (10 mL) was added TFA (2 mL) dropwise at room temperature under nitrogen atmosphere. The mixture was stirred at room temperature 2 h. Desired product could be detected by LCMS. The resulting mixture was concentrated under reduced pressure. The crude product (60 mg) was purified by Prep-HPLC with the following conditions (Column: XBridge Prep C18 OBD Column, 5 um, 19*150 mm; Mobile Phase A: Water (10 mmoL/L NH4HCO3), Mobile Phase B: ACN; Flow rate: 20 mL/min; Gradient: 25% B to 60% B in 7 min; 254 nm; Rt: 5.58 min) to afford 5-methyl-4-(3-oxo-4-[[2-(trifluoromethyl)phenyl]methyl]piperazin-1-yl)-2,3-dihydropyridazin-3-one (8.6 mg, 13.22%) as a white solid.

### Example 38. Synthesis of KX

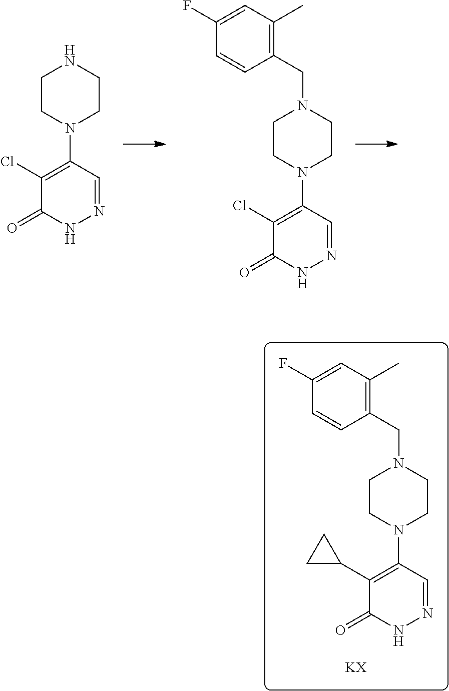

### Preparation of Compound KX

### 4-chloro-5-[4-[(4-fluoro-2-methylphenyl)methyl]piperazin-1-yl]-2,3-dihydropyridazin-3-one

To a stirred solution of 4-chloro-5-(piperazin-1-yl)-2,3-dihydropyridazin-3-one; trifluoroacetic acid (656 mg, 2.00 mmol, 1 equiv.) in DCM (10 mL) was added DIEA (515.9 mg, 3.99 mmol, 2 equiv.) and 1-(bromomethyl)-4-fluoro-2-methylbenzene (405.3 mg, 2.00 mmol, 1.00 equiv.) in portions at 0 degrees Celsius under nitrogen atmosphere. The mixture was stirred at room temperature overnight. Desired product could be detected by LCMS. The resulting mixture was concentrated under reduced pressure. The residue was purified by silica gel column chromatography, eluted with PE/EtOAc (5:1 to 1:1) to afford 4-chloro-5-[4-[(4-fluoro-2-methylphenyl)methyl]piperazin-1-yl]-2,3-dihydropyridazin-3-one (400 mg, 59.51%) as a white solid.

### Compound KX: 4-cylopropyl-5-[4-[(4-fluoro-2-methylphenyl)methyl]piperazin-1-yl]-2,3-dihydropyridazin-3-one

To a solution of 4-chloro-5-[4-[(4-fluoro-2-methylphenyl)methyl]piperazin-1-yl]-2,3-dihydropyridazin-3-one (120 mg, 0.36 mmol, 1 equiv.) and cyclopropylboronic acid (91.8 mg, 1.07 mol, 3.00 equiv.) in 1,4-dioxane (5 mL) and H2O (1 mL) were added Pd(AcO)2 (8.0 mg, 0.04 mmol, 0.10 equiv.), K2CO3 (98.5 mg, 0.71 mmol, 2.00 equiv.) and PCy3 (20.0 mg, 0.07 mmol, 0.20 equiv). The final reaction mixture was irradiated with microwave radiation for 3 h at 120 degrees Celsius under nitrogen atmosphere, the resulting mixture was concentrated under reduced pressure. The crude product (100 mg) was purified by Prep-HPLC with the following conditions (Column: XBridge Prep OBD C18 Column 30×150 mm 5 um; Mobile Phase A: Water (10 mmol/L NH4HCO3+0.1% NH3.H2O), Mobile Phase B: ACN; Flow rate: 60 mL/min; Gradient: 20% B to 45% B in 7 min; 254 nm; Rt: 6.73 min) to afford 4-cyclopropyl-5-[4-[(4-fluoro-2-methylphenyl)methyl]piperazin-1-yl]-2,3-dihydropyridazin-3-one (25.5 mg) as a white solid.

### Example 39. Synthesis of FA

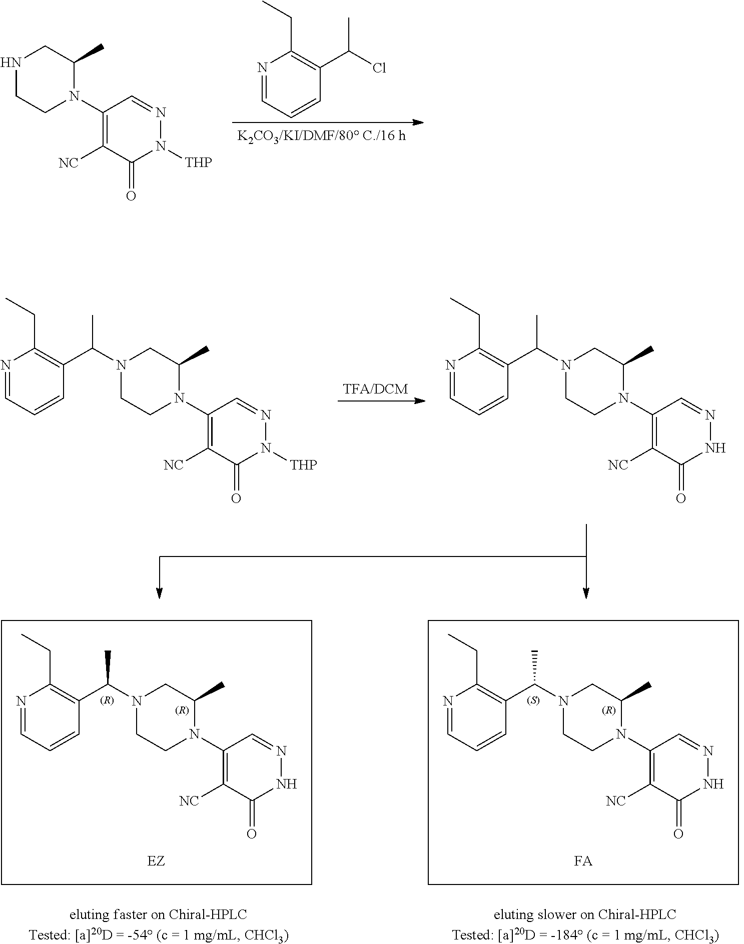

### Preparation of EZ and FA

### 3-(1-chloroethyl)-2-ethylpyridine was prepared by the methods and scheme described for 3-(1-chloropropyl)-2-ethylpyridine by using the corresponding pyridine

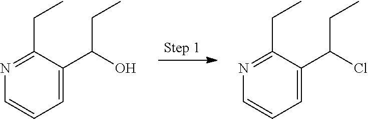

### 3-(1-chloropropyl)-2-ethylpyridine

To a stirred solution of 1-(2-ethylpyridin-3-yl)propan-1-ol (300 mg, 1.82 mmol, 1 equiv.) in DCM (20 mL) was added SOCl₂ (432.0 mg, 3.63 mmol, 2.00 equiv.) dropwise at 0 degrees C. under nitrogen atmosphere. The resulting mixture was stirred for 16 h at under nitrogen atmosphere. The reaction was monitored by LCMS. The resulting mixture was concentrated under vacuum. This resulted in 3-(1-chloropropyl)-2-ethylpyridine (350 mg, 104.95%) as a yellow oil.

### 5-[(3S)-1-[1-(2-ethylpyridin-3-yl)ethyl]-3-methylpiperidin-4-yl]-2-(oxan-2-yl)-3-oxo-2,3-dihydropyridazine-4-carbonitrile

To a stirred mixture of 3-(1-chloroethyl)-2-ethylpyridine (56.1 mg, 0.33 mmol, 1 equiv.) and 5-[(3 S)-3-methylpiperidin-4-yl]-2-(oxan-2-yl)-3-oxo-2,3-dihydropyridazine-4-carbonitrile (100 mg, 0.33 mmol, 1 equiv.) in ACN (20 mL) were added K2CO3 (68.6 mg, 0.50 mmol, 1.5 equiv.) and KI (109.8 mg, 0.66 mmol, 2 equiv.) in portions at room temperature. The reaction was stirred overnight at 80 degrees C. The resulting mixture was concentrated under reduced pressure. The residue was purified by silica gel column chromatography, eluted with PE:EA (50% to 100%) to afford 5-[(3S)-1-[1-(2-ethylpyridin-3-yl)ethyl]-3-methylpiperidin-4-yl]-2-(oxan-2-yl)-3-oxo-2,3-dihydropyridazine-4-carbonitrile (120 mg, 83.31%) as a yellow oil.

### 4-chloro-5-[(2R)-4-[(1S)-1-(2-ethylpyridin-3-yl)ethyl]-2-methylpiperazin-1-yl]-2,3-dihydropyridazin-3-one and 5-[(2R)-4-[(1R)-1-(2-ethylpyridin-3-yl)ethyl]-2-methylpiperazin-1-yl]-3-oxo-2,3-dihydropyridazine-4-carbonitrile

A mixture of 5-[(2R)-4-[1-(2-ethylpyridin-3-yl)-2,2,2-trifluoroethyl]-2-methylpiperazin-1-yl]-2-(oxan-2-yl)-3-oxo-2,3-dihydropyridazine-4-carbonitrile (120 mg, 0.24 mmol, 1 equiv.) and THF (3 mL, 37.03 mmol) in DCM (15 mL, 235.95 mmol) was stirred for 16 h at room temperature. The resulting mixture was concentrated under reduced pressure. The crude product was purified by reverse phase flash with the following conditions (Column: Spherical C18, 20˜40 um, 120 g; Mobile Phase A: Water (0.05% TFA), Mobile Phase B: ACN; Flow rate: 45 mL/min; Gradient (B %): 5%-15%, 4 min; 15%-45%, 20 min; 45%˜95%; 2 min; 95%, 5 min; Detector: 254 nm; Rt: 18 min.) to afford 4-chloro-5-[(2R)-4-[(1S)-1-(2-ethylpyridin-3-yl)ethyl]-2-methylpiperazin-1-yl]-2,3-dihydropyridazin-3-one (19 mg, 19.51%) as a white solid and 5-[(2R)-4-[(1R)-1-(2-ethylpyridin-3-yl)ethyl]-2-methylpiperazin-1-yl]-3-oxo-2,3-dihydropyridazine-4-carbonitrile (18.1 mg, 20.99%) as a white solid.

### Example 40. Synthesis of AO

Preparation of compound AO

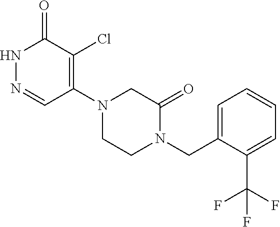 [SMILES: O=C1CN(c2cn[nH]c(=O)c2Cl)CCN1Cc1ccccc1C(F)(F)F]

follows the method and protocols described for the synthesis of AM starting with the appropriate benzylic bromide or chloride and using 4,5-dichloro-2,3-dihydropyridazin-3-one.

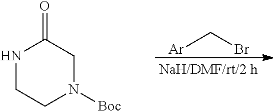

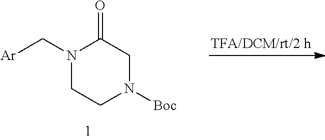

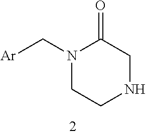

| Ar | Target ID |
|---|---|
| 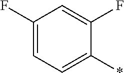 [SMILES: *c1ccc(F)cc1F] | AF |
| 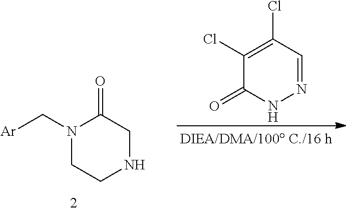 | 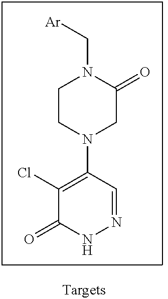 |
| Target ID | Ar |
| AM | 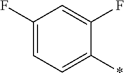 [SMILES: *c1ccc(F)cc1F] |

### tert-butyl 4-[(2,4-difluorophenyl)methyl]-3-oxopiperazine-1-carboxylate

To a solution of tert-butyl 3-oxopiperazine-1-carboxylate (300 mg, 1.50 mmol, 1 equiv.) in DMF (5 mL) was added NaH (89.9 mg, 2.25 mmol, 1.5 equiv., 60%) at room temperature. The resulting mixture was stirred for 0.5 h at room temperature. To the above mixture was added 1-(bromomethyl)-2,4-difluorobenzene (465.2 mg, 2.25 mmol, 1.5 equiv.) dropwise at room temperation. The resulting mixture was stirred for additional 16 h at room temperature. The reaction was monitored by LCMS. The reaction was quenched with water (100 mL). The resulting mixture was extracted with EtOAc (3×100 mL). The combined organic layers were washed with brine (100 mL), dried over anhydrous MgSO4. After filtration, the filtrate was concentrated under reduced pressure. The residue was purified by Prep-TLC (petroleum ether/EA 3:1) to afford tert-butyl 4-[(2,4-difluorophenyl)methyl]-3-oxopiperazine-1-carboxylate (410 mg, 83.86%) as a white solid.

### 1-[(2,4-difluorophenyl)methyl]piperazin-2-one

To a solution of tert-butyl 4-[(2,4-difluorophenyl)methyl]-3-oxopiperazine-1-carboxylate (410 mg, 1.26 mmol, 1 equiv.) in DCM (10 mL) was added TFA (2 mL, 26.93 mmol, 21.432 equiv.) at room temperature. The resulting mixture was stirred for 3 h at room temperature. The reaction was monitored by LCMS. The resulting mixture was concentrated under reduced pressure. The residue was basified to pH 8˜9 with saturated NaHCO₃(aq.). The resulting mixture was extracted with EtOAc (3×50 mL). The combined organic layers were washed with brine (100 mL), dried over anhydrous MgSO4. After filtration, the filtrate was concentrated under reduced pressure to afford 1-[(2,4-difluorophenyl)methyl]piperazin-2-one (220 mg, 77.41%) as a light yellow oil.

### Compound AM: 4-chloro-5-[4-[(2,4-difluorophenyl)methyl]-3-oxopiperazin-1-yl]-2,3-dihydropyridazin-3-one

To a solution of 4,5-dichloro-2,3-dihydropyridazin-3-one (65.6 mg, 0.40 mmol, 1 equiv.) in DMA (2 mL) were added 1-[(2,4-difluorophenyl)methyl]piperazin-2-one (90 mg, 0.40 mmol, 1 equiv.) and DIEA (102.8 mg, 0.80 mmol, 2 equiv.) at room temperation. The resulting mixture was stirred for 16 h at 100 degrees C. The reaction was monitored by LCMS. The product was purified by reverse phase flash with the following conditions (Column: spherical C18, 20-40 um, 120 g; Mobile Phase A: Water (5 mmol/L NH4HCO3), Mobile Phase B: MeCN; Flow rate: 45 mL/min; Gradient: 20% B to 40% B in 25 min; 220 nm) to afford 4-chloro-5-[4-[(2,4-difluorophenyl)methyl]-3-oxopiperazin-1-yl]-2,3-dihydropyridazin-3-one (28.6 mg, 20.27%) as a yellow solid.

### Example 41. TRPC4 Activity Assay

ICLN-1694 cells (HEK-TREx hTRPC4) expressing TRPC4 were generated as follows. Commercially available HekTrex-293 cells were seeded at 0.7×10⁶ cells/well in a 1×6-well plate 24 hrs prior to transfection using 2 mL cell growth media containing no antibiotics (lx DMEM/high glucose (Hyclone #SH30022.02); 10% fetal bovine serum (Sigma) 2 mM sodium pyruvate, 10 mM HEPES). The human codon-optimized TRPC4 coding sequence was cloned into pcDNA5/TO (Invitrogen; Cat No. V103320) using hygromycin as the resistance gene and the plasmid (SEQ ID NO:1) propagated using T-Rex-293 cells (Invitrogen; Cat No. R71007) following manufacturer's directions. On day 2, 2 μg of plasmid DNA plus 6 μl of Xtreme-GENE HP reagent in Optimem (200 μl total volume) was prepared and incubated for 15 min at room temperature. This plasmid solution was then gently overlayed dropwise onto each well and the plate was gently swirled to mix complex with the media for approximately 30 seconds. Transfected cells were incubated at 37° C. in a 10% CO₂ incubator for 24 hrs. The transfected cells were harvested and transferred into 2×150 mm dishes containing cell growth media with no antibiotics at 37° C.

The next day selection was initiated to generate a stable pool by adding cell growth media containing 150 μg/mL Hygromycin and 5 μg/mL Blasticidin and cells were allowed to grow. Media with the selection agent was changed every 1-2 days as needed to remove dead cells. After 7 days, the hygromycin concentration was reduced to 75 μg/mL and cells growth was allowed to continue.

Single clones were selected as follows. The stable pool was diluted to 10 cells/mL and seeded (100 μl/well) into 24×96 well plates (˜1 cell/well) and allowed to grow for 7 days in cell growth media. Fresh media (100 μl) was added and the cells allowed to grow for another 1-2 weeks and then stored frozen or used immediately.

Compounds were made up to, or supplied as, a 10 mM stock solution generally using DMSO as the vehicle. 10-point dose response curves were generated using the Echo-550 acoustic dispenser. Compound source plates were made by serially diluting compound stocks to create 10 mM, 1 mM, and 0.1 mM solutions in DMSO into Echo-certified LDV plates. The Echo then serially spotted 100% DMSO stock solutions into source dose response plates to generate a 4-fold dilution scheme. 100% DMSO was added to the spotted dose response plates to bring the final volume to 5 μl. 300 nl of the dose response stock plate was then spotted into pre-incubation and stimulation assay plates. 50 μl of pre-incubation buffer and 100 μl of stimulation buffer was then added to the plates resulting in a final assay test concentration range of 30 μM to 0.0001 μM with a final DMSO concentration of 0.3%.

ICLN-1694 cells (HEK-TREx hTRPC4) were plated onto 384 well, black pdl-coated microplates and maintained in cell growth media supplemented with 1 μg/mL tetracycline the day prior to use for experiments. TRPC4 expression was induced by the application of 1 μg/mL tetracycline at the time of plating. Media was removed from the plates and 10 μl of 4 μM of Fluo-4 AM (mixed with equal volume of Pluronic F-127) in EBSS (NaCl (142 mM), KCl (5.4 mM), glucose (10 mM), CaCl₂) (1.8 mM), MgCl2 (0.8 mM), HEPES (10 mM), pH 7.4) is added to the cells. Cells were incubated at room temperature, protected from light, for 60-90 minutes. After the incubation period, the dye was removed and replaced with 10 μl of EBSS. Cell, pre-incubation and stimulation plates were loaded onto the FLIPR-II and the assay was initiated. The FLIPR measured a 10 second baseline and then added 10 μl of 2× compounds (or controls). Changes in fluorescence were monitored for an additional 5 minutes. After a 5 minute pre-incubation, 20 μl of 2× Englerin A (with 1× compound or controls) was added to the cell plate. The final Engerlin A stimulation concentration in the assay was 100 nM. After the Englerin A addition, changes in fluorescence were monitored for an additional 5 minutes.

Compound modulation of TRPC4 calcium response was determined as follows. After the Englerin A, fluorescence was monitored for a 5-minute period. The maximum relative fluorescence response (minus the control response of 1 μM of an internal control compound known to maximally block TRPC4 calcium response, the “REF INHIB” in the formula below) was captured and exported from the FLIPR.

Compound effect is calculated as % inhibition using the following formula:

$$\% inhibition = \frac{\begin{matrix}RFU TEST AGENT - \\ Plate Average RFU REF INHIB\end{matrix}}{\begin{matrix}Plate Average RFU CONTROL - \\ Plate Average RFU REF INHIB\end{matrix}} \times 100$$

wherein “RFU” is the relative fluorescent units.

The results of these assays are shown in Table 2, below, wherein “A” indicates an IC₅₀ of less than or equal to 50 nM; “B” an IC₅₀ of greater than 50 nM and less than or equal to 500 nM; “C” an IC₅₀ of greater than 500 nM and less than 1 μM; “D” an IC₅₀ of 1 μM or greater; and “NT” indicates that the compound was not tested.

### Example 42. TRPC5 Activity Assay

ICLN-1633 cells (HEK-TREx hTRPC5) expressing TRPC5 were generated as follows. Commercially available HekTrex-293 cells were seeded at 0.7×10⁶ cells/well in a 1×6-well plate 24 hrs prior to transfection using 2 mL cell growth media containing no antibiotics (lx DMEM/high glucose (Hyclone #SH30022.02); 10% fetal bovine serum (Sigma) 2 mM sodium pyruvate, 10 mM HEPES). The human TRPC5 coding sequence (NM 012471 with a silent T478C mutation) was cloned into pcDNA5/TO (Invitrogen; Cat No. V103320) using hygromycin as the resistance gene and the plasmid (SEQ ID NO:2) propagated using T-Rex-293 cells (Invitrogen; Cat No. R71007) following manufacturer's directions. On day 2, 2 μg of plasmid DNA plus 6 μl of Xtreme-GENE HP reagent in Optimem (200 μl total volume) was prepared and incubated for 15 min at room temperature. This plasmid solution was then gently overlayed dropwise onto each well and the plate was gently swirled to mix complex with the media for approximately 30 seconds. Transfected cells were incubated at 37° C. in a 10% CO₂ incubator for 24 hrs. The transfected cells were harvested and transferred into 2×150 mm dishes containing cell growth media with no antibiotics at 37° C.

The next day selection was initiated to generate a stable pool by adding cell growth media containing 150 μg/mL Hygromycin and 5 μg/mL Blasticidin and cells were allowed to grow. Media with the selection agent was changed every 1-2 days as needed to remove dead cells. After 7 days, the hygromycin concentration was reduced to 75 μg/mL and cells growth was allowed to continue.

Single clones were selected as follows. The stable pool was diluted to 10 cells/mL and seeded (100 μl/well) into 24×96 well plates (˜1 cell/well) and allowed to grow for 7 days in cell growth media. Fresh media (100 μl) was added and the cells allowed to grow for another 1-2 weeks and then stored frozen or used immediately.

Compounds were made up to, or supplied as a 10 mM stock solution generally using DMSO as the vehicle. 10-point dose response curves were generated using the Echo-550 acoustic dispenser. Compound source plates were made by serially diluting compound stocks to create 10 mM, 1 mM, and 0.1 mM solutions in DMSO into Echo certified LDV plates. The Echo then serially spotted 100% DMSO stock solutions into source dose response plates to generate a 4-fold dilution scheme. 100% DMSO was added to the spotted dose response plates to bring the final volume to 5 μl. 300 nl of the dose response stock plate was then spotted into pre-incubation and stimulation assay plates. 50 μl of pre-incubation buffer and 100 μl of stimulation buffer was then added to the plates resulting in a final assay test concentration range of 30 μM to 0.0001 μM with a final DMSO concentration of 0.3%.

Human ICLN-1633 cells expressing were plated onto 384 well, black PDL-coated microplates and maintained in TRPC5 growth media the day prior to use for experiments. TRPC5 expression was induced by the application of 1 μg/mL tetracycline at the time of plating. Media is removed from the plates and 10 μl of 4 μM of Fluo-4 AM (mixed with equal volume of Pluronic F-127) in EBSS is added to the cells. Cells are incubated at room temperature, protected from light, for 60-90 minutes. After the incubation period, the dye is removed and replaced with 10 μl of EBSS. Cell, pre-incubation and stimulation plates are loaded onto the FLIPR-II and the assay is initiated. The FLIPR measures a 10 second baseline and then adds 10 μl of 2× compounds (or controls). Changes in fluorescence are monitored for an additional 5 minutes. After the 5 minute pre-incubation, 20 μl of 2× Riluzole (with 1× compound or controls) is added to the cell plate. The final Riluzole stimulation concentration in the assay is 30 μM. After the Riluzole addition, changes in fluorescence are monitored for an additional 5 minutes.

Compound modulation of TRPC5 calcium response was determined as follows. After the Englerin A, fluorescence was monitored for a 5-minute period. The maximum relative fluorescence response (minus the control response of 1 μM of an internal control compound known to maximally block TRPC5 calcium response, the “REF INHIB” in the formula below) was captured and exported from the FLIPR.

Compound effect is calculated as % inhibition using the following formula:

$$\% inhibition = \frac{\begin{matrix}RFU TEST AGENT - \\ Plate Average RFU REF INHIB\end{matrix}}{\begin{matrix}Plate Average RFU CONTROL - \\ Plate Average RFU REF INHIB\end{matrix}} \times 100$$

wherein “RFU” is the relative fluorescent units.

The results of these assays are shown in Table 2, below, wherein “A” indicates an IC₅₀ of less than or equal to 50 nM; “B” an IC₅₀ of greater than 50 nM and less than or equal to 500 nM; “C” an IC₅₀ of greater than 500 nM and less than 1 μM; “D” an IC₅₀ of 1 μM or greater; and “NT” indicates that the compound was not tested.

TABLE 2
TRPC4 and TRPC5 Activities of Exemplary Compounds

| Compound | TRPC5 | TRPC4 |
|---|---|---|
| 100 | A | A |
| 101 | A | A |
| 102 | A | A |
| 103 | A | A |
| 104 | A | A |
| 105 | A | A |
| 106 | A | B |
| 107 | A | B |
| 108 | B | C |
| 109 | A | B |
| 110 | B | A |
| 111 | B | NT |
| 112 | A | B |
| 113 | A | B |
| 114 | A | A |
| 115 | B | B |
| 116 | A | A |
| 117 | A | B |
| 117a | D | NT |
| 118 | B | B |
| 119 | A | A |
| 120 | A | A |
| 121 | A | NT |
| 122 | A | NT |
| 123 | B | NT |
| 124 | A | NT |
| 125 | A | NT |
| 126 | A | NT |
| 126a | B | NT |
| 127 | B | NT |
| 128 | A | NT |
| 129 | A | NT |
| 130 | A | NT |
| 131 | A | NT |
| 132 | B | NT |
| 133 | B | NT |
| 133a | C | NT |
| 134 | A | NT |
| 135 | A | NT |
| 136 | A | NT |
| 137 | A | NT |
| 138 | B | NT |
| 139 | B | NT |
| 140 | B | NT |

Exemplary Biological Assay Data

A: 0.00001 μM<IC₅₀<1 μM

B: 1 μM<IC₅₀<5 μM

C: 5 μM<IC₅₀<10 μM

D: 10 μM<IC₅₀<500 μM

TABLE 3
IC_{SO} values for representative compounds of the disclosure measured in an
automated patch clamp assay utilizing HEK293 cells overexpressing TRPC5 (see above),
with the readout as a current block utilizing whole cell automated patch following
stimulation with rosiglitazone at either 80 or 100 mV.

| | | TRPC5_QP_ | TRPC5_QP_ |
|---|---|---|---|
| Compound | Structure | X50_100 | XC50_80 |
| AO | 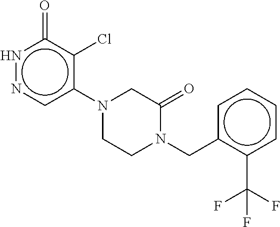 | A | A |

TABLE 4
IC_{SO} values for representative compounds
of the disclosure measured in a
Fluorescence assay—FLIPR format
utilizing cells expressing TRPC5 (HEK-TREx hTRPC5)

| | | TRPC5-FLIPR |
|---|---|---|
| Compound | Structure | IC50 (uM) |
| MS | 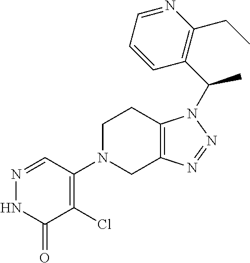 [SMILES: CCc1ncccc1[C@@H](C)n1nnc2c1CCN(c1cn[nH]c(=O)c1Cl)C2] | A |

TABLE 5
IC_{SO} values for representative compounds of the disclosure measured in a
Fluorescence assay—FLIPR format utilizing cells expressing TRPC4 (HEK-TREx
hTRPC4).

| | | TRPC4 FLIPR IC₅₀ |
|---|---|---|
| Compound | Structure | (uM) |
| MX | 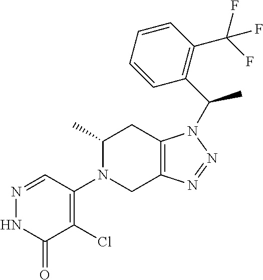 [SMILES: C[C@@H]1Cc2c(nnn2[C@H](C)c2ccccc2C(F)(F)F)CN1c1cn[nH]c(=O)c1Cl] | A |
| LY | 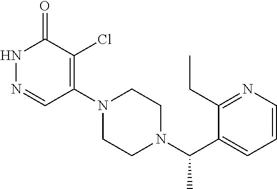 [SMILES: CCc1ncccc1[C@H](C)N1CCN(c2cn[nH]c(=O)c2Cl)CC1] | A |
| LW | 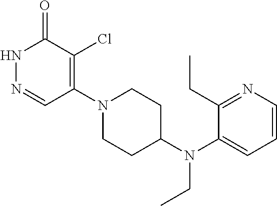 [SMILES: CCc1ncccc1N(CC)C1CCN(c2cn[nH]c(=O)c2Cl)CC1] | A |
| OP | 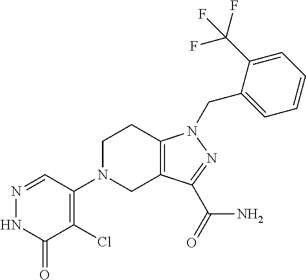 [SMILES: NC(=O)c1nn(Cc2ccccc2C(F)(F)F)c2c1CN(c1cn[nH]c(=O)c1Cl)CC2] | A |
| NL | 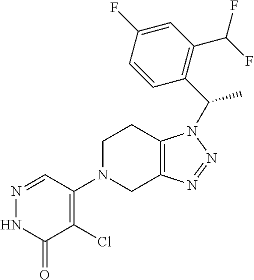 [SMILES: C[C@@H](c1ccc(F)cc1C(F)F)n1nnc2c1CCN(c1cn[nH]c(=O)c1Cl)C2] | A |
| QM | 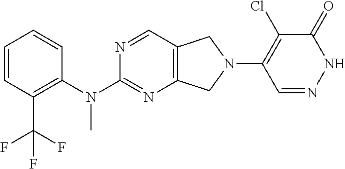 [SMILES: CN(c1ncc2c(n1)CN(c1cn[nH]c(=O)c1Cl)C2)c1ccccc1C(F)(F)F] | A |
| MD (single | | A |
| enantiomer; absolute | | |
| stereochemistry at | | |
| benzylic methine not | | |
| yet assigned) | | |
| PW | 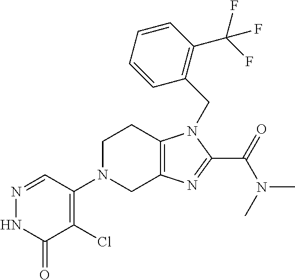 [SMILES: CN(C)C(=O)c1nc2c(n1Cc1ccccc1C(F)(F)F)CCN(c1cn[nH]c(=O)c1Cl)C2] | A |
| AO | 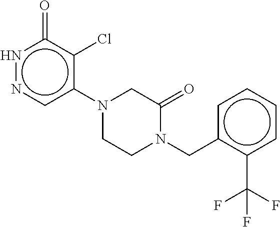 | A |

TABLE 6
ICso values for representative compounds of the disclosure measured in a
Fluorescence assay-FLIPR format utilizing cells expressing TRPC5 (HEK-TREx
hTRPC5) and TRPC4 (HEK-TREx hTRPC4).

| | | hTRPC5-FLIPR | hTRPC4-FLIPR |
|---|---|---|---|
| Compound | Structure | IC50 (uM) | IC50 (uM) |
| LY | 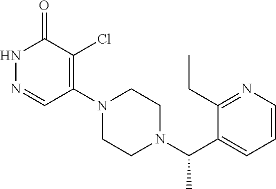 [SMILES: CCc1ncccc1[C@H](C)N1CCN(c2cn[nH]c(=O)c2Cl)CC1] | A | A |
| LW | 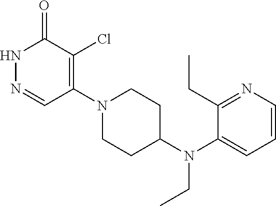 [SMILES: CCc1ncccc1N(CC)C1CCN(c2cn[nH]c(=O)c2Cl)CC1] | A | A |
| MF* | | A | A |

*Compound MF is a single stereoisomer (absolute stereochemistry not yet assigned).

TABLE 7
ICso values for representative compounds of the disclosure measured in a
Fluorescence assay-FLIPR format utilizing cells expressing TRPC5 (HEK-TREx
hTRPC5) and TRPC4 (HEK-TREx hTRPC4).

| | | TRPC5- | TRPC4- |
|---|---|---|---|
| | | FLIPR IC50 | FLIPR IC50 |
| Compound | Structure | (uM) | (uM) |
| MS | 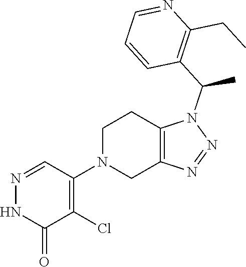 [SMILES: CCc1ncccc1[C@@H](C)n1nnc2c1CCN(c1cn[nH]c(=O)c1Cl)C2] | A | |
| MX | 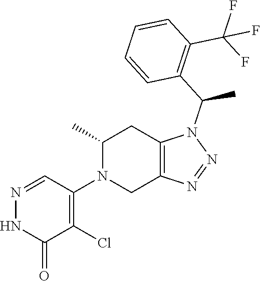 [SMILES: C[C@@H]1Cc2c(nnn2[C@H](C)c2ccccc2C(F)(F)F)CN1c1cn[nH]c(=O)c1Cl] | A | A |
| OM | 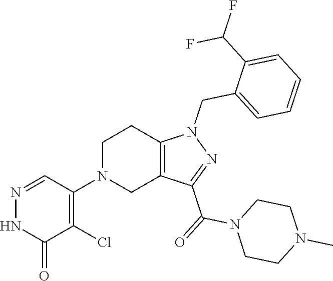 [SMILES: CN1CCN(C(=O)c2nn(Cc3ccccc3C(F)F)c3c2CN(c2cn[nH]c(=O)c2Cl)CC3)CC1] | A | A |
| OU | 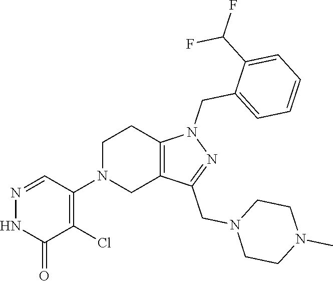 [SMILES: CN1CCN(Cc2nn(Cc3ccccc3C(F)F)c3c2CN(c2cn[nH]c(=O)c2Cl)CC3)CC1] | A | A |
| OP | 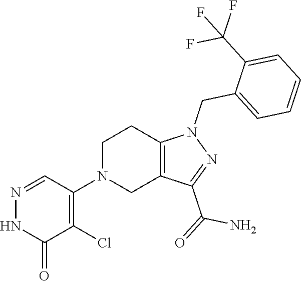 [SMILES: NC(=O)c1nn(Cc2ccccc2C(F)(F)F)c2c1CN(c1cn[nH]c(=O)c1Cl)CC2] | A | A |
| NL | 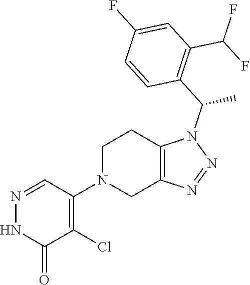 [SMILES: C[C@@H](c1ccc(F)cc1C(F)F)n1nnc2c1CCN(c1cn[nH]c(=O)c1Cl)C2] | A | A |
| QM | 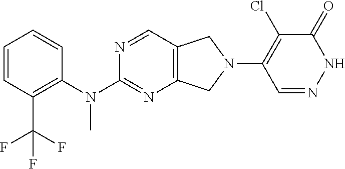 [SMILES: CN(c1ncc2c(n1)CN(c1cn[nH]c(=O)c1Cl)C2)c1ccccc1C(F)(F)F] | A | A |
| PW | 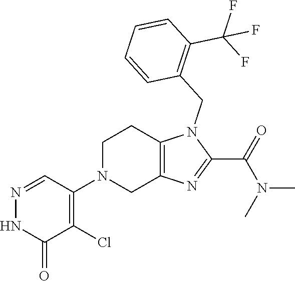 [SMILES: CN(C)C(=O)c1nc2c(n1Cc1ccccc1C(F)(F)F)CCN(c1cn[nH]c(=O)c1Cl)C2] | A | A |

### Example 43. Effects of Compound AO on Albuminuria in DOCA-Salt Hypertensive Rats

The aim of this study was to evaluate the effects of the TRCP5 inhibitor, AO, to attenuate the development and/or progression of albuminuria in deoxycorticosterone acetate (DOCA)-salt hypertensive rats.

The DOCA-salt hypertensive rat model is a well-established model of mineralocorticoid hypertension with renal dysfunction, characterized by increase levels of urinary protein and albumin excretion. [Schenk et al., “The pathogenesis of DOCA-salt hypertension,” J. Pharmacol. Toxicol. Methods (May 1992) 27(3):161-170; Gomez-Sanchez et al., “Mineralocorticoids, salt and high blood pressure,” Steroids (1996) 61:184-188.]

Six to seven weeks old Sprague Dawley rats were unilaterally nephrectomized; after one-week recovery, rats were implanted with a DOCA pellet (45 mg) and provided tap water containing 0.9% NaCl and 0.2% KCl (Day 1) for a 3 weeks treatment. On Day 1, DOCA-salt rats received one daily dose, subcutaneously (SC), of AO at 30 mg/kg for 3 weeks; control animals for DOCA treatment were administered vehicle or eplerenone, an aldosterone blocker; sham animals, implanted with a silicone-water pellet, were given tap water and received SC administration of the vehicle. Proteinuria, albuminuria and arterial blood pressure as well as body weight were recorded every week.

No adverse effects were observed in the animals administered AO. There was no significant difference in body weight and urinary creatinine excretion in rats treated with DOCA or DOCA-AO. Animals receiving DOCA and DOCA-AO had elevated mean arterial blood pressure (BP), diastolic and systolic BP, compared to sham animals, from week 1 to 3.

Water intake and urine volume produced per day were also elevated in animals receiving DOCA-salt treatment followed by vehicle or AO.

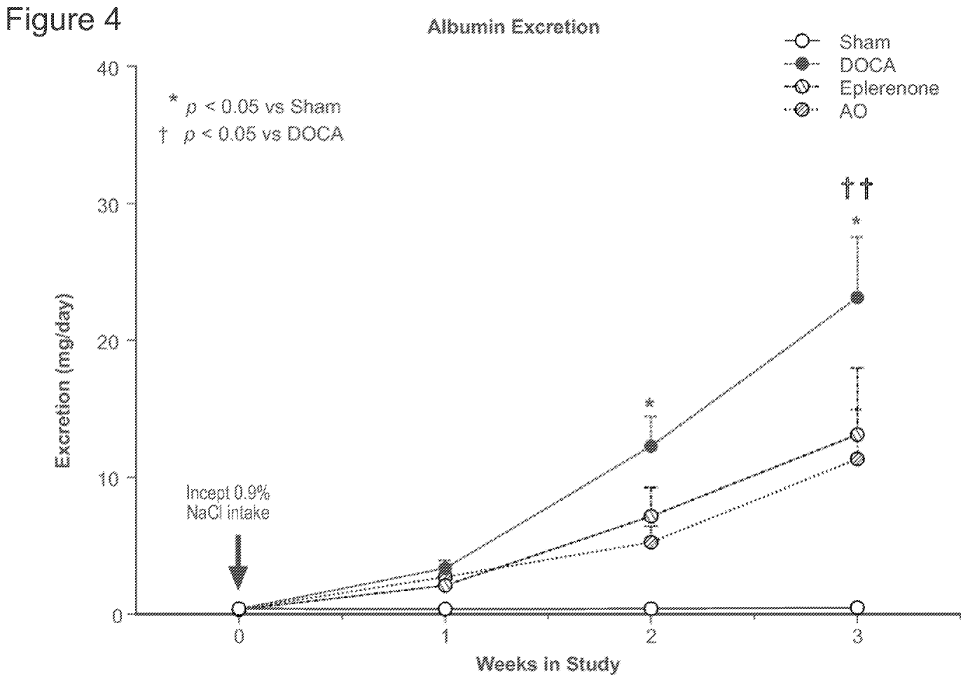

As shown in FIG. 4, AO attenuated urinary albumin excretion from week 1 to week 3 and the decrease reached significance at week 3, compared to DOCA-vehicle control rats (p value 0.0011). The albumin levels excreted in the urine were similar to the levels of the positive control animals that received eplerenone.

### Example 44. Effects of AO on Murine Podocytes with Protamine Sulfate Injury

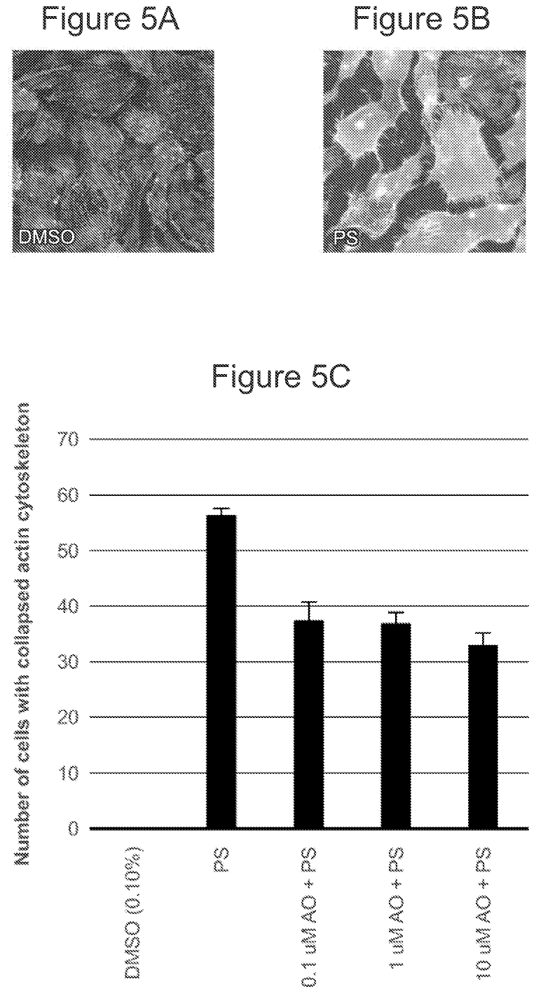

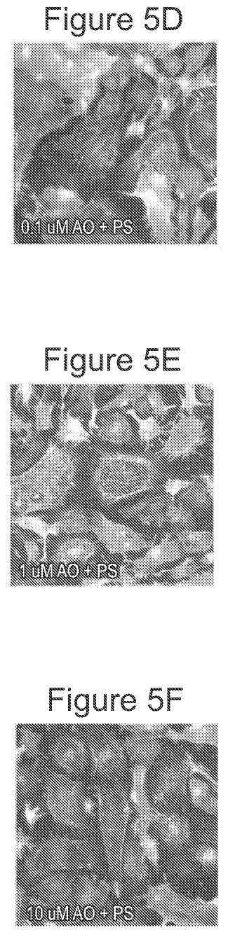

Conditionally immortalized murine podocytes were differentiated for 14 days in gamma-interferon-free media[Synaptopodin Is a Coincidence Detector of Tyrosine versus Serine/Threonine Phosphorylation for the Modulation of Rho Protein Crosstalk in Podocytes. Buvall L, Wallentin H, Sieber J, Andreeva S, Choi H Y, Mundel P, Greka A. J Am Soc Nephrol. 2017 March; 28(3):837-851. doi: 10.1681/ASN.2016040414. Epub 2016 Sep. 14.]. Murine podocyte cells were pretreated with 0.1, 1, 10 uM of AO or DMSO for 20 minutes then insulted with 300 ug/mL of protamine sulfate (PS) for 1 hour; 3 technical replicate plates were treated for each condition. Murine cells were washed with 1×DPBS −/−, fixed in 4% PFA+4% sucrose for 10 minutes at room temperature, washed 3 times with 1×DPBS −/−, permeabilized with 0.3% triton, and probed for phalloidin, synaptopodin, and DAPI (Proteasomal degradation of Nck1 but not Nck2 regulates RhoA activation and actin dynamics. Buvall L, Rashmi P, Lopez-Rivera E, Andreeva S, Weins A, Wallentin H, Greka A, Mundel P. Nat Commun. (2013) 4:2863. doi: 10.1038/ncomms3863.). Tiled images were acquired using a Zeiss LSM880 Airyscan super resolution confocal microscope using ZEN 2.3. Manual quantitation of cells with or without collapsed actin cytoskeleton were quantified. As shown in FIGS. 5A-5F, here we observe addition of AO protects ˜20% of murine cells from cytoskeletal collapse induced by protamine sulfate induced injury.

### Example 45. Effects of Compound AO on Human iPSC Derived Kidney Organoids with Protamine Sulfate Injury

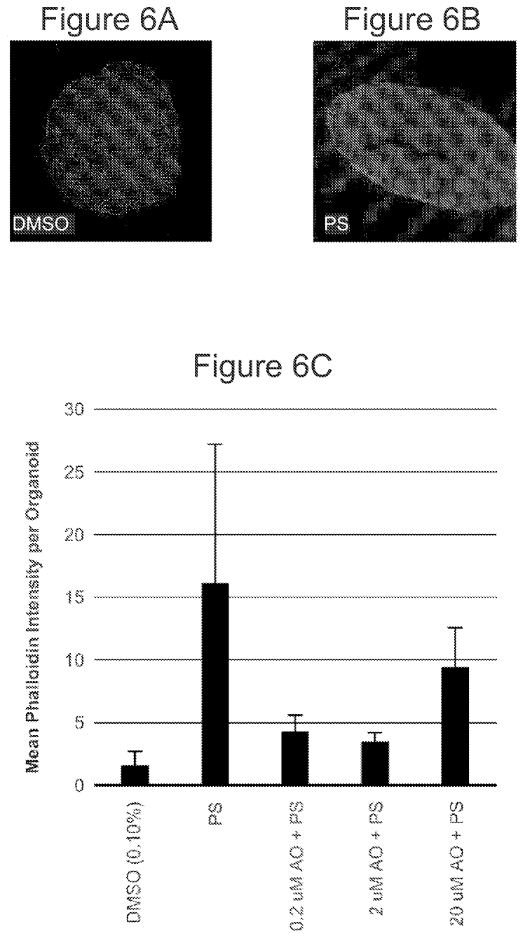

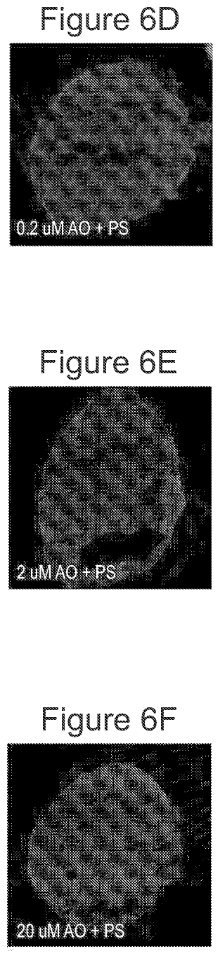

Human iPSC derived kidney organoids differentiated for 22 days [Generation of kidney organoids from human pluripotent stem cells. Takasato M, Er P X, Chiu H S, Little M H. Nat Protoc. 2016 September; 11(9):1681-92. doi: 10.1038/nprot.2016.098. Epub 2016 Aug. 18.] were pretreated with 0.2, 2, 20 uM of AO or DMSO for 20 minutes then insulted with 300 ug/mL of protamine sulfate for 1 hour; 3 technical replicate organoids were treated for each condition. Organoids were washed twice with 1×DPBS−/−, fixed in 4% PFA for 25 minutes at room temperature, washed twice with 1×DPBS−/−, and transferred to 30% sucrose at 4° C. overnight, then snap frozen in Tissue-Tek O.C.T. compound. Organoids were cryosectioned at 5 uM thickness and stained for phalloidin. Tiled images were acquired using a Zeiss LSM880 Airyscan super resolution confocal microscope using ZEN 2.3. Mean intensity values were quantified using Fiji/ImagJ1.52d. As shown in FIGS. 6A-6F, here we observe human iPSC derived kidney organoids have decreased injury from protamine sulfate injury as indicated by a decrease in mean phalloidin intensity per organoid with AO treatment compared to protamine sulfate alone.

¹H NMR and MS data for selected compounds is provided in the table below:

| Compound | Structure | NMR | MS |
|---|---|---|---|
| 100 | 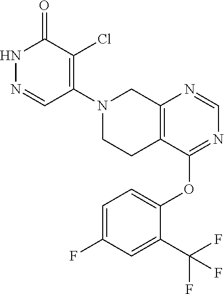 [SMILES: O=c1[nH]ncc(N2CCc3c(ncnc3Oc3ccc(F)cc3C(F)(F)F)C2)c1Cl] | 1H NMR (400 MHz, DMSO-d6) chemical shifts 13.00 (s, 1H), 8.57 (s, 1H), 8.04 (s, 1H), 7.79 (dd, J = 8.4, 3.0 Hz, 1H), 7.70 (s, 1H), 7.70- 7.59 (m, 1H), 4.68 (s, 2H), 3.79 (t, J = 5.6 Hz, 2H), 2.98 (s, 2H). | 442 |
| 101 | 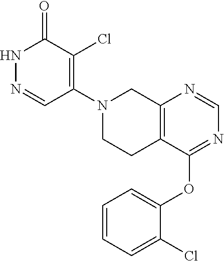 [SMILES: O=c1[nH]ncc(N2CCc3c(ncnc3Oc3ccccc3Cl)C2)c1Cl] | 1H NMR (400 MHz, DMSO-d6) chemical shifts 13.00 (s, 1H), 8.54 (s, 1H), 8.03 (s, 1H), 7.63 (dd, J = 8.0, 1.4 Hz, 1H), 7.51-7.40 (m, 2H), 7.36 (td, J = 7.4, 2.2 Hz, 1H), 4.67 (s, 2H), 3.80 (t, J = 5.7 Hz, 2H), 3.03 (t, J = 5.7 Hz, 2H). | 390 |
| 102 | 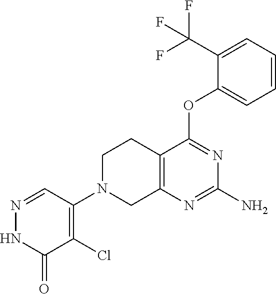 [SMILES: Nc1nc2c(c(Oc3ccccc3C(F)(F)F)n1)CCN(c1cn[nH]c(=O)c1Cl)C2] | 1H NMR (400 MHz, Methanol-d4) chemical shifts 8.00 (s, 1H), 7.79- 7.67 (m, 2H), 7.44 (dd, J = 13.1, 7.9 Hz, 2H), 4.51 (s, 2H), 3.87 (t, J = 5.8 Hz, 2H), 2.93 (t, J = 5.6 Hz, 2H). | 439 |
| 103 | 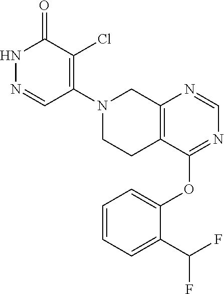 [SMILES: O=c1[nH]ncc(N2CCc3c(ncnc3Oc3ccccc3C(F)F)C2)c1Cl] | 1H NMR (400 MHz, DMSO-d6) chemical shifts 13.00 (s, 1H), 8.54 (s, 1H), 8.04 (s, 1H), 7.71 (d, J = 7.7 Hz, 1H), 7.65 (t, J = 7.9 Hz, 1H), 7.46 (t, J = 7.6 Hz, 1H), 7.40 (d, J = 8.2 Hz, 1H), 7.13 (t, J = 54.3 Hz, 1H), 4.67 (s, 2H), 3.79 (t, J = 5.7 Hz, 2H), 3.03 (t, J = 5.7 Hz, 2H). | 406 |
| 104 | 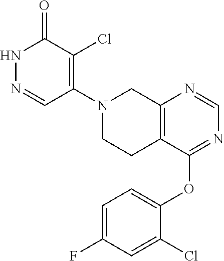 [SMILES: O=c1[nH]ncc(N2CCc3c(ncnc3Oc3ccc(F)cc3Cl)C2)c1Cl] | 1H NMR (400 MHz, DMSO-d6) chemical shifts 12.97 (s, 1H), 8.55 (s, 1H), 8.03 (s, 1H), 7.66 (dd, J = 8.3, 3.0 Hz, 1H), 7.51 (dd, J = 8.9, 5.3 Hz, 1H), 7.42-7.24 (m, 1H), 4.67 (s, 2H), 3.80 (t, J = 5.9 Hz, 2H), 3.02 (s, 2H). | 407 |
| 105 | 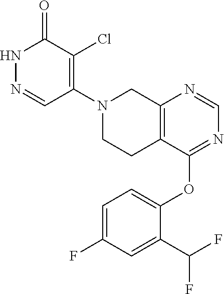 [SMILES: O=c1[nH]ncc(N2CCc3c(ncnc3Oc3ccc(F)cc3C(F)F)C2)c1Cl] | 1H NMR (400 MHz, DMSO-d6) chemical shifts 13.00 (s, 1H), 8.55 (s, 1H), 8.04 (s, 1H), 7.51 (ddd, J = 14.3, 10.8, 6.8 Hz, 3H), 7.13 (t, J = 53.9 Hz, 1H), 4.67 (s, 2H), 3.79 (t, J = 5.7 Hz, 2H), 3.03 (t, J = 5.8 Hz, 2H). | 424 |
| 106 | 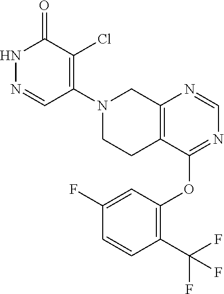 [SMILES: O=c1[nH]ncc(N2CCc3c(ncnc3Oc3cc(F)ccc3C(F)(F)F)C2)c1Cl] | 1H NMR (400 MHz, DMSO-d6) chemical shifts 13.00 (s, 1H), 8.61 (s, 1H), 8.03 (s, 1H), 7.95 (d, J = 8.4 Hz, 1H), 7.62 (d, J = 9.7 Hz, 1H), 7.41 (s, 1H), 4.69 (s, 2H), 3.80 (s, 2H), 2.98 (s, 2H). | 442 |
| 107 | 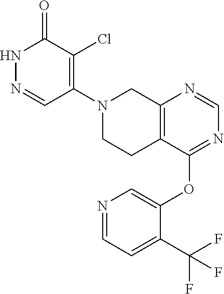 [SMILES: O=c1[nH]ncc(N2CCc3c(ncnc3Oc3cnccc3C(F)(F)F)C2)c1Cl] | 1H NMR (400 MHz, DMSO-d6) chemical shifts 13.00 (s, 1H), 8.91 (s, 1H), 8.79 (d, J = 4.9 Hz, 1H), 8.59 (s, 1H), 8.04 (s, 1H), 7.92 (d, J = 5.0 Hz, 1H), 4.70 (s, 2H), 3.81 (t, J = 5.7 Hz, 2H), 3.02 (s, 2H). | 425 |
| 108 | 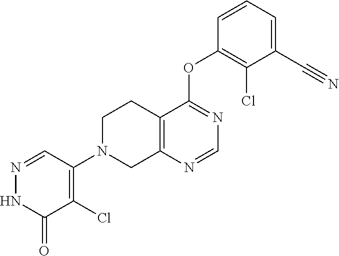 [SMILES: N#Cc1cccc(Oc2ncnc3c2CCN(c2cn[nH]c(=O)c2Cl)C3)c1Cl] | 1H NMR (400 MHz, DMSO-d6) chemical shifts 13.00 (s, 1H), 8.57 (s, 1H), 8.04 (s, 1H), 7.99 (dd, J = 7.8, 1.5 Hz, 1H), 7.86 (dd, J = 8.3, 1.5 Hz, 1H), 7.69 (t, J = 8.0 Hz, 1H), 4.69 (s, 2H), 3.81 (t, J = 5.7 Hz, 2H), 3.08-3.01 (m, 2H). | 415 |
| 109 | 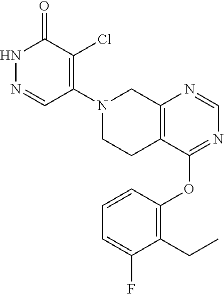 [SMILES: CCc1c(F)cccc1Oc1ncnc2c1CCN(c1cn[nH]c(=O)c1Cl)C2] | 1H NMR (400 MHz, DMSO-d6) chemical shifts 13.01 (s, 1H), 8.54 (s, 1H), 8.04 (s, 1H), 7.38-7.28 (m, 1H), 7.15 (t, J = 8.9 Hz, 1H), 7.06 (d, J = 8.2 Hz, 1H), 4.67 (s, 2H), 3.80 (t, J = 5.8 Hz, 2H), 3.03 (d, J = 5.8 Hz, 2H), 2.48 (d, J = 7.4 Hz, 2H), 1.05 (t, J = 7.5 Hz, 3H). | 402 |
| 110 | 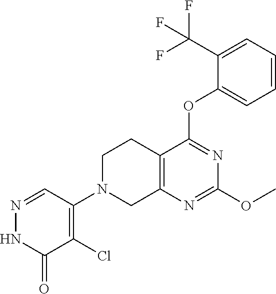 [SMILES: COc1nc2c(c(Oc3ccccc3C(F)(F)F)n1)CCN(c1cn[nH]c(=O)c1Cl)C2] | 1H NMR (400 MHz, DMSO-d6) chemical shifts 12.98 (s, 1H), 8.02 (s, 1H), 7.88-7.75 (m, 2H), 7.59- 7.48 (m, 2H), 4.60 (s, 2H), 3.77 (t, J = 5.7 Hz, 2H), 3.74 (s, 3H), 2.89 (t, J = 5.6 Hz, 2H). | 454 |
| 111 | 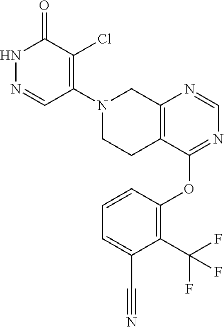 [SMILES: N#Cc1cccc(Oc2ncnc3c2CCN(c2cn[nH]c(=O)c2Cl)C3)c1C(F)(F)F] | 1H NMR (400 MHz, DMSO-d6) chemical shifts 13.01 (s, 1H), 8.60 (s, 1H), 8.13 (d, J = 7.4 Hz, 1H), 8.07-7.99 (m, 2H), 7.96 (d, J = 8.4 Hz, 1H), 4.70 (s, 2H), 3.81 (t, J = 5.6 Hz, 2H), 3.01 (s, 2H). | 449 |
| 112 | 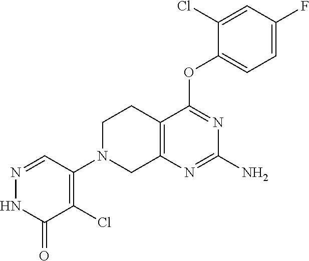 [SMILES: Nc1nc2c(c(Oc3ccc(F)cc3Cl)n1)CCN(c1cn[nH]c(=O)c1Cl)C2] | 1H NMR (400 MHz, DMSO-d6) chemical shifts 12.95 (s, 1H), 7.97 (s, 1H), 7.62 (dd, J = 8.5, 3.1 Hz, 1H), 7.45 (dd, J = 9.0, 5.3 Hz, 1H), 7.31 (td, J = 8.5, 2.9 Hz, 1H), 6.50 (s, 2H), 4.41 (s, 2H), 3.72 (d, J = 6.0 Hz, 2H), 2.80 (s, 2H). | 423 |
| 113 | 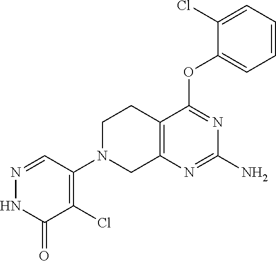 [SMILES: Nc1nc2c(c(Oc3ccccc3Cl)n1)CCN(c1cn[nH]c(=O)c1Cl)C2] | 1H NMR (400 MHz, DMSO-d6) chemical shifts 12.95 (s, 1H), 7.97 (s, 1H), 7.58 (dd, J = 7.9, 1.5 Hz, 1H), 7.47-7.38 (m, 1H), 7.41- 7.34 (m, 1H), 7.34-7.25 (m, 1H), 6.48 (s, 2H), 4.41 (s, 2H), 3.73 (t, J = 5.8 Hz, 2H), 2.81 (d, J = 5.7 Hz, 2H). | 405 |
| 114 | 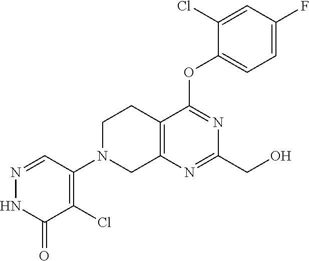 [SMILES: O=c1[nH]ncc(N2CCc3c(nc(CO)nc3Oc3ccc(F)cc3Cl)C2)c1Cl] | 1H NMR (400 MHz, DMSO-d6) chemical shifts 13.00 (s, 1H), 8.03 (s, 1H), 7.66 (dd, J = 8.4, 3.0 Hz, 1H), 7.51 (dd, J = 9.0, 5.3 Hz, 1H), 7.35 (td, J = 8.6, 3.0 Hz, 1H), 5.17 (t, J = 6.3 Hz, 1H), 4.67 (s, 2H), 4.32 (d, J = 6.3 Hz, 2H), 3.80 (t, J = 5.7 Hz, 2H), 3.00 (d, J = 6.4 Hz, 2H). | 438 |
| 115 | 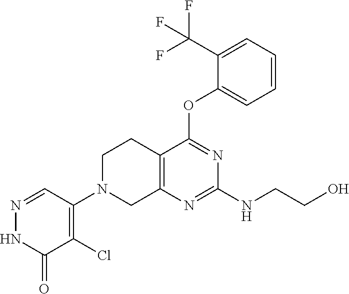 [SMILES: O=c1[nH]ncc(N2CCc3c(nc(NCCO)nc3Oc3ccccc3C(F)(F)F)C2)c1Cl] | 1H NMR (400 MHz, DMSO-d6) chemical shifts 12.95 (s, 1H), 7.98 (s, 1H), 7.83-7.72 (m, 2H), 7.48 (dd, J = 18.0, 8.2 Hz, 2H), 6.82 (s, 1H), 4.54 (s, 1H), 4.44 (s, 2H), 3.73 (t, J = 5.6 Hz, 2H), 3.33 (m, 4H), 2.78 (s, 2H). | 483 |
| 116 | 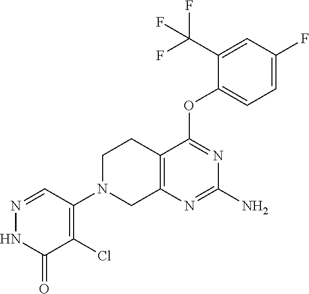 [SMILES: Nc1nc2c(c(Oc3ccc(F)cc3C(F)(F)F)n1)CCN(c1cn[nH]c(=O)c1Cl)C2] | 1H NMR (400 MHz, DMSO-d6) chemical shifts 12.96 (s, 1H), 7.97 (s, 1H), 7.79-7.71 (m, 1H), 7.66 (td, J = 8.5, 3.0 Hz, 1H), 7.56 (dd, J = 9.2, 4.6 Hz, 1H), 6.54 (s, 2H), 4.41 (s, 2H), 3.72 (t, J = 5.8 Hz, 2H), 2.76 (t, J = 5.7 Hz, 2H). | 457 |
| 117* | 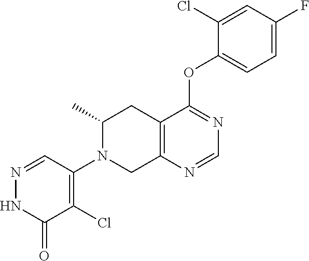 [SMILES: C[C@@H]1Cc2c(ncnc2Oc2ccc(F)cc2Cl)CN1c1cn[nH]c(=O)c1Cl] | 1H NMR (400 MHz, DMSO-d6) chemical shifts 13.04 (s, 1H), 8.56 (s, 1H), 8.02 (s, 1H), 7.67 (dd, J = 8.4, 2.5 Hz, 1H), 7.53 (dd, J = 9.2, 5.3 Hz, 1H), 7.41-7.31 (m, 1H), 4.75 (d, J = 18.1 Hz, 1H), 4.62- 4.34 (m, 2H), 3.23 (dd, J = 17.0, 6.0 Hz, 1H), 2.81 (d, J = 17.1 Hz, 1H), 1.20 (d, J = 6.6 Hz, 3H). | 422 |
| 117a* | 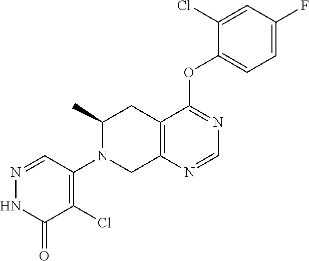 [SMILES: C[C@H]1Cc2c(ncnc2Oc2ccc(F)cc2Cl)CN1c1cn[nH]c(=O)c1Cl] | 1H NMR (400 MHz, DMSO-d6) chemical shifts 13.04 (s, 1H), 8.56 (s, 1H), 8.02 (s, 1H), 7.67 (dd, J = 8.6, 2.5 Hz, 1H), 7.58-7.50 (m, 1H), 7.36 (dd, J = 9.9, 7.3 Hz, 1H), 4.76 (d, J = 18.1 Hz, 1H), 4.63- 4.40 (m, 2H), 3.23 (dd, J = 17.3, 5.9 Hz, 1H), 2.81 (d, J = 17.0 Hz, 1H), 1.20 (d, J = 6.7 Hz, 3H). | 422 |
| 118 | 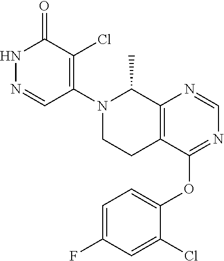 [SMILES: C[C@@H]1c2ncnc(Oc3ccc(F)cc3Cl)c2CCN1c1cn[nH]c(=O)c1Cl] | 1H NMR (400 MHz, DMSO-d6) chemical shifts 12.96 (s, 1H), 8.55 (s, 1H), 8.02 (s, 1H), 7.66 (dd, J = 8.4, 3.0 Hz, 1H), 7.50 (dd, J = 9.0, 5.3 Hz, 1H), 7.35 (td, J = 8.5, 3.0 Hz, 1H), 5.06 (q, J = 6.7 Hz, 1H), 4.00 (dd, J = 14.1, 5.7 Hz, 1H), 3.68 (ddd, J = 14.4, 11.1, 4.2 Hz, 1H), 3.06 (ddd, J = 17.1, 11.2, 6.0 Hz, 1H), 2.98-2.88 (m, 1H), 1.60 (d, J = 6.8 Hz, 3H). | 422 |
| 119 | 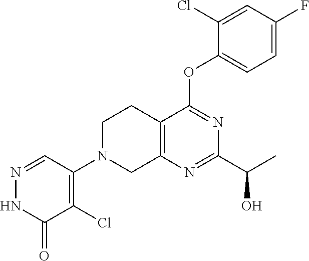 [SMILES: C[C@@H](O)c1nc2c(c(Oc3ccc(F)cc3Cl)n1)CCN(c1cn[nH]c(=O)c1Cl)C2] | 1H NMR (400 MHz, DMSO-d6) chemical shifts 12.98 (s, 1H), 8.03 (s, 1H), 7.66 (dd, J = 8.4, 3.0 Hz, 1H), 7.51 (dd, J = 9.1, 5.3 Hz, 1H), 7.35 (td, J = 8.5, 3.0 Hz, 1H), 5.06 (d, J = 5.3 Hz, 1H), 4.67 (s, 2H), 4.48 (p, J = 6.4 Hz, 1H), 3.80 (t, J = 5.5 Hz, 2H), 2.99 (s, 2H), 1.22 (t, J = 6.4 Hz, 3H). | 452 |
| 120 | 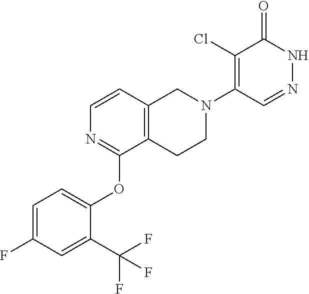 [SMILES: O=c1[nH]ncc(N2CCc3c(ccnc3Oc3ccc(F)cc3C(F)(F)F)C2)c1Cl] | 1H NMR (400 MHz, DMSO-d6) chemical shifts 13.00 (s, 1H), 7.98 (s, 1H), 7.90 (d, J = 5.2 Hz, 1H), 7.72 (dd, J = 8.6, 3.1 Hz, 1H), 7.63 (td, J = 8.6, 3.1 Hz, 1H), 7.49 (dd, J = 9.1, 4.6 Hz, 1H), 7.00 (d, J = 5.2 Hz, 1H), 4.69 (s, 2H), 3.78 (t, J = 5.8 Hz, 2H), 2.95 (t, J = 5.9 Hz, 2H). | 441 |
| 121 | 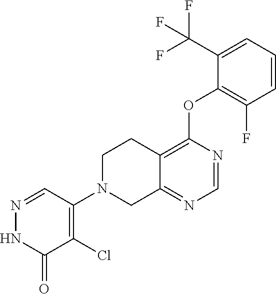 [SMILES: O=c1[nH]ncc(N2CCc3c(ncnc3Oc3c(F)cccc3C(F)(F)F)C2)c1Cl] | 1H NMR (400 MHz, DMSO-d6) chemical shifts 13.01 (s, 1H), 8.60 (s, 1H), 8.04 (s, 1H), 7.83 (t, J = 9.2 Hz, 1H), 7.73 (d, J = 7.9 Hz, 1H), 7.67-7.57 (m, 1H), 4.71 (s, 2H), 3.81 (t, J = 5.8 Hz, 2H), 3.01 (s, 2H). | 442 |
| 122 | 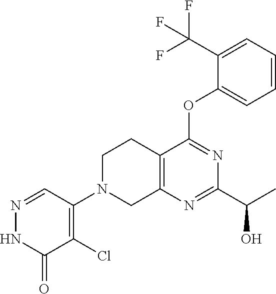 [SMILES: C[C@@H](O)c1nc2c(c(Oc3ccccc3C(F)(F)F)n1)CCN(c1cn[nH]c(=O)c1Cl)C2] | 1H NMR (400 MHz, DMSO-d6) chemical shifts 13.00 (s, 1H), 8.03 (s, 1H), 7.88-7.75 (m, 2H), 7.57 (d, J = 8.2 Hz, 1H), 7.51 (t, J = 7.6 Hz, 1H), 5.07 (d, J = 5.3 Hz, 1H), 4.67 (s, 2H), 4.50 (p, J = 6.2 Hz, 1H), 3.80 (t, J = 5.6 Hz, 2H), 2.96 (s, 2H), 1.23 (d, J = 6.5 Hz, 3H). | 468 |
| 123 | 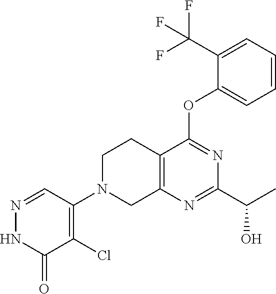 [SMILES: C[C@H](O)c1nc2c(c(Oc3ccccc3C(F)(F)F)n1)CCN(c1cn[nH]c(=O)c1Cl)C2] | 1H NMR (400 MHz, DMSO-d6) chemical shifts 13.00 (s, 1H), 8.03 (s, 1H), 7.88-7.75 (m, 2H), 7.57 (d, J = 8.3 Hz, 1H), 7.51 (t, J = 7.7 Hz, 1H), 5.07 (d, J = 5.2 Hz, 1H), 4.67 (s, 2H), 4.50 (p, J = 6.4 Hz, 1H), 3.80 (t, J = 5.6 Hz, 2H), 2.96 (s, 2H), 1.23 (d, J = 6.6 Hz, 3H). | 468 |
| 124 | 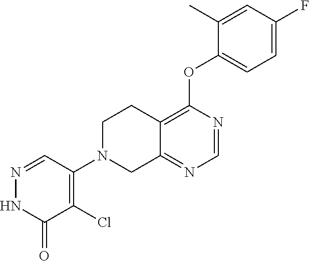 [SMILES: Cc1cc(F)ccc1Oc1ncnc2c1CCN(c1cn[nH]c(=O)c1Cl)C2] | 1H NMR (DMSO-d6) δ: 12.97 (br s, 1H), 8.49 (s, 1H), 8.01 (s, 1H), 7.15-7.24 (m, 2H), 7.05-7.12 (m, 1H), 4.63 (s, 2H), 3.78 (t, J = 5.7 Hz, 2H), 3.00 (s, 2H), 2.06 (s, 3H) | 388.3 |
| 125 | 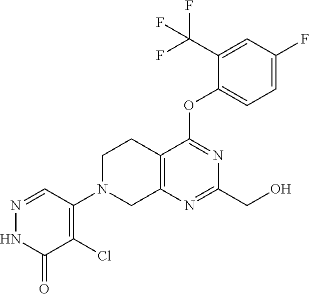 [SMILES: O=c1[nH]ncc(N2CCc3c(nc(CO)nc3Oc3ccc(F)cc3C(F)(F)F)C2)c1Cl] | 1H NMR (400 MHz, DMSO-d6) chemical shifts 13.00 (s, 1H), 8.03 (s, 1H), 7.81-7.74 (m, 1H), 7.74- 7.60 (m, 2H), 5.19 (t, J = 6.3 Hz, 1H), 4.67 (s, 2H), 4.34 (d, J = 6.3 Hz, 2H), 3.79 (t, J = 5.6 Hz, 2H), 2.95 (s, 2H). | 472 |
| 126* | 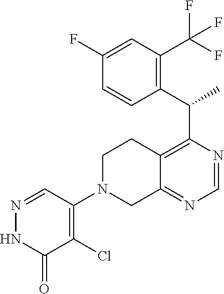 [SMILES: C[C@@H](c1ccc(F)cc1C(F)(F)F)c1ncnc2c1CCN(c1cn[nH]c(=O)c1Cl)C2] | 1H NMR (400 MHz, DMSO-d6) chemical shifts 12.98 (s, 1H), 9.02 (s, 1H), 7.97 (s, 1H), 7.63 (dd, J = 9.4, 2.6 Hz, 1H), 7.53 (p, J = 8.7 Hz, 2H), 4.71 (t, J = 7.1 Hz, 1H), 4.65 (s, 2H), 3.68 (t, J = 5.7 Hz, 2H), 3.08 (d, J = 16.5 Hz, 1H), 2.57 (s, 1H), 1.60 (d, J = 6.8 Hz, 3H). | 454 |
| 126a* | 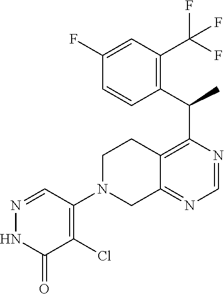 [SMILES: C[C@H](c1ccc(F)cc1C(F)(F)F)c1ncnc2c1CCN(c1cn[nH]c(=O)c1Cl)C2] | 1H NMR (400 MHz, DMSO-d6) chemical shifts 12.97 (s, 1H), 9.02 (s, 1H), 7.97 (s, 1H), 7.63 (d, J = 7.9 Hz, 1H), 7.60-7.49 (m, 2H), 4.72 (d, J = 6.9 Hz, 1H), 4.65 (s, 2H), 3.68 (s, 2H), 3.08 (d, J = 16.3 Hz, 1H), 2.58 (s, 1H), 1.60 (d, J = 6.8 Hz, 3H). | 454 |
| 127 | 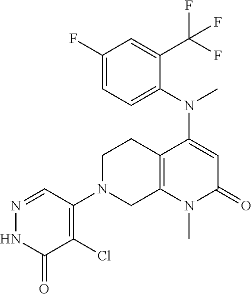 [SMILES: CN(c1ccc(F)cc1C(F)(F)F)c1cc(=O)n(C)c2c1CCN(c1cn[nH]c(=O)c1Cl)C2] | 1H NMR (400 MHz, DMSO-d6) chemical shifts 12.98 (s, 1H), 7.96 (s, 1H), 7.81-7.74 (m, 1H), 7.55 (d, J = 8.8 Hz, 1H), 7.35-7.27 (m, 1H), 5.85 (s, 1H), 4.52 (s, 2H), 3.39 (s, 3H), 3.39-3.34(m, 2H), 3.09 (s, 3H), 1.95 (s, 2H). | 484 |
| 128 | 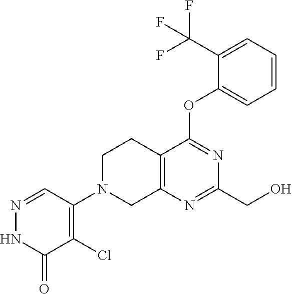 [SMILES: O=c1[nH]ncc(N2CCc3c(nc(CO)nc3Oc3ccccc3C(F)(F)F)C2)c1Cl] | 1H NMR (400 MHz, DMSO-d6) chemical shifts 13.01 (s, 1H), 8.03 (s, 1H), 7.88-7.75 (m, 2H), 7.56 (d, J = 8.3 Hz, 1H), 7.51 (t, J = 7.7 Hz, 1H), 5.19 (t, J = 6.3 Hz, 1H), 4.68 (s, 2H), 4.34 (d, J = 6.3 Hz, 2H), 3.80 (t, J = 5.7 Hz, 2H), 2.96 (s, 2H). | 454 |
| 129* | 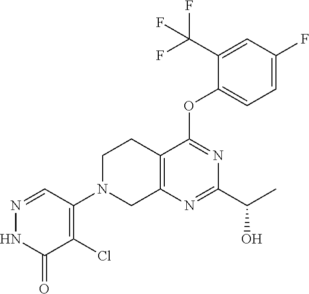 [SMILES: C[C@H](O)c1nc2c(c(Oc3ccc(F)cc3C(F)(F)F)n1)CCN(c1cn[nH]c(=O)c1Cl)C2] | 1H NMR (400 MHz, DMSO-d6) chemical shifts 13.00 (s, 1H), 8.03 (s, 1H), 7.78 (dd, J = 8.6, 2.9 Hz, 1H), 7.75-7.61 (m, 2H), 5.08 (d, J = 5.3 Hz, 1H), 4.67 (s, 2H), 4.50 (p, J = 6.5 Hz, 1H), 3.79 (t, J = 5.7 Hz, 2H), 2.94 (d, J = 6.1 Hz, 2H), 1.23 (d, J = 6.5 Hz, 3H). | 486 |
| 130* | 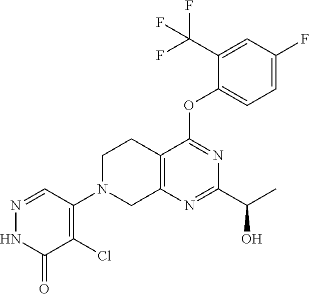 [SMILES: C[C@@H](O)c1nc2c(c(Oc3ccc(F)cc3C(F)(F)F)n1)CCN(c1cn[nH]c(=O)c1Cl)C2] | 1H NMR (400 MHz, DMSO-d6) chemical shifts 13.00 (s, 1H), 8.03 (s, 1H), 7.78 (dd, J = 8.6, 3.0 Hz, 1H), 7.67 (ddd, J = 19.1, 8.6, 3.8 Hz, 2H), 5.08 (d, J = 5.3 Hz, 1H), 4.67 (s, 2H), 4.50 (p, J = 6.5 Hz, 1H), 3.79 (t, J = 5.5 Hz, 2H), 2.95 (s, 2H), 1.23 (d, J = 6.6 Hz, 3H). | 486 |
| 131 | 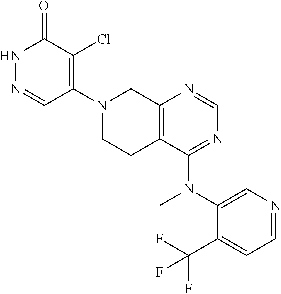 [SMILES: CN(c1cnccc1C(F)(F)F)c1ncnc2c1CCN(c1cn[nH]c(=O)c1Cl)C2] | 1H NMR (400 MHz, DMSO-d6) chemical shifts 12.07(s, 1H), 8.89- 8.79 (m, 2H), 8.59 (s, 1H), 7.94- 7.84 (m, 2H), 4.53 (s, 2H), 3.44 (d, J = 11.0 Hz, 2H), 3.41 (s, 3H), 1.96 (t, J = 5.6 Hz, 2H). | 438 |
| 132 | 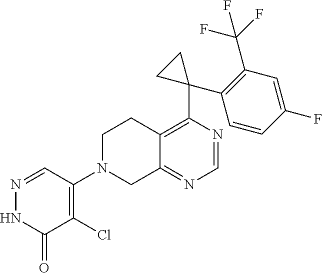 [SMILES: O=c1[nH]ncc(N2CCc3c(ncnc3C3(c4ccc(F)cc4C(F)(F)F)CC3)C2)c1Cl] | 1H NMR (400 MHz, DMSO-d6) chemical shifts 12.94 (s, 1H), 8.89 (s, 1H), 8.00 (d, J = 8.8 Hz, 1H), 7.87 (s, 1H), 7.62 (d, J = 8.6 Hz, 2H), 4.58 (s, 2H), 3.48 (s, 2H), 2.27 (s, 2H), 1.84 (s, 2H), 1.47 (s, 2H). | 466 |
| 133 | 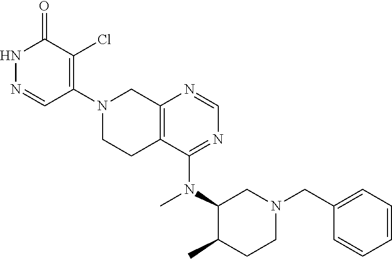 [SMILES: C[C@@H]1CCN(Cc2ccccc2)C[C@@H]1N(C)c1ncnc2c1CCN(c1cn[nH]c(=O)c1Cl)C2] | 1H NMR (400 MHz, Methanol-d4) chemical shifts 8.34 (s, 1H), 8.02 (s, 1H), 7.33 (q, J = 7.8 Hz, 4H), 7.25 (t, J = 7.0 Hz, 1H), 4.67 (d, J = 17.3 Hz, 1H), 4.58 (d, J = 17.2 Hz, 2H), 3.97 (d, J = 13.9 Hz, 1H), 3.55 (s, 2H), 3.47 (s, 1H), 3.39 (s, 3H), 2.94 (s, 1H), 2.81 (d, J = 15.8 Hz, 1H), 2.74-2.53 (m, 3H), 2.35 (s, 1H), 2.15 (s, 1H), 1.76 (s, 2H), 1.01 (d, J = 7.0 Hz, 3H). | 480 |
| 133a | 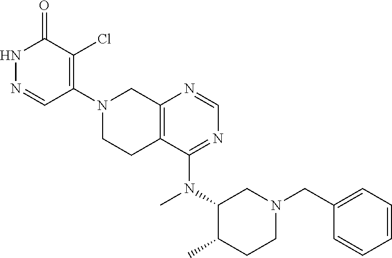 [SMILES: C[C@H]1CCN(Cc2ccccc2)C[C@H]1N(C)c1ncnc2c1CCN(c1cn[nH]c(=O)c1Cl)C2] | 1H NMR (400 MHz, Methanol-d4) chemical shifts 8.34 (s, 1H), 8.02 (s, 1H), 7.42-7.21 (m, 5H), 4.62 (q, J = 17.5 Hz, 3H), 3.97 (d, J = 13.3 Hz, 1H), 3.55 (s, 2H), 3.47 (s, 1H), 3.39 (s, 3H), 3.15 (s, 1H), 2.94 (s, 1H), 2.87-2.61 (m, 3H), 2.35 (s, 1H), 2.15 (s, 1H), 1.76 (s, 2H), 1.01 (d, J = 7.0 Hz, 3H). | 480 |
| 134 | 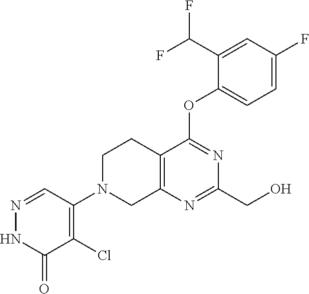 [SMILES: O=c1[nH]ncc(N2CCc3c(nc(CO)nc3Oc3ccc(F)cc3C(F)F)C2)c1Cl] | 1H NMR (400 MHz, DMSO-d6) chemical shifts 13.01 (s, 1H), 8.03 (s, 1H), 7.58-7.49 (m, 3H), 7.27- 7.00 (m, 1H), 5.17 (t, J = 6.3 Hz, 1H), 4.66 (s, 2H), 4.34 (d, J = 6.3 Hz, 2H), 3.79 (t, J = 5.7 Hz, 2H), 2.99 (s, 2H). | 454 |
| 135* | 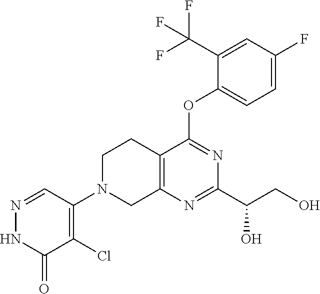 [SMILES: O=c1[nH]ncc(N2CCc3c(nc([C@@H](O)CO)nc3Oc3ccc(F)cc3C(F)(F)F)C2)c1Cl] | 1H NMR (400 MHz, DMSO-d6) chemical shifts 12.97 (s, 1H), 8.03 (s, 1H), 7.78 (dd, J = 8.5, 3.0 Hz, 1H), 7.75-7.61 (m, 2H), 5.12 (d, J = 5.7 Hz, 1H), 4.67 (s, 2H), 4.57 (t, J = 6.0 Hz, 1H), 4.37 (q, J = 5.8 Hz, 1H), 3.79 (t, J = 5.7 Hz, 2H), 3.57 (dt, J = 11.2, 5.7 Hz, 1H), 3.46 (dt, J = 10.8, 6.1 Hz, 1H), 2.96 (t, J = 5.6 Hz, 2H). | 502 |
| 136 | 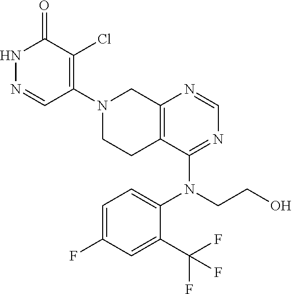 [SMILES: O=c1[nH]ncc(N2CCc3c(ncnc3N(CCO)c3ccc(F)cc3C(F)(F)F)C2)c1Cl] | 1H NMR (400 MHz, Methanol-d4) chemical shifts 8.54 (s, 1H), 7.87 (s, 1H), 7.75 (s, 1H), 7.63 (dd, J = 8.8, 3.0 Hz, 1H), 7.54 (s, 1H), 4.57 (m, 3H), 3.88 (s, 2H), 3.51 (m, 3H), 2.05 (s, 1H), 1.98 (s, 1H). | 485 |
| 137* | 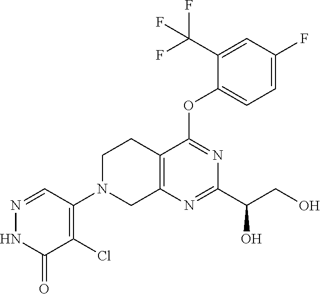 [SMILES: O=c1[nH]ncc(N2CCc3c(nc([C@H](O)CO)nc3Oc3ccc(F)cc3C(F)(F)F)C2)c1Cl] | 1H NMR (400 MHz, DMSO-d6) chemical shifts 12.94 (s, 1H), 8.03 (s, 1H), 7.78 (dd, J = 8.5, 3.0 Hz, 1H), 7.75-7.61 (m, 2H), 5.12 (d, J = 5.7 Hz, 1H), 4.67 (s, 2H), 4.57 (t, J = 6.0 Hz, 1H), 4.37 (q, J = 5.7 Hz, 1H), 3.79 (t, J = 5.7 Hz, 2H), 3.57 (dt, J = 11.2, 5.7 Hz, 1H), 3.52- 3.43 (m, 1H), 2.96 (t, J = 5.6 Hz, 2H). | 502 |
| 138 | 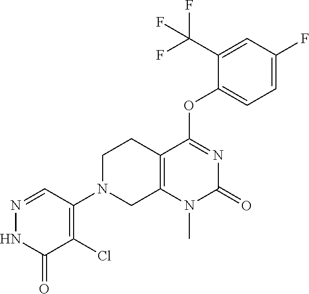 [SMILES: Cn1c2c(c(Oc3ccc(F)cc3C(F)(F)F)nc1=O)CCN(c1cn[nH]c(=O)c1Cl)C2] | 1H NMR (400 MHz, DMSO-d6) chemical shifts 13.05 (s, 1H), 8.11 (s, 1H), 7.78 (dd, J = 8.5, 3.1 Hz, 1H), 7.70 (td, J = 8.5, 3.2 Hz, 1H), 7.57 (dd, J = 9.1, 4.6 Hz, 1H), 4.65 (s, 2H), 3.66 (t, J = 5.7 Hz, 2H), 3.38 (s, 3H), 2.74 (t, J = 5.4 Hz, 2H). | 472 |
| 139 | 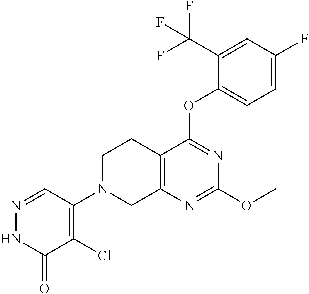 [SMILES: COc1nc2c(c(Oc3ccc(F)cc3C(F)(F)F)n1)CCN(c1cn[nH]c(=O)c1Cl)C2] | 1H NMR (400 MHz, DMSO-d6) chemical shifts 12.84 (s, 1H), 8.02 (s, 1H), 7.79 (dd, J = 8.6, 2.9 Hz, 1H), 7.68 (dtd, J = 18.8, 9.0, 3.8 Hz, 2H), 4.60 (s, 2H), 3.77 (d, J = 5.6 Hz, 2H), 3.75 (s, 3H), 2.88 (t, J = 5.7 Hz, 2H) | 472 |
| 140 | 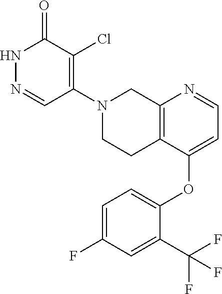 [SMILES: O=c1[nH]ncc(N2CCc3c(Oc4ccc(F)cc4C(F)(F)F)ccnc3C2)c1Cl] | 1H NMR (400 MHz, DMSO-d6) chemical shifts 12.97 (s, 1H), 8.30 (d, J = 5.6 Hz, 1H), 8.02 (s, 1H), 7.83 (dd, J = 8.5, 3.2 Hz, 1H), 7.68 (td, J = 8.6, 3.1 Hz, 1H), 7.50 (dd, J = 9.1, 4.5 Hz, 1H), 6.58 (d, J = 5.6 Hz, 1H), 4.69 (s, 2H), 3.77 (t, J = 5.7 Hz, 2H), 2.98 (t, J = 5.8 Hz, 2H). | 441 |
| AO | 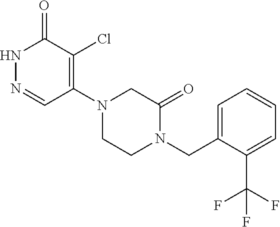 [SMILES: O=C1CN(c2cn[nH]c(=O)c2Cl)CCN1Cc1ccccc1C(F)(F)F] | 1H NMR (400 MHz, DMSO-d6) chemical shifts 12.98 (s, 1H), 7.91 (s, 1H), 7.78 (d, J = 7.8 Hz, 1H), 7.69 (t, J = 7.6 Hz, 1H), 7.52 (t, J = 7.6 Hz, 1H), 7.41 (d, J = 7.8 Hz, 1H), 4.77 (s, 2H), 4.25 (s, 2H), 3.77 (d, J = 5.6 Hz, 2H), 3.43 (d, J = 5.5 Hz, 2H). | 387 |
| JX | 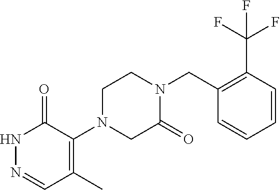 [SMILES: Cc1cn[nH]c(=O)c1N1CCN(Cc2ccccc2C(F)(F)F)C(=O)C1] | 1H NMR (400 MHz, DMSO-d6) chemical shifts 12.82 (s, 1H), 7.76 (d, J = 7.9 Hz, 1H), 7.71-7.63 (m, 2H), 7.51 (d, J = 7.0 Hz, 2H), 4.75 (s, 2H), 3.98 (s, 2H), 3.48 (t, J = 5.3 Hz, 2H), 3.27 (t, J = 5.3 Hz, 2H), 2.14 (s, 3H) | 367 |
| KX | 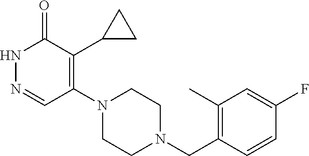 [SMILES: Cc1cc(F)ccc1CN1CCN(c2cn[nH]c(=O)c2C2CC2)CC1] | 1H NMR (400 MHz, DMSO-d6) chemical shifts 12.41 (s, 1H), 7.73 (s, 1H), 7.27 (dd, J = 8.3, 6.2 Hz, 1H), 7.12-6.85 (m, 2H), 3.47 (s, 2H), 3.20 (t, J = 4.6 Hz, 4H), 2.53-2.49 (m, 4H), 2.36 (s, 3H), 1.63 (ddd, J = 11.0, 8.6, 5.5 Hz, 1H), 1.08 (dq, J = 5.9, 3.6 Hz, 2H), 0.83 (dt, J = 8.6, 3.1 Hz, 2H). | 343 |
| FA | 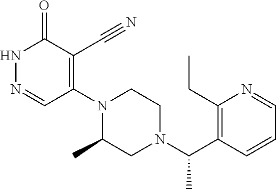 [SMILES: CCc1ncccc1[C@H](C)N1CCN(c2cn[nH]c(=O)c2C#N)[C@H](C)C1] | 1H NMR (400 MHz, DMSO-d6) chemical shifts 12.66 (s, 1H), 8.40 (dd, J = 4.7, 1.6 Hz, 1H), 7.98 (s, 1H), 7.77 (d, J = 7.4 Hz, 1H), 7.23 (dd, J = 7.8, 4.7 Hz, 1H), 4.49 (s, 1H), 4.17 (d, J = 13.8 Hz, 1H), 3.68 (q, J = 6.3 Hz, 1H), 3.47 (dd, J = 14.0, 11.0 Hz, 1H), 3.07 (d, J = 11.2 Hz, 1H), 2.85 (dh, J = 22.1, 7.3 Hz, 2H), 2.48 (s, 0H), 2.29 (dd, J = 11.5, 3.5 Hz, 1H), 2.26-2.15 (m, 1H), 1.29 (dd, J = 10.5, 6.6 Hz, 6H), 1.22 (t, J = 7.5 Hz, 3H). | 353 |
| MS | 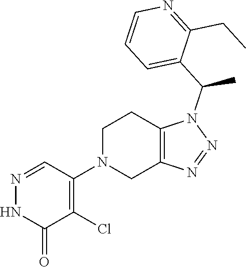 [SMILES: CCc1ncccc1[C@@H](C)n1nnc2c1CCN(c1cn[nH]c(=O)c1Cl)C2] | 1H NMR (400 MHz, DMSO-d6) chemical shifts 12.93 (s, 1H), 8.47 (dd, J = 4.8, 1.7 Hz, 1H), 7.92 (s, 1H), 7.52-7.46 (m, 1H), 7.25 (dd, J = 7.9, 4.7 Hz, 1H), 6.10 (d, J = 6.9 Hz, 1H), 4.58 (s, 2H), 3.65-3.55 (m, 2H), 2.85 (ddq, J = 31.1, 14.8, 7.5 Hz, 3H), 2.45 (d, J = 15.3 Hz, 1H), 1.89 (d, J = 6.9 Hz, 3H), 1.17 (t, J = 7.4 Hz, 3H). | 386 |
| LY | 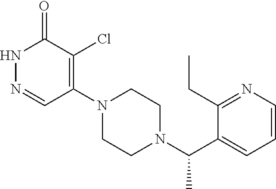 [SMILES: CCc1ncccc1[C@H](C)N1CCN(c2cn[nH]c(=O)c2Cl)CC1] | 1H NMR (400 MHz, DMSO-d6) chemical shifts 12.93 (s, 1H), 8.41 (s, 1H), 7.85 (s, 1H), 7.81 (s, 1H), 7.27 (s, 1H), 3.74 (s, 1H), 2.89 (t, J = 7.5 Hz, 2H), 2.59 (s, 4H), 2.43 (s, 4H), 1.32-1.19 (m, 6H). | 348 |
| LW | 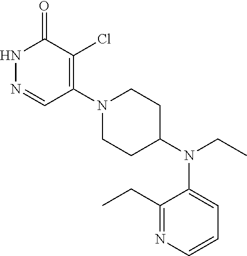 [SMILES: CCc1ncccc1N(CC)C1CCN(c2cn[nH]c(=O)c2Cl)CC1] | 1H NMR (400 MHz, DMSO-d6) chemical shifts 12.86 (s, 1H), 8.27 (d, J = 4.3 Hz, 1H), 7.83 (s, 1H), 7.59 (d, J = 8.0 Hz, 1H), 7.20 (dd, J = 8.0, 4.6 Hz, 1H), 3.76 (d, J = 12.6 Hz, 2H), 3.12-2.92 (m, 5H), 2.86 (q, J = 7.5 Hz, 2H), 1.81 (d, J = 12.5 Hz, 2H), 1.65-1.51 (m, 2H), 1.22 (t, J = 7.5 Hz, 3H), 0.81 (t, J = 7.0 Hz, 3H). | 362 |
| OM | 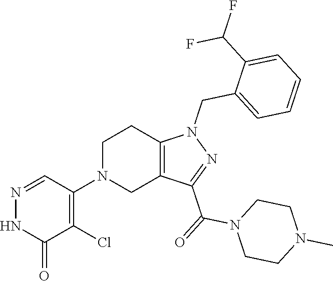 [SMILES: CN1CCN(C(=O)c2nn(Cc3ccccc3C(F)F)c3c2CN(c2cn[nH]c(=O)c2Cl)CC3)CC1] | 1H NMR (400 MHz, DMSO-d6) chemical shifts 12.90 (s, 1H), 7.87 (s, 1H), 7.67 (d, J = 7.4 Hz, 1H), 7.55- 7.45 (m, 2H), 7.40-7.38 (m, 1H), 6.95 (d, J = 7.3 Hz, 1H), 5.53 (s, 2H), 4.55 (s, 2H), 3.98 (s, 2H), 3.69 (t, J = 5.5 Hz, 2H), 3.60 (s, 2H), 2.85 (d, J = 5.5 Hz, 2H), 2.33 (s, 4H), 2.20 (s, 3H). | 518 |
| OU | 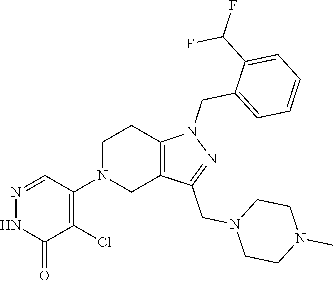 [SMILES: CN1CCN(Cc2nn(Cc3ccccc3C(F)F)c3c2CN(c2cn[nH]c(=O)c2Cl)CC3)CC1] | 1H NMR (400 MHz, DMSO-d6) chemical shifts 12.91 (s, 1H), 7.88 (s, 1H), 7.63 (d, J = 7.2 Hz, 1H), 7.46 (t, J = 7.8 Hz, 2H), 7.43-7.19 (m, 1H), 6.81 (d, J = 7.3 Hz, 1H), 5.42 (s, 2H), 4.45 (s, 2H), 3.68 (t, J = 5.7 Hz, 2H), 3.44 (s, 2H), 2.74 (t, J = 5.5 Hz, 2H), 2.38 (s, 8H), 2.19 (s, 3H). | 504 |
| OP | 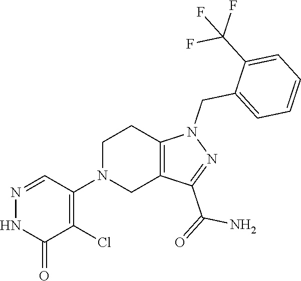 [SMILES: NC(=O)c1nn(Cc2ccccc2C(F)(F)F)c2c1CN(c1cn[nH]c(=O)c1Cl)CC2] | 1H NMR (400 MHz, DMSO-d6) chemical shifts 12.91 (s, 1H), 7.88 (s, 1H), 7.81 (d, J = 7.8 Hz, 1H), 7.64 (t, J = 7.6 Hz, 1H), 7.54 (t, J = 7.7 Hz, 1H), 7.45 (s, 1H), 7.29 (s, 1H), 6.72 (d, J = 7.8 Hz, 1H), 5.54 (s, 2H), 4.64 (s, 2H), 3.67 (t, J = 5.6 Hz, 2H), 2.79 (t, J = 5.6 Hz, 2H). | 453 |
| QM | 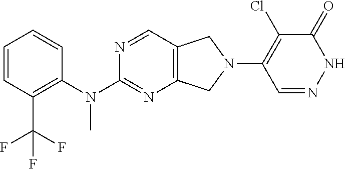 [SMILES: CN(c1ncc2c(n1)CN(c1cn[nH]c(=O)c1Cl)C2)c1ccccc1C(F)(F)F] | 1H NMR (400 MHz, DMSO-d6) chemical shifts 12.71 (s, 1H), 8.40- 8.33 (m, 1H), 7.87-7.76 (m, 3H), 7.59 (t, J = 7.7 Hz, 1H), 7.50 (d, J = 7.9 Hz, 1H), 5.10-4.81 (m, 4H), 3.38 (s, 3H). | 423 |
| MD (single enantiomer; absolute stereochemistry at benzylic methine not yet assigned) | 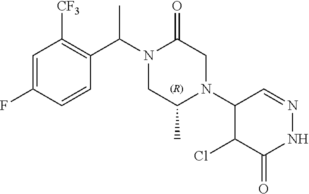 | 1H NMR (400 MHz, DMSO-d6) chemical shifts 12.98 (s, 1H), 7.85 (d, J = 9.7 Hz, 2H), 7.70-7.58 (m, 2H), 5.93 (d, J = 6.9 Hz, 1H), 4.35 (s, 1H), 4.15 (d, J = 17.0 Hz, 1H), 3.94 (d, J = 17.0 Hz, 1H), 3.66 (dd, J = 12.5, 4.3 Hz, 1H), 2.88 (dd, J = 12.5, 3.6 Hz, 1H), 1.51 (d, J = 7.0 Hz, 3H), 1.01 (d, J = 6.5 Hz, 3H). | 433 |
| MF (single enantiomer; absolute stereochemistry not yet assigned) | 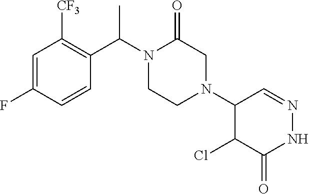 [SMILES: CC(c1ccc(F)cc1C(F)(F)F)N1CCN(C2C=NNC(=O)C2Cl)CC1=O] | 1H NMR (400 MHz, DMSO-d6) chemical shifts 12.93 (s, 1H), 7.86 (dd, J = 8.7, 5.4 Hz, 1H), 7.82 (s, 1H), 7.64 (ddd, J = 19.1, 8.9, 4.2 Hz, 2H), 5.93 (q, J = 6.9 Hz, 1H), 4.10 (s, 2H), 3.70-3.54 (m, 2H), 3.40 (dt, J = 10.8, 4.6 Hz, 1H), 2.89 (ddd, J = 11.9, 7.4, 4.1 Hz, 1H), 1.52 (d, J = 6.9 Hz, 3H). | 419 |
| PW | 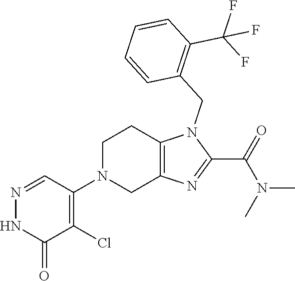 [SMILES: CN(C)C(=O)c1nc2c(n1Cc1ccccc1C(F)(F)F)CCN(c1cn[nH]c(=O)c1Cl)C2] | 1H NMR (400 MHz, DMSO-d6) chemical shifts 12.91 (s, 1H), 7.94 (s, 1H), 7.79 (d, J = 7.8 Hz, 1H), 7.62 (t, J = 7.6 Hz, 1H), 7.51 (t, J = 7.6 Hz, 1H), 6.63 (d, J = 7.9 Hz, 1H), 5.61 (s, 2H), 4.49 (s, 2H), 3.67 (t, J = 5.2 Hz, 2H), 3.39 (s, 3H), 2.91 (s, 3H), 2.59 (s, 2H). | 481 |
| PR | 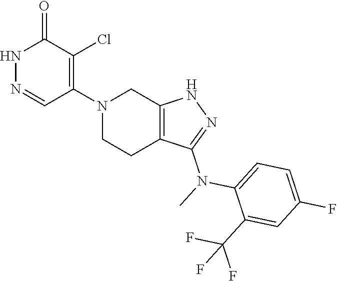 [SMILES: CN(c1ccc(F)cc1C(F)(F)F)c1n[nH]c2c1CCN(c1cn[nH]c(=O)c1Cl)C2] | 1H NMR (400 MHz, DMSO-d6) chemical shifts 12.88 (s, 1H), 11.75 (s, 1H), 7.80 (s, 1H), 7.69 (dd, J = 8.9, 3.0 Hz, 1H), 7.56 (s, 1H), 7.38 (s, 1H), 4.47 (s, 2H), 3.07 (s, 3H), 1.71 (s, 2H). | 443 |

### Example 46 Effect of Compound 100 on Puromycin Aminonucleoside (PAN)-Induced Glomerular Injury in Rats

### Objective:

The objective of this study is to evaluate the dose-dependent effects of compound 100 on PAN induced glomerular kidney injury as indexed by albuminuria.

### Methods:

Eighty (80), male Sprague-Dawley rats weighing approximately 125-150 g and approximately 5-6 weeks of age were acquired from Charles River. They were fed a standard chow diet (Harlan 8640), housed under standard conditions, and allowed to acclimate for at least 5 days prior to study inception.

On D-2, rats were placed into weight-matched treatment groups and were placed, individually housed, into metabolic cages for the balance of the study.

A 24 hour baseline (Day 0) urine was collected followed by a baseline blood collection via conscious tail venous puncture. Rats were then administered vehicle or test article.

Two (2) hours following administration of vehicle or test agent on Day 0, rats received an administration of (5 mL/kg, s.c.) vehicle (sterile saline) or puromycin aminonucleoside (PAN; challenge agent; 75 mg/kg) dissolved in vehicle.

Intermittent (Day 4, 7 and 10) 24 hour urine volumes were determined and samples (4 samples/animal/time point; 0.5 mL/sample) were obtained. Additionally, intermittent (Day 4, 7 and 10) blood samples were collected via conscious tail venous puncture 2 hours±1 minute post-AM dose.

Immediately following the last blood collection, rats were anesthetized with isoflurane, tissues harvested, and animals sacrificed. Endpoint kidney weights and indices were obtained.

Urine samples were immediately flash-frozen in liquid N₂ and stored at −80° C. until analyzed.

Whole blood samples collected on K₃EDTA were processed appropriately for the production of plasma for PK measurements.

### Results:

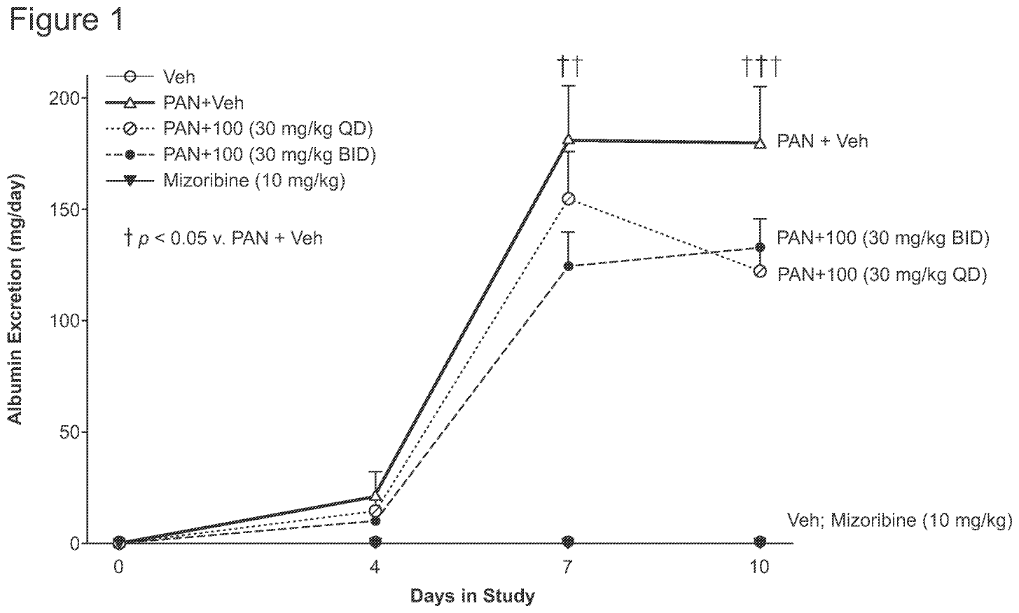

As shown in FIG. 1, treatment with compound 100 at 30 mg/kg once—(QD) or twice—(BID) daily resulted in reduced urinary albumin excretion on following injury with PAN. Significant reductions were seen at 7 and 10 days with BID dosing, and at 10 days with QD dosing of compound 100. Mizoribine, the positive control compound, was also efficacious in reducing albuminuria.

### Conclusion:

Compound 100 is effective in reducing albuminuria in the PAN model of glomerular injury in rats.

### Example 47. Compound 100 is Efficacious in the AT1R Transgenic Rat Model of FSGS

The AT1R transgenic rat model of FSGS is characterized by podocyte-specific expression of human AT1R. Males have been shown to have substantially worse pathology compared to females. The efficacy of TRPC5 inhibitors in the AT1R model has been demonstrated with a tool compound. See Zhou et al., Science (2017), vol. 358 (6368), 1332-1336.

In the present study, pathophysiology in AT1R transgenic rats was accelerated with unilateral nephrectomy (UniNX) and minipump AngII infusion. Compound 100 was dosed orally once daily at 3 mg/kg or 10 mg/kg, and the urine protein creatinine ratio was determined at −1, 0, 1, 2, and 3 weeks of treatment.

The results show that compound 100 is efficacious in the AT1R transgenic rat model of FSGS.

### Example 48. Kidney Organoid Differentiation

### Reagents & Materials

| Reagent | Vendor | Cat# | Lot# | Storage/Expiration |
|---|---|---|---|---|
| hiPSCs | ThermoFisher | A18945 | 1938075 | LN2, Oct. 31, 2027 |
| T25 flask | VWR | BD354277 | | |
| 6-well transwells | Sigma | CLS3450 | | |
| Serological | VWR | 89130-896, | | |
| pipettes | | 89130-898, | | |
| | | 89130-900, | | |
| | | 89130-902 | | |
| Aspirator | VWR | 93000-694 | | |
| P20 tips | VWR | | | |
| P200 tips | VWR | 53510-106 | | |
| Wide bore tips | Phenix | T-061BRS | | |
| 15 mL tube | Falcon | 352097 | | |
| 50 mL tube | Falcon | 352070 | | |
| 1.5 mL tubes | Eppendorf | 022431021 | | |
| Forceps | VWR | 82027-384 | | |
| 6-well transwell | Sigma | Z363359-1EA | | |
| plate holder | | | | |
| Cell counter slide | Nexcelom | CHT4-PD100- | | |
| | | 003 | | |
| Nexcelom Auto | Nexcelom | | | |
| T4 Cell Counter | | | | |
| 37° C. incubator, | | | | |
| 5.0% CO2 | | | | |
| 4C refrigerator | | | | |
| Autoclave | | | | |
| Centrifuge | Eppendorf | 5424R (24 | | |
| | | well rotor FA- | | |
| | | 45-24-11) | | |
| Matrigel Matrix | Corning | 354277 | 7282005, | −20 C., |
| (hESC qualified) | | | 7268012, | Dec. 2, 2019 |
| | | | 8015323 | Nov. 19, 2019 |
| | | | | Mar. 10, 2020 |
| X Dulbecco's PBS | ThermoFisher | 14190-144 | | |
| (DPBS) without | | | | |
| Calcium and | | | | |
| Magnesium | | | | |
| mTeSR | StemCell | 85851 | AC11627264 | 4 C., May 31, 2019 |
| | Technologies | | | |
| mTeSR Plus 5X | StemCell | 05827 | SLBV7960 | −20 C., Jun. 30, 2019 |
| supplement | Technologies | | | |
| STEMDiff APEL2 | StemCell | 05275 | 17L85156, | −20 C., May 2019 |
| | Technologies | | 18C88822 | September 2019 |
| Accutase | StemCell | 07920 | 7D1845A | −20 C., December 2019 |
| | Technologies | | | |
| FGF9 (100 ug/mL) | R&D Systems | 273-F9/CF | ON2317071 | −20 C., Mar. 30, 2019 |
| Heparin Solution | StemCell | 07980 | 18AB6733 | 4 C., Jan. 31, 2020 |
| | Technologies | | | |
| Rock inhibitor | Tocris | 1254 | 31A/208776 | RT, August 2018 |
| CHIR | Tocris | 4423 | 8A/211242 | −20, October 2018 |
| Trypan blue | Gibco | 15250-061 | | |

### Day −1: Initial Plating of iPS Single Cells

Reagents:
1. Accutase (warm at 37° C.)
2. mTeSR-1 (RT)
3. Rock inhibitor (thaw at RT)
4. 1×PBS (RT)
5. T25 flasks coated with hES Matrigel (warm at 37° C.)

Procedure:

Fresh T25 flasks were prepared for differentiation. hES Matrigel was aspirated, to which 5 mL of mTeSR-1 containing Rock inhibitor (1:1000; Rock inhibitor to mTeSR) was added. The flasks were kept at room temperature, and mTeSR-1 medium was aspirated from a starter T25 flask containing iPSC colonies cultured on hES Matrigel. The iPSCs were washed with 5 L of of 1×PBS and aspirated. Next was added 2 mL of warm Accutase before incubating the flask at 37° C. for 3-5 min. (If cells are still attached to the flask, incubate at 37° C. for an additional minute; 8 min. max for incubation in Accutase. If Accutase is slightly cold upon addition to iPSCs, incubate flask at 37° C. for 6 min. initially.) The Accutase was neutralized with 8 mL of mTeSR-1 medium, gently pipetting up and down a few times to help break up cell aggregates. Cells were transferred to a new falcon tube and centrifuged at 400×g for 3 min. The mTeSR-1 medium was aspirated, and cells were resuspended in mTeSR-1 medium containing Rock inhibitor (1:1000; Rock inhibitor to mTeSR). Cells were counted at least two times with Trypan Blue (1:1); viability should be at least 85-90%. Single cells were plated at an initial seeding density that has been optimized for each iPS line. The flask was placed an 37° C. incubator and moved in a FIG. 8 motion, back and forth, then side to side; repeat movements in the opposite direction. The flask was not disturbed for the first ˜20-24 hrs after plating.

### Day 0: Begin Differentiation (Addition of OL1 Medium)

mTeSR-1 medium containing Rock inhibitor was aspirated, and 8 mL of OL1-A (10 uM CHIR) was added to each T25 flask.

### Day 2: Differentiation (Addition of OL1 Medium)

OL1 medium was aspirated, and 8 mL of OL1-B (8 uM CHIR) was added to each T25 flask.

### Day 4: Differentiation (Addition of OL2 Medium)

OL1 medium was aspirated, and 8 mL of OL2 was added to each T25 flask.

### Day 6: Differentiation (Addition of OL2 Medium)

OL2 medium was aspirated, and 8 mL of OL2 was added to each T25 flask.

### Day 7: Generation of 3D Organoids (Passaging to Transwells)

Reagents:

1. 1×PBS

2. Accutase (warm at 37° C.)

3. Apel2 medium

Procedure:

Cells were washed with 5 mL of 1×PBS and then with aspirated OL2 medium. 2 mL of warm Accutase were added, and the flask was incubated at 37° C. for 5 min. (If cells are still attached to the flask, the flask can be incubated for an additional minute, up to a maximum of 8 minutes for incubation in Accutase. Cells can be washed off the flask later. If the Accutase is slightly cold upon addition to the iPSCs, the flask can be incubated at 37° C. initially.) Accutase was neutralized with 8 mL of Apel2 medium, breaking up cell aggregates by gently pipetting up and down a few times. Cells were transferred to a new falcon tube and centrifuged at 400×g for 3 min. (Cells can be washed off the flask here if not completely detached in the earlier Accutase addition.). Fresh Apel 2 medium was aspirated, and cells were resuspended in an appropriate amount of Apel2 medium using a p1000 pipet (e.g., for ˜10 million cells, 4 mL of medium can be used). Cells were counted at least twice with Trypan Blue (1:1); viability should be at least 85-90%. Poritons of about 500 k cells (100-200 μL) were transferred into 1.5 mL Eppendorf tubes, with occasional mixing of the stock cell suspension while making 500 k Eppendorf tubes. The tubes were centrifuged at 350×g for 2 min using an Eppendorf 5424R centrifuge with a 24-well rotor only. The tubes were rotated 180° and centrifuged again at 350×g for 2 min, and the resulting pellets were allowed to settle for about 30 sec. (The tubes can be centrifuged up to 4 times, but the resulting pellet should not be too loosely or tightly compact after spinning; additional spins should only be performed if the pellet falls apart easily during plating to transwells.) Using a wide bore pipet tip, the cell pellet was transferred in a small amount of medium and carefully placed on a 6-well transwell plate, with 4-6 cell pellets per transwell. The Transwells should be dry, and the organoids can be left on a dry transwell for about 10 mins.

1 hour CHIR pulse: After all organoids have been plated, 1.2 mL of Apel2 medium, containing 5 uM of CHIR (OL-trans), was added, and transwell plates were incubated at 37 C for 1 hour. Medium was aspirated, and OL2 medium was added of the bottom of the transwell: 1.2 mL for 2 organoids per well, or 1.5 L for 4 organoids per well.

### Day 9: Differentiation

OL2 medium was aspirated and added to the bottom of transwells: 1.2 mL for 2 organoids per well, and 1.5 mL for 4 organoids per well.

By Day 10, nephrogenesis should be observed in developing organoids.

### Day 11: Differentiation

OL2 medium was aspirated and added to the bottom of the transwells: 1.2 mL for 2 organoids per well, and 1.5 mL for 4 organoids per well.

### Day 13: Differentiation

Medium was aspirated, and OL3 medium was added to the bottom of the transwells, to a final concentration of 1 mg/mL Heparin: 1.2 mL for 2 organoids per well, and 1.5 mL for 4 organoids per well.

### Days 14-25: Differentiation

Medium was changed with OL4 medium every 2-3 days.

Media recipes:
OL1-A (Apel 2+10 μM CHIR)
   15 mL Apel2
   7.5 μL CHIR (20 mM stock)
OL1-B (Apel 2+8 μM CHIR)
   15 mL Apel2
   6 μL CHIR (20 mM stock)
OL2 (Apel2+200 ng/mL FGF9+1 mg/mL Heparin)
   10 mL Apel2
   20 μL FGF9 (100 μg/mL stock)
   5 μL Heparin (2 mg/mL stock)
OL-trans (Apel2+5 μM CHIR)
   8 mL Apel2
   2 μL CHIR (20 mM stock)
OL3 (APEL2+1 mg/mL Heparin)
   10 mL APEL2
   5 μL Heparin (2 mg/mL stock)
OL4
   APEL2 media

### Example 49. Transplanting Kidney Organoids Under the Rat Kidney Capsule

### Reagents & Materials

| | | | | Storage/ |
|---|---|---|---|---|
| Reagent | Vendor | Cat# | Lot# | Expiration |
| 6-well transwells | Sigma | CL53450 | | |
| containing kidney | | | | |
| organoids (derived | | | | |
| from ehiPSC, | | | | |
| ThermoFisher, | | | | |
| cat# A18945) | | | | |
| Fine forceps | Fisherbrand | 16-100-122 | | |
| STEMDiff APEL2 | StemCell | 05275 | 17L85156, | −20 C., |
| | Technologies | | 18C88822 | May 2019 |
| | | | | September |
| | | | | 2019 |
| Tissue culture | Falcon | 353001 | | |
| dish, | | | | |
| 35 × 10 mm | | | | |
| P1000 pipetman | RAININ | | | |
| P1000 tips | VWR | 53225-782 | | |
| Scissors | | | | |
| Parafilm | | | | |
| Small Styrofoam | | | | |
| box | | | | |
| Medium | | | | |
| Styrofoam box | | | | |
| Aluminum blocks, | | | | |
| warmed at 37 C. | | | | |
| overnight | | | | |
| Ice packs, frozen | | | | |
| at −20 C. | | | | |
| Ice packs, warmed | | | | |
| at 37 C. | | | | |
| 37° C. incubator, | | | | |
| 5.0% CO2 | | | | |
| 4C refrigerator | | | | |
| Plastic feeding | Instech | FTP-18-30- | | |
| tubes, 18G | Laboratories | 50 | | |
| Syringe, 1 mL | BD | BD309628 | | |

Surgical procedures were performed by Biomere (Worcester, Mass., USA).

### Preparing Organoids for Transport

Organoids were prepared for transport by first taking representative images of organoids on a transwell (TW) plate. The TW plates were parafilmed and stored in a biosafety cabinet before packing into a Styrofoam box sprayed with ethanol and containing warmed aluminum blocks and ice packs.

### Preparing Organoids for Transplant

In a biosafety cabinet (Biomere, first floor), a P1000 tip was cut with scissors so that the resulting circumference of the wide bore tip was approximately the size of the organoid. About 100-200 uL of APEL2 medium was aspirated with this wide bore P1000 tip. The tip was then placed around one organoid, and the organoid was gently scraped off of the transwell while simultaneously releasing some of the APEL2 medium. (If needed, the edges of the organoid can be worked loose from the transwell plate by gentle scraping with the outside of the pipet tip.) The organoid and the APEL2 medium were aspirated from the well of the TW plate, and the organoid was placed in a 35×10 mm tissue culture dish containing 2 mL of APEL2, using 2 organoids (plus 1 spare) per rat. The dish was parafilmed, and the organoids were transferred to the surgical room.

### Bilateral Transplants of Kidney Organoids Under the Rat Kidney Capsule ˜25-30 Min/Rat (1 Organoid/Kidney, 2 Kidney Transplants/Rat)

After completion of the second kidney capsule transplant, closing of the animal was begun, and the procedure of removing the organoids from the TW plate for the next animal was begun; this timing typically enables the arrival of the organoids from the hood to the surgical room by the time the first kidney is exposed for transplant in the next animal. Organoids can be removed from TW as needed for each animal, to help ensure that the organoids are not detached from the TW for longer than necessary.

Once the first rat kidney was exposed, a small incision was made on the kidney capsule with a 25-26G needle, ensuring that the incision did not penetrate into the kidney cortex. Initially a space was created under the kidney capsule with angled fine forceps. A 22G blunt needle was used to further open the space under the kidney capsule. An angled cut was made at the tip of the 18G feeding tube attached to a 1 mL syringe. The 18G feeding tube was pre-wet with APEL2 medium, and one organoid from the dish containing APEL2 with the 18G feeding tube was carefully aspirated, keeping the entire organoid close to the tip of the 18G feeding tube, and not aspirating organoid into the syringe. An 18G feeding tube containing the organoid was inserted into the space created under the kidney capsule, and the organoid was injected towards the back end of the created space. The animal was closed, and then the procedure was repeated on the second kidney. After the second transplant, the animal was closed again and allowed to recover from anesthesia. The next organoid was started to be removed from TW (2+1 spare organoids) upon closing of animal after second kidney capsule transplant.

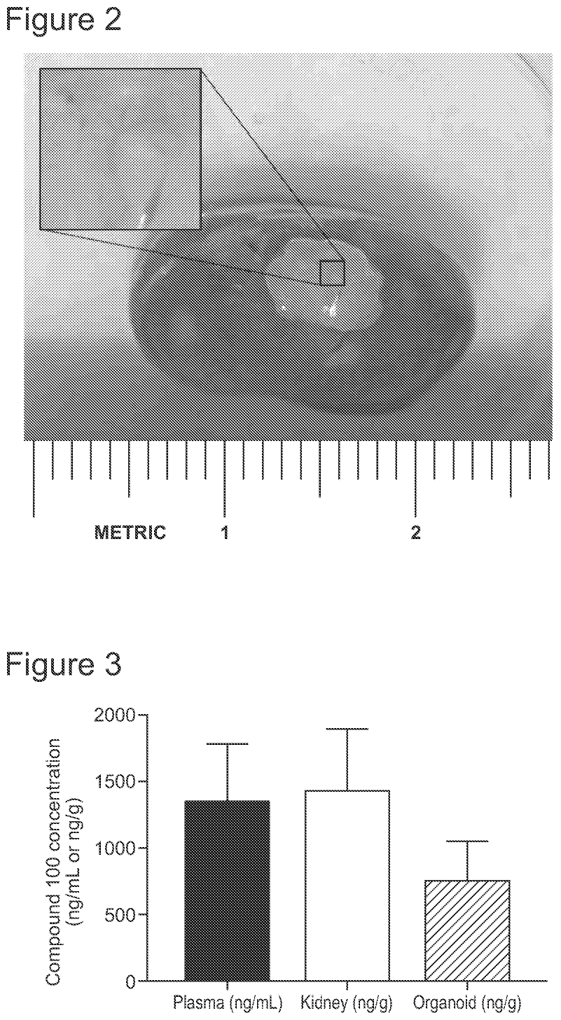

FIG. 2 shows an organoid that was differentiated for 14 days, then transplanted into a rat, and then removed for analysis after 4 weeks.

Compound 100 was administered to animals after transplant orally, once-daily as follows:

| | | Dose |
|---|---|---|
| Treatment | Dose Level | Volume |
| Regimen (PO) | (mg/kg) | (mL/kg) |
| Cmpd 100 dosing | 10 mg/kg of Cmpd 100 | 5 mL/kg |
| QD for 3 | formulated in Solutol HS- | |
| consecutive days | 15/Vit E-TPGS/PEG 300 | |
| (final dose | (40/20/40; w/w/w) | |
| administered the AM | | |
| of necropsy) | | |

After 3 days, necropsy was performed to study distribution of compound 100 in the animal. As shown in FIG. 3, oral dosing of compound 100 results in drug exposure in the implanted organoid.

### INCORPORATION BY REFERENCE

All of the U.S. patents and U.S. and PCT published patent applications cited herein are hereby incorporated by reference.

### EQUIVALENTS

The foregoing written specification is sufficient to enable one skilled in the art to practice the invention. The present invention is not to be limited in scope by examples provided, since the examples are intended as a single illustration of one aspect of the invention and other functionally equivalent embodiments are within the scope of the invention. Various modifications of the invention in addition to those shown and described herein will become apparent to those skilled in the art from the foregoing description and fall within the scope of the appended claims. The advantages and objects of the invention are not necessarily encompassed by each embodiment of the invention.

TRPC4 Plasmid Sequence
The DNA sequence of the TRPC4 plasmid used
in Example 41 is included below.
Underlined nucleic acids represent those
encoding human TRPC4.
SEQ ID NO: 1
GACGGATCGGGAGATCTCCCGATCCCCTATGGTGCACTCTCAGTACAATC
TGCTCTGATGCCGCATAGTTAAGCCAGTATCTGCTCCCTGCTTGTGTGTT
GGAGGTCGCTGAGTAGTGCGCGAGCAAAATTTAAGCTACAACAAGGCAAG
GCTTGACCGACAATTGCATGAAGAATCTGCTTAGGGTTAGGCGTTTTGCG
CTGCTTCGCGATGTACGGGCCAGATATACGCGTTGACATTGATTATTGAC
TAGTTATTAATAGTAATCAATTACGGGGTCATTAGTTCATAGCCCATATA
TGGAGTTCCGCGTTACATAACTTACGGTAAATGGCCCGCCTGGCTGACCG
CCCAACGACCCCCGCCCATTGACGTCAATAATGACGTATGTTCCCATAGT
AACGCCAATAGGGACTTTCCATTGACGTCAATGGGTGGAGTATTTACGGT
AAACTGCCCACTTGGCAGTACATCAAGTGTATCATATGCCAAGTACGCCC
CCTATTGACGTCAATGACGGTAAATGGCCCGCCTGGCATTATGCCCAGTA
CATGACCTTATGGGACTTTCCTACTTGGCAGTACATCTACGTATTAGTCA
TCGCTATTACCATGGTGATGCGGTTTTGGCAGTACATCAATGGGCGTGGA
TAGCGGTTTGACTCACGGGGATTTCCAAGTCTCCACCCCATTGACGTCAA
TGGGAGTTTGTTTTGGCACCAAAATCAACGGGACTTTCCAAAATGTCGTA
ACAACTCCGCCCCATTGACGCAAATGGGCGGTAGGCGTGTACGGTGGGAG
GTCTATATAAGCAGAGCTCTCCCTATCAGTGATAGAGATCTCCCTATCAG
TGATAGAGATCGTCGACGAGCTCGTTTAGTGAACCGTCAGATCGCCTGGA
GACGCCATCCACGCTGTTTTGACCTCCATAGAAGACACCGGGACCGATCC
AGCCTCCGGACTCTAGCGTTTAAACTTAAGCTTGGTACCGAGCTCGGATC
CGCCACCATGGCCCAGTTCTACTATAAGAGAAACGTGAATGCCCCTTACC
GCGACAGAATCCCCCTGAGAATCGTGAGGGCAGAGTCCGAGCTGAGCCCA
TCCGAGAAGGCCTACCTGAACGCCGTGGAGAAGGGCGACTATGCCAGCGT
GAAGAAGTCCCTGGAGGAGGCCGAGATCTACTTTAAGATCAACATCAATT
GCATCGATCCTCTGGGCAGAACCGCCCTGCTGATCGCCATCGAGAACGAG
AATCTGGAGCTGATCGAGCTGCTGCTGAGCTTCAACGTGTATGTGGGCGA
TGCCCTGCTGCACGCCATCAGGAAGGAGGTGGTGGGAGCAGTGGAGCTGC
TGCTGAATCACAAGAAGCCAAGCGGAGAGAAGCAGGTGCCACCTATCCTG
CTGGACAAGCAGTTCTCCGAGTTTACCCCAGATATCACACCCATCATCCT
GGCCGCCCACACCAACAATTACGAGATCATCAAGCTGCTGGTGCAGAAGG
GCGTGTCCGTGCCTCGCCCACACGAGGTGCGGTGCAACTGCGTGGAGTGC
GTGAGCTCCTCTGACGTGGATTCTCTGAGGCACAGCCGGAGCCGGCTGAA
CATCTATAAGGCCCTGGCCTCCCCATCTCTGATCGCCCTGAGCTCCGAGG
ACCCCTTCCTGACCGCCTTTCAGCTGTCTTGGGAGCTGCAGGAGCTGAGC
AAGGTGGAGAACGAGTTTAAGAGCGAGTACGAGGAGCTGTCCAGACAGTG
CAAGCAGTTCGCCAAGGACCTGCTGGATCAGACACGCTCTAGCCGGGAGC
TGGAGATCATCCTGAACTATAGGGACGATAATTCTCTGATCGAGGAGCAG
AGCGGAAACGACCTGGCACGCCTGAAGCTGGCCATCAAGTACCGGCAGAA
GGAGTTCGTGGCCCAGCCTAATTGTCAGCAGCTGCTGGCCTCCCGCTGGT
ATGATGAGTTTCCAGGATGGCGGAGAAGGCACTGGGCAGTGAAGATGGTG
ACCTGCTTCATCATCGGCCTGCTGTTCCCCGTGTTCAGCGTGTGCTACCT
GATCGCCCCTAAGTCTCCACTGGGCCTGTTTATCCGGAAGCCTTTCATCA
AGTTTATCTGCCACACCGCCAGCTATCTGACATTCCTGTTTCTGCTGCTG
CTGGCCTCCCAGCACATCGACAGATCTGATCTGAACAGGCAGGGCCCACC
CCCTACCATCGTGGAGTGGATGATCCTGCCATGGGTGCTGGGCTTCATCT
GGGGCGAGATCAAGCAGATGTGGGACGGCGGCCTGCAGGACTACATCCAC
GATTGGTGGAACCTGATGGATTTTGTGATGAATTCCCTGTACCTGGCCAC
AATCTCTCTGAAGATCGTGGCCTTCGTGAAGTATAGCGCCCTGAATCCCA
GAGAGTCCTGGGACATGTGGCACCCTACCCTGGTGGCAGAGGCCCTGTTC
GCAATCGCCAACATCTTTTCCTCTCTGCGCCTGATCAGCCTGTTTACAGC
CAATTCCCACCTGGGACCACTGCAGATCTCCCTGGGACGGATGCTGCTGG
ATATCCTGAAGTTCCTGTTTATCTACTGCCTGGTGCTGCTGGCCTTCGCC
AACGGCCTGAATCAGCTGTACTTCTACTATGAGGAGACCAAGGGCCTGAC
ATGCAAGGGCATCCGCTGTGAGAAGCAGAACAATGCCTTCAGCACCCTGT
TCGAGACACTGCAGTCTCTGTTCTGGAGCATCTTTGGCCTGATCAACCTG
TACGTGACCAATGTGAAGGCCCAGCACGAGTTCACAGAGTTTGTGGGCGC
CACCATGTTCGGCACATACAACGTGATCTCTCTGGTGGTGCTGCTGAATA
TGCTGATCGCCATGATGAACAATAGCTATCAGCTGATCGCCGACCACGCC
GATATCGAGTGGAAGTTCGCCCGGACCAAGCTGTGGATGTCCTACTTTGA
GGAGGGCGGCACCCTGCCCACACCTTTCAACGTGATCCCATCCCCCAAGT
CTCTGTGGTATCTGATCAAGTGGATCTGGACACACCTGTGCAAGAAGAAG
ATGCGCCGGAAGCCTGAGAGCTTTGGCACCATCGGCGTGCGCACACAGCA
CAGAAGGGCAGCAGACAACCTGCGCCGGCACCACCAGTACCAGGAAGTGA
TGCGCAATCTGGTGAAGCGGTATGTGGCCGCCATGATCAGGGACGCAAAG
ACCGAGGAGGGACTGACAGAGGAGAACTTCAAGGAGCTGAAGCAGGATAT
CAGCTCCTTCAGATTTGAGGTGCTGGGCCTGCTGAGGGGCAGCAAGCTGT
CCACCATCCAGTCCGCCAACGCCTCTAAGGAGTCTAGCAATTCTGCCGAC
AGCGATGAGAAGAGCGACTCCGAGGGCAACTCTAAGGATAAGAAGAAGAA
CTTCAGCCTGTTTGACCTGACCACACTGATCCACCCACGCAGCGCCGCAA
TCGCATCCGAGCGGCACAACATCTCCAATGGCTCTGCCCTGGTGGTGCAG
GAGCCACCAAGAGAGAAGCAGAGGAAGGTGAACTTTGTGACAGATATCAA
GAATTTCGGCCTGTTTCACAGAAGGAGCAAGCAGAACGCCGCCGAGCAGA
ACGCCAATCAGATCTTCTCTGTGAGCGAGGAGGTGGCAAGACAGCAGGCA
GCAGGACCACTGGAGAGGAATATCCAGCTGGAGAGCCGGGGACTGGCAAG
CAGGGGCGACCTGTCCATCCCAGGACTGTCTGAGCAGTGCGTGCTGGTGG
ACCACAGGGAGCGGAACACCGATACACTGGGACTGCAAGTGGGCAAGCGG
GTGTGCCCTTTCAAGAGCGAGAAGGTCGTGGTGGAGGACACCGTGCCCAT
CATCCCTAAGGAGAAGCACGCCAAGGAGGAGGATTCCTCTATCGACTACG
ATCTGAATCTGCCAGACACCGTGACACACGAGGATTATGTGACCACAAGG
CTGTGAGCGGCCGCTCTAGAGGGCCCGTTTAAACCCGCTGATCAGCCTCG
ACTGTGCCTTCTAGTTGCCAGCCATCTGTTGTTTGCCCCTCCCCCGTGCC
TTCCTTGACCCTGGAAGGTGCCACTCCCACTGTCCTTTCCTAATAAAATG
AGGAAATTGCATCGCATTGTCTGAGTAGGTGTCATTCTATTCTGGGGGGT
GGGGTGGGGCAGGACAGCAAGGGGGAGGATTGGGAAGACAATAGCAGGCA
TGCTGGGGATGCGGTGGGCTCTATGGCTTCTGAGGCGGAAAGAACCAGCT
GGGGCTCTAGGGGGTATCCCCACGCGCCCTGTAGCGGCGCATTAAGCGCG
GCGGGTGTGGTGGTTACGCGCAGCGTGACCGCTACACTTGCCAGCGCCCT
AGCGCCCGCTCCTTTCGCTTTCTTCCCTTCCTTTCTCGCCACGTTCGCCG
GCTTTCCCCGTCAAGCTCTAAATCGGGGGCTCCCTTTAGGGTTCCGATTT
AGTGCTTTACGGCACCTCGACCCCAAAAAACTTGATTAGGGTGATGGTTC
ACGTAGTGGGCCATCGCCCTGATAGACGGTTTTTCGCCCTTTGACGTTGG
AGTCCACGTTCTTTAATAGTGGACTCTTGTTCCAAACTGGAACAACACTC
AACCCTATCTCGGTCTATTCTTTTGATTTATAAGGGATTTTGCCGATTTC
GGCCTATTGGTTAAAAAATGAGCTGATTTAACAAAAATTTAACGCGAATT
AATTCTGTGGAATGTGTGTCAGTTAGGGTGTGGAAAGTCCCCAGGCTCCC
CAGCAGGCAGAAGTATGCAAAGCATGCATCTCAATTAGTCAGCAACCAGG
TGTGGAAAGTCCCCAGGCTCCCCAGCAGGCAGAAGTATGCAAAGCATGCA
TCTCAATTAGTCAGCAACCATAGTCCCGCCCCTAACTCCGCCCATCCCGC
CCCTAACTCCGCCCAGTTCCGCCCATTCTCCGCCCCATGGCTGACTAATT
TTTTTTATTTATGCAGAGGCCGAGGCCGCCTCTGCCTCTGAGCTATTCCA
GAAGTAGTGAGGAGGCTTTTTTGGAGGCCTAGGCTTTTGCAAAAAGCTCC
CGGGAGCTTGTATATCCATTTTCGGATCTGATCAGCACGTGATGAAAAAG
CCTGAACTCACCGCGACGTCTGTCGAGAAGTTTCTGATCGAAAAGTTCGA
CAGCGTCTCCGACCTGATGCAGCTCTCGGAGGGCGAAGAATCTCGTGCTT
TCAGCTTCGATGTAGGAGGGCGTGGATATGTCCTGCGGGTAAATAGCTGC
GCCGATGGTTTCTACAAAGATCGTTATGTTTATCGGCACTTTGCATCGGC
CGCGCTCCCGATTCCGGAAGTGCTTGACATTGGGGAATTCAGCGAGAGCC
TGACCTATTGCATCTCCCGCCGTGCACAGGGTGTCACGTTGCAAGACCTG
CCTGAAACCGAACTGCCCGCTGTTCTGCAGCCGGTCGCGGAGGCCATGGA
TGCGATCGCTGCGGCCGATCTTAGCCAGACGAGCGGGTTCGGCCCATTCG
GACCGCAAGGAATCGGTCAATACACTACATGGCGTGATTTCATATGCGCG
ATTGCTGATCCCCATGTGTATCACTGGCAAACTGTGATGGACGACACCGT
CAGTGCGTCCGTCGCGCAGGCTCTCGATGAGCTGATGCTTTGGGCCGAGG
ACTGCCCCGAAGTCCGGCACCTCGTGCACGCGGATTTCGGCTCCAACAAT
GTCCTGACGGACAATGGCCGCATAACAGCGGTCATTGACTGGAGCGAGGC
GATGTTCGGGGATTCCCAATACGAGGTCGCCAACATCTTCTTCTGGAGGC
CGTGGTTGGCTTGTATGGAGCAGCAGACGCGCTACTTCGAGCGGAGGCAT
CCGGAGCTTGCAGGATCGCCGCGGCTCCGGGCGTATATGCTCCGCATTGG
TCTTGACCAACTCTATCAGAGCTTGGTTGACGGCAATTTCGATGATGCAG
CTTGGGCGCAGGGTCGATGCGACGCAATCGTCCGATCCGGAGCCGGGACT
GTCGGGCGTACACAAATCGCCCGCAGAAGCGCGGCCGTCTGGACCGATGG
CTGTGTAGAAGTACTCGCCGATAGTGGAAACCGACGCCCCAGCACTCGTC
CGAGGGCAAAGGAATAGCACGTGCTACGAGATTTCGATTCCACCGCCGCC
TTCTATGAAAGGTTGGGCTTCGGAATCGTTTTCCGGGACGCCGGCTGGAT
GATCCTCCAGCGCGGGGATCTCATGCTGGAGTTCTTCGCCCACCCCAACT
TGTTTATTGCAGCTTATAATGGTTACAAATAAAGCAATAGCATCACAAAT
TTCACAAATAAAGCATTTTTTTCACTGCATTCTAGTTGTGGTTTGTCCAA
ACTCATCAATGTATCTTATCATGTCTGTATACCGTCGACCTCTAGCTAGA
GCTTGGCGTAATCATGGTCATAGCTGTTTCCTGTGTGAAATTGTTATCCG
CTCACAATTCCACACAACATACGAGCCGGAAGCATAAAGTGTAAAGCCTG
GGGTGCCTAATGAGTGAGCTAACTCACATTAATTGCGTTGCGCTCACTGC
CCGCTTTCCAGTCGGGAAACCTGTCGTGCCAGCTGCATTAATGAATCGGC
CAACGCGCGGGGAGAGGCGGTTTGCGTATTGGGCGCTCTTCCGCTTCCTC
GCTCACTGACTCGCTGCGCTCGGTCGTTCGGCTGCGGCGAGCGGTATCAG
CTCACTCAAAGGCGGTAATACGGTTATCCACAGAATCAGGGGATAACGCA
GGAAAGAACATGTGAGCAAAAGGCCAGCAAAAGGCCAGGAACCGTAAAAA
GGCCGCGTTGCTGGCGTTTTTCCATAGGCTCCGCCCCCCTGACGAGCATC
ACAAAAATCGACGCTCAAGTCAGAGGTGGCGAAACCCGACAGGACTATAA
AGATACCAGGCGTTTCCCCCTGGAAGCTCCCTCGTGCGCTCTCCTGTTCC
GACCCTGCCGCTTACCGGATACCTGTCCGCCTTTCTCCCTTCGGGAAGCG
TGGCGCTTTCTCATAGCTCACGCTGTAGGTATCTCAGTTCGGTGTAGGTC
GTTCGCTCCAAGCTGGGCTGTGTGCACGAACCCCCCGTTCAGCCCGACCG
CTGCGCCTTATCCGGTAACTATCGTCTTGAGTCCAACCCGGTAAGACACG
ACTTATCGCCACTGGCAGCAGCCACTGGTAACAGGATTAGCAGAGCGAGG
TATGTAGGCGGTGCTACAGAGTTCTTGAAGTGGTGGCCTAACTACGGCTA
CACTAGAAGAACAGTATTTGGTATCTGCGCTCTGCTGAAGCCAGTTACCT
TCGGAAAAAGAGTTGGTAGCTCTTGATCCGGCAAACAAACCACCGCTGGT
AGCGGTTGGTTTTTTGTTTGCAAGCAGCAGATTACGCGCAGAAAAAAAGG
ATCTCAAGAAGATCCTTTGATCTTTTCTACGGGGTCTGACGCTCAGTGGA
ACGAAAACTCACGTTAAGGGATTTTGGTCATGAGATTATCAAAAAGGATC
TTCACCTAGATCCTTTTAAATTAAAAATGAAGTTTTAAATCAATCTAAAG
TATATATGAGTAAACTTGGTCTGACAGTTACCAATGCTTAATCAGTGAGG
CACCTATCTCAGCGATCTGTCTATTTCGTTCATCCATAGTTGCCTGACTC
CCCGTCGTGTAGATAACTACGATACGGGAGGGCTTACCATCTGGCCCCAG
TGCTGCAATGATACCGCGAGACCCACGCTCACCGGCTCCAGATTTATCAG
CAATAAACCAGCCAGCCGGAAGGGCCGAGCGCAGAAGTGGTCCTGCAACT
TTATCCGCCTCCATCCAGTCTATTAATTGTTGCCGGGAAGCTAGAGTAAG
TAGTTCGCCAGTTAATAGTTTGCGCAACGTTGTTGCCATTGCTACAGGCA
TCGTGGTGTCACGCTCGTCGTTTGGTATGGCTTCATTCAGCTCCGGTTCC
CAACGATCAAGGCGAGTTACATGATCCCCCATGTTGTGCAAAAAAGCGGT
TAGCTCCTTCGGTCCTCCGATCGTTGTCAGAAGTAAGTTGGCCGCAGTGT
TATCACTCATGGTTATGGCAGCACTGCATAATTCTCTTACTGTCATGCCA
TCCGTAAGATGCTTTTCTGTGACTGGTGAGTACTCAACCAAGTCATTCTG
AGAATAGTGTATGCGGCGACCGAGTTGCTCTTGCCCGGCGTCAATACGGG
ATAATACCGCGCCACATAGCAGAACTTTAAAAGTGCTCATCATTGGAAAA
CGTTCTTCGGGGCGAAAACTCTCAAGGATCTTACCGCTGTTGAGATCCAG
TTCGATGTAACCCACTCGTGCACCCAACTGATCTTCAGCATCTTTTACTT
TCACCAGCGTTTCTGGGTGAGCAAAAACAGGAAGGCAAAATGCCGCAAAA
AAGGGAATAAGGGCGACACGGAAATGTTGAATACTCATACTCTTCCTTTT
TCAATATTATTGAAGCATTTATCAGGGTTATTGTCTCATGAGCGGATACA
TATTTGAATGTATTTAGAAAAATAAACAAATAGGGGTTCCGCGCACATTT
CCCCGAAAAGTGCCACCTGACGTC
TRPC5 Plasmid Sequence
The DNA sequence of the TRPC5 plasmid used
in Example 42 is included below.
Underlined nucleic acids represent those
encoding human TRPC5.
SEQ ID NO: 2
GACGGATCGGGAGATCTCCCGATCCCCTATGGTGCACTCTCAGTACAATC
TGCTCTGATGCCGCATAGTTAAGCCAGTATCTGCTCCCTGCTTGTGTGTT
GGAGGTCGCTGAGTAGTGCGCGAGCAAAATTTAAGCTACAACAAGGCAAG
GCTTGACCGACAATTGCATGAAGAATCTGCTTAGGGTTAGGCGTTTTGCG
CTGCTTCGCGATGTACGGGCCAGATATACGCGTTGACATTGATTATTGAC
TAGTTATTAATAGTAATCAATTACGGGGTCATTAGTTCATAGCCCATATA
TGGAGTTCCGCGTTACATAACTTACGGTAAATGGCCCGCCTGGCTGACCG
CCCAACGACCCCCGCCCATTGACGTCAATAATGACGTATGTTCCCATAGT
AACGCCAATAGGGACTTTCCATTGACGTCAATGGGTGGAGTATTTACGGT
AAACTGCCCACTTGGCAGTACATCAAGTGTATCATATGCCAAGTACGCCC
CCTATTGACGTCAATGACGGTAAATGGCCCGCCTGGCATTATGCCCAGTA
CATGACCTTATGGGACTTTCCTACTTGGCAGTACATCTACGTATTAGTCA
TCGCTATTACCATGGTGATGCGGTTTTGGCAGTACATCAATGGGCGTGGA
TAGCGGTTTGACTCACGGGGATTTCCAAGTCTCCACCCCATTGACGTCAA
TGGGAGTTTGTTTTGGCACCAAAATCAACGGGACTTTCCAAAATGTCGTA
ACAACTCCGCCCCATTGACGCAAATGGGCGGTAGGCGTGTACGGTGGGAG
GTCTATATAAGCAGAGCTCTCCCTATCAGTGATAGAGATCTCCCTATCAG
TGATAGAGATCGTCGACGAGCTCGTTTAGTGAACCGTCAGATCGCCTGGA
GACGCCATCCACGCTGTTTTGACCTCCATAGAAGACACCGGGACCGATCC
AGCCTCCGGACTCTAGCGTTTAAACTTAAGCCCAAGCTGGCTAGACCGCC
ATGGCCCAACTGTACTACAAAAAGGTCAACTACTCACCGTACAGAGACCG
CATCCCCCTGCAAATTGTGAGGGCTGAGACAGAGCTCTCTGCAGAGGAGA
AGGCCTTCCTCAATGCTGTGGAGAAGGGGGACTATGCCACTGTGAAGCAG
GCCCTTCAGGAGGCTGAGATCTACTATAATGTTAACATCAACTGCATGGA
CCCCTTGGGCCGGAGTGCCCTGCTCATTGCCATTGAGAACGAGAACCTGG
AGATCATGGAGCTACTGCTGAACCACAGCGTGTATGTGGGTGATGCATTG
CTCTATGCCATACGCAAGGAAGTGGTGGGCGCTGTGGAGCTTCTGCTCAG
CTACAGGCGGCCCAGCGGAGAGAAGCAGGTCCCCACTCTGATGATGGACA
CGCAGTTCTCTGAATTCACACCGGACATCACTCCCATCATGCTGGCTGCC
CACACCAACAACTACGAAATCATCAAACTGCTTGTCCAAAAACGGGTCAC
TATCCCACGGCCCCACCAGATCCGCTGCAACTGTGTGGAGTGTGTGTCTA
GTTCAGAGGTAGACAGCCTGCGCCACTCTCGCTCCCGACTGAACATCTAT
AAGGCTCTGGCAAGCCCCTCACTCATTGCCTTATCAAGTGAGGACCCCAT
CCTAACTGCCTTCCGTCTGGGCTGGGAGCTCAAGGAGCTCAGCAAGGTGG
AGAATGAGTTCAAGGCCGAGTATGAGGAGCTCTCTCAGCAGTGCAAGCTC
TTTGCCAAAGACCTGCTGGACCAAGCTCGGAGCTCCAGGGAACTGGAGAT
CATCCTCAACCATCGAGATGACCACAGTGAAGAGCTTGACCCTCAGAAGT
ACCATGACCTGGCCAAGTTGAAGGTGGCAATCAAATACCACCAGAAAGAG
TTTGTTGCTCAGCCCAACTGCCAACAGTTGCTTGCCACCCTGTGGTATGA
TGGCTTCCCTGGATGGCGGCGGAAACACTGGGTAGTCAAGCTTCTAACCT
GCATGACCATTGGGTTCCTGTTTCCCATGCTGTCTATAGCCTACCTGATC
TCACCCAGGAGCAACCTTGGGCTGTTCATCAAGAAACCCTTTATCAAGTT
TATCTGCCACACAGCATCCTATTTGACCTTCCTCTTTATGCTTCTCCTGG
CTTCTCAGCACATTGTCAGGACAGACCTTCATGTACAGGGGCCTCCCCCA
ACTGTCGTGGAATGGATGATATTGCCTTGGGTTCTAGGTTTCATTTGGGG
TGAGATTAAGGAAATGTGGGATGGTGGATTTACTGAATACATCCATGACT
GGTGGAACCTGATGGATTTTGCAATGAACTCCCTCTACCTGGCAACTATT
TCCCTGAAGATTGTGGCCTATGTCAAGTATAATGGTTCTCGTCCAAGGGA
GGAATGGGAAATGTGGCACCCGACTCTGATTGCGGAAGCACTCTTCGCAA
TATCCAACATTTTAAGTTCGTTGCGTCTCATATCCCTGTTCACAGCCAAC
TCCCACTTAGGACCTCTGCAGATCTCTTTGGGACGCATGCTGCTTGATAT
CCTCAAATTCCTCTTTATCTACTGCCTGGTACTACTAGCTTTTGCCAATG
GACTGAACCAGCTTTACTTCTATTATGAAACCAGAGCTATCGATGAGCCT
AACAACTGCAAGGGGATCCGATGTGAGAAACAGAACAATGCCTTCTCCAC
GCTCTTTGAGACTCTTCAGTCACTCTTCTGGTCTGTATTTGGCCTTTTAA
ATCTATATGTCACCAATGTGAAAGCCAGACACGAATTCACCGAGTTTGTA
GGAGCTACCATGTTTGGAACATACAATGTCATCTCCCTGGTAGTGCTGCT
GAACATGCTGATTGCTATGATGAACAACTCCTATCAGCTTATTGCCGATC
ATGCTGATATCGAGTGGAAGTTTGCAAGGACGAAGCTCTGGATGAGTTAC
TTTGATGAAGGTGGCACCTTGCCACCTCCTTTCAACATCATCCCCAGCCC
CAAGTCATTTCTATACCTTGGTAACTGGTTCAACAACACCTTCTGCCCCA
AAAGAGACCCTGACGGTAGACGGAGAAGGCGCAACTTGAGAAGTTTCACA
GAACGCAATGCTGACAGCCTGATACAAAATCAACATTATCAGGAAGTTAT
CAGGAATTTAGTCAAAAGATATGTGGCTGCTATGATAAGAAATTCCAAAA
CACATGAGGGACTTACAGAAGAAAATTTTAAGGAATTAAAGCAAGACATC
TCCAGCTTTCGGTATGAAGTGCTTGACCTCTTGGGAAATAGAAAACATCC
AAGGAGCTTTTCCACTAGCAGCACTGAACTGTCTCAGAGAGACGATAATA
ATGATGGCAGTGGTGGGGCTCGGGCCAAATCCAAGAGTGTCTCTTTTAAT
TTAGGCTGCAAGAAAAAGACTTGCCATGGGCCACCTCTCATCAGAACCAT
GCCAAGGTCCAGTGGTGCCCAAGGAAAGTCAAAAGCTGAGTCATCAAGCA
AACGCTCCTTCATGGGTCCTTCTCTCAAGAAACTGGGTCTCCTATTCTCC
AAATTTAATGGTCATATGTCTGAACCCAGTTCAGAGCCAATGTACACAAT
TTCTGATGGAATTGTTCAGCAGCACTGTATGTGGCAGGACATCAGATATT
CTCAGATGGAGAAAGGGAAAGCAGAGGCCTGTTCTCAAAGTGAAATTAAC
CTCAGTGAGGTAGAATTAGGTGAAGTCCAGGGCGCTGCTCAGAGCAGTGA
ATGCCCTCTAGCCTGTTCCAGCTCTCTTCACTGTGCATCCAGCATCTGCT
CCTCAAATTCTAAACTTTTAGACTCCTCAGAGGATGTATTTGAAACTTGG
GGAGAGGCTTGTGACTTGCTCATGCACAAATGGGGTGATGGACAGGAAGA
ACAAGTTACAACTCGCCTCTAATGACTCGAGTCTAGAGGGCCCGTTTAAA
CCCGCTGATCAGCCTCGACTGTGCCTTCTAGTTGCCAGCCATCTGTTGTT
TGCCCCTCCCCCGTGCCTTCCTTGACCCTGGAAGGTGCCACTCCCACTGT
CCTTTCCTAATAAAATGAGGAAATTGCATCGCATTGTCTGAGTAGGTGTC
ATTCTATTCTGGGGGGTGGGGTGGGGCAGGACAGCAAGGGGGAGGATTGG
GAAGACAATAGCAGGCATGCTGGGGATGCGGTGGGCTCTATGGCTTCTGA
GGCGGAAAGAACCAGCTGGGGCTCTAGGGGGTATCCCCACGCGCCCTGTA
GCGGCGCATTAAGCGCGGCGGGTGTGGTGGTTACGCGCAGCGTGACCGCT
ACACTTGCCAGCGCCCTAGCGCCCGCTCCTTTCGCTTTCTTCCCTTCCTT
TCTCGCCACGTTCGCCGGCTTTCCCCGTCAAGCTCTAAATCGGGGGCTCC
CTTTAGGGTTCCGATTTAGTGCTTTACGGCACCTCGACCCCAAAAAACTT
GATTAGGGTGATGGTTCACGTAGTGGGCCATCGCCCTGATAGACGGTTTT
TCGCCCTTTGACGTTGGAGTCCACGTTCTTTAATAGTGGACTCTTGTTCC
AAACTGGAACAACACTCAACCCTATCTCGGTCTATTCTTTTGATTTATAA
GGGATTTTGCCGATTTCGGCCTATTGGTTAAAAAATGAGCTGATTTAACA
AAAATTTAACGCGAATTAATTCTGTGGAATGTGTGTCAGTTAGGGTGTGG
AAAGTCCCCAGGCTCCCCAGCAGGCAGAAGTATGCAAAGCATGCATCTCA
ATTAGTCAGCAACCAGGTGTGGAAAGTCCCCAGGCTCCCCAGCAGGCAGA
AGTATGCAAAGCATGCATCTCAATTAGTCAGCAACCATAGTCCCGCCCCT
AACTCCGCCCATCCCGCCCCTAACTCCGCCCAGTTCCGCCCATTCTCCGC
CCCATGGCTGACTAATTTTTTTTATTTATGCAGAGGCCGAGGCCGCCTCT
GCCTCTGAGCTATTCCAGAAGTAGTGAGGAGGCTTTTTTGGAGGCCTAGG
CTTTTGCAAAAAGCTCCCGGGAGCTTGTATATCCATTTTCGGATCTGATC
AGCACGTGATGAAAAAGCCTGAACTCACCGCGACGTCTGTCGAGAAGTTT
CTGATCGAAAAGTTCGACAGCGTCTCCGACCTGATGCAGCTCTCGGAGGG
CGAAGAATCTCGTGCTTTCAGCTTCGATGTAGGAGGGCGTGGATATGTCC
TGCGGGTAAATAGCTGCGCCGATGGTTTCTACAAAGATCGTTATGTTTAT
CGGCACTTTGCATCGGCCGCGCTCCCGATTCCGGAAGTGCTTGACATTGG
GGAATTCAGCGAGAGCCTGACCTATTGCATCTCCCGCCGTGCACAGGGTG
TCACGTTGCAAGACCTGCCTGAAACCGAACTGCCCGCTGTTCTGCAGCCG
GTCGCGGAGGCCATGGATGCGATCGCTGCGGCCGATCTTAGCCAGACGAG
CGGGTTCGGCCCATTCGGACCGCAAGGAATCGGTCAATACACTACATGGC
GTGATTTCATATGCGCGATTGCTGATCCCCATGTGTATCACTGGCAAACT
GTGATGGACGACACCGTCAGTGCGTCCGTCGCGCAGGCTCTCGATGAGCT
GATGCTTTGGGCCGAGGACTGCCCCGAAGTCCGGCACCTCGTGCACGCGG
ATTTCGGCTCCAACAATGTCCTGACGGACAATGGCCGCATAACAGCGGTC
ATTGACTGGAGCGAGGCGATGTTCGGGGATTCCCAATACGAGGTCGCCAA
CATCTTCTTCTGGAGGCCGTGGTTGGCTTGTATGGAGCAGCAGACGCGCT
ACTTCGAGCGGAGGCATCCGGAGCTTGCAGGATCGCCGCGGCTCCGGGCG
TATATGCTCCGCATTGGTCTTGACCAACTCTATCAGAGCTTGGTTGACGG
CAATTTCGATGATGCAGCTTGGGCGCAGGGTCGATGCGACGCAATCGTCC
GATCCGGAGCCGGGACTGTCGGGCGTACACAAATCGCCCGCAGAAGCGCG
GCCGTCTGGACCGATGGCTGTGTAGAAGTACTCGCCGATAGTGGAAACCG
ACGCCCCAGCACTCGTCCGAGGGCAAAGGAATAGCACGTGCTACGAGATT
TCGATTCCACCGCCGCCTTCTATGAAAGGTTGGGCTTCGGAATCGTTTTC
CGGGACGCCGGCTGGATGATCCTCCAGCGCGGGGATCTCATGCTGGAGTT
CTTCGCCCACCCCAACTTGTTTATTGCAGCTTATAATGGTTACAAATAAA
GCAATAGCATCACAAATTTCACAAATAAAGCATTTTTTTCACTGCATTCT
AGTTGTGGTTTGTCCAAACTCATCAATGTATCTTATCATGTCTGTATACC
GTCGACCTCTAGCTAGAGCTTGGCGTAATCATGGTCATAGCTGTTTCCTG
TGTGAAATTGTTATCCGCTCACAATTCCACACAACATACGAGCCGGAAGC
ATAAAGTGTAAAGCCTGGGGTGCCTAATGAGTGAGCTAACTCACATTAAT
TGCGTTGCGCTCACTGCCCGCTTTCCAGTCGGGAAACCTGTCGTGCCAGC
TGCATTAATGAATCGGCCAACGCGCGGGGAGAGGCGGTTTGCGTATTGGG
CGCTCTTCCGCTTCCTCGCTCACTGACTCGCTGCGCTCGGTCGTTCGGCT
GCGGCGAGCGGTATCAGCTCACTCAAAGGCGGTAATACGGTTATCCACAG
AATCAGGGGATAACGCAGGAAAGAACATGTGAGCAAAAGGCCAGCAAAAG
GCCAGGAACCGTAAAAAGGCCGCGTTGCTGGCGTTTTTCCATAGGCTCCG
CCCCCCTGACGAGCATCACAAAAATCGACGCTCAAGTCAGAGGTGGCGAA
ACCCGACAGGACTATAAAGATACCAGGCGTTTCCCCCTGGAAGCTCCCTC
GTGCGCTCTCCTGTTCCGACCCTGCCGCTTACCGGATACCTGTCCGCCTT
TCTCCCTTCGGGAAGCGTGGCGCTTTCTCATAGCTCACGCTGTAGGTATC
TCAGTTCGGTGTAGGTCGTTCGCTCCAAGCTGGGCTGTGTGCACGAACCC
CCCGTTCAGCCCGACCGCTGCGCCTTATCCGGTAACTATCGTCTTGAGTC
CAACCCGGTAAGACACGACTTATCGCCACTGGCAGCAGCCACTGGTAACA
GGATTAGCAGAGCGAGGTATGTAGGCGGTGCTACAGAGTTCTTGAAGTGG
TGGCCTAACTACGGCTACACTAGAAGAACAGTATTTGGTATCTGCGCTCT
GCTGAAGCCAGTTACCTTCGGAAAAAGAGTTGGTAGCTCTTGATCCGGCA
AACAAACCACCGCTGGTAGCGGTTGGTTTTTTGTTTGCAAGCAGCAGATT
ACGCGCAGAAAAAAAGGATCTCAAGAAGATCCTTTGATCTTTTCTACGGG
GTCTGACGCTCAGTGGAACGAAAACTCACGTTAAGGGATTTTGGTCATGA
GATTATCAAAAAGGATCTTCACCTAGATCCTTTTAAATTAAAAATGAAGT
TTTAAATCAATCTAAAGTATATATGAGTAAACTTGGTCTGACAGTTACCA
ATGCTTAATCAGTGAGGCACCTATCTCAGCGATCTGTCTATTTCGTTCAT
CCATAGTTGCCTGACTCCCCGTCGTGTAGATAACTACGATACGGGAGGGC
TTACCATCTGGCCCCAGTGCTGCAATGATACCGCGAGACCCACGCTCACC
GGCTCCAGATTTATCAGCAATAAACCAGCCAGCCGGAAGGGCCGAGCGCA
GAAGTGGTCCTGCAACTTTATCCGCCTCCATCCAGTCTATTAATTGTTGC
CGGGAAGCTAGAGTAAGTAGTTCGCCAGTTAATAGTTTGCGCAACGTTGT
TGCCATTGCTACAGGCATCGTGGTGTCACGCTCGTCGTTTGGTATGGCTT
CATTCAGCTCCGGTTCCCAACGATCAAGGCGAGTTACATGATCCCCCATG
TTGTGCAAAAAAGCGGTTAGCTCCTTCGGTCCTCCGATCGTTGTCAGAAG
TAAGTTGGCCGCAGTGTTATCACTCATGGTTATGGCAGCACTGCATAATT
CTCTTACTGTCATGCCATCCGTAAGATGCTTTTCTGTGACTGGTGAGTAC
TCAACCAAGTCATTCTGAGAATAGTGTATGCGGCGACCGAGTTGCTCTTG
CCCGGCGTCAATACGGGATAATACCGCGCCACATAGCAGAACTTTAAAAG
TGCTCATCATTGGAAAACGTTCTTCGGGGCGAAAACTCTCAAGGATCTTA
CCGCTGTTGAGATCCAGTTCGATGTAACCCACTCGTGCACCCAACTGATC
TTCAGCATCTTTTACTTTCACCAGCGTTTCTGGGTGAGCAAAAACAGGAA
GGCAAAATGCCGCAAAAAAGGGAATAAGGGCGACACGGAAATGTTGAATA
CTCATACTCTTCCTTTTTCAATATTATTGAAGCATTTATCAGGGTTATTG
TCTCATGAGCGGATACATATTTGAATGTATTTAGAAAAATAAACAAATAG
GGGTTCCGCGCACATTTCCCCGAAAAGTGCCACCTGACGTC

What is claimed is:

1. A method of treating a kidney disease comprising the step of co-administering to a subject in need thereof:
a. a TRPC5 inhibitory compound of structural Formula (A), or a tautomer or a pharmaceutically acceptable salt thereof: 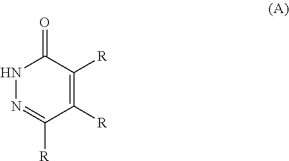
wherein
each R is independently selected from the group consisting of H, alkyl, alkenyl, alkynyl, aryl, heterocyclyl, heteroaryl, halogen, —OH, CN, cycloalkyl, —O-alkyl, —O— cycloalkyl, —O-aryl, -aryl-O-aryl, —CF₃, —C(H)F₂, alkylene-CF₃, alkylene-C(H)F₂, —SO₂-alkyl, —O-alkylene-O-alkyl, -heterocyclyl-L-R⁴, and heteroaryl-L-R⁴;
R⁴ is absent or selected from the group consisting of alkyl, cycloalkyl, polycyclyl, aryl, heterocyclyl, heteroaryl, —C(O)N(R⁵)₂, and CF₃;
R⁵ is independently H or alkyl;
R⁶ is selected from the group consisting of alkyl, cycloalkyl, aryl, heterocyclyl, heteroaryl, alkylene-aryl, —C(O)N(R⁵)₂, and CF₃;
L is absent or selected from the group consisting of methylene, —C(O)—, —SO₂—, —CH₂N(Me)-, —N(R⁵)(R⁶)—, —C(R⁵)(R⁶)—, and —O—R⁶; and
one and only one R is -heterocyclyl-L-R⁴ or -heteroaryl-L-R⁴; and
b. a second therapeutic agent selected from an immunomodulator, a calcineurin inhibitor, a renin angiotensin aldosterone system inhibitor, an antiproliferative agent, an alkylating agent, a corticosteroid, an angiotensin converting enzyme inhibitor, an adrenocorticotropic hormone stimulant, an angiotensin receptor blocker, a sodium-glucose transport protein 2 inhibitor, a dual sodium-glucose transport protein 1/2 inhibitor, a nuclear Factor-1 (erythroid-derived 2)-like 2 agonist, a chemokine receptor 2 inhibitor, a chemokine receptor 5 inhibitor, an endothelin 1 receptor antagonist, a beta blocker, a mineralocorticoid receptor antagonist, a loop or thiazide diuretic, a calcium channel blocker, a statin, a short-intermediate or long-acting insulin, a dipeptidyl peptidase 4 inhibitor, a glucagon-like peptide 1 receptor agonist, a sulfonylurea, an apoptosis signal-regulating kinase-1, a chymase inhibitor, a selective glycation inhibitor, a renin inhibitor, an interleukin-33 inhibitor, a farnesoid X receptor agonist, a soluble guanylate cyclase stimulator, a thromboxane receptor antagonist, a xanthine oxidase inhibitor, an erythropoietin receptor agonist, a cannabinoid receptor type 1 inverse agonist, a NADPH oxidase inhibitor, an anti-vascular endothelial growth factor B, an anti-fibrotic agent, a neprilysin inhibitor, a dual CD80/CD86 inhibitor, a CD40 antagonist, a cellular cholesterol and lipid blocker, a PDGFR antagonist, a Slit guidance ligand 2, an APOL1 inhibitor, an Nrl2 activator/NF-κB inhibitor, a somatostatin receptor agonist, a PPAR gamma agonist, a AMP activated protein kinase stimulator, a tyrosine kinase inhibitor, a glucosylceramide synthase inhibitor, an arginine vasopressin receptor 2 antagonist, a xanthine oxidase inhibitor, and a vasopressin receptor 2 antagonist.

2. The method of claim 1, wherein the TRPC5 inhibitory compound is represented by structural Formula (A-I), (A-II), or (A-III), or a tautomer or a pharmaceutically acceptable salt thereof; 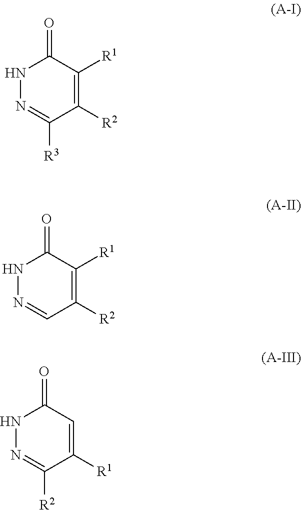
wherein
R¹ and R³ are independently selected from the group consisting of H, alkyl, alkenyl, alkynyl, aryl, heterocyclyl, heteroaryl, halogen, —OH, —CN, -cycloalkyl, —O-alkyl, —O-cycloalkyl, —O-aryl, -aryl-O-aryl, —CF₃, —C(H)F₂, alkylene-CF₃, alkylene-C(H)F₂, —SO₂-alkyl, and —O-alkylene-O-alkyl, -heterocyclyl-L-R⁴, and -heteroaryl-L-R⁴;
R² is -heterocyclyl-L-R⁴;
R⁴ is absent or selected from the group consisting of alkyl, cycloalkyl, aryl, alkylene-aryl, alkylene-heteroaryl, heteroaryl, heterocyclyl, —C(O)N(R⁵)₂, and CF₃;
R⁵ is independently H or alkyl;
R⁶ is selected from the group consisting of alkyl, cycloalkyl, aryl, heterocyclyl, heteroaryl, alkylene-aryl, —C(O)N(R⁵)₂, and CF₃;
L is absent or selected from the group consisting of methylene, —C(O)—, —SO₂—, —CH₂N(Me)-, —N(R⁵)(R⁶)—, —C(R⁵)(R⁶)—, and —O—R⁶; and
one and only one of R¹, R², and R³ is -heterocyclyl-L-R⁴ or -heteroaryl-L-R⁴.

3. The method of claim 1, wherein the TRPC5 inhibitory compound has structural formula (I): 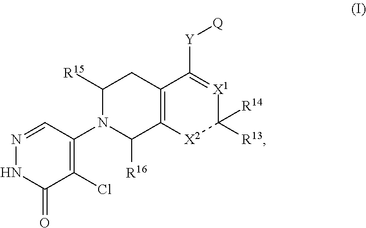
or a pharmaceutically acceptable salt thereof; wherein:
“ [symbol] ” is a single bond or a double bond
X¹ is CH or N;
when “ [symbol] ” is a double bond, X² is CH or N;
when “ [symbol] ” is a single bond, X² is N(CH₃),
when X¹ is CH, X² is N or N(CH₃);
Y is —O—, —N(CH₃)—, —N(CH₂CH₂OH)—, cyclopropan-1,1-diyl, or —CH(CH₃)—;
Q is 2-trifluoromethyl-4-fluorophenyl, 2-difluoromethyl-4-fluorophenyl, 2-trifluoromethylphenyl, 2-methyl-4-fluorophenyl, 2-chloro-4-fluorophenyl, 2-chlorophenyl, 1-(benzyl)-4-methylpiperidin-3-yl, 4-trifluoromethylpyridin-3-yl, 2-trifluoromethyl-6-fluorophenyl, 2-trifluoromethyl-3-cyanophenyl, 2-ethyl-3-fluorophenyl, 2-chloro-3-cyanophenyl, 2-trifluoromethyl-5-fluorophenyl, or 2-difluoromethylphenyl;
when “ [symbol] ” is a double bond, R¹³ is hydrogen, —CH₂OH, —CH(OH)—CH₂OH, —NH₂, —CH(OH)CH₃, —OCH₃, or —NH—(CH₂)₂OH; and R¹⁴ is absent; or
when “ [symbol] ” is a single bond, R¹³ and R¹⁴ are taken together to form ═O; and
each of R⁵ and R⁶ is independently hydrogen or —CH₃.

4. The method of claim 2, wherein the TRPC5 inhibitory compound has the structural formula (II): 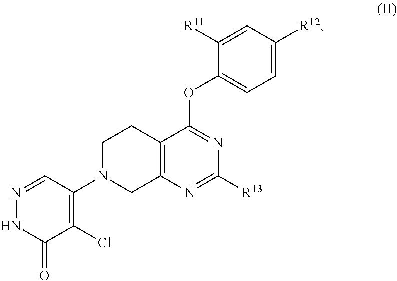
or a pharmaceutically acceptable salt thereof; wherein:
R¹¹ is chloro, —CF₃, —CHF₂, or —CH₃;
R¹² is hydrogen or fluoro; and
R¹³ is hydrogen, —NH₂, —CH₂OH, or CH(OH)—CH₂OH.

5. The method of claim 4, wherein R¹¹ is —CHF₂; and R¹² is fluoro.

6. The method of claim 3, wherein the TRPC5 inhibitory compound is selected from any one of the following compounds, or a pharmaceutically acceptable salt thereof:

| Com- | |
|---|---|
| pound | Structure |
| 100 | 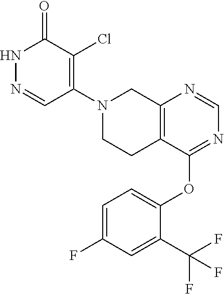 [SMILES: O=c1[nH]ncc(N2CCc3c(ncnc3Oc3ccc(F)cc3C(F)(F)F)C2)c1Cl] |
| 101 | 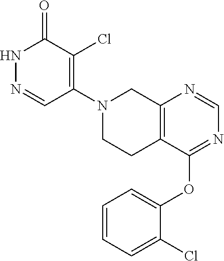 [SMILES: O=c1[nH]ncc(N2CCc3c(ncnc3Oc3ccccc3Cl)C2)c1Cl] |
| 102 | 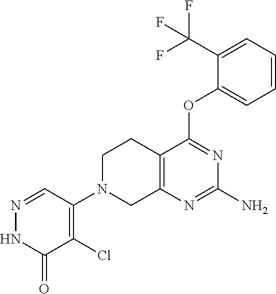 [SMILES: Nc1nc2c(c(Oc3ccccc3C(F)(F)F)n1)CCN(c1cn[nH]c(=O)c1Cl)C2] |
| 103 | 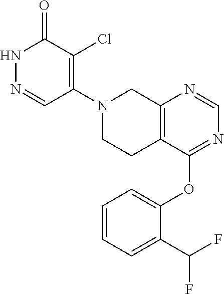 [SMILES: O=c1[nH]ncc(N2CCc3c(ncnc3Oc3ccccc3C(F)F)C2)c1Cl] |
| 104 | 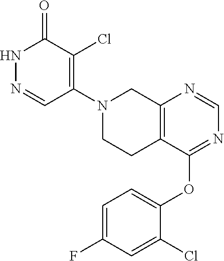 [SMILES: O=c1[nH]ncc(N2CCc3c(ncnc3Oc3ccc(F)cc3Cl)C2)c1Cl] |
| 105 | 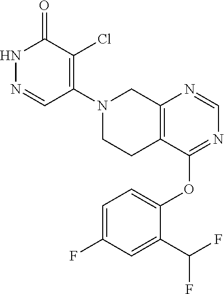 [SMILES: O=c1[nH]ncc(N2CCc3c(ncnc3Oc3ccc(F)cc3C(F)F)C2)c1Cl] |
| 106 | 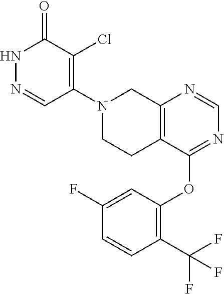 [SMILES: O=c1[nH]ncc(N2CCc3c(ncnc3Oc3cc(F)ccc3C(F)(F)F)C2)c1Cl] |
| 107 | 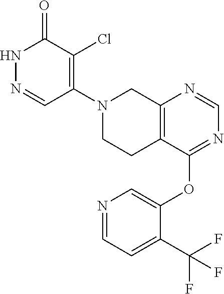 [SMILES: O=c1[nH]ncc(N2CCc3c(ncnc3Oc3cnccc3C(F)(F)F)C2)c1Cl] |
| 108 | 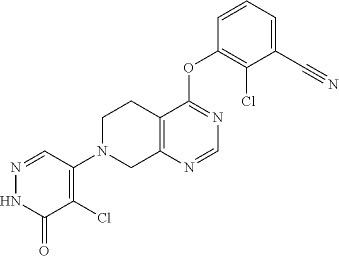 [SMILES: N#Cc1cccc(Oc2ncnc3c2CCN(c2cn[nH]c(=O)c2Cl)C3)c1Cl] |
| 109 | 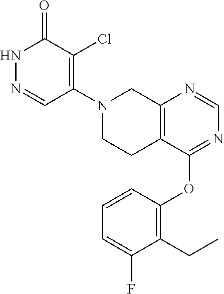 [SMILES: CCc1c(F)cccc1Oc1ncnc2c1CCN(c1cn[nH]c(=O)c1Cl)C2] |
| 110 | 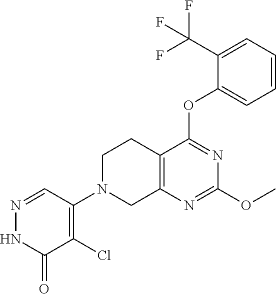 [SMILES: COc1nc2c(c(Oc3ccccc3C(F)(F)F)n1)CCN(c1cn[nH]c(=O)c1Cl)C2] |
| 111 | 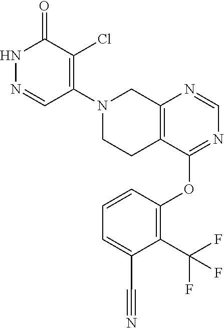 [SMILES: N#Cc1cccc(Oc2ncnc3c2CCN(c2cn[nH]c(=O)c2Cl)C3)c1C(F)(F)F] |
| 112 | 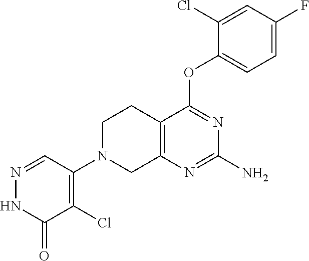 [SMILES: Nc1nc2c(c(Oc3ccc(F)cc3Cl)n1)CCN(c1cn[nH]c(=O)c1Cl)C2] |
| 113 | 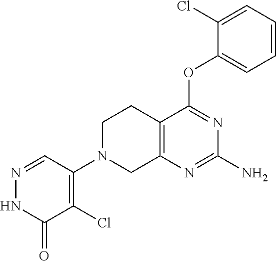 [SMILES: Nc1nc2c(c(Oc3ccccc3Cl)n1)CCN(c1cn[nH]c(=O)c1Cl)C2] |
| 114 | 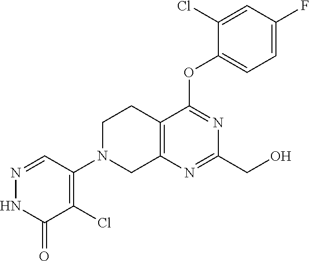 [SMILES: O=c1[nH]ncc(N2CCc3c(nc(CO)nc3Oc3ccc(F)cc3Cl)C2)c1Cl] |
| 115 | 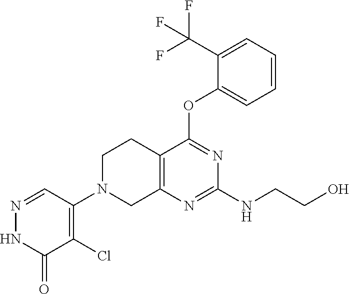 [SMILES: O=c1[nH]ncc(N2CCc3c(nc(NCCO)nc3Oc3ccccc3C(F)(F)F)C2)c1Cl] |
| 116 | 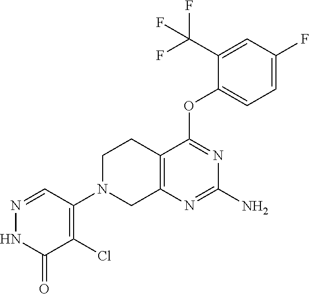 [SMILES: Nc1nc2c(c(Oc3ccc(F)cc3C(F)(F)F)n1)CCN(c1cn[nH]c(=O)c1Cl)C2] |
| 117 | 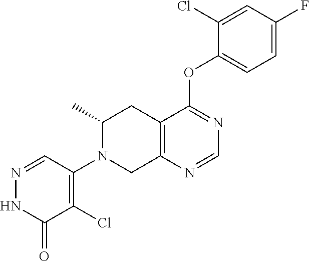 [SMILES: C[C@@H]1Cc2c(ncnc2Oc2ccc(F)cc2Cl)CN1c1cn[nH]c(=O)c1Cl] |
| 117a | 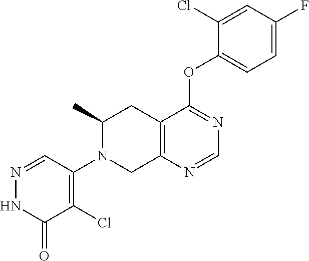 [SMILES: C[C@H]1Cc2c(ncnc2Oc2ccc(F)cc2Cl)CN1c1cn[nH]c(=O)c1Cl] |
| 118 | 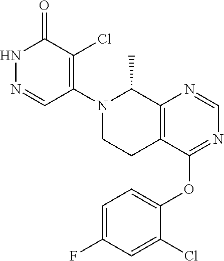 [SMILES: C[C@@H]1c2ncnc(Oc3ccc(F)cc3Cl)c2CCN1c1cn[nH]c(=O)c1Cl] |
| 119 | 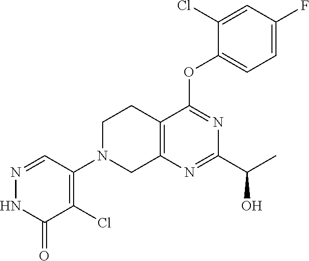 [SMILES: C[C@@H](O)c1nc2c(c(Oc3ccc(F)cc3Cl)n1)CCN(c1cn[nH]c(=O)c1Cl)C2] |
| 120 | 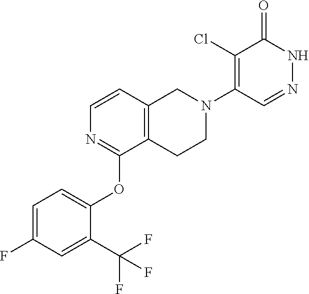 [SMILES: O=c1[nH]ncc(N2CCc3c(ccnc3Oc3ccc(F)cc3C(F)(F)F)C2)c1Cl] |
| 121 | 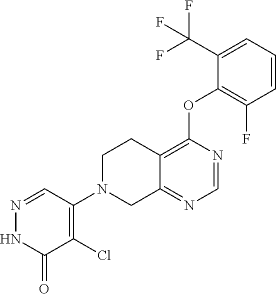 [SMILES: O=c1[nH]ncc(N2CCc3c(ncnc3Oc3c(F)cccc3C(F)(F)F)C2)c1Cl] |
| 122 | 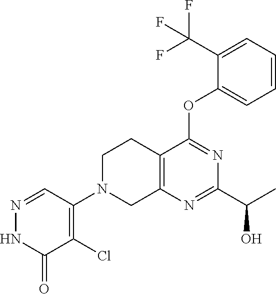 [SMILES: C[C@@H](O)c1nc2c(c(Oc3ccccc3C(F)(F)F)n1)CCN(c1cn[nH]c(=O)c1Cl)C2] |
| 123 | 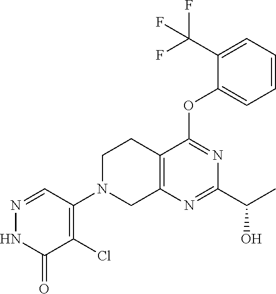 [SMILES: C[C@H](O)c1nc2c(c(Oc3ccccc3C(F)(F)F)n1)CCN(c1cn[nH]c(=O)c1Cl)C2] |
| 124 | 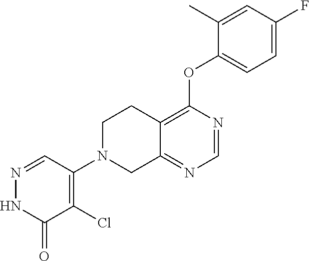 [SMILES: Cc1cc(F)ccc1Oc1ncnc2c1CCN(c1cn[nH]c(=O)c1Cl)C2] |
| 125 | 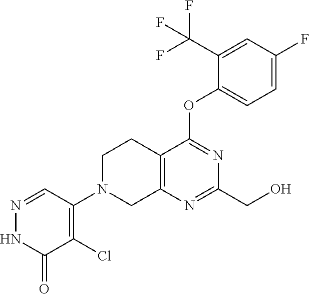 [SMILES: O=c1[nH]ncc(N2CCc3c(nc(CO)nc3Oc3ccc(F)cc3C(F)(F)F)C2)c1Cl] |
| 126 | 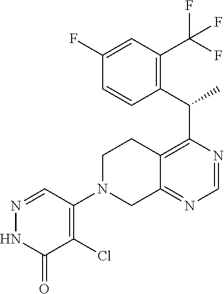 [SMILES: C[C@@H](c1ccc(F)cc1C(F)(F)F)c1ncnc2c1CCN(c1cn[nH]c(=O)c1Cl)C2] |
| 126a | 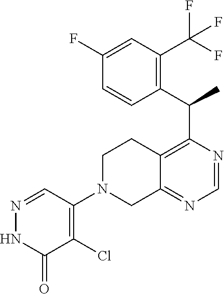 [SMILES: C[C@H](c1ccc(F)cc1C(F)(F)F)c1ncnc2c1CCN(c1cn[nH]c(=O)c1Cl)C2] |
| 127 | 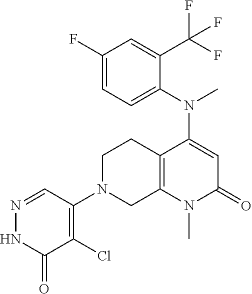 [SMILES: CN(c1ccc(F)cc1C(F)(F)F)c1cc(=O)n(C)c2c1CCN(c1cn[nH]c(=O)c1Cl)C2] |
| 128 | 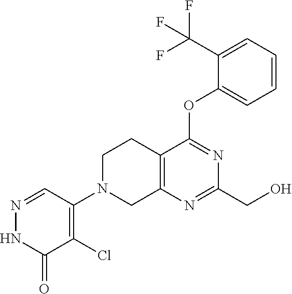 [SMILES: O=c1[nH]ncc(N2CCc3c(nc(CO)nc3Oc3ccccc3C(F)(F)F)C2)c1Cl] |
| 129 | 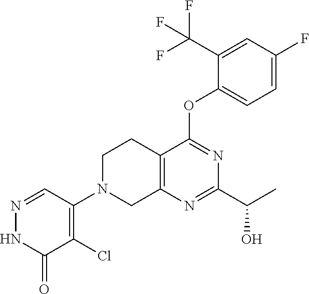 [SMILES: C[C@H](O)c1nc2c(c(Oc3ccc(F)cc3C(F)(F)F)n1)CCN(c1cn[nH]c(=O)c1Cl)C2] |
| 130 | 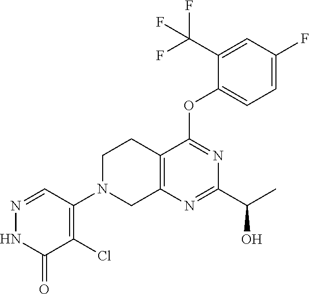 [SMILES: C[C@@H](O)c1nc2c(c(Oc3ccc(F)cc3C(F)(F)F)n1)CCN(c1cn[nH]c(=O)c1Cl)C2] |
| 131 | 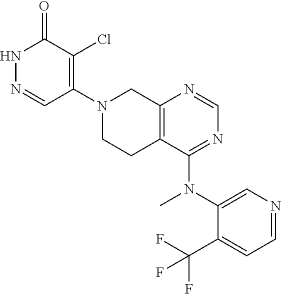 [SMILES: CN(c1cnccc1C(F)(F)F)c1ncnc2c1CCN(c1cn[nH]c(=O)c1Cl)C2] |
| 132 | 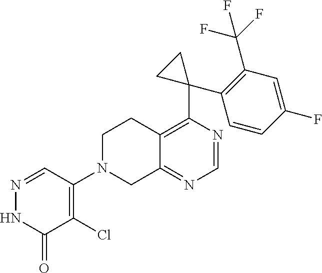 [SMILES: O=c1[nH]ncc(N2CCc3c(ncnc3C3(c4ccc(F)cc4C(F)(F)F)CC3)C2)c1Cl] |
| 133 | 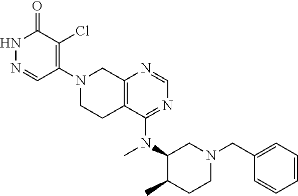 [SMILES: C[C@@H]1CCN(Cc2ccccc2)C[C@@H]1N(C)c1ncnc2c1CCN(c1cn[nH]c(=O)c1Cl)C2] |
| 133a | 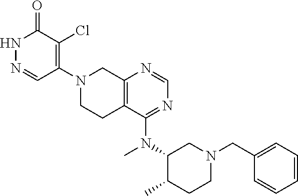 [SMILES: C[C@H]1CCN(Cc2ccccc2)C[C@H]1N(C)c1ncnc2c1CCN(c1cn[nH]c(=O)c1Cl)C2] |
| 134 | 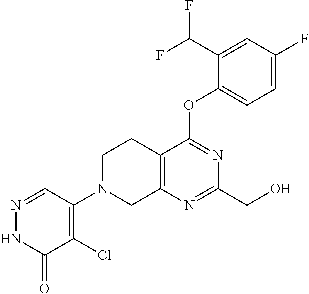 [SMILES: O=c1[nH]ncc(N2CCc3c(nc(CO)nc3Oc3ccc(F)cc3C(F)F)C2)c1Cl] |
| 135 | 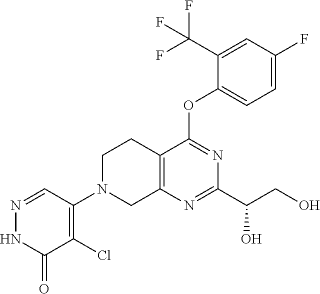 [SMILES: O=c1[nH]ncc(N2CCc3c(nc([C@@H](O)CO)nc3Oc3ccc(F)cc3C(F)(F)F)C2)c1Cl] |
| 136 | 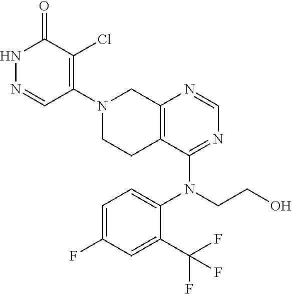 [SMILES: O=c1[nH]ncc(N2CCc3c(ncnc3N(CCO)c3ccc(F)cc3C(F)(F)F)C2)c1Cl] |
| 137 | 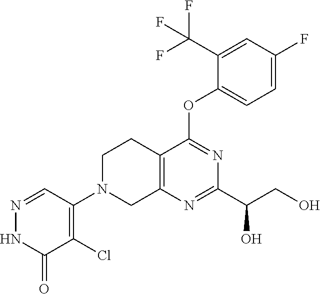 [SMILES: O=c1[nH]ncc(N2CCc3c(nc([C@H](O)CO)nc3Oc3ccc(F)cc3C(F)(F)F)C2)c1Cl] |
| 138 | 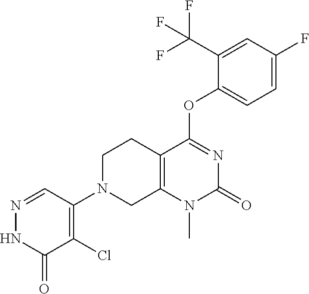 [SMILES: Cn1c2c(c(Oc3ccc(F)cc3C(F)(F)F)nc1=O)CCN(c1cn[nH]c(=O)c1Cl)C2] |
| 139 | 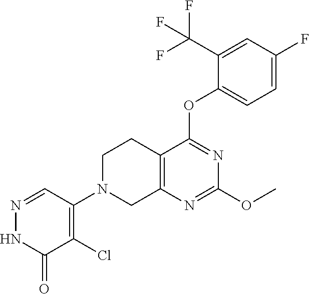 [SMILES: COc1nc2c(c(Oc3ccc(F)cc3C(F)(F)F)n1)CCN(c1cn[nH]c(=O)c1Cl)C2] |
| 140 | 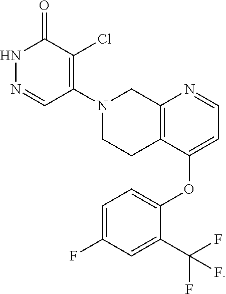 [SMILES: O=c1[nH]ncc(N2CCc3c(Oc4ccc(F)cc4C(F)(F)F)ccnc3C2)c1Cl] |

7. The method of claim 6, wherein the TRPC5 inhibitory compound is selected from any one of the following compounds, or a pharmaceutically acceptable salt thereof:

| Compound | Structure |
|---|---|
| 100 | 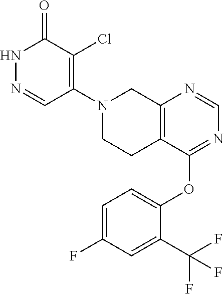 [SMILES: O=c1[nH]ncc(N2CCc3c(ncnc3Oc3ccc(F)cc3C(F)(F)F)C2)c1Cl] |
| 101 | 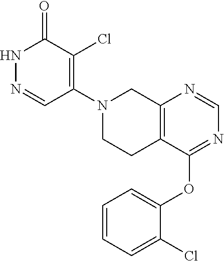 [SMILES: O=c1[nH]ncc(N2CCc3c(ncnc3Oc3ccccc3Cl)C2)c1Cl] |
| 102 | 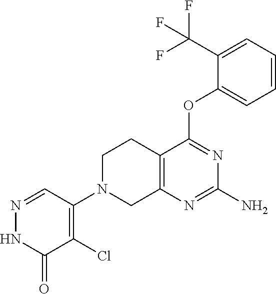 [SMILES: Nc1nc2c(c(Oc3ccccc3C(F)(F)F)n1)CCN(c1cn[nH]c(=O)c1Cl)C2] |
| 104 | 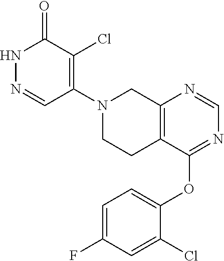 [SMILES: O=c1[nH]ncc(N2CCc3c(ncnc3Oc3ccc(F)cc3Cl)C2)c1Cl] |
| 105 | 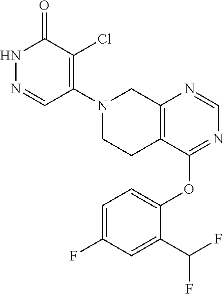 [SMILES: O=c1[nH]ncc(N2CCc3c(ncnc3Oc3ccc(F)cc3C(F)F)C2)c1Cl] |
| 114 | 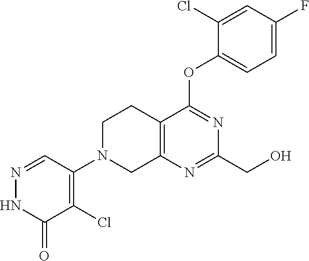 [SMILES: O=c1[nH]ncc(N2CCc3c(nc(CO)nc3Oc3ccc(F)cc3Cl)C2)c1Cl] |
| 116 | 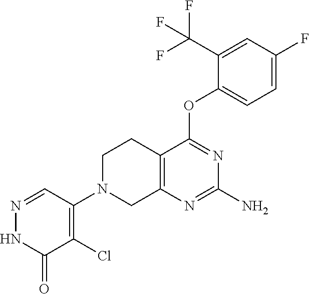 [SMILES: Nc1nc2c(c(Oc3ccc(F)cc3C(F)(F)F)n1)CCN(c1cn[nH]c(=O)c1Cl)C2] |
| 124 | 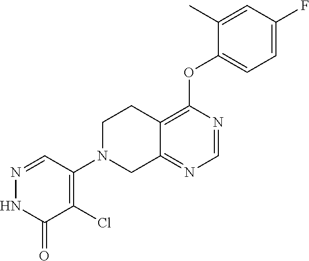 [SMILES: Cc1cc(F)ccc1Oc1ncnc2c1CCN(c1cn[nH]c(=O)c1Cl)C2] |
| 125 | 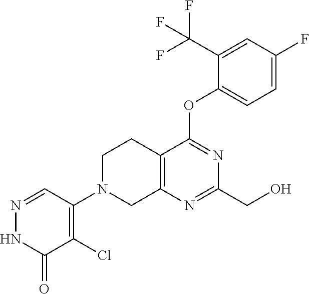 [SMILES: O=c1[nH]ncc(N2CCc3c(nc(CO)nc3Oc3ccc(F)cc3C(F)(F)F)C2)c1Cl] |
| 128 | 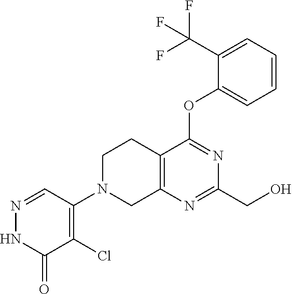 [SMILES: O=c1[nH]ncc(N2CCc3c(nc(CO)nc3Oc3ccccc3C(F)(F)F)C2)c1Cl] |
| 134 | 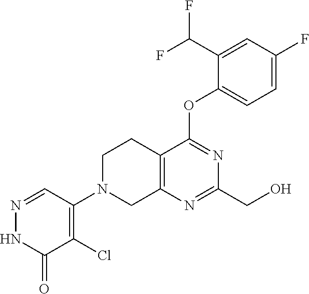 [SMILES: O=c1[nH]ncc(N2CCc3c(nc(CO)nc3Oc3ccc(F)cc3C(F)F)C2)c1Cl] |
| 135 | 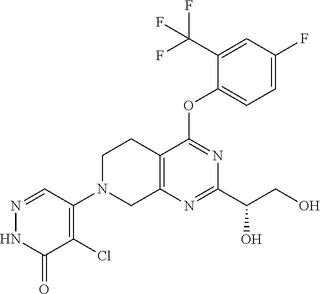 [SMILES: O=c1[nH]ncc(N2CCc3c(nc([C@@H](O)CO)nc3Oc3ccc(F)cc3C(F)(F)F)C2)c1Cl] |
| 137 | 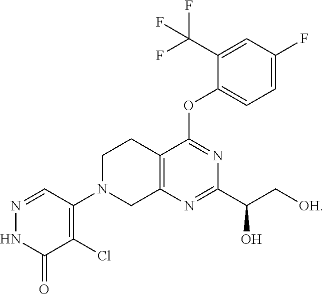 [SMILES: O=c1[nH]ncc(N2CCc3c(nc([C@H](O)CO)nc3Oc3ccc(F)cc3C(F)(F)F)C2)c1Cl] |

8. The method of claim 7, wherein the TRPC5 inhibitory compound is the following compound, or a pharmaceutically acceptable salt thereof:

| Compound | Structure |
|---|---|
| 100 | 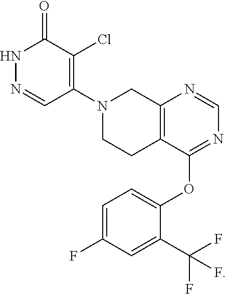 [SMILES: O=c1[nH]ncc(N2CCc3c(ncnc3Oc3ccc(F)cc3C(F)(F)F)C2)c1Cl] |

9. The method of any one of claims 1-8, wherein the immunomodulator is rituximab.

10. The method of any one of claims 1-8, wherein the angiotensin converting enzyme inhibitor is captopril, zofenopril, enalapril, ramipril, quinapril, perindopril, lisinopril, benazepril, imidapril, trandolapril, or cilazapril.

11. The method of any one of claims 1-8, wherein the angiotensin receptor blocker is losartan, candesartan, valsartan, irbesartan, telmisartan, eprosartan, olmesartan, azilsartan, or fimasartan.

12. The method of any one of claims 1-8, wherein the renin angiotensin aldosterone system inhibitor is aliskiren.

13. The method of any one of claims 1-8, wherein the endothelin 1 receptor antagonist is ambrisentan, atrasentan, bosentan, or sparsentan.

14. The method of any one of claims 1-8, wherein the anti-proliferative agent is mycophenolate mofetil.

15. The method of any one of claims 1-8, wherein the SGLT2 inhibitor is canagliflozin, dapagliflozin, empagliflozin, a combination of empagliflozin and linagliptin, a combination of empagliflozin and metformin, or a combination of dapagliflozin and metformin.

16. The method of any one of claims 1-8, wherein the calcineurin inhibitor is cyclosporine A or tacrolimus.

17. The method of any one of claims 1-8, wherein the nuclear Factor-1 (erythroid-derived 2)-like 2 agonist is bardoxolone or CXA-10.

18. The method of any one of claims 1-8, wherein the chemokine receptor 2 inhibitor is PF-04136309 or ccx140.

19. The method of any one of claims 1-8, wherein the second therapeutic agent is tacrolimus, cyclosporine A, rituximab, mycophenolate mofetil, a corticosteroid, sparsentan, enalapril, or losartan.

20. The method of claim 19, wherein the second therapeutic agent is enalapril, losartan, or cyclosporine A.

21. The method of any one of claims 1-20, wherein the disease or condition is Focal Segmental Glomerulosclerosis (FSGS), Primary Focal Segmental Glomerulosclerosis, genetic Focal Segmental Glomerulosclerosis, secondary Focal Segmental Glomerulosclerosis, Diabetic nephropathy, Alport syndrome, hypertensive kidney disease, nephrotic syndrome, steroid-resistant nephrotic syndrome, minimal change disease, membranous nephropathy, idiopathic membranous nephropathy, membranoproliferative glomerulonephritis (MPGN), immune complex-mediated MPGN, complement-mediated MPGN, Lupus nephritis, postinfectious glomerulonephritis, thin basement membrane disease, mesangial proliferative glomerulonephritis, amyloidosis (primary), c1q nephropathy, rapidly progressive glomerulonephritis (GN), anti-GBM disease, C3 glomerulonephritis, hypertensive nephrosclerosis, IgA nephropathy, autosomal recessive polycystic kidney disease, or autosomal dominant polycystic kidney disease.

22. The method of claim 21 wherein the disease or condition is Focal Segmental Glomerulosclerosis (FSGS), Primary Focal Segmental Glomerulosclerosis, genetic Focal Segmental Glomerulosclerosis, transplant-related FSGS, or secondary Focal Segmental Glomerulosclerosis.

23. The method of claim 21 wherein the kidney disease is proteinuric kidney disease.

24. The method of claim 21, wherein the kidney disease is microalbuminuria or macroalbuminuria kidney disease.

25. The method of any one of claims 1-24, wherein the subject is a human.

26. The method of claim 22 or 25, wherein the disease or condition is focal segmental glomerulosclerosis.
